(19) United States
(12) Patent Application Publication
(10) Pub. No.: US 2021/0317479 A1
(43) Pub. Date: Oct. 14, 2021

(54) NUCLEIC ACID ASSEMBLIES FOR USE IN TARGETED DELIVERY

(71) Applicants: The Broad Institute, Inc., Cambridge, MA (US); Massachusetts Institute of Technology, Cambridge, MA (US)

(72) Inventors: Feng Zhang, Cambridge, MA (US); Tyson Shepherd, Cambridge, MA (US); Rémi Veneziano, Cambridge, MA (US); Mark Bathe, Cambridge, MA (US); Ian Slaymaker, Cambridge, MA (US); Bernd Zetsche, Cambridge, MA (US)

(21) Appl. No.: 17/273,999

(22) PCT Filed: Sep. 6, 2019
(86) PCT No.: PCT/US2019/050029

§ 371 Date: Mar. 5, 2021

Related U.S. Application Data
(60) Provisional application No. 62/727,959, filed on Sep. 6, 2018.

(51) Int. Cl. C12N 15/88; C12N 15/11; C12N 9/22; A61K 9/51

(52) U.S. Cl. CPC C12N 15/88; C12N 15/11; C12N 2800/80; A61K 9/513; C12N 2310/20; C12N 9/22

(57) ABSTRACT

Disclosed are compositions and methods involving nucleic acid assemblies that enclose and/or protect cargo. Disclosed are compositions that include a nucleic acid assembly comprising one or more nucleic acid molecules and cargo comprising two or more cargo molecules. The nucleic acid assembly can have physiochemical properties that: (i) enhance targeting of the composition to one or more types of cells, tissues, organs, or microenvironments relative to other types of cells, tissues, organs, or microenvironments in vivo; (ii) enhance stability and/or half-life of the composition in vivo; and/or (iii) reduce immunogenicity of the composition. The nucleic acid assembly and/or cargo can have features that enhance intracellular trafficking of nucleic acid assembly and/or its cargo. The cargo can be enclosed and/or protected by the nucleic acid assembly. Some or all of the cargo molecules in the composition can be present in a defined stoichiometric ratio.

### CROSS-REFERENCE TO RELATED APPLICATIONS

This application claims the benefit of and priority to U.S. Provisional Application No. 62/727,959 filed Sep. 6, 2018, which is hereby incorporated by reference in its entirety.

### STATEMENT REGARDING FEDERALLY SPONSORED RESEARCH

This invention was made with Government support under Grant No. N00014-16-1-2953 awarded by Office of Naval Research, Grant No. MH110049 awarded by the National Science Foundation, and Grant No. HL141201 awarded by the National Institutes of Health. The Government has certain rights in the invention.

### REFERENCE TO SEQUENCE LISTING

The Sequence Listing submitted Sep. 6, 2019 as a text file named “BROAD_10372_PCT_ST25.txt,” created on Sep. 6, 2019, and having a size of 50,350 bytes is hereby incorporated by reference pursuant to 37 C.F.R. § 1.52(e)(5).

### FIELD OF THE INVENTION

The disclosed invention is generally in the field of nucleic acid assemblies and specifically in the area of nucleic acid assemblies for targeting cargo to cells, tissues, organs, and microenvironments and for and trafficking cargo intracellularly.

### BACKGROUND OF THE INVENTION

Synthetic biology aims to solve complex biotechnological problems such as meeting industrial-scale production needs for biomolecules and metabolites; detection of toxins or pathogens with ‘organ-on-a-chip’ devices; antibody engineering; and the establishment of in vitro disease models (Li, F., et al., MAbs 2, 466-79 (2010); Wurm, F. M., et al., Nat Biotechnol 22, 1393-8 (2004); Bhatia, S. N., et al., Sci Transl Med 6, 245sr2 (2014); Griffith, L. G., et al., Hepatology 60, 1426-34 (2014); Powell, J. D., et al., J Appl Microbiol 119, 711-23 (2015); Eklund, S. E., et al., Sensors 9, 2117-33 (2009)). Genetic engineering represents a key enabling technology for synthetic biology, offering the optimization or de novo introduction of cellular processes and products over a broad array of organisms. The recent emergence of clustered regularly interspaced short palindromic repeats (CRISPR) method for precise, targeted gene editing has translated into major advances such as mammalian cell line gene editing to optimize the industrial production of biomolecules, as well as high-throughput screens to elucidate gene function, as well as discover new drug targets (Lee, J. S., et al., Sci Rep 5, 8572 (2015); Weber, J., et al., Proc Natl Acad Sci USA 112, 13982-7 (2015); Housden, B. E., et al., Sci Signal 8, rs9 (2015)). These advances have already provided benefits, including the editing of animal livestock and crops to establish pathogen resistance in agriculture (Wang, K., et al., Sci Rep 5, 16623 (2015); Ali, Z., et al., Mol Plant 8, 1288-91 (2015)). Traditional gene editing methods typically rely on transient transfection followed by rare random insertion events, recombinase-mediated cassette exchange or viral vectors (Recillas-Targa, F., Mol Biotechnol 34, 337-54 (2006)). By comparison, the use of CRISPR/CRISPR-associated protein (Cas) ribonucleoproteins (RNPs) provides vastly improved versatility and fidelity based on sequence-specific RNA-guided DNA cleavage (Jinek, M., et al., Science 337, 816-21 (2012)). The cleavage initiates the cellular DNA repair machinery either leading to insertion or deletion events through non-homologous end joining (NHEJ) or controlled insertion through homology-directed repair (HDR). However, full realization of the potential for CRISPR-mediated gene editing requires targeted, efficient nuclear delivery of intact RNPs consisting of Cas proteins and single-guide RNA (sgRNA), particularly for difficult-to-transfect cells (Dowdy, S. F., Nat Biotechnol 35, 222-229 (2017); Kim, S., et al., Genome Res 24, 1012-9 (2014)). Additionally, HDR can be promoted by co-formulating single-stranded template DNA (Savic, N., et al., bioRxiv (2017)). In contrast, the co-delivery of sgRNA and DNA encoding for Cas proteins using viral or liposomal delivery platforms has been observed to result in off-target editing and genome instability due to Cas protein overexpression (Ran, F. A. et al., Nature 520, 186-91 (2015); Yin, H. et al., Nat Biotechnol 34, 328-33 (2016); Fu, Y. et al., Nat Biotechnol 31, 822-6 (2013)). Recent advances have increased the efficiency of intact RNP delivery in vitro (Kim, S., et al., Genome Res 24, 1012-9 (2014); Han, X. et al., Sci Adv 1, e1500454 (2015); Ramakrishna, S., et al., Genome Res 24, 1020-7 (2014). Zuris, J. A. et al., Nat Biotechnol 33, 73-80 (2015)). Yet, current techniques suffer from cytotoxicity due to the disruption of the plasma membrane or the use of toxic lipids and polymers. Moreover, they neither provide control over the stoichiometry of RNPs with different sgRNAs to facilitate multiplexed gene editing nor over RNP release and intracellular trafficking (Barbieri, E. M., et al., Cell., 171(6):1453-1467 (2017)).

Efficient delivery of proteins, protein complexes, and multicomponent cargo to cells and tissues remains a difficult problem. Problems include size, stability, and ratio of components in multicomponent systems. CRISPR is a prime example of such a multicomponent cargo. Efficient, high-fidelity gene editing of human cells using CRISPR offers transformative potential for synthetic biology. Conventional approaches to gene editing rely on transient transfection followed by rare, random insertion events, recombinase-mediated cassette exchange, or viral vectors. In contrast, nuclear delivery of CRISPR-Cas RNPs offers precise, targeted genomic modifications. The delivery of intact CRISPR-RNPs rather than the DNA encoding for Cas proteins has been shown to achieve high efficiency, low off-target editing needed for next-generation biotechnology applications. However, the delivery of RNPs to the nucleus remains challenging using conventional transfection techniques. At present, no delivery platform offers full control over RNP stoichiometry and programmed intracellular release.

Accordingly, it is an object of the present invention to provide compositions that allow targeting, delivery, and intracellular trafficking of multicomponent cargo to cells and tissues.

It is also an object of the present invention to provide compositions comprising assemblies where the compositions allow targeting, delivery, and intracellular trafficking of multicomponent cargo to cells and tissues.

It is also an object of the present invention to provide compositions comprising nucleic acid assemblies where the compositions allow targeting, delivery, and intracellular trafficking of multicomponent cargo to cells and tissues.

It is also an object of the present invention to provide assemblies that allow targeting, delivery, and intracellular trafficking of multicomponent cargo to cells and tissues.

It is also an object of the present invention to provide nucleic acid assemblies that allow targeting, delivery, and intracellular trafficking of multicomponent cargo to cells and tissues.

It is also an object of the present invention to provide nucleic acid assemblies that allow targeting, delivery, and intracellular trafficking of CRISPR-Cas effector proteins, guide molecules, and template oligonucleotides to cells and tissues.

It is also an object of the present invention to provide nucleic acid assemblies that allow targeting, delivery, and intracellular trafficking of multicomponent cargo to cells and tissues, where the components of the cargo are delivered in a defined stoichiometric ratio.

It is also an object of the present invention to provide nucleic acid assemblies that allow targeting, delivery, and intracellular trafficking of CRISPR-Cas effector proteins, guide molecules, and template oligonucleotides to cells and tissues, where the CRISPR-Cas effector proteins, guide molecules, and template oligonucleotides are delivered in a defined stoichiometric ratio.

It is also an object of the present invention to provide compositions with physiochemical properties that enhance targeting of the compositions to one or more types of cells, tissues, organs, or microenvironments relative to other types of cells, tissues, organs, or microenvironments in vivo.

It is also an object of the present invention to provide compositions with physiochemical properties that enhance stability and/or half-life of the compositions in vivo.

It is also an object of the present invention to provide compositions with physiochemical properties that reduce immunogenicity of the compositions.

It is also an object of the present invention to provide compositions with physiochemical properties that enhance targeting of the compositions to one or more types of cells, tissues, organs, or microenvironments relative to other types of cells, tissues, organs, or microenvironments in vivo; (ii) enhance stability and/or half-life of the compositions in vivo; and/or (iii) reduce immunogenicity of the compositions.

It is also an object of the present invention to provide compositions with features that enhance intracellular trafficking of the composition and/or its cargo.

It is also an object of the present invention to provide such compositions comprising assemblies and cargo where the cargo comprising two or more cargo molecules enclosed and/or protected by the nucleic acid assembly in a defined stoichiometric ratio.

It is also an object of the present invention to provide methods of attaching cargo, targeting molecules, effector molecules, and/or intracellular trafficking molecules to assemblies.

It is also an object of the present invention to provide methods of treating or affecting subjects by administering assemblies, such as nucleic acid assemblies, to subjects.

It is also an object of the present invention to provide methods of formulating assemblies and/or compositions for administration to subjects.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each claim of this application.

Throughout this specification the word “comprise,” or variations such as “comprises” or “comprising,” will be understood to imply the inclusion of a stated element, integer or step, or group of elements, integers or steps, but not the exclusion of any other element, integer or step, or group of elements, integers or steps.

### BRIEF SUMMARY OF THE INVENTION

Disclosed are compositions and methods involving nucleic acid assemblies that enclose or protect cargo. In some forms, the nucleic acid assemblies have useful physiochemical properties. In some forms, the compositions and methods are used for targeting of the composition to one or more types of cells, tissues, organs, or microenvironments relative to other types of cells, tissues, organs, or microenvironments in vivo. In some forms, the compositions and methods are used for intracellular trafficking of the composition and/or its cargo. In some forms, the physiochemical properties enhance stability and/or half-life of the compositions in vivo. In some forms, the physiochemical properties reduce immunogenicity of the compositions.

In particular, disclosed are compositions that include a nucleic acid assembly comprising one or more nucleic acid molecules and cargo comprising two or more cargo molecules. In some forms, the nucleic acid assembly comprises physiochemical properties that: (i) enhance targeting of the composition to one or more types of cells, tissues, organs, or microenvironments relative to other types of cells, tissues, organs, or microenvironments in vivo; (ii) enhance stability and/or half-life of the composition in vivo; and/or (iii) reduce immunogenicity of the composition. In some forms, the nucleic acid assembly and/or cargo comprise features that enhance intracellular trafficking of nucleic acid assembly and/or its cargo. In some forms, the cargo is enclosed and/or protected by the nucleic acid assembly. In some forms, some or all of the cargo molecules in the composition are present in a defined stoichiometric ratio.

In some forms, one or more of the nucleic acid molecules comprising the nucleic acid assembly hybridize together. In some forms, the one or more nucleic acid molecules comprising the nucleic acid assembly hybridize together. In some forms, one or more of the nucleic acid molecules comprising the nucleic acid assembly partially or completely comprise RNA. In some forms, the one or more nucleic acid molecules comprising the nucleic acid assembly partially or completely comprise RNA. In some forms, one or more of the nucleic acid molecules comprising the nucleic acid assembly comprise RNA. In some forms, the one or more nucleic acid molecules comprising the nucleic acid assembly comprise RNA.

In some forms, the composition further comprises a plurality of bridging molecules. Generally, each bridging molecule is part of or directly or indirectly attached to either or both the nucleic acid assembly and one or more of the cargo molecules. Cargo molecules and bridging molecules that are part of or attach to each other are said to correspond to each other. These relationships are preferably selected and designed so that the bridging molecules collectively attach cargo molecules to the nucleic acid assembly in the defined stoichiometric ratio for the cargo molecules having a defined stoichiometric ratio.

In some forms, two or more of the bridging molecules constitute one or more pairs of bridging molecules that specifically bind to the other bridging molecule in the pair. Generally for each pair, one bridging molecule of the pair is part of or is directly or indirectly attached to the nucleic acid assembly and the other bridging molecule of the pair is part of or directly or indirectly attached to a corresponding cargo molecule. Based on this, specific binding of the pair of bridging molecules specifically attaches the corresponding cargo molecule to the nucleic acid assembly.

In some forms, at least one of the bridging molecules is part of the nucleic acid assembly, where the bridging molecule that is part of the nucleic acid assembly attaches directly or indirectly to a corresponding cargo molecule. Based on this, the bridging molecule that is part of the nucleic acid assembly specifically attaches the corresponding cargo molecule to the nucleic acid assembly.

In some forms, at least one of the bridging molecules is part of a cargo molecule, where the bridging molecule that is part of the cargo molecule attaches directly or indirectly to the nucleic acid assembly. Based on this, bridging molecule that is part of the cargo molecule specifically attaches the cargo molecule to the nucleic acid assembly.

In some forms, at least one of the bridging molecules is part of a cargo molecule, where the bridging molecule that is part of the cargo molecule is part of the nucleic acid assembly. Based on this, the bridging molecule that is part of the cargo molecule specifically attaches the cargo molecule to the nucleic acid assembly.

In some forms, direct attachments of bridging molecules to the nucleic acid assembly and/or cargo molecules each comprise a covalent bond, a non-covalent bond, or both a covalent bond and a non-covalent bond. In some forms, a plurality of the non-covalent bonds are involved in nucleic acid hybridization, whereby specificity of the attachment of the cargo molecule to the nucleic acid assembly is provided by the specificity of the nucleic acid hybridization. In some forms, at least one of the covalent bonds is formed by a click chemistry reaction, whereby specificity of the attachment of the cargo molecule to the nucleic acid assembly is provided by the specificity of the click chemistry reaction. In some forms, at least one of the non-covalent bonds is formed by specific binding molecules in a specific binding molecule pair, whereby specificity of the attachment of the cargo molecule to the nucleic acid assembly is provided by the specificity of the specific binding molecule pair.

In some forms, the defined stoichiometric ratio of the cargo molecules is based on the stoichiometric ratio at which the cargo molecules function together. In some forms, the defined stoichiometric ratio of the cargo molecules is based on a desired relative effect of the cargo molecules.

In some forms, the physiochemical properties are selected from structural properties, electric properties, biological properties, or a combination thereof.

In some forms, the cargo comprises one or more CRISPR-Cas effector proteins. In some forms, the cargo comprises one or more guide molecules. In some forms, the cargo comprises one or more template oligonucleotides. In some forms, the cargo comprises one or more CRISPR-Cas effector proteins, one or more guide molecules, and/or one or more template oligonucleotides. In some forms, one or more of the guide molecules is part of the nucleic acid assembly. In some forms, one or more of the template oligonucleotides is part of the nucleic acid assembly. In some forms, one or more of the template oligonucleotides is an HDR template. In some forms, one or more of the template oligonucleotides is an mRNA.

In some forms, the cargo comprises two or more CRISPR-Cas effector proteins, two or more guide molecules, two or more template oligonucleotides, or a combination thereof. In some forms, the cargo comprises three or more CRISPR-Cas effector proteins, three or more guide molecules, three or more template oligonucleotides, or a combination thereof.

In some forms, at least one of the one or more CRISPR-Cas systems is a Cas9 system, a Cas12 system, a Cas13 system, a dCas system, a nickase system, a paired nickase system, an Alt-R CRISPR system, a proxy-CRISPR system, an Alt-R dCas system, an Alt-R nickase system, an Alt-R paired nickase system, an Alt-R proxy-CRISPR system, a proxy-dCas system, a proxy-nickase system, a proxy-paired nickase system, an Alt-R proxy-dCas system, an Alt-R proxy-nickase system, or an Alt-R proxy-paired nickase system.

In some forms, at least one of the one or more CRISPR-Cas systems comprises one or more CRISPR-Cas effector proteins, wherein at least one of the CRISPR-Cas effector proteins is SpCas9, dCas9, Cas nickase, Cas9 nickase, FnCas9, StCas9, SaCas9, LpCas9, FnCas12, Cas12 nickase, AsCas12, LbCas12, Cas12a, Cas12b, Cas12c, Cas13, or Cas13d.

In some forms, at least one of the one or more CRISPR-Cas systems comprises a paired Cas9 nickase system, a paired Cas9 nickase system, a dCas/Cas proxy-CRISPR system, a dCas9/Cas9 proxy-CRISPR system, or an SpdCas9/FnCas9 proxy-CRISPR system.

In some forms, the proxy-CRISPR system comprises two first dCas ribonucleoproteins targeted to different sites flanking and on opposite sides of a main target site and a Cas ribonucleoprotein targeted to the main target site. In some forms, the proxy-CRISPR system further comprises two additional dCas ribonucleoproteins targeted to different sites flanking and on opposite sides of the main target site, wherein the sites to which the additional dCas ribonucleoproteins are targeted are not the same as the target sites to which the first dCas ribonucleoproteins are targeted.

In some forms, the Alt-R CRISPR system comprises a separate, shortened gRNA, a separate, shortened tracrRNA, a Cas9 protein. In some forms, the paired nickase system leaves 5′ overhangs. In some forms, the cargo comprises one or more components of two or more CRISPR-Cas systems.

In some forms, the cargo does not comprise a CRISPR-Cas effector protein, guide molecule, or HDR template. In some forms, the cargo comprises an anti-sense nucleic acid, mRNA, miRNA, piRNA, siRNA, or a combination thereof.

In some forms, the composition further comprises one or more targeting molecules that specifically targets the nucleic acid assembly to one or more types of cells, tissues, organs, or microenvironments relative to other types of cells, tissues, organs, or microenvironments in vivo. In some forms, the targeting molecules are selected from the group consisting of, for example, aptamers, antibodies, and lectins.

In some forms, the nucleic acid assembly forms a container. In some forms, the cargo is inside the container.

In some forms, the nucleic acid assembly comprises one or more RNA/DNA hybrid regions. In some forms, the RNA/DNA hybrid region includes at least part of an RNA scaffold and DNA staple or at least part of a DNA scaffold and RNA staple. In some forms, one or more of the bridging molecules or bound bridging molecule pairs comprises an RNA/DNA hybrid region. In some forms, one or more of the RNA/DNA hybrid regions facilitates release of one or more cargo molecules in the presence of an RNA/DNA hybrid specific nuclease.

In some forms, the nucleic acid assembly comprises a plurality of effector molecules, wherein the effector molecules produce or contribute to the physiochemical properties. In some forms, the effector molecules comprise, for example, polyethylene glycol molecules, lipids, polar groups, charged groups, amphipathic groups, and albumin binding molecules.

Disclosed are methods of making and using the disclosed compositions. For example, disclosed are methods of assembling the disclosed nucleic acid assemblies, including, for example, methods of attaching cargo to nucleic acid assemblies, attaching of targeting moieties, effector molecules, and intracellular trafficking molecules to nucleic acid assemblies. As another example, disclosed are methods of administering the disclosed nucleic acid assembly compositions to subjects, including, for example, methods of treating subjects by administering the disclosed nucleic acid assembly compositions to subjects and methods of affecting subjects by administering the disclosed nucleic acid assembly compositions to subjects. Also disclosed are methods of formulating the disclosed nucleic acid assembly compositions for administration to subjects. Also disclosed are methods of producing components of the disclosed nucleic acid assembly compositions to subjects, including, for example, methods of producing nucleic acid molecules, methods of producing CRISPR-Cas effector proteins, and methods of producing bridging molecules.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIGS. 1A, 1B, 1C, and 1D are diagrams of examples of nucleic acid assemblies and nucleic acid assembly compositions. FIG. 1A shows the conceptualization (1) and (2) and actualization (3) of an example of a nucleic acid assembly (exemplified by an icosahedral shape). The inset in (3) shows single-stranded overhangs, which are an example of attachment molecules (e.g., bridging molecules) that can be used to attach components (e.g., cargo, targeting molecules, effector molecule) to the nucleic acid assemblies. FIG. 1B shows a nucleic acid assembly composition with PEG (an example of an effector molecule) attached to the outside as shielding and intact CRISPR RNPs (an example of cargo) attached to and enclosed by the nucleic acid assembly. FIG. 1C shows a nucleic acid assembly composition with multiplexed complexation of components. Targeting molecules (illustrated as squares attached via nucleic acid stalks) are shown attached to the outside of the nucleic acid assembly as shielding and intact CRISPR RNPs (an example of cargo) are shown attached to and enclosed by the nucleic acid assembly. The patterns, clustering, density, and distribution of targeting can be used to optimize cell and tissue targeting. FIG. 1D shows a nucleic acid assembly composition with multiplexed complexation of components. Targeting molecules (illustrated as squares attached via nucleic acid stalks), cell penetrating peptides (an example of an effector molecule; illustrated as spirals attached via nucleic acid stalks), and PEG (an example of an effector molecule; illustrated as twisted lines) are shown attached to the outside of the nucleic acid assembly.

FIGS. 2A and 2B are diagrams of examples nucleic acid assembly compositions. FIG. 2A shows the complexation (attachment) of cargo (intact CRISPR RNPs are illustrated) with a nucleic acid assembly. A gel shift assay demonstrated the complexation. FIG. 2B illustrates release of the cargo (an intact CRISPR RNP is illustrated) and breakup of segments of the nucleic acid assembly (a double stranded DNA segment is illustrated) through action of an RNA/DNA hybrid nuclease (RNAse H is illustrated) and strand displacement. A gel shift assay demonstrated the release of cargo and breakup of the nucleic acid assembly.

FIGS. 3A, 3B, and 3C are diagrams of examples of nucleic acid assemblies illustrating examples of properties that can be varied or selected. FIG. 3A shows examples of selection of size of the nucleic acid assemblies. FIG. 3B shows examples of different geometries of nucleic acid assemblies that can be used. FIG. 3C shows examples of controlled attachment of functional molecules (e.g., targeting molecules, effector molecules, cargo molecules) to nucleic acid assemblies, with different patterns and densities controlled by design of the attachment molecules (e.g., bridging molecules).

FIG. 4 is a diagram illustrating an example of how features combined in nucleic acid assembly compositions enhance targeting and effective delivery of cargo to a cell, including receptor-mediated endocytosis (1), endosomal escape (2) and controlled cargo release (3). These functions solve the three central challenges for nuclear RNP delivery and gene editing.

FIGS. 5A and 5B are diagrams of examples nucleic acid assembly compositions. FIG. 5A shows the complexation (attachment) of cargo (intact CRISPR RNPs are illustrated) with a nucleic acid assembly. The cargo includes HDR ssDNA that is used as a scaffold nucleic acid in the nucleic acid assembly. FIG. 5B illustrates release of the cargo (an intact CRISPR RNP is illustrated) and breakup of segments of the nucleic acid assembly (to release the HDR ssDNA) through action of an RNA/DNA hybrid nuclease (RNAse H is illustrated) and strand displacement.

FIG. 6 is plasmid map of an exemplary plasmind for all-in-one production of a Crispr enzyme (e.g., Cas9), sgRNA, staple strands (e.g., RNA staples) and M13 phage genes. As illustrated, pET21a-biobrick—with M13 gene 2 and gene 5 BioBrick parts were cloned together to be independent inducible expression cistrons.

### DETAILED DESCRIPTION OF THE INVENTION

The disclosed method and compositions may be understood more readily by reference to the following detailed description of particular embodiments and the Example included therein and to the Figures and their previous and following description.

The delivery to, and intracellular effects in, cells, organs, and tissues of large and multicomponent cargo (such as CRISPR RNPs) is a continuing problem in both basic research and medical treatments. For example, gene editing of mammalian cells represents an important process for basic research, applied biotechnology, and medical treatment. Improving the fidelity and efficiency of editing, particularly for difficult-to-transfect and -reach cells, will impact the industrial production of biomolecules, antibody engineering, the development of ‘organ-on-a-chip’ devices, and medical treatments involving gene editing. The transformative method here provides unique opportunities to address the limitations of traditional delivery methods. Disclosed is an adaptable nucleic acid assembly-based delivery platform to facilitate effective delivery of large and multicomponent cargo (such as CRISPR RNPs). The disclosed compositions and methods allow for the multiplexed complexation of multicomponent cargo and of multiple different cargos to nucleic acid assemblies at controlled stoichiometry, simultaneously eliminating the requirement for toxic liposomal formulations. The internalization of nucleic acid assemblies can be promoted via receptor-mediated endocytosis by functionalization with targeting ligands such as those for endocytic lectins. The nucleic acid assemblies can also be programmed to escape the endosome using CPPs and NLSs to target the nucleus, for example, by releasing the RNPs in the cytoplasm. The disclosed synthetic procedures to functionalize nucleic acid assemblies enables the controlled spatial orientation of targeting ligands, CPPs, and other modalities on nucleic acid assemblies of varying size, geometry and mechanics. Further, carefully designed nucleic acid assembly libraries can be leveraged to investigate the molecular and cellular mechanisms governing receptor-mediated endocytosis, endosomal escape, and nuclear translocation of RNPs. Thus, the disclosed compositions and methods find wide use from basic research up to clinical therapies and treatments. Accordingly, the disclosed compositions and methods will have a broad translational impact.

It has been discovered that programmable nucleic acid assemblies provide unique opportunities to engineer solutions to the central challenges of large and multicomponent cargo delivery. The spatial and stoichiometric control over their functionalization with cargo, targeting ligands, and other modalities enables leveraging of the cellular and molecular processes governing endocytosis, endosomal escape, and nuclear translocation of the cargo for superior effectiveness. The disclosed compositions represent an adaptable delivery platform for CRISPR-mediated gene editing (and other large and multicomponent cargo), useful for both medical applications and basic research. The disclosed compositions can be targeted to a large variety of cells and tissues based on the wide array of targeting molecules that are known.

Disclosed are compositions and methods involving nucleic acid assemblies that enclose or protect cargo. In some forms, the nucleic acid assemblies have useful physiochemical properties. In some forms, the compositions and methods are used for targeting of the composition to one or more types of cells, tissues, organs, or microenvironments relative to other types of cells, tissues, organs, or microenvironments in vivo. In some forms, the compositions and methods are used for intracellular trafficking of the composition and/or its cargo. In some forms, the physiochemical properties enhance stability and/or half-life of the compositions in vivo. In some forms, the physiochemical properties reduce immunogenicity of the compositions.

The disclosed compounds, components, compositions, and methods can be formulated and practiced in a variety of ways and modes. For example, in some forms, the disclosed compositions allow targeting, delivery, and intracellular trafficking of multicomponent cargo to cells and tissues. In some forms, the disclosed assemblies allow targeting, delivery, and intracellular trafficking of multicomponent cargo to cells and tissues. In some forms, the disclosed nucleic acid assemblies allow targeting, delivery, and intracellular trafficking of multicomponent cargo to cells and tissues. In some forms, the disclosed nucleic acid assemblies allow targeting, delivery, and intracellular trafficking of CRISPR-Cas effector proteins, guide molecules, and template oligonucleotides to cells and tissues.

In some forms, the disclosed compositions comprise assemblies where the compositions allow targeting, delivery, and intracellular trafficking of multicomponent cargo to cells and tissues. In some forms, the disclosed compositions comprise nucleic acid assemblies where the compositions allow targeting, delivery, and intracellular trafficking of multicomponent cargo to cells and tissues.

In some forms, the disclosed nucleic acid assemblies allow targeting, delivery, and intracellular trafficking of multicomponent cargo to cells and tissues, where the components of the cargo are delivered in a defined stoichiometric ratio. In some forms, the disclosed nucleic acid assemblies allow targeting, delivery, and intracellular trafficking of CRISPR-Cas effector proteins, guide molecules, and template oligonucleotides to cells and tissues, where the CRISPR-Cas effector proteins, guide molecules, and template oligonucleotides are delivered in a defined stoichiometric ratio.

In some forms, the disclosed compositions have physiochemical properties that enhance targeting of the compositions to one or more types of cells, tissues, organs, or microenvironments relative to other types of cells, tissues, organs, or microenvironments in vivo. In some forms, the compositions comprise features that enhance intracellular trafficking of the composition and/or its cargo. In some forms, the disclosed compositions have physiochemical properties that enhance stability and/or half-life of the compositions in vivo. In some forms, the disclosed compositions have physiochemical properties that reduce immunogenicity of the compositions. In some forms, the disclosed compositions have physiochemical properties that enhance targeting of the compositions to one or more types of cells, tissues, organs, or microenvironments relative to other types of cells, tissues, organs, or microenvironments in vivo; (ii) enhance stability and/or half-life of the compositions in vivo; and/or (iii) reduce immunogenicity of the compositions. In some forms, the compositions comprise features that enhance intracellular trafficking of the composition and/or its cargo.

In some forms, the disclosed compositions comprise assemblies and cargo where the cargo comprises two or more cargo molecules enclosed and/or protected by the nucleic acid assembly in a defined stoichiometric ratio.

In some forms, the disclosed methods provide for attachment of cargo, targeting molecules, effector molecules, and/or intracellular trafficking molecules to assemblies.

In some forms, the disclosed methods involve treating or affecting subjects by administering assemblies, such as nucleic acid assemblies, to subjects.

In some forms, the disclosed methods involve formulating assemblies and/or compositions for administration to subjects.

Preferred forms of the disclosed compositions are non-viral, structured nucleic acid assemblies useful for efficiently delivering multiplexed CRISPR-Cas RNPs (and/or other large or multicomponent cargo) to the nucleus of mammalian cells in vitro and in vivo. These nucleic acid assemblies hold particular value for gene editing in difficult-to-transfect cells such as primary hepatocytes, which may aid in the development of ‘liver-on-a-chip’ devices for toxin sensing. In addition, biotechnological processes that require screening large numbers of genetic constructs will benefit from the disclosed approach, including antibody engineering and the industrial production of biomolecules and metabolites. Three major challenges to nuclear delivery of CRISPR-RNP (and/or other large or multicomponent cargo) are overcome by the disclosed compositions and methods. First, the size of RNPs and single-stranded DNA templates required for homology-directed repair that typically prohibit the use of viral and nanoparticle approaches. Second, efficient cellular uptake via endocytosis to ensure high transfection efficiency with minimal cytotoxicity. And third, optimal intracellular cargo trafficking for gene editing, including endosomal escape, controlled cargo release in the cytoplasm, followed by nuclear translocation.

The disclosed compositions and methods address these challenges by formulating CRISPR-RNPs (and/or other large or multicomponent cargo) packaged within fully synthetic structured nucleic acid assemblies. RNPs can be complexed with, for example, single-stranded overhangs (i.e., bridging molecules) on the nucleic acid assemblies, allowing for controlled stoichiometry, multiplexing, and release. Second, use of targeting molecules (such as lectins) to promote endocytosis of the nucleic acid assembly, using the target of the targeting molecule (such as lectin asialoglycoprotein receptor (ASGPR) expressed in primary hepatocytes). Third, endosomal escape can be controlled to achieve programmed cytoplasmic release of CRISPR-RNP (and/or other large or multicomponent cargo) with minimal cytotoxicity. The effectiveness of such compositions and methods can be assessed by, for example, evaluating CRISPR-mediated gene editing efficiency in target cells (such as primary hepatocytes) via sequence-specific amplification and cleavage. The effectiveness of other cargo can be assess using techniques appropriate for their expected effects.

The disclosed compositions and methods can be used for a variety of delivery goals and targets, such as gene editing of various mammalian cells relevant for antibody engineering and industrial production of biomolecules, as well as in vivo delivery and effect of CRISPR-RNP and other large or multicomponent cargo.

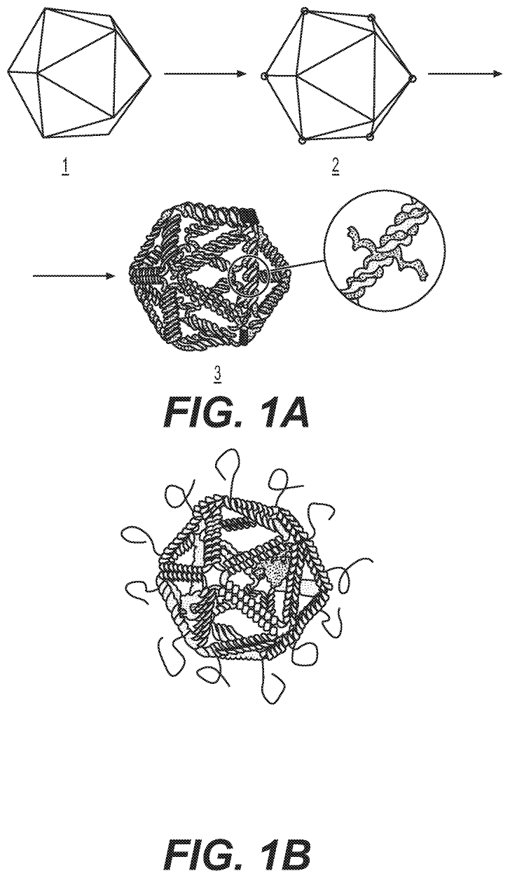

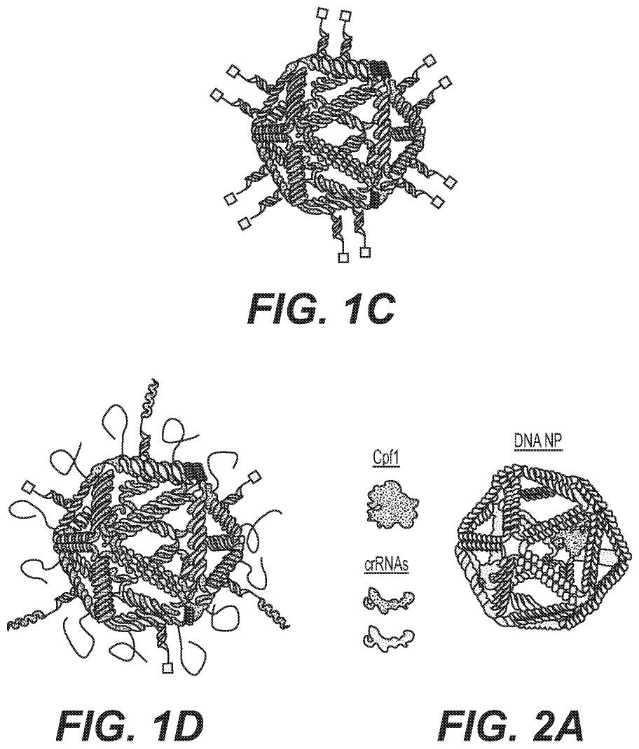

The size of intact CRISPR RNPs has generally impeded the use of synthetic nanoparticles (NPs) as delivery platforms. DNA-based materials represent a viable alternative, allowing for the construction of nucleic acid assemblies at the 100 nm scale and have previously been used to deliver drug-like small molecules and siRNA in vivo (Zhang, Q., et al., ACS Nano 8, 6633-43 (2014); Lee, H., et al., Nat Nanotechnol 7, 389-93 (2012)). General DNA origami methods were developed to design and fold wireframe nucleic acid assemblies of arbitrary geometry and size by annealing of oligonucleotide staples to scaffold ssDNA (FIG. 1A; Rothemund, P. W., Nature 440, 297-302 (2006); Veneziano, R., et al., Science 352, 1534 (2016)). These methods include the use of asymmetric polymerase chain reactions (aPCR) to synthesize single-stranded HDR template and scaffold DNA in large quantities (Veneziano, R. et al., bioRxiv (2017)), as well as more recently fully biological production (Veneziano, R. et al., bioRxiv (2017); Shepherd, et al., bioRxiv, (2019), Shepherd, et al., Sci Rep., 9: 6121 (2019), (29)). Disclosed herein is the development of nucleic acid assemblies as an adaptable delivery platform, addressing the limitations discussed above to facilitate, for example, CRISPR-mediated gene editing for synthetic biology and other effects provided by other cargo. The sequence-specific functionalization of nucleic acid assemblies based on, for example, single-stranded overhangs or chemical modifications provides stoichiometric and spatial control over delivered cargo, targeting ligands and other modalities.

In some forms, cargo, such as CRISPR RNPs and single-stranded HDR template DNA, can be complexed with nucleic acid assemblies (FIG. 1B). As part of the development of particular nucleic acid assemblies, the structural integrity and activity of the nucleic acid assemblies can be assessed or monitored in presence of nucleases and proteases typically found the cellular environment. This provides assessment of stability of the nucleic acid assemblies, cell-proximate release of cargo, or both. RNPs and nucleic acid assemblies can also be modified to limit enzymatic degradation. Examples include chemical modifications of the sgRNA and the functionalization of nucleic acid assemblies with shielding molecules (Yin, H., et al., Nat Biotechnol., 35(12):1179-1187 (2017)). The first cellular barrier nucleic acid assemblies encounter is the plasma membrane. While the non-specific internalization of nucleic acid assemblies by most cell types is inefficient, receptor-mediated endocytosis can be leveraged to optimize this process. Lectins have been recognized for their capacity to promote endocytosis and have emerged as target receptors for the therapeutic delivery of antigens, siRNA and small-molecule drugs (Johannssen, T. & Lepenies, B., Trends Biotechnol 35, 334-346 (2017); Angata, T., et al., Trends Pharmacol Sci 36, 645-60 (2015); D'Souza, A. A. & Devarajan, P. V., J Control Release 203, 126-39 (2015)). Many endocytic lectins share biochemical profiles that increase the efficiency of nanoparticle internalization and simultaneously prevent lysosomal degradation of both nanoparticles and cargo (Onizuka, T., et al., FEBS J 279, 2645-56 (2012); Hanske, J., et al., J Am Chem Soc 138, 12176-86 (2016); Schwartz, A. L., et al., J Cell Biol 98, 732-8 (1984); O'Reilly, M. K., et al., J Immunol 186, 1554-63 (2011)). In mammals, lectins expressed on various immune cells, including dendritic cells, macrophages, and B cells, as well as non-immune cells such as keratinocytes, epithelial cells, and hepatocytes. Moreover, lectins display increased expression levels in many cancer cells and transformed cell lines (Esko, J. D., et al. Proteins That Bind Sulfated Glycosaminoglycans. In Essentials of Glycobiology (eds. Varki, A. et al.) (Cold Spring Harbor (N.Y.), 2015)). The broad expression profile, in combination with their capacity to promote endocytosis, renders endocytic lectins viable target receptors for the disclosed nucleic acid assemblies for delivery of cargo, such as CRISPR RNP.

In the context of in vitro delivery, primary hepatocytes represent useful target cells for the disclosed compositions. First, they represent biotechnologically relevant target cells for genetic engineering, such as for the development of ‘liver-on-a-chip’ devices to study the metabolism of toxins and drugs (Bhatia, S. N., et al., Sci Transl Med 6, 245sr2 (2014); Griffith, L. G., et al., Hepatology 60, 1426-34 (2014); Guye, P., et al., Nat Commun 7, 10243 (2016); Dong, J., et al., Cell 130, 1120-33 (2007)). Second, they display low viability in vitro and thus are difficult to transfect (Han, X. et al., Sci Adv 1, e1500454 (2015); Gresch, O. & Altrogge, L., Methods Mol Biol 801, 65-74 (2012).). Finally, they express the endocytic lectin asialoglycoprotein receptor (ASGPR), which represents an established target receptor the in vivo delivery of siRNA and other cargo (Prakash, T. P., et al., J Med Chem 59, 2718-33 (2016); Sanhueza, C. A., et al., J Am Chem Soc 139, 3528-3536 (2017)). For these and other suitable target cells, the disclosed nucleic acid assemblies can be functionalized with natural glycans such as N-acetylgalactosamine (GalNAc) as well as glycomimetic ASGPR ligands to promote endocytosis by hepatocytes (FIG. 1C; Sanhueza, C. A., et al., J Am Chem Soc 139, 3528-3536 (2017)). The spatial organization of ligands as well as the size and geometry of nucleic acid assemblies can affect endocytosis (Huang, X., et al., Bioconjug Chem 28, 283-295 (2017); Agarwal, R. et al., Proc Natl Acad Sci USA 110, 17247-52 (2013); Bujold, K. E., et al., Chemical Science 5, 2449-2455 (2014)) and so these features of the disclosed compositions should be designed and engineered to produce efficient delivery. Once the nucleic acid assemblies have been internalized, the intracellular trafficking of the cargo can be important for the effectiveness of the cargo. For example, effective endosomal release and entry into the nucleus is important for the efficiency of gene editing by a CRISPR RNP cargo. Various features can be used to optimize endosomal escape, cargo release, and nuclear translocations (FIG. 1D). Programmed endosomal escape of the nucleic acid assemblies represents a useful way to limit lysosomal degradation of nucleic acid assemblies and their cargo. Cell-penetrating peptides (CPPs) have been demonstrated to destabilize endosomal membranes and to induce nanoparticles translocation into the cytoplasm (Bechara, C. & Sagan, S., FEBS Lett 587, 1693-702 (2013)). Thus, functionalizing nucleic acid assemblies with CPPs, with, for example, controlled spatial organization of the CPPs on the nucleic acid assemblies, is one strategy for promoting endosomal escape and minimizing cytotoxicity. Combination of these strategies (targeted endocytosis, endosomal escape, and controlled release of cargo from the nucleic acid assemblies) can provide the most efficient delivery and effectiveness of the cargo.

The disclosed nucleic acid assembly-based delivery platform provides particular benefits for national security and improved patient care in several ways. Broadly, efficient high-fidelity gene editing for industrial biomolecule and metabolite production will translate into short process optimization cycles following terrorism threats. For example, the availability of antibodies can be vital for the detection and neutralization of toxins encountered in infectious diseases as well biological and chemical warfare. The adaptability of the disclosed delivery platform to hybridoma cells offers unique opportunities for antibody engineering. Thus, the disclosed compositions and methods can contribute to the development of point-of-care diagnostic tools and antidotes for patients in emerging and rapidly developing medical crises. Moreover, CRISPR-mediated gene editing of difficult-to-transfect cells such as primary cells or induced pluripotent stem cells (iPSCs) will facilitate the use of improved in vitro models for basic research including drug or toxin screens, functional genomics, and systems biology of cell networks. The disclosed delivery platform will also have immediate impact on the development of in vitro liver models and ‘liver-on-a-chip’ devices. Consequently, the disclosed compositions and methods will contribute to the understanding of liver-associated diseases such as Malaria infections prevalent in tropical or subtropical areas.

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

### Materials

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if an assembly is disclosed and discussed and a number of modifications that can be made to a number of components including the assembly are discussed, each and every combination and permutation of assembly and the modifications that are possible are specifically contemplated unless specifically indicated to the contrary. Thus, if a class of molecules or components A, B, and C are disclosed as well as a class of molecules or components D, E, and F and an example of a combination molecule or component, A-D, is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. Further, each of the materials, compositions, components, etc. contemplated and disclosed as above can also be specifically and independently included or excluded from any group, subgroup, list, set, etc. of such materials. These concepts apply to all aspects of this application including, but not limited to, steps in methods of making and using the disclosed compositions and assemblies. Thus, if there are a variety of additional steps that can be performed it is understood that each of these additional steps can be performed with any specific embodiment or combination of embodiments of the disclosed methods, and that each such combination is specifically contemplated and should be considered disclosed.

The terms “nucleic acid molecule,” “nucleic acid sequence,” “nucleic acid fragment,” “oligonucleotide,” and “polynucleotide” are used interchangeably and are intended to include, but not limited to, a polymeric form of nucleotides that may have various lengths, either deoxyribonucleotides (DNA) or ribonucleotides (RNA), or analogs or modified nucleotides thereof, including, but not limited to locked nucleic acids (LNA), peptide nucleic acids (PNA), and morpholinos. An oligonucleotide is typically composed of a specific sequence of four nucleotide bases: adenine (A); cytosine (C); guanine (G); and thymine (T) (uracil (U) for thymine (T) when the polynucleotide is RNA). Thus, the term “oligonucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Oligonucleotides may optionally include one or more non-standard nucleotide(s), nucleotide analog(s) and/or modified nucleotides. In some cases nucleotide sequences are provided using character representations recommended by the International Union of Pure and Applied Chemistry (IUPAC) or a subset thereof. IUPAC nucleotide codes used herein include, A=Adenine, C=Cytosine, G=Guanine, T=Thymine, U=Uracil, R=A or G, Y=C or T, S=G or C, W=A or T, K=G or T, M=A or C, B=C or G or T, D=A or G or T, H=A or C or T, V=A or C or G, N=any base, “.” or “-”=gap. In some forms the set of characters is (A, C, G, T, U) for adenosine, cytidine, guanosine, thymidine, and uridine respectively. In some forms the set of characters is (A, C, G, T, U, I, X, Ψ, R, Y, N) for adenosine, cytidine, guanosine, thymidine, uridine, inosine, uridine, xanthosine, pseudouridine respectively. The modified sequences, non-natural sequences, or sequences with modified binding, may be in the genomic, the guide or the tracr sequences.

The terms “staple strands” or “helper strands” are used interchangeably. “Staple strands” or “helper strands” refer to oligonucleotides that work as glue to hold the scaffold nucleic acid assembly in its three-dimensional geometry. Additional nucleotides can be added to the staple strand at either 5′ end or 3′ end, and those are referred to as “staple overhangs.” Staple overhangs can be functionalized to have desired properties such as a specific sequence to hybridize to a target nucleic acid sequence, or a targeting element. In some instances, the staple overhang is biotinylated for capturing the DNA assembly on a streptavidin-coated bead. In some instances, the staple overhang can be also modified with chemical moieties. Non-limiting examples include Click-chemistry groups (e.g., azide group, alkyne group, DIBO/DBCO), amine groups, and Thiol groups. In some instances some bases located inside the oligonucleotide can be modified using base analogs (e.g., 2-Aminopurine, Locked nucleic acids, such as those modified with an extra bridge connecting the 2′ oxygen and 4′ carbon) to serve as linker to attach functional moieties (e.g., lipids, proteins). Alternatively DNA-binding proteins or guide RNAs can be used to attach secondary molecules to the DNA scaffold.

The terms “scaffolded origami,” “origami,” or “nucleic acid assembly” are used interchangeably. They refer to a long, single strand of polynucleotide (scaffold strand) that is folded into desired shapes on the order of about 10 nm to a micron, or more. In some forms, nucleic acid scaffold sequences are folded into nucleic acid assemblies by hybridization to small nucleic acid “staple sequences.” Alternatively, single-stranded nucleic acid scaffolds can be designed to fold into an origami object without helper strands, for example, using parallel or paranemic crossover motifs. The scaffolded origami or origami can be composed of deoxyribonucleotides (DNA) or ribonucleotides (RNA), or analogs or modified nucleotides thereof, including, but not limited to locked nucleic acids (LNA), peptide nucleic acids (PNA), and morpholinos. A scaffold or origami composed of DNA can be referred to as, for example a scaffolded DNA origami or DNA origami, etc. It will be appreciated that where compositions, methods, and systems herein are exemplified with DNA (e.g., DNA origami), other nucleic acid molecules can be substituted. Typically, the nucleic acid assemblies are nucleic acid objects made from scaffold nucleic acid with or without staple nucleic acid sequences, or from encapsulated nucleic acid of any arbitrary length/form, or any combinations thereof. The nucleic acid assembly can be composed of deoxyribonucleotides (DNA) or ribonucleotides (RNA), or analogs or modified nucleotides thereof, including, but not limited to locked nucleic acids (LNA), peptide nucleic acids (PNA), and morpholinos.

The term “Single Stranded Nucleic Acid Scaffold Sequence” refers to a single-stranded nucleic acid sequence that is routed throughout the entire structure of a nucleic acid assembly. The nucleic acid structure assemblies optionally include oligonucleotide staple strands that hybridize to the scaffold sequence and create the polyhedral structure. When the polyhedral nucleic acid assemblies do not include staple strands, the scaffold sequence hybridizes to itself to create the nucleic acid assembly.

The terms “nucleic acid overhang,” “DNA overhang tag,” “staple overhang tag,” and “address tag” are used interchangeably to refer to any additional nucleotides added to the nucleic acid assemblies that can be functionalized. In some forms, these additional nucleotides are added to the staple strand. In some instances, the overhang tag contains one or more nucleic acid sequences that encode metadata for the associated nucleic acid assemblies. In some instances, the overhang tag contains sequences designed to hybridize other nucleic acid sequences such as those on tags of other nucleic acid assemblies. In other instances, the overhang contains one or more sites for conjugation to a molecule. For example, the overhang tag can be conjugated to a protein, or non-protein molecule, for example, to enable affinity-binding of the nucleic acid assemblies. Exemplary proteins for conjugating to overhang tags include biotin and antibodies, or antigen-binding fragments of antibodies.

The term “bit stream encoded sequence” is any nucleic acid sequence that encodes for data to be stored. Bit stream-encoded nucleic acid can be in the form of a linear nucleic acid sequence, a two-dimensional nucleic acid object or a three-dimensional nucleic acid object. Bit stream-encoded nucleic acid can include a sequence that is synthesized, or naturally occurring The term “bit” is a contraction of “binary digit.” Commonly “bit” refers to a basic capacity of information in computing and telecommunications. A “bit” conventionally represents either 1 or 0 (one or zero) only, though other codes can be used with nucleic acids that contain 4 nucleotide possibilities (ATGC) at every position, and higher-order codecs including sequential 2-, 3-, 4-, etc. nucleotides can alternatively be employed to represent bits, letters, or words.

### A. Compositions

The disclosed compositions generally include a nucleic acid assembly and cargo. The purpose and use of the disclosed compositions is, for example, hold, protect, transport, and/or deliver the cargo to cells, tissues, organs, and/or microenvironments. Thus, preferred forms of the disclosed compositions are designed and adapted to these purposes and uses.

1. Nucleic Acid Assemblies

Disclosed are nucleic acid assemblies. Generally, the disclosed nucleic acid assemblies are structures primarily assembled from and composed of nucleic acid molecules. Generally, the disclosed nucleic acid assemblies are assembled, structured, and/or held together by nucleic acid hybridization. The nucleic acid molecules can be any form of nucleic acid, including, for example, DNA, RNA mixtures of DNA and RNA, nucleic acids including or composed of modified nucleotides and/or modified nucleic acids, such as peptide nucleic acids. The composition of nucleic acid molecules used in the nucleic acid assemblies can be chosen based on and/or to aid, for example, design of, assembly of, and/or cargo attachment to or release from the nucleic acid assemblies. As just one example, the use of RNA/DNA hybrids as part of the nucleic acid assemblies can facilitate release of cargo, break-up of the nucleic acid assembly, or both (through the action of RNA/DNA specific nuclease).

Many designs and methods are known for making different types of nucleic acid assemblies, such as DNA tile-based structurers, and scaffolded DNA origami structures. Many of these methods and designs can be used with and adapted to the disclosed nucleic acid assemblies.

Exemplary methods include those described by Benson E et al (Benson E et al., Nature 523, 441-444 (2015)), Rothemund P W et al (Rothemund P W et al., Nature. 440, 297-302 (2006)), Douglas S M et al., (Douglas S M et al., Nature 459, 414-418 (2009)), Ke Y et al (Ke Y et al., Science 338: 1177 (2012)), Zhang F et al (Zhang F et al., Nat. Nanotechnol. 10, 779-784 (2015)), Dietz H et al (Dietz H et al., Science, 325, 725-730 (2009)), Liu et al (Liu et al., Angew. Chem. Int. Ed., 50, pp. 264-267 (2011)), Zhao et al (Zhao et al., Nano Lett., 11, pp. 2997-3002 (2011)), Woo et al (Woo et al., Nat. Chem. 3, pp. 620-627 (2011)), and Torring et al (Torring et al., Chem. Soc. Rev. 40, pp. 5636-5646 (2011)), which are incorporated here in the entirety by reference.

DNA nucleic acid assemblies are assemblies of any arbitrary geometric shapes. DNA nucleic acid assemblies can be of two-dimensional shapes, three dimensional shapes, or non-spherical morphologies.

i. Types of Nucleic Acid Assemblies

Nucleic acid assemblies, such as DNA nucleic acid assemblies, are assemblies of any arbitrary geometric shapes. DNA nucleic acid assemblies can be of two-dimensional shapes, for example plates, or any other 2-D shape of arbitrary sizes and shapes. In some forms, the nucleic acid assemblies are simple DX-tiles, with two DNA duplexes connected by staples. DNA double crossover (DX) motifs are examples of small tiles (approximately 4 nm×approximately 16 nm) that have been programmed to produce 2D crystals (Winfree E et al., Nature. 394:539-544(1998)); often these tiles contain pattern-forming features when more than a single tile constitutes the crystallographic repeat.

In some forms, the nucleic acid assemblies are 2-D crystalline arrays by parallel double helical domains with sticky ends on each connection site (Winfree E et al., Nature, 6; 394(6693):539-44 (1998)). In some forms, the nucleic acid assemblies are 2-D crystalline arrays by parallel double helical domains, held together by crossovers (Rothemund P W K et al., PLoS Biol. 2:2041-2053 (2004)). In some forms, the nucleic acid assemblies are 2-D crystalline arrays by an origami tile whose helix axes propagate in orthogonal directions (Yan H et al., Science. 301:1882-1884 (2003)).

DNA nucleic acid assemblies can be any solid in three dimensions that can be rendered with flat polygonal faces, straight edges and sharp corners or vertices. Exemplary basic target structures include cuboidal structures, icosahedral structures, tetrahedral structures, cuboctahedral structures, octahedral structures, and hexahedral structures. In some forms, the target structure is a convex polyhedron, or a concave polyhedron. For example, in some forms, the nucleic acid assemblies are of a uniform polyhedron that has regular polygons as faces and is isogonal. In other forms, nucleic acid assemblies are of an irregular polyhedron that has unequal polygons as faces. In some forms, the target structure is a truncated polyhedral structure, such as truncated cuboctahedron.

Platonic polyhedrons include polyhedrons with multiple faces, for example, 4 faces (tetrahedron), 6 faces (cube or hexahedron), 8 faces (octahedron), 12 faces (dodecahedron), 20 faces (icosahedron).

“Scaffolded DNA origami” is a highly versatile approach to program rigid nanometer-scale 3D molecular structures of arbitrary size and symmetry on the 5 to 100 nm scale.

Typically, scaffolded DNA origami objects, or DNA nucleic acid assemblies having the desired shape, are produced by folding a long single-stranded polynucleotide, referred to as a “scaffold strand,” into a desired shape or structure. In some forms, the scaffold strand is folded by hybridizing to a number of small “staple strands,” which act as a glue to hold the scaffold in place. Typically, when staple strands are employed, the number of staple strands will depend upon the size of the scaffold strand and the complexity of the shape or structure. For example, for relatively short scaffold strands (e.g., about 150 to 1,500 bases in length) and/or simple structures the number of staple strands are small (e.g., about 5, 10, 50 or more). For longer scaffold strands (e.g., greater than 1500 bases) and/or more complex structures, the number of staple strands can be several hundreds to thousands (e.g., 50, 100, 300, 600, 1000 or more helper strands). The choice of staple strands determines the pattern. In some forms, a software program is used to identify the staple strands needed to form a given design. In some forms, the target structure is a nucleic acid assembly that has a non-spherical geometry. Therefore, in some forms, the target structure has geometry with holes. Exemplary non-spherical geometries include toroidal polyhedra and nested shapes. Exemplary toroidal polyhedra include a torus, and double torus. Exemplary topologies of nested shapes include nested cube, nested octahedron. In other forms, target structures can be a combination of one or more of the same or different polyhedral forms, linked by a common contiguous edge. Staple strands can also include, function as, or be attached to bridging molecules.

In some forms, the target structure is a reinforced structure. Reinforced structures are structures that share the same polyhedral form as the equivalent, non-reinforced structure, and include one or more additional edges spanning between two vertices. Typically, the reinforced structure contains at least one or more edges than the corresponding non-reinforced structure. In some forms, additional structural elements that appear as “cross-bars” spanning between two vertices are introduced. Generally, edge lengths for a chosen geometry, satisfies the 10.5 bp/turn rule.

The size of arbitrary structured DNA assemblies ranges from about 5 nanometers to about 100 nanometers. In some form, the size of the DNA nucleic acid assemblies is about 10, 20, 30, 40, 50, 60, 70, 80, 90, 100, or more than 100 nanometers.

In some forms, pore size of any desired geometric shape is varied to accommodate large cargos. For example, the face in radius being 14 nm in the dodecahedron is large enough for the prokaryotic ribosome to diffuse inside.

a. Design and Assembly of Nucleic Acid Structures

Nucleic acid assemblies can be produced according to methods known for the design and assembly of nucleic acid assemblies. Generally, methods for preparation of nucleic acid assemblies include methods for design and methods for production of the assemblies.

Exemplary methods for design include templated building block-based design, and user-defined structure-based design. Therefore, in some forms, nucleic acid assemblies are produced by templated building block-based methods. In some forms, nucleic acid assemblies are designed according to user-defined structure-based methods. As described in greater detail below, the user-defined structure-based methods can provide design parameters for subsequent assembly of nucleic acid assemblies. For example, in some forms, user-defined structure-based methods provide the sequence of one or more nucleic acids that can be combined to form a nucleic acid assembly having a user-defined size, shape and three-dimensional structure. In exemplary forms, user-defined structure based design methods provide the sequence of a single-stranded nucleic acid “scaffold” sequence that routes throughout the user-defined structure, and the sequences of smaller nucleic acids that can be hybridized with the scaffold sequence to form a the user-defined three-dimensional structure.

When output is in the form of a single-stranded nucleic acid polymer that is a scaffold sequence that is routed several times throughout every edge of the assembly providing a double-stranded nucleic acid structure of the desired form without the need for staples, or as few staples as desired, by allowing self-hybridization.

“Scaffolded DNA origami” or “DNA origami” are nucleic acid assemblies that can include numerous short single strands of nucleic acids (staple strands) (e.g., DNA) to direct the folding of a long, single strand of polynucleotide (scaffold strand) into desired shapes on the order of about 10-nm to a micron or more, and the structures form therefrom. Exemplary scaffolded nucleic acid structures include three-dimensional solid figures, in which each side is a flat surface. The flat surfaces are typically polygons, and are joined at their edges. The scaffolded assemblies include “staple strand” or “helper strand” oligonucleotides that hold the scaffold DNA in its three-dimensional wireframe geometry. Additional nucleotides can be added to the staple strand at either 5′ end or 3′ end, and those are referred to as “staple overhangs.” Staple overhangs can be functionalized to have desired properties such as a specific sequence to hybridize to a target nucleic acid sequence, or a targeting element. In some instances, the staple overhang is biotinylated for capturing the DNA assembly on a streptavidin-coated bead. In some instances, the staple overhang can be also modified with chemical moieties. Non-limiting examples include Click-chemistry groups (e.g., azide group, alkyne group, DIBO/DBCO), amine groups, and Thiol groups. In some instances some bases located inside the oligonucleotide can be modified using base analogs (e.g., 2-Aminopurine, Locked nucleic acids, such as those modified with an extra bridge connecting the 2′ oxygen and 4′ carbon) to serve as linker to attach functional moieties (e.g., lipids, proteins). Alternatively DNA-binding proteins or guide RNAs can be used to attach secondary molecules to the DNA scaffold.

b. Templated Building Block-Based Nucleic Acid Assemblies

In some forms, the sequence and structure of the nucleic acid assembly is designed manually, or using nucleic acid “tile” based computational sequence design procedures (e.g., Templated Building Block-Based Design). Templated Building Block-Based Design methods typically include fabricating assemblies from combining pre-formed templates, or “building blocks,” for example, 2-dimensional nucleic acid structures. These methods typically include fabricating assemblies from combining pre-formed templates, or “building blocks,” for example, 2-dimensional nucleic acid structures. Exemplary design strategies that can be incorporated for making and using NMOs include single-stranded tile-based DNA origami (Ke Y, et al., Science 2012); brick-like DNA origami, for example, including a single-stranded scaffold with helper strands (Rothemund, et al., and Douglas, et al.); and purely single-stranded DNA that folds onto itself in PX-origami, for example, using paranemic crossovers.

Structured nucleic acid assemblies include bricks, bricks with holes or cavities, assembled using DNA duplexes packed on square or honeycomb lattices (Douglas et al., Nature 459, 414-418 (2009); Ke Y et al., Science 338: 1177 (2012)). Paranemic-crossover (PX)-origami in which the nucleic acid assembly is formed by folding a single long scaffold strand onto itself can alternatively be used, provided bait sequences are still included in a site-specific manner. Further diversity can be introduced such as using different edge types, including 6-, 8-, 10, or 12-helix bundle. Further topology such as ring structure is also useable for example a 6-helix bundle ring.

c. User-Defined Polyhedral Nucleic Acid Assemblies

Systems and methods for the automated, step-wise design of a nucleic acid assemblies having arbitrary geometries are known in the art (see, for example, WO 2017/189870, and Veneziano, et al., Science, V. 352, (6293), pp. 1534 (2016); and WO 2017/189914).

Structure-based design algorithms provide a “top-down” approach for developing a polyhedral nucleic acid structure of user-defined geometry, from only a target structure/shape as input. Nucleic acid structures produced by structure-based design algorithms typically include a long single-stranded nucleic acid sequence that is routed throughout the entire structure, and optionally includes one or more “staple” nucleic acid sequences that hybridize to the scaffold sequence in such a way as to determine a three-dimensional structure.

Systems and methods for design of nucleic acid structures of user-defined geometry involve rendering the geometric parameters of a desired polyhedral form as a node-edge network, and determining the nucleic acid scaffold route and staple design parameters necessary to form the desired polyhedral structure. Nucleic acid assemblies are designed by methods that typically generate the sequences of a long single-stranded nucleic acid scaffold and the nucleic acid sequence of staple strands that combine to form a nucleic acid assembly having the desired shape.

For example, nucleic acid assemblies are designed by methods that described in WO 2017/189870, and Veneziano, et al., Science, V. 352, (6293), pp. 1534 (2016) provide the nucleic acid sequences of a single-stranded scaffold, and the oligonucleotide staple sequences that can be combined to form complete three-dimensional nucleic acid assemblies of a desired form and size.

Typically, nucleic acid assemblies are designed by methods that that convert the information provided as geometric parameters corresponding to the desired form and the desired dimensions into the sequences of oligonucleotides that can be synthesized using any means for the synthesis of nucleic acids known in the art. These systems and methods are generally useful for predicting the design parameters that produce a nucleic acid assembly having a desired polyhedral shape.

The route of a single-stranded nucleic acid scaffold that traces throughout the entire target structure and can hybridize to itself is typically identified by a method including: (i) producing a node-edge network representing the three-dimensional structure; (ii) determining a spanning tree of the network corresponding to the three-dimensional structure, for example, where the vertices and lines of the structure are the nodes and edges of the network, respectively; (iii) classifying each edge as one of four types, based on its membership in the spanning tree and the crossover motif employed: if it is not a member of the spanning tree, each fragment of the scaffold exits the edge from the vertex it starts from, if it is a member of the spanning tree, each fragment of the scaffold exits the edge from the vertex it did not start from, and each edge can employ either anti-parallel or parallel crossover motifs; (iv) splitting the edges that are not members of the spanning tree into two edges, each containing a pseudo-node at the point of the scaffold crossover and each node at each of the vertices being split into two pseudo-nodes; and (v) determining the route of a single-stranded nucleic acid scaffold that traces throughout the entire target structure from the Eulerian cycle of the network by superimposing and connecting units of partial scaffold routing within an edge based on its classification and length.

This is fundamentally different from bottom-up design methods. For example, the “bottom-up approach” does not produce the sequences of staple strands, but requires manual intervention via an heuristic approach, using multiple duplex arms combined together to form the structure (i.e., may not use a single scaffold sequence throughout). The “top-down” methods start with the desired output, i.e. the final structure and the use of a specific scaffold, and generate the sequences required to synthesize that output, using a single ssDNA scaffold that is routed throughout the entire structure. The scaffold can be a user-defined scaffold sequence, and the staple sequences are varied accordingly.

The approach is extremely powerful because it can exploit the single scaffold strand to enable down-stream applications, such as DNA RAM storage (i.e., a single strand of DNA is folded into each object), as well as other applications. The formula uses a maximum-breadth spanning tree to determine positions of the scaffold crossovers for the scaffold routing. Any spanning tree, however, will lead to a valid scaffold routing. The nucleic acid assemblies themselves are distinct in having a continuous single stranded nucleic acid sequence routed through each edge of the structure.

Exemplary methods for designing a nucleic acid assembly having a desired polyhedral form include selecting a desired 3D polyhedral or 2D polygon form as a target structure; providing geometric parameters and physical dimensions of the a target structure for a selected 3D polyhedral or 2D polygon form; identifying the route of a single-stranded nucleic acid scaffold that traces throughout the entire target structure; and generating the sequences of the single-stranded nucleic acid scaffold and/or the nucleic acid sequence of staple strands that combine to form a nucleic acid assembly having the desired shape. DNA nucleic acid assemblies having the desired shape are produced by folding a long single stranded polynucleotide, referred to as a “scaffold strand,” into a desired shape or structure using a number of small “staple strands” as glue to hold the scaffold in place. Typically, the number of staple strands will depend upon the size of the scaffold strand, the complexity of the shape or structure, the types of crossover motifs employed, and the number of helices per edge. For example, for relatively short scaffold strands (e.g., about 150 to 1,500 base in length) and/or simple structures the number of staple strands are small (e.g., about 5, 10, 50 or more). For longer scaffold strands (e.g., greater than 1,500 bases) and/or more complex structures, the number of staple strands can be several hundreds to thousands (e.g., 50, 100, 300, 600, 1,000 or more helper strands). Using parallel crossover motifs, however, the number of staples can be reduced, even to zero. The choice of staple strands and, in some instances, the programmed self-hybridization of the scaffold strand, determine the pattern. In some forms, a software program is used to identify the staple strands needed to form a given design.

Typically, nucleic acid assemblies are designed by methods that include one or more of the following steps:

(a) Selecting a target polyhedral structure;

(b) Choosing the cross-section geometry of the edge of 2 helices, 4 helices on a square or honeycomb lattice, 6 helices on a square or honeycomb lattice, or any even number of helices on a square or honeycomb lattice.

(c) Determining the spatial coordinates of all vertices, the edge connectivity between vertices, and the faces to which vertices belong in the target structure;

(d) Identifying the route of a single-stranded nucleic acid scaffold sequence that traces throughout the entire target polyhedral structure, and

(e) Determining the nucleic acid sequence of the single-stranded nucleic acid scaffold and, optionally, the nucleic acid sequence of corresponding staple strands.

Typically, the route of the scaffold nucleic acid is identified by

(i) Determining edges that form the spanning tree of the node-edge network (for example, using the Prim's Formula);

(ii) Bisecting each edge that does not form the spanning tree to form two split edges;

(iii) Determining an Eulerian circuit that passes twice along each edge of the spanning tree. The direction of the continuous scaffold sequence is reversed at the bisecting point of the node-edge network in a DX-anti-parallel crossover, and the Eulerian circuit defines the route of a single-stranded nucleic acid scaffold sequence that passes throughout the entire structure. Staple strands are located at the vertices and edges of the route of the single-stranded nucleic acid scaffold sequence determined in (d).

Typically, for the origami nucleic acid assemblies that incorporate parallel crossovers, the route of the scaffold nucleic acid is identified by determining an Eulerian circuit that passes twice or more than twice along each edge of the wireframe. Based on the length and spanning tree classification, units of partial scaffold routing are superimposed and connected to complete the circuit.

In some forms nucleic acid assemblies are designed by methods that further include the steps by

(i) Detaching and scaling each edge of the initial geometry to represent the number of helixes as lines indicating their lengths and endpoints;

(ii) Generating the loop-crossover structure joining endpoints and finding double crossovers between two loops;

(iii) Generating the dual graph of the loop-crossover by converting each loop to a node and each double crossover to edge;

(iv) Computing the spanning tree of the dual graph of the loop-structure (for example, using the Prim's Formula);

(v) Inverting the dual graph back to the loop-crossover structure but without the double-crossovers corresponding to the non-member's spanning tree; Edges that are members of the spanning tree correspond to the subset of crossovers required to complete the Eulerian circuit.

In some forms nucleic acid assemblies are designed by methods that further include the step of

(f) Modelling the 3-Dimensional co-ordinates of each nucleic acid according to the parameters determined in (c) and (d).

In some forms, nucleic acid assemblies are designed by methods that further include the step of

(g) Assembling and optionally purifying the nucleic acid assemblies designed by nucleic acid assemblies are designed by methods that of any of the steps (a) through (d). Each of these steps is discussed in more detail, below.

The method described herein is a “top-down approach” of the structure (i.e., only input is a “shape” and the number and geometry of helices per edge). Nothing else is required, except for optional selection of a size and an input sequence (otherwise, default parameters can be used for both).

Default parameters for input scaffold size, nucleic acid type, input scaffold sequence, edge length, number of helices per edge, cross-sectional morphology of edges and vertex geometry (i.e., beveled or non-beveled edges) can be used as necessary to generate the sequences of staples and/or scaffold nucleic acid when no value is specified. For example, in some forms, the default nucleic acid is B-DNA, and the default edge-length is 31 bases, with 2 helices per edge. In some forms, the default nucleic acid scaffold sequence is the 7,249 nt M13pm18 bacteriophage DNA. In some forms, when the number of helices per edge is specified, but the vertex morphology is not specified, the default vertex geometry is to use honeycomb morphology with beveled edges.

In some forms, the assembly process includes mixing the nucleic acid scaffold, the core staples, and the functionalized staple strands, which are then annealed by slowly changing the temperature down (annealing) over the course of 1 to 48 hours. This process allows the staple strands to guide the folding of the scaffold into the final DNA nucleic acid assemblies.

In some forms, methods for user-defined structure-based design of nucleic acid assemblies produce structures having anti-parallel scaffold crossover motifs, for example, to provide a structure through hybridization with oligonucleotide staple strands.

In other forms, methods for user-defined structure-based design of nucleic acid assemblies produce structures having at least one edge having one “PX” (parallel paranemic scaffold crossover) motif. The differences in these methods, and the resulting structures, are described in greater detail, below.

(A) Nucleic Acid Assemblies with Antiparallel Double Crossover (DX) Motifs

In some forms, nucleic acid assemblies are polyhedral structures including at least one edge having one “DX” (anti-parallel scaffold crossover) motif. The edges with zero DX scaffold crossovers meet the definition of a spanning tree of a network. Therefore, a single DX anti-parallel scaffold crossover is positioned along every edge that does not form part of the spanning tree of the graph, preferably as close to the center of the edge as possible.

The scaffold strand is routed by a method that identifies the Eulerian circuit through the entire network, such that the strand enters each vertex from a first edge and exits the vertex from an adjacent edge that shares a face with the first edge. The route of the scaffold strand is determined according to the rules that the scaffold strand does not enter and exit from the same edge, and the scaffold strand does not exit from an edge that is not-adjacent to the edge it enters. Therefore, the scaffold routing process does not allow for the intersection of DNA strands and the process produces only edges that are connected to the vertex.

Nucleic acid assemblies are designed by methods that include using the spanning tree to identify the route of the scaffold sequences through the target structure. For example, nucleic acid assemblies are designed by methods that that identify the location of anti-parallel DX cross-overs within the target structure by classifying each edge.

Determination of a spanning tree including all nodes of the network enables the identification of edges that are within the spanning tree and edges that are not within the spanning tree. Therefore, nucleic acid assemblies are designed by methods that that include identifying edges that are within the spanning tree and edges that are not within the spanning tree. Edges within a spanning tree represent continuous stretches for the route of the single-stranded nucleic acid scaffold in both directions (i.e., 5′-3′ and 3′-5′). Edges not within a spanning tree include anti-parallel DX cross-over motifs. Therefore, for each edge that is not in the spanning tree, a pair of pseudo-nodes is added to split the edge into two halves, each corresponding to one side of a scaffold crossover. At each anti-parallel DX cross-over motif, the single-stranded nucleic acid scaffold reverses the direction it travels along.

Nucleic acid assemblies are designed by methods that include assigning anti-parallel DX cross-over motifs at the center of each edge that is not within a spanning tree. Because a single scaffold crossover is assigned to each edge that is not within a spanning tree, and edges with zero scaffold crossovers must connect to every vertex, there can be no cycles of edges with zero scaffold crossovers, meaning that there are V−1 edges with zero scaffold crossovers, where V is the number of vertices, and the rest have one scaffold crossover.

Locating the DX crossovers within each possible spanning tree corresponds to a unique scaffold routing.

Nucleic acid assemblies are designed by methods that include the identification of the nucleic acid sequences of staples corresponding to the sequence of the single-stranded nucleic acid scaffold.

The length of the scaffold sequence is determined from the Eulerian circuit calculated from the input geometry, modified according to the input size, for example, as determined by the user-defined size of one or more of the edges of the structure. Typically, the sequence of the scaffold is based on a template sequence, for example, a user-defined sequence, or a known sequence, such as a bacteriophage sequence (e.g., M13mp18). If the sequence length required to provide the desired structure according to nucleic acid assemblies designed by top-down methods is smaller than that of the default sequence, a subset of the default sequence will be output. Alternatively, if the sequence length required to provide the desired structure according to nucleic acid assemblies designed by top-down methods is larger than that of the default sequence, a sequence will be generated.

Nucleic acid assemblies are designed by methods that include the placement of all staple sequences. After all the staples are placed, each staple is converted to a vector of numbers, each value corresponding to the scaffold nucleotide to which it is base paired. Then, the input or generated scaffold sequence is used, matching a base identity (A, T, G, or C) to a scaffold number. If no sequence is provided, a segment of M13pm18 is used by default if the required scaffold length is less than 7249 nucleotides, and a sequence is randomly generated if the required length is greater. The complementary nucleotide via Watson-Crick base pairing is then be computed and assigned to the corresponding staple nucleotides. Finally, this list of staple sequences is output for synthesis.

Nucleic acid assemblies are designed by methods that identify the routing of the staple strands based on the spatial location, including the edge, the duplex, and the position from the 5′ end. For example, information contained within the set of numbers that indicate the spatial location, including the edge, the duplex, and the position from the 5′ end, is used to identify which bases in the staples are paired with which bases in the scaffold, then the former index number is assigned to the staples accordingly.

Typically, the number of staple strands varies depending upon the complexity of the structure. For structures with small scaffold strands that are of minimal complexity, such as simple tetrahedra, cubes, etc., the number of staple strands is typically about 5, 10, 50 or more than 50. For longer scaffold strands (e.g., greater than 1500 bases) and/or more complex structures, the number of staple strands can be several hundreds to thousands. For example, in some forms, the number of staple strands is up to 50, 100, 300, 600, 1,000 or more than 1,000.

There are three categories of staple strands, each with their own prescribed pattern: staples on vertices, staples on edges with scaffold crossovers, and staples on edges without scaffold crossovers.

Nucleic acid assemblies are designed by top-down methods include a minimum edge length of 31 bp. A 31/32-bp edge has 21 bp occupied by vertex staples, leaving 10 or 11 bp for edge staples. Therefore, in both types of edges, a 20- or 22-bp staple is placed with a single crossover on one side, because a staple nick in the middle would conflict with the scaffold crossover. Therefore, nucleic acid assemblies designed by top-down methods include a double-crossover vertex staple design in any structure with a 31- or 32-bp edge present.

The pattern of staple routing depends on the degree of the vertex, ensuring that each staple length is 52- or 78-nucleotides (nt) long for ease of synthesis.

$$\begin{matrix}a = \{ \begin{matrix}0 , & if n mod 3 = 0 \\ 2 , & if n mod 3 = 1 \\ 1 , & if n mod 3 = 2\end{matrix} & ( Eq . 1 ) \\ b = \frac{n - 2 a}{3} & ( Eq . 2 )\end{matrix}$$

### Where

a is the number of 52-nt staples at the vertex,

b is the number of 78-nt staples at the vertex, and

n is the degree of the vertex.

(1) Staples on Vertices

The staples on vertices pair with the first 10-11 nucleotides of each duplex abutting the vertex, with poly-T bulges of length 5 crossing between edges. There are two varieties of vertex staple designs implemented: one system uses single crossovers in some places to ensure that there is 10-11 bp of continuous duplex for high specificity and binding strength, and the other, more traditional, system uses double crossovers everywhere, leading to a minimum of 5 bp of continuous duplex. For the structures synthesized and characterized in this work, the former paradigm is used, as the higher binding strength was found to create a more cooperative transition at a higher temperature (FIGS. 9A-9L). The pattern of staple routing depends on the degree of the vertex, ensuring that each staple length is 52- or 78-nucleotides (nt) long for ease of synthesis.

(2) Staples on Edges with Scaffold Crossovers

The edge staples pair with the intermediate nucleotides between vertex staples. For the edges with scaffold crossovers, two 31-32-nt staples are placed across the scaffold crossover, together occupying a 15-16-nt region on either side of the crossover for sufficiently strong binding. The remainder of scaffold has 42-nt staples placed to create staple crossovers every 21 base pairs, with a 20- or 22-nt staple in the case of a 10- or 11-nt remainder.

(3) Staples on Edges without Scaffold Crossovers

The edges without scaffold crossovers follow the same pattern, filling with as many 42-nt staples that can fit and using a 20- or 22-nt staple when necessary.

Nucleic acid assemblies are designed by methods that provide the nucleic acid sequences of staple strands corresponding to the desired target sequence, edge size(s) and optionally a template nucleic acid sequence.

After all the staples are placed according to top-down design methods, each staple is a vector of numbers, each value corresponding to the scaffold nucleotide to which it is base paired. Then, the input or generated scaffold sequence is used, matching a base identity (A, T, G, or C) to a scaffold number.

If no sequence is provided, a default sequence is used. For example, in some forms, if the required scaffold length is less than 7249 nucleotides, a segment of M13pm18 nucleic acid sequence is used. In other forms, a sequence is randomly generated. Nucleic acid assemblies are designed by methods that determine complementary nucleotides via Watson-Crick base pairing and assign sequences to the corresponding staple nucleotides. Typically, nucleic acid assemblies are designed by methods that produce this list of staple sequences as output. Therefore, in some forms, nucleic acid assemblies are designed by methods that also include the step of synthesizing the staple sequences. In some forms, nucleic acid assemblies are designed by methods that include the step of synthesizing the scaffold sequence. In some forms, nucleic acid assemblies are designed by methods that include the step of synthesizing the scaffold sequence and the staple sequences. Therefore, nucleic acid assemblies are designed by methods that include converting the undirected graph into a directed graph to implement this directional choice.

In some forms, methods to generate staple strand sequences given a scaffold sequence can be inverted, so that the user provides staple strand sequences that are used to generate a scaffold sequence.

Methods for custom-design of an assembly having desired geometric parameters can also be used to determine the nucleic acid sequence of a scaffold sequence that will fold into the desired shape based on hybridization with one or more user-defined staple sequences. Therefore, in some forms, nucleic acid assemblies are designed by methods that provide the nucleic acid scaffold sequence, based on the input of user-defined staple strands, desired target structure and optionally edge size(s).

User-defined design methods provide a custom scaffold sequence that based on user-defined staple sequences. Typically, the number and size of scaffold sequences that are required by the user will vary according to the desired geometry of the assembly. In some forms, at least one, two or three staple sequences are required as input. In some forms, one or more staple sequences are required as input, and nucleic acid assemblies are designed by methods that provide the sequence(s) of one or more remaining, or undefined staple sequences.

Nucleic acid assemblies having polyhedral morphology designed and produced according to the described top-down design methods are described. The polyhedral nucleic acid assemblies include a single stranded nucleic acid scaffold routed through the entire polyhedral structure. Typically, the number of staple strands varies depending upon the complexity of the structure. For structures with small scaffold strands that are of minimal complexity, such as simple tetrahedra, cubes, etc., the number of staple strands is typically about 5, 10, 50 or more than 50. For longer scaffold strands (e.g., greater than 1500 bases) and/or more complex structures, the number of staple strands can be several hundreds to thousands. For example, in some forms, the number of staple strands is up to 50, 100, 300, 600, 1,000 or more than 1,000.

There are three categories of staple strands, each with their own prescribed pattern: staples on vertices, staples on edges with scaffold crossovers, and staples on edges without scaffold crossovers.

The nucleic acid assemblies can be of any desired shape that can be rendered as a three-dimensional wire-frame mesh with sharp angles and non-curved edges. The nucleic acid assemblies include a single-stranded nucleic acid scaffold that is routed throughout the entire structure. The route of the single-stranded nucleic acid scaffold throughout every face of the structure is the Eulerian circuit through the node-edge network of the planar graph of the structure. Preferably, the Eulerian circuit that defines the path of the single-stranded scaffold sequence throughout the entire structure is the A-trail Eulerian circuit.

In some forms, the nucleic acid assemblies include at least one edge having a DX crossover motif located within the center of the edge. In other forms, the nucleic acid assemblies include at least one edge having a PX crossover motif located within the center of the edge. Typically, the nucleic acid assemblies include zero or one scaffold crossover structures per edge. The placement of DX scaffold cross-overs is defined using by the maximum-breadth spanning-tree of the node-edge network of the planar graph of the structure. Edges that form part of the maximum-breadth spanning tree are the only edges that do not include a DX scaffold crossover. Edges that form part of the maximum-breadth spanning tree are the only edges that include a single DX scaffold crossover.

Nucleic acid assemblies produced according to nucleic acid assemblies are designed by methods that include two nucleic acid anti-parallel helices along each edge to strengthen the rigidity of the structure.

The nucleic acid assemblies are typically less than 1 micron in diameter, for example, 10 nm-1,000 nm, inclusive. In some forms, the nucleic acid assemblies have overall dimensions of 50-500 nm, 60-200 nm, or 60-100 nm, for example, 60 nm, 70 nm, 80 nm, 90 nm, 100 nm or leger than 100 nm. The molecular weight of the nucleic acid assembly is typically defined by the size and complexity of the polyhedral shape of the nucleic acid assembly. Typically, the nucleic acid assemblies have a molecular weight of between 200 kilo daltons (kDa) and 1 mega dalton (1 mDa). The volume encapsulated by the nucleic acid assemblies is defined by the size and shape of the nucleic acid assemblies, and can be determined from the dimensions.

Typically the nucleic acid assemblies are stable in physiological concentrations of salt, for example, in PBS, and DMEM.

(B) User-Defined Nucleic Acid Assemblies with Parallel Crossover Motifs

In some forms, nucleic acid assemblies include at least one edge having one “PX” (parallel paranemic scaffold crossover) motif. Therefore, in some forms, there are two double helices per edge oriented in parallel vertically, that is, one of the duplexes is closer to the interior of the object than the other. In some forms, the scaffold cannot be an arbitrary sequence, because self-hybridization must occur to complete the structure. Self-hybridizing regions replace the need for staple strands, so in some forms, one nucleic acid strand can fold and hybridize to itself to form an origami assembly without any other oligonucleotides.

The scaffold strand is routed by a method that identifies the Eulerian circuit through the entire network, such that the strand enters each vertex from a first edge and exits the vertex from an adjacent edge that shares a face with the first edge. The route of the scaffold strand is determined according to the rules that the scaffold strand does not enter and exit from the same edge, and the scaffold strand does not exit from an edge that is not-adjacent to the edge it enters. Therefore, the scaffold routing process does not allow for the intersection of DNA strands and the process produces only edges that are connected to the vertex.

In some forms, the wire-frame model of a desired polyhedral structure is rendered as a node-edge network. Typically, the nodes and edges of the network correspond to the vertices and lines of the polyhedron. In some forms, a node-edge network corresponding to a structure can be represented by the planar graph of the corresponding polyhedron, or by other means. For example, in some forms, the planar graph of the corresponding polyhedron is a Schlegel diagram. The Schlegel diagram is a projection of the desired polyhedral form from R^{d} into R^{d-1} through a point beyond one of its facets or faces. The resulting entity is a polytopal subdivision of the facet in R^{d-1} that is combinatorially equivalent to the original polyhedral form. Formulas and methods for generating a Schlegel diagram of a polyhedral form are known in the art. In other forms, a node-edge network is calculated for a corresponding structure without the use of a planar graph.

Therefore, in some forms, nucleic acid assemblies are designed by methods that that include the step of providing a node-edge network of the target structure. Typically, each of the vertices corresponds to a node in the network, and each line between any two vertices represents an edge in the network.

In some forms, the node-edge network is used to establish connectivity amongst all of the vertices. Exemplary representations of connectivity through the node-edge network include by producing one or more spanning trees. The spanning tree is the set of edges that connect all nodes within the network without circuits. In some forms, the spanning tree is determined using one or more formulas. Formulas for determining the spanning tree for a network are known in the art. Exemplary methods for determining the spanning tree for the node-edge network corresponding to the desired shape include Prim's Formula. Therefore, in some forms, identifying scaffold routing includes creating one or more spanning trees for the node-edge network. In some forms, the spanning tree is the spanning tree produced using a maximum-breadth search. If, as in this case, all edges are weighted the same, Prim's formula will generate a breadth-first search spanning tree, one with the most branches. Therefore, in some forms, identifying scaffold routing includes the selection of one or more spanning trees that have the most branches.

It has been shown that branching trees self-assemble more reliably than more linear trees, however, any spanning tree will provide a valid route.

Nucleic acid assemblies are designed by methods that include using the spanning tree to classify the edges, culminating in the final Eulerian circuit the scaffold strand takes through the target structure.

There are four classifications the edges can have, based on choosing between two options for two traits. One trait is the crossover motif of the edge. Each edge can employ either anti-parallel (DX) or parallel (PX) crossovers. The second trait is determined by membership in the spanning tree. Edges that are members of the spanning tree must have each scaffold fragment, that is, the portion of the scaffold strand within the edge, start and end at different vertices. Edges that are not members of the spanning tree must have each scaffold fragment start and end at the same vertices. Note that this is an extension of the classification used for the two-helix-per-edge DX structures; the classifications and choice of scaffold crossover location follow the same start and end rules as described above.

Based on the classification (crossover motif, spanning tree membership) and the length of the edge, a set of scaffold fragments, and in some forms, staple strands, with routing within the edge already determined, is superimposed on the edge. In some forms, this is represented by an M×4 matrix, where M is the length of the edge, and each of the four columns represents one strand, e.g. Column 1 represents the nucleotides 3′ to 5′ from the vertex at the top to the vertex at the bottom in the duplex closer to the interior of the object, Column 2 represents the nucleotides 5′ to 3′ from the vertex at the top to the vertex at the bottom in the interior duplex, Column 3 represents the nucleotides 5′ to 3′ from the top vertex to the bottom vertex in the duplex closer to the exterior of the object for PX edges and 3′ to 5′ for DX edges, and Column 4 represents the nucleotides 3′ to 5′ from the top vertex to the bottom vertex in the exterior duplex for PX edges and 5′ to 3′ for DX edges. Nucleotides in Columns 1 and 2 are complementary via Watson-Crick base pairing, and nucleotides in Columns 3 and 4 are complementary in the same manner Nucleotides in the same row are the same interpolated distance between the two vertices.

In some forms, the elements of the matrix determine the route of the scaffold and enforce the crossover motif; for PX edges, the major/minor groove pattern is also enforced. Elements that are consecutive in number, e.g., 4 and 5, or i and i+1, represent nucleotides that share a covalent phosphodiester bond, and elements that are in the same row and are in paired columns (1 and 2, 3 and 4) are base paired. For PX edges, the major/minor groove pattern is the number of bases that lie in the major and minor grooves of the double helix. In some forms, the number of bases in a major groove can be less than 5, 5, 6, 7, 8, 9, or more than 9, and the number of bases in a minor groove can be less than 4, 4, 5, 6, or more than 6. The major/minor groove pattern also determines where parallel crossovers can occur. In some forms, this is reflected in the matrix as when consecutive nucleotides are not in the same column, e.g. nucleotide 4 is in Column 1 and nucleotide 5 is in Column 4.

When all of the edges have been superimposed, the first and last rows of Columns 1 and 2 of each edge matrix represent the 5′ and 3′ ends that must be joined to neighboring edges at the vertex. The connection is enforced by updating each nucleotide's number to uniquely identify its position in the complete scaffold strand, maintaining that consecutive numbers indicate connection along the phosphodiester backbone.

Nucleic acid assemblies are designed by methods that include the identification of the nucleic acid sequences of scaffold and staples corresponding to the hybridization pattern set by the routing described above.

In regions of parallel crossovers, the sequence must be customized such that Watson-Crick base pairing is followed. In regions of anti-parallel crossovers, the scaffold sequence can be arbitrary, and the staple sequences that hybridize to it must follow Watson-Crick base pairing.

In some forms, the scaffold nick is chosen to be placed at the end of a farther-from-center duplex. This may be on PX or DX edge. The 5′ end of the nick is marked as base #1, and the 3′ end is the last base of the scaffold. Some scaffold nucleotides may be part of hairpin loops and do not have bases paired to them; the numbering of the scaffold strand remains unchanged, but these regions may be marked as single-stranded nucleic acid strands.

For these custom sequences, in some forms, a random number generator choosing between 1 and 4 inclusive, which can map to A, C, G, T for DNA and A, C, G, U for RNA can produce the sequences of one member of each base pair, and its partner's sequence is found via canonical Watson-Crick base pairing. If certain staple sequences are to be incorporated, perhaps for example if they have been functionalized and need to bind to the larger origami structure, then those sequences of those regions are determined from the target staple sequences.

With this, nucleic acid assemblies are designed by methods that ascribe (1) an index number to indicate its position on the scaffold strand; and (2) a set of numbers to indicate its spatial location, including the edge, the duplex, and the position from the 5′ end.

In edges with anti-parallel crossovers, staples may be necessary to bring together the portions of scaffold within the edge. In some forms, the superimposed edges contain regions where the staples lie based on their numbers being non-consecutive with the rest of the bases in the edges. In this form, vertex staples are not required because only one duplex from each edge meets at the vertex.

Nucleic acid assemblies are designed by methods that provide the nucleic acid sequences of scaffold and staple strands corresponding to the desired target edge size(s) and geometry. Unlike the forms that only contains DX motifs, the scaffold sequence is, in part or in whole, a custom sequence.

In some forms, the nucleic acid assemblies produced by user-defined structure-based design methods include at least one edge having one “PX” (parallel paranemic scaffold crossover) motif. Therefore, in some forms, nucleic acid assemblies produced by user-defined structure-based design methods include two double helices per edge oriented in parallel vertically, that is, one of the duplexes is closer to the interior of the object than the other. In some forms, the scaffold cannot be an arbitrary sequence, because self-hybridization must occur to complete the structure. Self-hybridizing regions replace the need for staple strands, so in some forms one nucleic acid strand can fold and hybridize to itself to form an origami nucleic acid assembly without any other oligonucleotides.

Compositions of polyhedral nucleic acid assemblies designed according to user-defined structure-based methods are provided. In some forms, the polyhedral nucleic acid assemblies include two nucleic acid anti-parallel helices spanning each edge of the structure. In other forms, the polyhedral nucleic acid assemblies include 4, 6, 8, or more than 8 anti-parallel helices spanning each edge of the structure. The three-dimensional structure is formed from single stranded nucleic acid staple sequences hybridized to a single stranded nucleic acid scaffold sequence. The scaffold sequence is routed through a Eulerian cycle of the network defined by the vertices and lines of the polyhedral structure. The locations of double-stranded crossovers are determined by the spanning tree of the polyhedral structure. The staple sequences are hybridized to the vertices, edges and double-stranded crossovers of the scaffold sequence to define the shape of the assembly. In some forms, the polyhedral nucleic acid assemblies include 2 or more than 2 parallel helices spanning each edge of the structure. The three-dimensional structure is formed from single stranded nucleic acid sequences hybridized to itself. The scaffold sequence is routed through the Eulerian cycle of the network defined by the vertices and edges of the polyhedral structure. In other forms, the polyhedral nucleic acid assemblies include a combination of 2 or more than 2 parallel or anti-parallel helices spanning each edge of the structure. In some forms, the polyhedral nucleic acid assembly further includes one or more of a therapeutic, diagnostic or prophylactic agent, or combinations. For example, in some forms, the assemblies encapsulate one or more therapeutic, diagnostic or prophylactic agent. In other forms, secondary molecules are either covalently or non-covalently attached to the DNA structural scaffold or oligonucleotides with resulting full control over their 3D organization. In exemplary forms, messenger RNA (mRNA) encoding a protein is non-covalently attached to the DNA assembly using single-stranded DNA extensions from the oligonucleotides and complementary to the mRNA.

In some forms, the nucleic acid assembly comprises a single stranded nucleic acid scaffold sequence hybridized to itself, to single stranded nucleic acid staple sequences, or both, wherein the scaffold sequence is routed through the Euler cycle of the network defined by vertices and lines of a node-edge network of the nucleic acid assembly. In some forms, the scaffold sequence hybridizes to itself in at least one edge using parallel crossovers, the nucleic acid assembly comprises at least one edge including a double-strand crossover, or both. In some forms, the location of the double strand crossover, if present, is determined by a spanning tree of the dual graph of the network of the polyhedral or polygonal structure. In some forms, the nucleic acid assembly comprises two or more nucleic acid anti-parallel helices spanning each edge of the structure. In some forms, the nucleic acid staple sequences, if any, are hybridized to the edges and double strand crossovers of the scaffold sequence to define the shape of the nucleic acid assembly.

In some forms, the single stranded nucleic acid scaffold sequence is hybridized to single stranded nucleic acid staple sequences. In some forms, the nucleic acid assembly comprises four or more nucleic acid anti-parallel helices spanning each edge of the structure. In some forms, the nucleic acid assembly comprises at least one edge including a double-strand crossover. In some forms, the helices comprising an edge are arranged as a square lattice of four or more helices, or honeycomb lattice of six or more helices. In some forms, the helices meeting at a vertex can be beveled or non-beveled.

ii. Production of Nucleic Acid Assemblies

Based on the nucleotide sequences generated in the previous steps, nucleic acid assemblies are designed by methods that typically produce this list of staple sequences and scaffold sequence as output. Therefore, in some forms, nucleic acid assemblies are designed by methods that also include the step of synthesizing the staple sequences. In some forms, nucleic acid assemblies are designed by methods that include the step of synthesizing the scaffold sequence. In some forms, nucleic acid assemblies are designed by methods that include the step of synthesizing the scaffold sequence and the staple sequences.

Typically, following design according to the described methods, the nucleic acid assemblies are synthesized, folded and purified prior to structural validation. Therefore, methods for the design of nucleic acid assemblies having a desired form optionally include the step of producing the nucleic acid assembly. In some forms, producing the assembly includes synthesizing nucleic acids having the sequence of the scaffold and staples according to the designed form; hybridizing the staple sequences to the scaffold; folding the assembly; purifying the assembly; performing structural analysis of the assembly; validating the structure; and combinations.

Nucleic acid sequences of the single-stranded scaffold and the oligonucleotide staple sequences are combined via hybridization to form complete three-dimensional nucleic acid assemblies of a desired form and size. Typically, nucleic acid assemblies are designed by methods that convert the information provided as geometric parameters corresponding to the desired form and the desired dimensions into the sequences of oligonucleotides that can be synthesized using any means for the synthesis of nucleic acids known in the art.

Scaffold nucleic acid sequences and oligonucleotide staple sequences can be synthesized or purchased from numerous commercial sources. In some forms, the scaffold nucleic acid sequence is the M13mp18 single-stranded DNA scaffold. The M13mp18 ss DNA can be purchased from multiple commercial sources, including New England Biolabs (Cat #N4040S) or from Guild Biosciences for various M13mp18 size.

Typically, scaffold DNA of the desired length is produced using polymerase chain reaction (PCR) methodologies. Standard methods for PCR are known in the art. In some forms, the nucleic acid assemblies are produced using asymmetric PCR (aPCR). When aPCR amplification is used, oligonucleotide primers can be designed to generate many different scaffold lengths. Therefore, in some forms, the scaffold having a desired length is produced using one or more custom oligonucleotides. When the template scaffold nucleic acid is known, a set of known oligonucleotides can be used. In some forms, modified dNTPs (examples of modified dNTPs include, but are not limited to dUTP, Cy5-dNTP, biotin-dNTPs, alpha-phosphate-dNTPs) are used for amplification of the ssDNA scaffold. In other forms, the template use is the Lambda phage that can be purchased from different commercial sources, including New England Biolabs (Cat #N3011S). In other forms, the nucleic acid assemblies are produced using digestion of the template DNA to form a scaffold nucleic acid of the desired length. In some forms, a combination of PCR and digestion methods is used to produce scaffold single-stranded nucleic acid of the desired length. In some preferred forms, nucleic acids for the nucleic acid assemblies and other components can be produced via in vivo production of highly-pure single-stranded DNAs isolated within bacteriophage. Such bacteriophage particles can be produced by engineered bacteria containing both a phagemid and a helper plasmid.

When nucleic acid scaffold sequences are required to be synthesized, the scaffolds can be synthesized using the asymmetric PCR, for example, using GBLOCK® DNA commercially available from Integrated DNA Technologies as a template.

Single-stranded nucleic acid scaffold and corresponding staple sequences are assembled into nucleic acid assemblies of the desired shape and size.

In some forms, asymmetric polymerase chain reaction (aPCR) is used to synthesize long single-stranded DNA used as a scaffold.

Typically, an aPCR reaction is composed of two primers flanking the region of interest to be amplified, a template DNA to replicate from, buffers, nucleotides, and polymerase enzymes, where one of the primers is in excess over the other. In some forms, one primer is in 50- or 65-fold molar excess over the second primer. In some forms, the length of the scaffold is 500 nucleotides in length; 1000 nucleotides in length; 1500 nucleotides in length; 2000 nucleotides in length; 2500 nucleotides in length; 3281 nucleotides in length; 10,000 nucleotides in length; 12,000 nucleotides in length; or greater than 12,000 nucleotides. Typically, Taq-based polymerases or commercial blend of enzymes [LONGAMP®] are used as the enzyme in aPCR. In some forms, the nucleic acid polymer can be modified by introduction of modified nucleotides into the solution, including fluorescent nucleotides, radio-labeled nucleotides, alternative bases, and modified backbone. In some exemplary forms, alternative nucleotides are used in the DNA polymer synthesis with nucleotides modified with Cy5 fluorophore-modified nucleotides, phosphorothioate-modified nucleotides, and deoxyuridines. In some exemplary forms, modified primers including additional 5′ sequences to add to the amplicons are used to increase or modify the ssDNA final product or to hybridize to other ssDNA produced by standard synthesis or through aPCR. In some exemplary forms, the primers can be phosphorylated for ligation.

Pure single-stranded DNA (ssDNA) can be produced directly from bacteria using an engineered M13 phage that is produced from a plasmid that only encodes double-stranded DNA (dsDNA) and a phagemid plasmid that only contains a single f1 origin of replication and the packaging signal. The phage particles are produced containing only the ssDNA that has the f1 origin. The phagemid DNA can additionally contain an insert of DNA of any user-defined size and sequence that will be produced with the f1 origin as pure ssDNA and released as phage particles into the media.

In vivo production of highly-pure single-stranded DNAs isolated within bacteriophage generally uses engineered bacteria containing both a phagemid and a helper plasmid to produce bacteriophage particles loaded with DNAs. In some forms, the helper plasmid does not include the f1 origin of replication, and is under the control of a selection factor. An exemplary selection factor is exposure to chloramphenicol. This plasmid is still under the control of the p15A origin of replication for medium copy number (˜10 copies per cell) and produces all 10 M13 phage proteins, but does not get packaged into the phage particle because the packaging signal does not reside within the helper plasmid sequence and the helper plasmid sequence is not single-stranded. Thus, the particles that are produced contain genetically pure ssDNA. To achieve gram-scale quantity of ssDNA composed of an arbitrary sequence that is also genetically pure and produced by bacteria, the helper system M13cp can be used to clone and produce phage particles that meet these specifications.

Synthesis of single-stranded DNA (ssDNA) can be scaled up by using a helper-strain Escherichia coli (E. coli) system to produce highly pure ssDNA exported from the E. coli without the need for additional biochemical purifications away from the contaminating dsDNA and other non-target ssDNA. The target ssDNA can be composed of custom sequence and size ranging from 427 nt to 10,000 nt or longer than 10,000 nt, and any number of nucleotides between these sizes (e.g. 428, 429, 9,998, 9,999, etc.). The target ssDNA need only contain the f1 origin of replication and the packaging sequence. To achieve this goal, a variation of the M13cp helper strain E. coli transformed with phagemids containing only the 427 nt f1 origin of replication and either biological or purely synthetic sequences can be used, for example. Because these phagemids do not contain any origins besides the f1 origin, they are only capable of being replicated within the helper plasmid-transformed E. coli, and are packaged within the produced phage particles. By combining centrifugation or filtration with DNA extraction techniques, this strategy enables complete purification of ssDNA without the requirement of additional purification steps to remove contaminating DNA. This approach can be used, for example, to produce purified ssDNA for folding scaffolded DNA origami assemblies, synthetic DNA encoding paranemic crossover origami, and binding sites for CRISPR proteins, single guide or CRISPR RNAs, or siRNAs for packaging of pure biomolecules. Typically the ssDNA produced by these methods is isolated from dsDNA and/or other sources of ssDNA. For example, the isolated ssDNA can be present in a bacteriophage that includes no dsDNA, or includes only a small amount of dsDNA. For example, the amount of dsDNA present in the bacteriophage can be less than 10% by weight of the total, less ten a 5% by weight, less than 4%, 3%, 2%, 1%, or less than 0.1% by weight of the total DNA within the bacteriophage. Typically, the ssDNA of user-defined size and sequence is of sufficient purity to facilitate folding into a DNA origami assembly without the need for further purification. Generally, any dsDNA or other contaminants are not present in sufficient quantity to prevent or disrupt the hybridization or folding of the ssDNA into a DNA origami assembly.

The method of producing long single-stranded nucleic acids in vivo in bacteriophage can produce sequences of between 1 and 1,000,000 nucleotides in length. In some forms, the methods include one or more of the steps of producing a long single-stranded nucleic acid sequence that is a scaffold for a nucleic acid assembly formation within a microorganism; packaging the long single-stranded nucleic acid scaffold sequence within a bacteriophage particle within the microorganism; and isolating the long single-stranded nucleic acid scaffold sequence from the bacteriophage particle. Isolating the long single-stranded nucleic acid sequence from the bacteriophage particle can include harvesting phage particles directly from clarified growth media. Harvesting phage from the media can typically include buffer-exchanging the clarified growth media; and concentrating the phage particles. Typically, the methods do not require removal of double-stranded DNA from the bacterially-produced single-stranded nucleic acid scaffold prior to folding into a nucleic acid assembly.

Typically, the method includes one or more of the following steps:

(A) Providing a target nucleic acid scaffold sequence in the form of a long single stranded nucleic acid;

(B) Forming a phagemid molecule including the long single stranded target nucleic acid sequence;

(C) Packaging the phagemid into a bacteriophage particle in the absence of contaminating nucleic acid, such as double-stranded DNA;

(D) Isolating the bacteriophage;

(E) Optionally isolating the long single stranded target nucleic acid sequence from the bacteriophage; and

(F) Creating a nucleic acid assembly including the target sequence.

The method can include assembling nucleic acid “target” sequences into a phagemid. Typically, the phagemid includes the f1 origin of replication, the target scaffold nucleic acid sequence, and optionally one or more selection markers. Phagemids can be produced using any techniques known in the art. In some forms, the phagemid can be produced using asymmetric Polymerase chain reaction (aPCR).

In some forms, single stranded DNA can be generated by first amplifying the synthetic f1 sequence with, for example, Phusion™ polymerase, followed by gel purification and silica column cleanup. Asymmetric polymerase chain reaction can subsequently applied using, for example, 200 ng of purified dsDNA and 1 μM of the 5′-phosphorylated 3′ reverse primer with QuantaBio Accustart HiFi polymerase.

In some forms, the beta-lactamase (bla) ampicillin resistance gene (ApR) and its promoter and terminator sequences can be amplified from the widely available pUC19 plasmid using Phusion™ polymerase using a 3′ reverse primer and a 5′ primer that is additionally extended on the 5′ side by the reverse complement of the reverse primer of the f1 fragment. The amplicon can then be gel- and column-purified.

In some forms, asymmetric PCR can be used to amplify single-stranded DNA using, for example, 200 ng of purified amplicon as a template with QuantaBio AccuStart HiFi buffer and enzyme and 1 μM 5′-phosphorylated reverse primer. The two single-stranded DNA products can then mixed in a 1:1 molar ratio and the ssDNA was converted to dsDNA using, for example, Phusion polymerase, followed by amplification using the flanking forward and reverse phosphorylated primers, and subsequently purified. Blunt-end ligation using, for example, T4 DNA ligase (NEB) in 1× T4 DNA ligation buffer with 30 ng of amplified DNA incubated at room temperature overnight can then be used to close the plasmid.

A suitable E. coli strain, such as DH5aF, can be made competent by washing log-phase grown cells in ice cold 100 mM CaCl₂. Competent cells can be transformed with, for example, 1 ng of helper plasmid DNA and 2 μL of plasmid DNA ligation mix were added to 20 μL of cells. Cells can then be incubated on ice for 30 minutes, heat shocked at 42° C. for 45 seconds, put back on ice before adding pre-warmed SOB media and shaking at 37° C. for 1 hour. 100 μL can be plated evenly across a Luria Agar (LA) media plate made with, for example, 100 μg/mL ampicillin and 15 μg/mL chloramphenicol.

Single, individual colonies can be selected and grown in 5 mL of Terrific Broth (TB) supplemented with 1% glycerol for 36 hours at 37° C. 1 mL of the growth can then removed to a 1.5 mL spin column and spun in a centrifuge at 4,000 rpm for 5 minutes. Supernatant can be removed and placed in a new 1.5 mL spin column and spun at 4,000 rpm for an additional 10 minutes. 1 μL of the supernatant can be added to 20 μL of nuclease-free water and heated to 95° C. for 5 minutes. 1 μL of the heated solution can be added to a Phusion PCR mix containing enzyme, buffer, nucleotides, and forward and reverse primers used to generate the plasmid. Positive colonies can be determined by the presence of the amplicon from the media as determined by agarose gel. Positive colonies were sent for Sanger sequencing.

Synthetic phage (sPhage) producing colonies (as judged by, for example, positive PCR, gel visualization, and sequencing) can be grown in 5 mL TB supplemented with glycerol, as recommended by the manufacturer (Sigma-Aldrich, Inc.), inoculated by a single colony from an Luria-Agar plate. The colony can be grown in a 15 mL culture tube shaken at 200 RPMs at 37° C. for 36-48 hours. The culture can then spun down in 2 mL centrifuge tubes at 4,000 RPMs for 5 minutes and the supernatant removed to a fresh tube and spun at 4,000 RPMs for an additional 10 minutes. The supernatant (approximately 5 mL) can then be refrigerated until ready for DNA preparation.

SPhage particles containing the f1 origin can be precipitated by adding, for example, 10% acetate pH 5.2 and 2.5 volumes of 100% ethanol and freezing at −20° C. for at least 1 hour, or, alternatively, by adding 6% polyethylene glycol 8000 (PEG 8000) final concentration and shaking at 37° C. for 30 minutes. Precipitated sPhage can be pelleted by centrifugation at 13,000 RPMs for 10 minutes in PEG 8000 or at 4° C. at 13,000 RPMs for 30 minutes in ethanol. Supernatant can be removed and the sPhage pellet brought up in Tris-buffered 2% sodium dodecyl sulfate (SDS) and heated to 70° C. for 30 minutes.

The lysed sPhage can be run through a silica-based column (Qiagen EndoFree MaxiPrep, ThermoFisher HiPure) to purify the DNA following the manufacturers' protocols. DNA can be eluted in 10 mM Tris-HCl elution buffer.

In some forms, the M13 system can be engineered to facilitate direct extrusion of the target scaffold ssDNA into the growth media without a phage intermediate. For these systems, high-throughput testing of media-exported ssDNA can be carried using qPCR, or by capillary electrophoresis. In some forms, high-throughput testing of media-exported ssDNA can be carried using qPCR, or by capillary electrophoresis.

In an exemplary form, 88 or 376 colonies can be individually selected and placed in a 96-well or 384-well plate containing 50 μL of media, and grown for 8 hours at 37° C. while shaking. After 5 to 8 hours, the plate can be centrifuged for 10 minutes at 4000 RPMs. The media supernatant can be pipetted to a different 96- or 384-well plate compatible with the qPCR machine (Roche Lightcycler or ThermoFisher QuantStudio 6 or 7). 1-20 μL of the cleared media is added with 1× to 2× final concentration of SybrGreen I, SybrGreen II, SybrGold, or similar DNA or RNA fluorescent stain. The remaining 8 wells of the plate can be used as a ssDNA standard curve in the same media but without bacterial culture. The plate can be heated to release the ssDNA from the sPhage, and fluorescence measurement be used to identify colonies with high DNA concentrations in the clear media. Those colonies with high fluorescence can also be tested for satisfying the other conditions (agarose gel visualization, sequencing).

If desired, 1 μL of the cleared media can be put in 19 μL of nuclease-free water and boiled at 95° C. for 5 minutes. 1 μL of the boiled solution can be placed in each well of a TaqMan® or similar assay for quantitative measurement of the ssDNA amounts per colony for a specific sequence. Positive colonies can be selected from the plate and grown up for large-scale production.

In some forms, high-throughput testing of media-exported ssDNA can be carried using qPCR, or by capillary electrophoresis. In an exemplary form, machines such as the Fragment Analyzer which rely on capillary electrophoresis can be used to quantitatively determine DNA amounts and sizes in 12 and 96 sample formats. DNA from the cleared media can be loaded to the Fragment Analyzer and visualized to determine colonies that are producing ssDNA of the expected size.

In some forms, mutagenesis of f1 origin and M13 helper strain plasmid can be carried out to increase ssDNA production. In an exemplary form, clones containing the f1 origin can be mutagenized by, for example, incubation with caffeine or subjecting the clone to UV light. In some forms, mutagenic clones can be generated by using mutagenic PCR with Manganese replacing some of the Magnesium in the PCR reaction. aPCR can be carried out with 1.8 mM MgSO4 and 200 μM MnSO4, or some variation of Mg and Mn concentration to allow for high yield ssDNA but with lower or higher mutation numbers per amplicon, or by production in E. coli XL-1 red or other mutagenic strains. Assembly of the phagemid and helper strain plasmid can be carried out as described above. Mutagenized M13 helper strain and f1 origin phagemid can be tested in high throughput using the techniques of purification of functionalized ssDNA and ssDNA production with removal of partial components towards two goals: (1) increasing sPhage production by testing for higher concentrations of DNA in the media, and (2) the direct export of the phagemid DNA into the media without the intermediate assembly of phage particles, without the intermediate step of heating.

In some forms, the method can include purification of functionalized ssDNA. In an exemplary form, the method can use cells containing the expression plasmid for a gene editing protein Cas9 or Cpf1 and the transcription unit for the single-guide or crispr RNA (crRNA) containing a 3′ extension overhang from the crRNA that is complementary to the ssDNA of the phagemid. By producing these in a cell that also contains the helper strain plasmid and the phagemid with only the f1 origin and an additional sequence that contains the sequence complementary to the overhang of the crRNA, the gene editing protein can be loaded directly to the sPhage. This strategy enables both purification of the CRISPR particle, or in vivo delivery of gene editing ribonucleoprotein complex.

In some forms, ssDNA can be produced with removal of partial components. In an exemplary form, LoxP forward and reverse sites can be introduced into the sequence of the phage-produced ssDNA surrounding sites targeted for removal. Cre recombinase enzyme can then be introduced in vitro to induce recombination and splitting of the ssDNA into two circular separate strands. The method can facilitate sequence removal and nucleic acid assembly partitioning.

Synthetic ssDNA produced by the bacteria can be isolated from phage particles or from the bacteria, or from the growth media. Typically, single stranded nucleic acid scaffold sequences can be produced within phage particles in quantities far greater than can be achieved in the absence of a phage or helper microorganism. In some forms, single stranded nucleic acid scaffold sequences can be isolated from phage particles in a two-step process, including buffer-exchanging the media and lysing the phage, for example, by exposure to heat. In some forms, single stranded nucleic acid scaffold sequences can be concentrated and immediately folded into nucleic acid assemblies. In preferred forms, the isolated nucleic acid is sufficiently free of contaminants, such as bacterial dsDNA, that folding can be achieved without the need for any purification of the nucleic acid.

In an exemplary method, phage particles can be collected from the media by first purifying away from bacteria by 2 rounds of centrifugation at 4,000 RPMs for 30 minutes. The supernatant can be concentrated on a 100 kDa MWCO spin concentrator (Amicon) and brought to equivalent volumes with 1×TAE buffer with 12 mM MgCl₂ 3 times. 20 nM phage material can be combined with 400 nM staples in 1×TAE buffer with 12 mM MgCl₂ and 0.2% sodium dodecyl sulfate (SDS) in 50 μL total volume. The solution can be annealed over 13 hours from 95° C. to 24° C. and the folded particle was run on an agarose gel with the ssDNA scaffold for reference.

Typically, the assembly is carried out by hybridization of the staples to the scaffold sequence. Therefore, in some forms, the nucleic acid assemblies are assembled by DNA origami annealing reactions. For example, the oligonucleotide staples are mixed in the appropriate quantities in an appropriate reaction volume. In preferred forms, the staple strand mixes are added in an amount effective to maximize the yield and correct assembly of the assembly. For example, in some forms, the staple strand mixes are added in molar excess of the scaffold strand. In some exemplary forms, the staple strand mixes are added at a 10-20× molar excess of the scaffold strand.

Annealing can be carried out according to the specific parameters of the staple and scaffold sequences.

The assembled nucleic acid assemblies are purified to separate the assembled structures from the substrates and buffers required during the assembly process. Typically, purification is carried out according to the physical characteristics of assemblies. For example, the use of filters and/or chromatographic processes (FPLC, etc.) is carried out according to the size and shape of the assemblies.

In some exemplary forms, nucleic acid assemblies are purified using filtration, such as by centrifugal filtration, or gravity filtration. In some forms, filtration is carried out using an Amicon Ultra-0.5 mL centrifugal filter (MWCO 100 kDa).

Following purification, nucleic acid assemblies can be placed into an appropriate buffer for storage, and/or subsequent structural analysis and validation. Storage can be carried out at room temperature (i.e., 25° C.), 4° C., or below 4° C., for example, at −20° C. Suitable storage buffers include PBS, TAE-Mg²⁺ or DMEM.

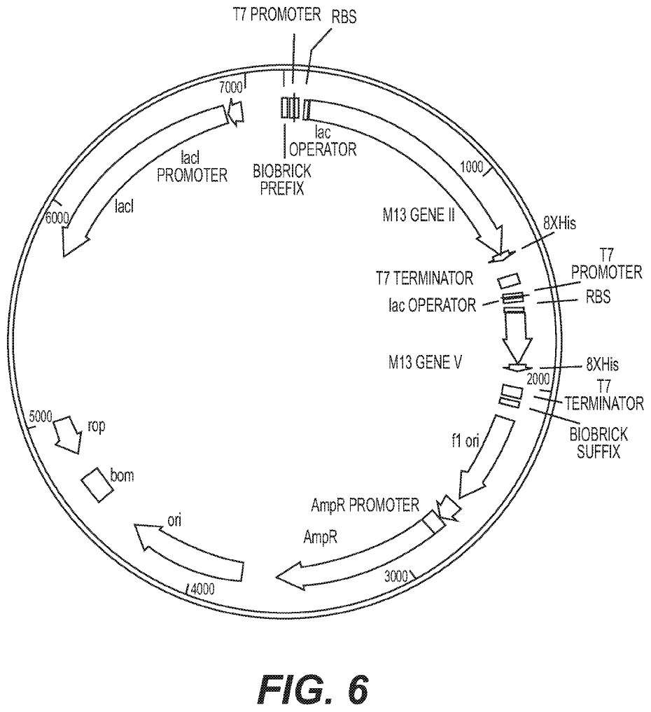

An exemplary plasmid that can be one of the required components for production of ssDNA is illustrated in FIG. 6. The plasmid allows for all-in-one production of a Crispr enzyme (e.g., Cas9) (not shown), sgRNA(s), staple strands (e.g., RNA staples) and M13 phage Genes 2, 10 and 5. Gene 2 (which would include gene 10) and gene 5 of M13 phage were cloned into a pET21a expression vector that was previously modified to be compatible with biobrick cloning. BioBrick cloning cistrons encoding a 5′-T7 promoter, a ribosome binding site, the gene2 or gene 5 coding sequence, followed by T7 terminators, flanked on each side by BioBrick cloning sites, allowed for cloning together the two genes into the pET vector. This construct can be one of the components of a ssDNA production scheme with the bacteria producing the scaffold (e.g., ssDNA scaffold) internally (e.g., via a separate plasmid) without export.

iii. Modified Nucleotides

In some forms, the nucleotides of the scaffolded DNA sequences are modified. For example, in some forms, one or more of the nucleotides of the DNA staple sequences are modified, or one or more of the nucleotides of scaffold sequence are modified, or both nucleotides in the DNA staple sequences and nucleotides in the scaffold sequence are modified.

When modified nucleotides are incorporated into nucleic acid scaffold strands or oligonucleotide staple strands, the modified nucleotides can be incorporated as a percentage or ratio of the total nucleotides used in the preparation of the nucleic acids. In some forms, the modified nucleotides represent 0.1% or more than 0.1% of the total number of nucleotides in the sequence, up to or approaching 100% of the total nucleotides present. For example, the relative amount of modified nucleotides can be between 0.1% and 100% inclusive, such as 0.1%-0.5%, 1%-2%, 1%-5%, 1%-10%, 10%-20%, 20%-30%, 30%-40%, 40%-50%, or more than 50% of the total, up to and including 100%, such as 60%, 70%, 75%, 80%, 85%, 90%, 95% or 99% of the total. In some forms, a sequence of nucleic acids includes a single modified nucleotide, or two, or three modified nucleotides. In some forms, nucleic acid assemblies contain one, or more than one, up to 100 modified nucleotides in every edge. In other forms, the number of modified nucleotides correlates with the size of the assembly, or the shape, or the number of faces or edges, or vertices of the assembly. For example, in some forms, nucleic acid assemblies include the same or different numbers of modified nucleotides within every edge or vertex. In some forms, the modified nucleotides are present at the equivalent position in every structurally-equivalent edge of the assembly. In some forms, nucleic acid assemblies include modified nucleotides at precise locations and in specific numbers or proportions as determined by the design process. Therefore, in some forms, nucleic acid assemblies include a defined number or percentage of modified nucleotides at specified positions within the structure. In some forms, nucleic acid assemblies produced according to the described methods include more than a single type of modified nucleic acid. In some exemplary forms, nucleic acid assemblies include one type of modified nucleic acid on every edge, or mixtures of two or more different modified nucleic acids on every edge. Therefore, when a single type of modified nucleic acid is present at an edge of the structure, each edge can include a different type of modification relative to every other edge.

Non-naturally occurring nucleic acids can include, for example, mixtures of naturally and non-naturally occurring nucleotides. Non-naturally occurring nucleotides and/or nucleotide analogs may be modified at the ribose, phosphate, and/or base moiety. In some forms, nucleic acids can comprise ribonucleotides and non-ribonucleotides. In some such forms, nucleic acids can comprise one or more ribonucleotides and one or more deoxyribonucleotides. In some forms, nucleic acids can comprise one or more non-naturally occurring nucleotide or nucleotide analog such as a nucleotide with phosphorothioate linkage, boranophosphate linkage, a locked nucleic acid (LNA) nucleotides comprising a methylene bridge between the 2′ and 4′ carbons of the ribose ring, peptide nucleic acids (PNA), bridged nucleic acids (BNA), or morpholinos. Other examples of modified nucleotides include 2′-O-methyl analogs, 2′-deoxy analogs, 2-thiouridine analogs, N6-methyladenosine analogs, or 2′-fluoro analogs. Further examples of modified nucleotides include linkage of chemical moieties at the 2′ position, including but not limited to peptides, nuclear localization sequence (NLS), peptide nucleic acid (PNA), morpholino, polyethylene glycol (PEG), triethylene glycol, or tetraethyleneglycol (TEG). Further examples of modified bases include, but are not limited to, 2-aminopurine, 5-bromo-uridine, pseudouridine (Ψ), N¹-methylpseudouridine (me¹Ψ), 5-methoxyuridine (5moU), inosine, 7-methylguanosine. Examples of nucleic acid chemical modifications include, without limitation, incorporation of 2′-O-methyl (M), 2′-O-methyl-3′-phosphorothioate (MS), phosphorothioate (PS), S-constrained ethyl (cEt), 2′-O-methyl-3′-thioPACE (MSP), or 2′-O-methyl-3′-phosphonoacetate (MP) at one or more terminal nucleotides. Such chemically modified nucleic acids can comprise increased stability and increased activity (for active nucleic acids) as compared to unmodified nucleic acids.

Examples of modified nucleotides (such as non-naturally occurring nucleotides) that can be included within the described assemblies include, but are not limited to, diaminopurine, S²T, 5-fluorouracil, 5-bromouracil, 5-chlorouracil, 5-iodouracil, hypoxanthine, xantine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-D46-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Nucleic acid molecules may also be modified at the base moiety (e.g., at one or more atoms that typically are available to form a hydrogen bond with a complementary nucleotide and/or at one or more atoms that are not typically capable of forming a hydrogen bond with a complementary nucleotide), sugar moiety or phosphate backbone. Nucleic acid molecules may also contain amine-modified groups, such as aminoallyl-dUTP (aa-dUTP) and aminohexhylacrylamide-dCTP (aha-dCTP) to allow covalent attachment of amine reactive moieties, such as N-hydroxy succinimide esters (NHS).

In some forms, phosphorothioate modified backbone on the DNA nucleotide staples or on the scaffold is used to improve stability of the DNA assemblies to degradation by exonuclease. For example, in some forms, the nucleic acid assemblies include modified nucleic acids that protect one or more regions of the assembly from enzymic degradation or disruption in vivo. In some forms, nucleic acid assemblies include modified nucleic acids at specific locations within the structure that direct the timing of the enzymic degradation of specific parts of the structure. For example, modifications can be designed to prevent degradation, or to enhance the likelihood of degradation of one or more edges before or after different edges within the same structure. In this way, modifications that enhance or reduce protection or enzymic degradation of one or more parts of an assembly in vivo can drive or facilitate structural changes in the structure, for example, for example to enhance or alter the half-life of a given structure in vivo.

Locked nucleic acid (LNA) is a family of conformationally locked nucleotide analogues which, amongst other benefits, imposes truly unprecedented affinity and very high nuclease resistance to DNA and RNA oligonucleotides (Wahlestedt C, et al., Proc. Natl Acad. Sci. USA, 975633-5638 (2000); Braasch, D A, et al., Chem. Biol. 81-7 (2001); Kurreck J, et al., Nucleic Acids Res. 301911-1918 (2002)). In some forms, the nucleic acids are synthetic RNA-like high affinity nucleotide analogue, locked nucleic acids. In some forms, the scaffolded DNAs are locked nucleic acids. In other forms, the staple strands are locked nucleic acids.

Peptide nucleic acid (PNA) is a nucleic acid analog in which the sugar phosphate backbone of natural nucleic acid has been replaced by a synthetic peptide backbone usually formed from N-(2-amino-ethyl)-glycine units, resulting in an achiral and uncharged mimic (Nielsen P E et al., Science 254, 1497-1500 (1991)). It is chemically stable and resistant to hydrolytic (enzymatic) cleavage. In some forms, the scaffolded DNAs are PNAs. In other forms, the staple strands are PNAs.

In some forms, nucleic acid can comprise morpholino oligonucleotides. Morpholino oligonucleotides are typically composed of two more morpholino monomers containing purine or pyrimidine base-pairing moieties effective to bind, by base-specific hydrogen bonding, to a base in a polynucleotide, which are linked together by phosphorus-containing linkages, one to three atoms long, joining the morpholino nitrogen of one monomer to the 5′ exocyclic carbon of an adjacent monomer. The purine or pyrimidine base-pairing moiety is typically adenine, cytosine, guanine, uracil or thymine. The synthesis, structures, and binding characteristics of morpholino oligomers are detailed in U.S. Pat. Nos. 5,698,685, 5,217,866, 5,142,047, 5,034,506, 5,166,315, 5,521,063, and 5,506,337.

Important properties of the morpholino-based subunits typically include: the ability to be linked in a oligomeric form by stable, uncharged backbone linkages; the ability to support a nucleotide base (e.g. adenine, cytosine, guanine, thymidine, uracil or inosine) such that the polymer formed can hybridize with a complementary-base target nucleic acid, including target RNA, with high Tₘ, even with oligomers as short as 10-14 bases; the ability of the oligomer to be actively transported into mammalian cells; and the ability of an oligomer:RNA heteroduplex to resist RNAse degradation.

In some forms, PNAs, DNAs, RNAs, morpholinos, or LNAs are used for capture, or proteins or other small molecules of interest to target, or otherwise interact with complementary binding sites on structured RNAs, or DNAs. In other forms, a combination of PNAs, DNAs, RNAs, morpholinos, and/or LNAs is used in the formation of structured nucleic acid assemblies.

In some forms, the structured assemblies include a combination of PNAs, DNAs, and/or LNAs. In some forms, a combination of PNAs, DNAs, morpholinos, and/or LNAs is used for the staple strands.

In some forms, the nucleic acids produced according to the described methods are modified to incorporate fluorescent molecules. Exemplary fluorescent molecules include fluorescent dyes and stains, such as Cy5 modified CTP.

In some forms, nucleic acid assemblies include one or more nucleic acids conjugated to polymers. Exemplary polymers that can be conjugated to nucleic acids include biodegradable polymers, non-biodegradable polymers, cationic polymers and dendrimers. For example, a non-limiting list of polymers that can be coupled to nucleic acids within the nucleic acid assemblies includes poly(beta-amino esters); aliphatic polyesters; polyphosphoesters; poly(L-lysine) containing disulfide linkages; poly(ethylenimine) (PEI); disulfide-containing polymers such as DTSP or DTBP cross-linked PEI; PEGylated PEI cross-linked with DTSP; Cross-linked PEI with DSP; Linear SS-PEI; DTSP-Cross-linked linear PEI; branched poly(ethylenimine sulfide) (b-PEIS). Typically, the polymer has a molecular weight of between 500 Da and 20,000 Da, inclusive, for example, approximately 1,000 Da to 10,000 Da, inclusive. In some forms, the polymer is ethylene glycol. In some forms, the polymer is polyethylene glycol. In some exemplary forms, one or more polymer are conjugated to the nucleic acids within one or more of the staples. Therefore, in some forms, one or more types of polymers conjugated to staple strands are used to coat the nucleic acid assembly with the one or more polymers. In some forms, one or more types of polymers conjugated to nucleic acids in the scaffold sequence are used to coat the used to coat the DNA nucleic acid assembly with the one or more polymers.

iv. Modified Assemblies

Nucleic acid assemblies designed and produced according to the described methods can be modified to include nucleic acids having a known function, or molecules other than nucleic acids. Exemplary additional elements include small molecules, proteins, peptides, nucleic acids, lipids, saccharides, or polysaccharides. For example, nucleic acid assemblies can be modified to include proteins or RNAs having a known function, such as antibodies or RNA aptamers having an affinity to one or more target molecules. Therefore, the nucleic acid assemblies designed and produced according to the described methods can be functionalized nucleic acid assemblies.

Nucleic acid assemblies can include one or more functional molecules at one or more locations on or within the structure. In some forms, the functional group is located at one or more staple strands. In other forms, the functional moiety is located directly within the scaffold sequence of the assembly. In other forms, assemblies include one or more functional moieties located within the scaffold sequence and within one or more staple sequences. When assemblies include two or more functional moieties, the functional moieties can be the same, or different.

a. Interaction with Functional Molecules

Typically, nucleic acid assemblies are modified by chemical or physical association with one or more functional molecules. Exemplary methods of conjugation include covalent or non-covalent linkages between the assembly and the functional molecule. In some forms, conjugation with functional molecules is through click-chemistry. In some forms, conjugation with functional molecules is through hybridization with one or more of the nucleic acid sequences present on the assembly. In some forms, conjugation with functional molecules is through click-chemistry. In some forms, functional molecules can be part of the structure of the nucleic acid assembly.

(A) Modified Staple Sequences

In some forms, nucleic acid assemblies include one or more functional groups located at one or more staple strands. For example, in some forms, the nucleic acid assemblies include modified staple strands include single-stranded overhang sequences. In some forms, the overhang sequences are between 4 and 60 nucleotides. In preferred forms, the overhang sequences are between 4 and 25 nucleotides. In some forms, the overhang sequences contain 4, 5, 6, 7, 8, 9, 10, 11, 12, 13, 14, 15, 16, 17, 18, 19, 20, 21, 22, 23, 24, 25, 30, 35, 40, 45, 50 nucleotides in length.

In some forms, assemblies include oligonucleotide staples extended at either the 5′ or 3′ ends by an unpaired region of nucleic acid, such as DNA, RNA, PNA, morpholino, or LNA of known sequence. For example, in some forms, the single-stranded nucleic acid includes a binding site for one or more functional moieties, such as nucleic acids, proteins or small molecules. Therefore, nucleic acid assemblies including staple strands extended to include one or more single-stranded nucleic acid binding sites for a functional nucleic acid, protein or small molecule are described. Nucleic acid assemblies including functional RNA, small molecules, or proteins are also described. The functionalized assemblies can include functional moieties displayed at the surface of the assembly, or located within the inner volume of the assembly. Typically, the location of the functional moiety is determined by the desired biological function of the assembly.

Nucleic acid assemblies functionalized with one or more nucleic acid or non-nucleic acid moieties having a known biological function are provided.

In some forms, nucleic acid assemblies include staple strands extended to include one or more single-stranded nucleic acid sequences that are complementary to the loop region of an RNA, such as an mRNA. Loop regions of mRNA targets can be identified using methods known in the art. When sequences complementary to these loop regions are appended to one or more assembly staple strands, the assembly is capable of capturing the target RNA. Assemblies specifically bound to target RNA can be identified from those that are not bound to the target RNA using any assay known in the art, such as by gel mobility shift, and/or imaging by cryo-EM.

(B) Modified Scaffold Sequences

In some forms, nucleic acid assemblies include a single-stranded scaffold nucleic acid sequence that is modified to include one or more sequences of nucleic acids that bind one or more functional moieties, such as nucleic acids, proteins or small molecules. In some forms, the scaffold includes an overhang sequence that includes one or more functionalizing sequences or moieties at the 5′ or 3′ ends. In other forms, the scaffold includes an internal functionalizing sequence or moiety, for example, within one or more nucleic acids that form part of an edge of the assembly.

b. Functional Molecules

Functional molecules are molecules that have one or more properties and/or functions of interest, especially when associated with a nucleic acid assembly. For example, functional molecules include targeting molecules, effector molecules, and cargo molecules. In the context of nucleic acid assemblies, functional molecules can be attached to, incorporated into, and/or contained or encapsulated by nucleic acid assemblies. For example, nucleic acid assemblies that have nucleic acid overhang sequences can capture one or more functional moieties, including, but not limited to, single-guide- or crispr-RNAs (crRNA), anti-sense DNA, anti-sense RNA, DNA coding for proteins, mRNA, miRNA, piRNA and siRNA, DNA-interacting proteins (such as CRISPR, TAL effector proteins, or zinc-finger proteins), lipids, and carbohydrates. In other forms, nucleic acid assemblies are modified with naturally or non-naturally occurring nucleotides having a known biological function. Exemplary functional groups include targeting elements, immunomodulatory elements, chemical groups, biological macromolecules, and combinations thereof.

In some forms, functionalized nucleic acid assemblies include one or more single-strand overhang or scaffold DNA sequences that are complementary to the loop region of an RNA, such as an mRNA. Nucleic acid assemblies functionalized with mRNAs encoding one or more proteins are described. In some exemplary forms, a tetrahedron (but could be any other object that can be designed from the procedure) can be functionalized with 3 (or 1 or 2 or more than 3) single-strand overhang DNA sequences that are complementary to the loop region of an RNA, for example an mRNA, for example an mRNA expressing a protein.

(A) Targeting Elements

Targeting elements can be added to the staple strands of the DNA assemblies, to enhance targeting of the assemblies to one or more cells, tissues or to mediate specific binding to a protein, lipid, polysaccharide, nucleic acid, etc. For example, for use as biosensors, additional nucleotide sequences are included as overhang sequences on the staple strands.

Exemplary targeting elements include proteins, peptides, nucleic acids, lipids, saccharides, or polysaccharides that bind to one or more targets associated with an organ, tissue, cell, or extracellular matrix, or specific type of tumor or infected cell. The degree of specificity with which the nucleic acid assemblies are targeted can be modulated through the selection of a targeting molecule with the appropriate affinity and specificity. For example, antibodies, or antigen-binding fragments thereof are very specific.

Typically, the targeting moieties exploit the surface-markers specific to a biologically functional class of cells, such as antigen presenting cells. Dendritic cells express a number of cell surface receptors that can mediate endocytosis. In some forms, overhang sequences include nucleotide sequences that are complementary to nucleotide sequences of interest, for example HIV-1 RNA viral genome.

Additional functional groups can be introduced on the staple strand for example by incorporating biotinylated nucleotide into the staple strand. Any streptavidin-coated targeting molecules are therefore introduced via biotin-streptavidin interaction. In other forms, non-naturally occurring nucleotides are included for desired functional groups for further modification. Exemplary functional groups include targeting elements, immunomodulatory elements, chemical groups, biological macromolecules, and combinations thereof.

Typically, the targeting moieties exploit the surface-markers specific to a group of cells to be targeted. Exemplary targeting elements include proteins, peptides, nucleic acids, lipids, saccharides, or polysaccharides that bind to one or more targets associated with cell, or extracellular matrix, or specific type of tumor or infected cell. The degree of specificity with which the delivery vehicles are targeted can be modulated through the selection of a targeting molecule with the appropriate affinity and specificity. For example, antibodies, or antigen-binding fragments thereof are very specific.

(1) Antibodies

In some forms, nucleic acid assemblies are modified to include one or more antibodies. Antibodies that function by binding directly to one or more epitopes, other ligands, or accessory molecules at the surface of cells can be coupled directly or indirectly to the assemblies. In some forms, the antibody or antigen binding fragment thereof has affinity for a receptor at the surface of a specific cell type, such as a receptor expressed at the surface of macrophage cells, dendritic cells, or epithelial lining cells. In some forms, the antibody binds one or more target receptors at the surface of a cell that enables, enhances or otherwise mediates cellular uptake of the antibody-bound assembly, or intracellular translocation of the antibody-bound assembly, or both.

Any specific antibody can be used to modify the nucleic acid assemblies. For example, antibodies can include an antigen binding site that binds to an epitope on the target cell. Binding of an antibody to a “target” cell can enhance or induce uptake of the associated nucleic acid assemblies by the target cell protein via one or more distinct mechanisms.

In some forms, the antibody or antigen binding fragment binds specifically to an epitope. The epitope can be a linear epitope. The epitope can be specific to one cell type or can be expressed by multiple different cell types. In other forms, the antibody or antigen binding fragment thereof can bind a conformational epitope that includes a 3-D surface feature, shape, or tertiary structure at the surface of the target cell.

In some forms, the antibody or antigen binding fragment that binds specifically to an epitope on the target cell can only bind if the protein epitope is not bound by a ligand or small molecule.

Various types of antibodies and antibody fragments can be used to modify nucleic acid assemblies, including whole immunoglobulin of any class, fragments thereof, and synthetic proteins containing at least the antigen binding variable domain of an antibody. The antibody can be an IgG antibody, such as IgG1, IgG2, IgG3, or IgG4 subtypes. An antibody can be in the form of an antigen binding fragment including a Fab fragment, F(ab′)₂ fragment, a single chain variable region, and the like. Antibodies can be polyclonal, or monoclonal (mAb). Monoclonal antibodies include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they specifically bind the target antigen and/or exhibit the desired biological activity (U.S. Pat. No. 4,816,567; and Morrison, et al., Proc. Natl. Acad. Sci. USA, 81: 6851-6855 (1984)). The antibodies can also be modified by recombinant means, for example by deletions, additions or substitutions of amino acids, to increase efficacy of the antibody in mediating the desired function. Substitutions can be conservative substitutions. For example, at least one amino acid in the constant region of the antibody can be replaced with a different residue (see, e.g., U.S. Pat. Nos. 5,624,821; 6,194,551; WO 9958572; and Angal, et al., Mol. Immunol. 30:105-08 (1993)). In some cases changes are made to reduce undesired activities, e.g., complement-dependent cytotoxicity. The antibody can be a bi-specific antibody having binding specificities for at least two different antigenic epitopes. In some forms, the epitopes are from the same antigen. In other forms, the epitopes are from two different antigens. Bi-specific antibodies can include bi-specific antibody fragments (see, e.g., Hollinger, et al., Proc. Natl. Acad. Sci. U.S.A., 90:6444-48 (1993); Gruber, et al., J. Immunol., 152:5368 (1994)).

Antibodies that target the nucleic acid assemblies to a specific epitope can be generated by any means known in the art. Exemplary descriptions of techniques for antibody generation and production include Delves, Antibody Production: Essential Techniques (Wiley, 1997); Shephard, et al., Monoclonal Antibodies (Oxford University Press, 2000); Goding, Monoclonal Antibodies: Principles And Practice (Academic Press, 1993); and Current Protocols In Immunology (John Wiley & Sons, most recent edition). Fragments of intact Ig molecules can be generated using methods well known in the art, including enzymatic digestion and recombinant means.

(2) Capture Tags

In some forms, assemblies include one or more sequences of nucleic acids that act as capture tags, or “Bait” sequences to specifically bind one or more targeted molecules. For example, in some forms, overhang sequences include nucleotide “bait” sequences that are complementary to any target nucleotide sequence, for example HIV-1 RNA viral genome. In some forms, functional groups are present on one or more staple strands to act as capture tags. For example, in some forms, one or more biotinylated nucleotides are incorporated into the staple strand. Streptavidin-coated molecules are therefore introduced via biotin-streptavidin interaction.

Typically, targeting moieties exploit the surface-markers specific to a group of cells to be targeted. Exemplary targeting elements include proteins, peptides, nucleic acids, lipids, saccharides, or polysaccharides that bind to one or more targets associated with cell, or extracellular matrix, or specific type of tumor or infected cell. Targeting molecules can be selected based on the desired physical properties, such as the appropriate affinity and specificity for the target. Exemplary targeting molecules having high specificity and affinity include antibodies, or antigen-binding fragments thereof. Therefore, in some forms, nucleic acid assemblies include one or more antibodies or antigen binding fragments specific to an epitope. The epitope can be a linear epitope. The epitope can be specific to one cell type or can be expressed by multiple different cell types. In other forms, the antibody or antigen binding fragment thereof can bind a conformational epitope that includes a 3-D surface feature, shape, or tertiary structure at the surface of the target cell.

v. Containers

In some forms, nucleic acid assemblies or structures are designed to have a shape or three dimensional form that encloses a volume suitable to contain one or more functional molecules. Such nucleic acid assemblies or structures can be referred to as containers. In some forms, the nucleic acid assemblies are designed to have the shape of a cup, box, vase or other open structure enclosing a volume, into which one or more functional molecules can be loaded or inserted. In some forms, insertion or loading of functional molecules to within the inner space of the nucleic acid assembly is directed through the presence of capture tags within or near the interior space of eth structure. In some forms, functional molecules that are locate within the inner space of the structure are maintained within the structure by the addition of one or more additional molecules, for example, to “block” or otherwise sterically prevent the release of the contained molecule. Therefore, in some forms, nucleic acid assemblies are designed to include a “lid” or other structured nucleic acid form that encapsulates a loaded or “captured” functional molecule with in the inner-space of the nucleic acid assembly. In some forms the access to the inner space of nucleic acid assemblies is mediated by a structural or conformational change in the structure. Therefore, in some forms, the encapsulation of a functional molecule and/or release of the functional molecule from the inner space is controlled by one or more external factors that induce a conformational change in the nucleic acid assembly.

Nucleic acid assemblies are suitable as a delivery vehicle for therapeutic, prophylactic and/or diagnostic agents. Since they are nucleic acid based, DNA nucleic acid assemblies are entirely biocompatible and elicit minimal immune response in the host. The automated design of any desired geometry of DNA nucleic acid assembly further allows manipulation of DNA structure tailored for individual drugs, dose, site of target and desired rate of degradation etc.

Any prophylactic, therapeutic, or diagnostic agent can be incorporated into the DNA origami nucleic acid assemblies via a variety of interactions, non-covalent or covalent. Some exemplary non-covalent interactions for attachment include intercalation, via biotin-streptavidin interaction, chemical linkers (e.g., using Click-chemistry groups), or via hybridization between complementary nucleotide sequences.

In some forms, the agents to be delivered are simply captures inside the DNA origami nucleic acid assemblies. In these cases, pore size of the DNA polyhedron is a key consideration, i.e., they are small enough so that the agent captured does not leak out. In some forms, the DNA polyhedron are assembled in two halves to allow the capture of agent prior to the completion of the polyhedron nucleic acid assemblies.

Prior work has shown that DNA origami as a carrier for anti-cancer drugs such as doxorubicin had increased cellular internalization and increased target cell killing as well as circumvented drug resistance (Jiang Q et al., Journal of the American Chemical Society 134.32: 13396-13403 (2012)). Small molecules, such the anti-cancer drug doxorubicin, can attach to the DNA origami structures through intercalation.

Exemplary agents to be delivered include proteins, peptides, carbohydrates, nucleic acid molecules, polymers, small molecules, and combinations thereof. In some forms, the nucleic acid assemblies are used for the delivery of a peptide drug, a dye, an antibody, or antigen-binding fragment of an antibody.

Therapeutic agents can include anti-cancer, anti-inflammatories, or more specific drugs for inhibition of the disease or disorder to be treated. These may be administered in combination, for example, a general anti-inflammatory with a specific biological targeted to a particular receptor. For example, one can administer an agent in treatment for ischemia that restores blood flow, such as an anticoagulant, anti-thrombotic or clot dissolving agent such as tissue plasminogen activator, as well as an anti-inflammatory. A chemotherapeutic which selectively kills cancer cells may be administered in combination with an anti-inflammatory that reduces swelling and pain or clotting at the site of the dead and dying tumor cells. Suitable genetic therapeutics include anti-sense DNA and RNA as well as DNA coding for proteins, mRNA, miRNA, piRNA and siRNA. In some forms, the nucleic acid that forms the assemblies include one or more therapeutic, prophylactic, diagnostic, or toxic agents.

vi. Physiochemical Properties

Disclosed are compositions involving nucleic acid assemblies that enclose or protect cargo. In some forms, the nucleic acid assembly have useful physiochemical properties that: (i) enhance targeting of the composition to one or more types of cells, tissues, organs, or microenvironments relative to other types of cells, tissues, organs, or microenvironments in vivo; (ii) enhance stability and/or half-life of the composition in vivo; and/or (iii) reduce immunogenicity of the composition. As used herein, physiochemical properties refer to, for example, structures, components, moieties, features, characteristics, and bulk properties that confer on the disclosed compositions selected or desired physiochemical effects. Such selected or desired physiochemical effects include targeting of the composition to one or more types of cells, tissues, organs, or microenvironments, stability and/or half-life of the composition in vivo; and immunogenicity of the composition. In some forms, the nucleic acid assembly and/or cargo comprise features that enhance intracellular trafficking of nucleic acid assembly and/or its cargo.

a. Enhanced Targeting

In some forms, the compositions are targeted to one or more types of cells, tissues, organs, or microenvironments relative to other types of cells, tissues, organs, or microenvironments in vivo. One of skill in the art would recognize that there are multiple ways to preferentially target the compositions. For example, the compositions may be actively targeted to specific cells, tissues, organs, or microenvironments by different forms of molecular interaction (e.g., ligand-receptor, antigen-antibody). To facilitate preferential targeting, the nucleic acid assemblies may be functionalized (e.g., conjugated by an suitable method) with targeting ligands specific to surface components that are unique to, differentially expressed or upregulated in, the cells, tissues, organs, or microenvironments of interest. Exemplary classes of targeting ligands include small molecules, polypeptide-based ligands, and nucleic acid-based aptamers (Friedman A D, et al., Curr Pharm Des., 19(35):6315-29 (2013)). In some forms, the nucleic acid assemblies described herein are conjugated with small molecules, polypeptide-based ligands, nucleic acid-based aptamers, or combinations thereof that contribute to their preferential targeting to one or more types of cells, tissues, organs, or microenvironments.

(A) Small Molecules

The use of small molecules is an attractive targeting strategy given: (1) the availability of a range of facile coupling chemistries for their conjugation, (2) achievement of higher ligand densities on surfaces due to the small size of ligands, (3) availability of a wide range of targeting ligands with variable solubilities and functional groups, as facilitated by advances in diversity-oriented synthesis, (4) less immunogenic effects in vivo (compared to macromolecular ligands) and, (5) reproducible and scalable manufacturing (Kamaly N, et al., Chem Soc Rev., 41(7):2971-3010 (2012)). Examples of small molecules that can be used for targeting of the described nucleic acid assemblies include, but are not limited to, folic acid or folate (a high affinity ligand of endogenous folate receptor), benzamide (e.g., anisamide; a sigma receptor ligand), carbohydrates or sugar moieties (e.g., mannose, glucose, galactose and their derivatives, dimannose, mannosylated lipoarabinomannan (ManLAM), Lewis-X (Lex); ligands of carbohydrate binding proteins such as lectin), myristic acid, RR-11a, and derivatives and/or conjugates thereof (Friedman A D, et al.; Kamaly N, et al.).

(B) Polypeptide-Based Ligands

In some forms, the nucleic acid assemblies described herein are conjugated with polypeptide-based ligands which contribute to their preferential targeting to one or more types of cells, tissues, organs, or microenvironments. Non-limiting examples of polypeptide-based ligands include homing peptides, protein domains, and antibodies (including antibody fragments and derivatives, e.g., Fab, Fab′, F(ab′)₂, Fv fragments, diabodies, affibodies, nanobodies, linear antibodies, and, single-chain antibody molecules). Smaller than antibodies but larger than small molecules, short homing peptides offer additional targeting options. Some non-limiting examples of homing peptides contemplated herein include, ATWLPPR (SEQ ID NO:164), EGF, NGR, S2P, I4R, AH1, TRP2₁₈₀₋₁₈₈, PAn DR epitope, HA₁₁₀₋₁₂₀, iRGD, CANF, CSK, TRAIL, Angiopep-2, tLyp-1, Pep 1, CLL1-L1, RGD, OA02, Tet-1, and, RGD, as disclosed in Friedman A D, et al. Some non-limiting examples of protein domain based ligands that are suitable for targeted delivery of the disclosed nucleic acid assemblies include FN3-based ligands (monobody) that recognize VEGF receptor and integrin αvβ3, Z domain based ligands (affibody) that recognize EGFR and HER2, DARPin based ligands that recognize HER2, transferrin which recognizes the transferrin receptor, adiponectin globular domain, apolipoprotein B-100 LDLR binding domain, C-termini of clostridium/botulinum neurotoxins, hepatitis B surface antigen preS1 domain, LFA-1 I domain, 2Rb18a nanobody, and N7 nanobody (Friedman A D, et al.).

The specificity of antibodies lends particularly well to the active targeting of the compositions/nucleic acid assemblies described herein. One of skill in the art will appreciate that any antibody that specifically binds to a desired target/antigen can be used in accordance with the disclosed compositions. As such, antibodies which specifically recognize one or more types of cells, tissues, organs, or microenvironments are known in the art (e.g., Friedman A D, et al.), and their use in the preferential targeting of the nucleic acid assemblies is contemplated herein. Non-limiting examples of antibodies that can be used include trastuzumab, rituximab, cetuximab, AZN-D1, and AZN-D2.

(C) Nucleic Acid-Based Aptamers

Aptamers are short single-stranded DNA or RNA oligonucleotides (6˜26 kDa) that fold into well-defined 3D structures that recognize a variety of biological molecules including transmembrane proteins, sugars and nucleic acids with high affinity and specificity (Yu B, et al., Mol Membr Biol., 27(7):286-98 (2010)). The high sequence and conformational diversity of naïve aptamer pools (not yet selected against a target) makes the discovery of target binding aptamers highly likely. The selection of aptamers capable of binding a target of interest is called ‘Systematic Evolution of Ligands by EXponential enrichment’ (SELEX). SELEX involves iterative rounds of target binding, partitioning binding from non-binding sequences, and amplification of the enriched binding sequences. Given their unique conformations with ligand-binding characteristics, typical non-immunogenicity and non-toxicity, and ability to be modified for stability in circulation, aptamers are suited to the active targeting of the nucleic acid assemblies described herein (Friedman A D, et al.).

In some forms, the nucleic acid assemblies described herein are conjugated with nucleic acid-based aptamers which contribute to their preferential targeting to one or more types of cells, tissues, organs, or microenvironments. In some forms, the aptamer specifically binds to surface or transmembrane proteins, such as, for example, integrin αvβ3, VEGF receptor, EGF receptor, HER2, HER3, MUC1, PSMA, platelet-derived growth factor receptor (e.g., PDGFRβ), Axl, and receptor tyrosine kinase RET. The aptamer may comprise modified or unmodified DNA or RNA. In some forms, the aptamers are nuclease resistant. In some forms, the aptamer is an RNA aptamer that is 2′-modified (e.g., 2′-fluro and 2′-O-methyl). In some forms, the aptamer (e.g., RNA aptamer) exhibits fluorescence upon binding small molecules. For example, the Spinach and Spinach2 aptamers bind and activate the fluorescence of fluorophores similar to that found in green fluorescent protein, and Broccoli is a 49-nt-long aptamer that exhibits bright green fluorescence upon binding DFHBI or DFHBI-1T (Filonov G S, et al., J Am Chem Soc., 136(46):16299-308 (2014)). Non-limiting examples of aptamers contemplated for use in accordance with the disclosed compositions and methods that have been recently used to target nanoparticles are provided in Friedman A D, et al. (see Table 7).

b. Enhanced Stability and/or Half-Life

In some forms, the nucleic acid assemblies enhance stability and/or half-life of the composition in vivo. Particle size, size distribution, shape, and, surface characteristic are important characteristics of nucleic acid assemblies. These features impact the in vivo distribution, biological fate, toxicity, clearance, uptake and targeting ability of delivery systems (e.g., nanoparticles, nucleic acid assemblies). In addition, they can influence cargo (e.g., drug) loading and release, and stability of the composition (Singh R and Lillard J W Jr. Exp Mol Pathol. 86(3):215-23 (2009); Bamrungsap S, et al., Nanomedicine. 7(8):1253-1271 (2012)). Apart from size, recent studies have shown that the shape of particles can also have an intriguing effect on particle functions, especially in biological processes, including internalization, transport through the blood vessels and targeting sites (Bamrungsap S, et al.). Accordingly, in some forms, the size, size distribution, shape, geometry, surface characteristics (e.g., surface charge, surface chemistry) of the nucleic acid assemblies, or combinations thereof, are modified and/or selected to enhance stability and/or half-life of the compositions in vivo.

c. Reduced Immunogenicity

In some forms, the nucleic acid assemblies reduce immunogenicity of the composition. It is accepted in the art that the physiochemical properties such as particle size and shape, surface charge, hydrophobicity/hydrophilicity, and the steric effects of the coating of compositions (e.g., nanoparticles, nucleic acid assemblies) can dictate compatibility with the immune system (Guo S, et al., Mol Ther Nucleic Acids., 15(9):399-4080 (2017); Zolnik B S, et al., Endocrinology., 151(2): 458-465 (2010)). Notably, the physicochemical properties of nucleic acid based assemblies are tunable, making them an attractive nanomaterial. Their size, shape, sequence, stoichiometry, and other properties can be controlled at ease, and the procedures and process for nucleic acid based assembly construction is reproducible (Guo S, et al.). Therefore, their immunomodulatory effect can be controlled precisely through rational design. Contemplated herein, is the modification/variation of the size, shape, surface charge, hydrophobicity/hydrophilicity, or coating of the disclosed nucleic acid assemblies in order to reduce immunogenicity. For example, in some forms, nucleic acid assemblies are coated with PEG or other types of polymers to provide a hydrophilic environment, thereby shielding them from immune recognition. In some forms, the shape of the nucleic acid assembly is modified or selected (for example, including but not limited to, triangle, square, pentagon, or tetrahedron) in order to reduce immunogenicity. In some forms, the surface charge, or hydrophobicity/hydrophilicity of the nucleic acid assembly is modified or selected in order to reduce immunogenicity.

d. Effector Molecules

Besides size and shape, surface characteristics of compositions can also determine their lifespan during circulation in the blood stream. A major discovery was the finding that compositions (e.g., nanoparticles, nucleic acid assemblies) coated with hydrophilic polymer molecules, such as polyethylene glycol (PEG), can resist serum protein adsorption, prolonging the systemic circulation of the particle. Since, numerous variations of PEG and other hydrophilic polymers have been tested for improved circulation. The surface charge on the particle also affects other functions, such as internalization by macrophages. Positively charged particles have been shown to exhibit higher internalization by macrophages and dendritic cells compared with neutral or negatively charged particles, although surface charge effect could also be cell-type dependent (Bamrungsap S, et al.). Effector molecules can include polyethylene glycol molecules, lipids, polar groups, charged groups, amphipathic groups, and albumin binding molecules.

To enhance half-life of the disclosed nucleic acid assemblies, one may minimize their opsonization and prolong their circulation in vivo. This can be achieved, for example, by coating the nucleic acid assemblies with hydrophilic polymers/surfactants or formulating the nucleic acid assemblies with biodegradable copolymers with hydrophilic characteristics, e.g., PEG, polyethylene oxide, polyoxamer, poloxamine, and polysorbate 80 (Tween 80). Studies show that PEG on nanoparticle surfaces prevents opsonization by complement and other serum factors. PEG molecules with brush-like and intermediate configurations reduce phagocytosis and complement activation, whereas surfaces comprised of PEG with mushroom-like structures are potent complement activators and favor phagocytosis (Singh R and Lillard J W Jr. Exp Mol Pathol. 86(3):215-23 (2009)).

In some forms, the nucleic acid assemblies are coated with a hydrophilic layer (e.g., PEG, polyethylene oxide, polyoxamer, poloxamine, and polysorbate 80 (Tween 80)) to enhance stability and/or half-life of the compositions in vivo. Non-PEG based alternatives such as polyoxazolines, poly(amino acids), polybetaines, polyglycerols, and polysaccharide derivatives may also be used to enhance stability and/or half-life (Amoozgar Z, and Yeo Y. wWiley Interdiscip Rev Nanomed Nanobiotechnol., 4(2):219-33 (2012)). In some forms, the nucleic acid assemblies are coated with polyoxazolines (POZ), poly(amino acids) such as poly(hydroxyethyl 1-glutamine) and poly(hydroxyethyl-1-asparagine), N-(2-hydroxypropyl)methacrylamide (HPMA) and its derivatives, polybetaines such as sulfobetaine and carboxybetaine, polyglycerols (also known as polyglycidols), and polysaccharides such as, derivatives of chitosan, dextran, hyaluronic acid, and heparin.

In some forms, the nucleic acid assemblies comprise a plurality of effector molecules which may contribute to their physiochemical properties (e.g., enhanced stability and/or half-life). In some forms, an effector molecule is any of the above-mentioned molecules that can be used to coat the nucleic acid assembly (e.g., PEG, polyethylene oxide, polyoxamer, poloxamine, and polysorbate 80 (Tween 80), polyoxazolines, poly(amino acids), HPMA, polybetaines, polyglycerols, and polysaccharide derivatives). In some forms, an effector molecule is a polyethylene glycol molecule, lipid, polar group, charged group, amphipathic group, or albumin binding molecule.

2. Cargo

The disclosed nucleic acid assemblies are useful for carrying and delivering cargo. The nucleic acid assemblies are particularly suited for carrying and delivering sensitive cargo, such as nucleic acid molecules, and multicomponent cargo, where the components of the multicomponent cargo work best when delivered together or in a useful or functional stoichiometric ratio. Such stoichiometric ratios are useful, for example, in providing a desired relative effect of different components of the cargo.

Cargo for the disclosed compositions can be any molecules, materials, and compositions desired to be attached to the disclosed nucleic acid assemblies. In some forms, the cargo can include therapeutic, prophylactic, toxic, diagnostic, or other agents. Exemplary agents for use as cargo include proteins, peptides, carbohydrates, nucleic acid molecules, polymers, small molecules, and combinations thereof. In some forms, the cargo can include a peptide drug, a dye, an antibody, or antigen-binding fragment of an antibody. Therapeutic agents for use as cargo can include anti-cancer, anti-inflammatories, or more specific drugs for inhibition of the disease or disorder to be treated. Genetic therapeutics for use as cargo can include anti-sense DNA and RNA as well as DNA coding for proteins, mRNA, miRNA, piRNA and siRNA.

The disclosed compositions are particularly suited to containing and delivering multicomponent cargo, especially multicomponent cargo where the components of the multicomponent cargo operates or is best when present in defined stoichiometric ratio. Multicomponent cargo can include, for example, multiple therapeutic compounds that work best in concert, compounds that affect expression of multiple genes, the coordinated regulation of which is useful, and enzyme(s) and substrate(s) of the enzyme(s). Multiple different cargo molecules that are not part of a multicomponent cargo can also be attached to the disclosed nucleic acid assemblies.

In some forms, the multicomponent cargo can be a DNA or RNA editing system or components of a DNA or RNA editing system. A preferred multicomponent cargo is a CRISPR-Cas system or components of a CRISPR-Cas system. Such systems and components are benefited by protection and delivery in defined stoichiometric ratios.

Cargo that includes more than one cargo molecule, whether part of a multicomponent cargo or not, can be attached to a nucleic acid assembly in a defined stoichiometric ratio. A defined stoichiometric ratio can be any stoichiometric ratio of interest. Preferred forms of stoichiometric ratios include functional stoichiometric ratios and relative effect stoichiometric ratios. As used here, a functional stoichiometric ratio is a stoichiometric ratio at which the components function together. For example, CRISPR-Cas effector proteins, guides (such as sgRNAs), and HDR templates typically function at a 1:1:1 ratio and so a 1:1:1 ratio of a CRISPR-Cas effector protein, a guide for a target, and an HDR template for that target is a functional stoichiometric ratio for these components. As another example, CRISPR-Cas effector proteins, gRNA, tracrRNA, and HDR templates typically function at a 1:1:1:1 ratio and so a 1:1:1:1 ratio of a CRISPR-Cas effector protein, a gRNA for a target, a tracrRNA, and an HDR template for that target is a functional stoichiometric ratio for these components. As another example, CRISPR-Cas nickases (i.e., partially disabled Cas nucleases), gRNA, tracrRNA, and HDR templates are typically used at either a 1:1:1:1 ratio or a 2:2:2:1 ratio and so a 1:1:1:1 ratio or a 2:2:2:1 ratio of a CRISPR-Cas nickase, a gRNA for a target, a tracrRNA, and an HDR template for that target is a functional stoichiometric ratio for these components. Multiplex use of CRISPR systems (i.e., use of CRIPR systems targeted to two or more different target sequences) can extend the functional stoichiometric ratio to the ratio of all of the CRISPR components for all of the targets. This would typically be 1:1:1:1:etc.

As used herein, a relative effect stoichiometric ratio is a stoichiometric ratio at which the components will have a desired relative effect. For example, if it is desired to reduce the amount of two different enzyme substrates to different extents, the stoichiometric ratio of the enzymes that act on the substrates can be used in a stoichiometric ratio such that the enzymes have this desired relative effect on the substrates. As another example, if it is desired that the expression of different genes are modulated to different extents, the stoichiometric ratio of the modulators that act on the genes can be used in a stoichiometric ratio such that the modulators have this desired relative effect on the genes. Multiplex use of CRISPR systems can extend the relative effect stoichiometric ratio to the ratio of all of the CRISPR components for all of the targets. Thus, although the relative effect stoichiometric ratio would typically be 1:1:1:1:etc., differences in efficiencies of different CRISPR systems (if used) and differences in the susceptibility of the different targets can result in functional stoichiometric ratios that are not typical.

i. CRISPR-Cas Systems

In some forms, the cargo may comprise one or more CRISPR-Cas effector proteins and one or more corresponding guide sequences. Alternatively, the cargo may comprise one or more formed CRISPR-Cas complexes, each complex comprising a CRISPR-Cas effector protein and a guide molecule existing as a ribonucleoprotein complex (RNP).

As used herein, the term “Cas” generally refers to an effector protein of a CRISPR-Cas system or complex. The term “Cas” may be used interchangeably with the terms “CRISPR” protein, “CRISPR-Cas protein,” “CRISPR effector,” CRISPR-Cas effector,” “CRISPR enzyme,” “CRISPR-Cas enzyme” and the like, unless otherwise apparent. In general, a “CRISPR system,” “CRISPR-Cas system,” and “CRISPR complex” as used herein and in documents, such as WO 2014/093622 (PCT/US2013/074667), refers collectively to transcripts and other elements involved in the expression of or directing the activity of CRISPR-associated (“Cas”) genes, including sequences encoding a Cas gene, and where applicable, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or “RNA(s)” as that term is herein used (e.g., RNA(s) to guide Cas, such as Cas9, e.g. CRISPR RNA and transactivating (tracr) RNA or a single guide RNA (sgRNA) (chimeric RNA)) or other sequences and transcripts from a CRISPR locus. In general, a CRISPR system is characterized by elements that promote the formation of a CRISPR complex at the site of a target sequence (also referred to as a protospacer in the context of an endogenous CRISPR system). See, e.g., Shmakov et al. (2015) “Discovery and Functional Characterization of Diverse Class 2 CRISPR-Cas Systems,” Molecular Cell, DOI: dx.doi.org/10.1016/j.molcel.2015.10.008.

The Cas effector protein may be without limitation a type II, type V, or type VI Cas effector protein.

a. Cas9

Example Type II Cas effector proteins include Cas9 and orthologs thereof. In some forms, the Type II CRISPR enzyme is a Cas9 enzyme such as disclosed in International Patent Application Publication No. WO/2014/093595. In some forms, the Cas9 enzyme is S. pneumoniae, S. pyogenes or S. thermophilus Cas9, and may include mutated Cas9 derived from these organisms. The enzyme may be a Cas9 homolog or ortholog. Additional orthologs include, for example, Cas9 enzymes from Corynebacter diptheriae, Eubacterium ventriosum, Streptococcus pasteurianus, Lactobacillus farciminis, Sphaeroachaeta globus, Azospirillum B510, Gluconacetobacter diazotrophicus, Neisseria cinereal, Roseburia intestinalis, Parvibaculum lavamentivorans, Staphylococcus aureus, Nitratifractor salsuginis DSM 16511, Camplyobacter lari CF89-12, and Streptococcus thermophilus LMD-9.

In some forms, the Cas9 effector protein and orthologs thereof may be modified for enhanced function. For example, improved target specificity of a CRISPR-Cas9 system may be accomplished by approaches that include, but are not limited to, designing and preparing guide RNAs having optimal activity, selecting Cas9 enzymes of a specific length, truncating the Cas9 enzyme making it smaller in length than the corresponding wild-type Cas9 enzyme by truncating the nucleic acid molecules coding therefor and generating chimeric Cas9 enzymes wherein different parts of the enzyme are swapped or exchanged between different orthologs to arrive at chimeric enzymes having tailored specificity. In some forms, the disclosed methods involve improving the target specificity of a Cas9 ortholog enzyme or of designing a CRISPR-Cas9 system comprising designing or preparing guide RNAs having optimal activity and/or selecting or preparing a Cas9 ortholog enzyme having a smaller size or length than the corresponding wild-type Cas9 whereby packaging a nucleic acid coding therefor into a delivery vector is advanced as there is less coding sequence therefor in the delivery vector than for the corresponding wild-type Cas9 and/or generating chimeric Cas9 enzymes.

A Cas9 enzyme may comprise one or more mutations and may be used as a generic DNA binding protein with or without fusion to or being operably linked to a functional domain. The mutations may be artificially introduced mutations and may include but are not limited to one or more mutations in a catalytic domain. Examples of catalytic domains with reference to a Cas9 enzyme may include but are not limited to RuvC I, RuvC II, RuvC III and HNH domains. Preferred examples of suitable mutations are the catalytic residue(s) in the N-term RuvC I domain of Cas9 or the catalytic residue(s) in the internal HNH domain. In some forms, the Cas9 is (or is derived from) the Streptococcus pyogenes Cas9 (SpCas9). In such forms, preferred mutations are at any or all of positions 10, 762, 840, 854, 863 and/or 986 of SpCas9 or corresponding positions in other Cas9 orthologs with reference to the position numbering of SpCas9 (which may be ascertained for instance by standard sequence comparison tools, e.g. ClustalW or MegAlign by Lasergene 10 suite). In particular, any or all of the following mutations are preferred in SpCas9: D10A, E762A, H840A, N854A, N863A and/or D986A; as well as conservative substitution for any of the replacement amino acids is also envisaged. The same mutations (or conservative substitutions of these mutations) at corresponding positions with reference to the position numbering of SpCas9 in other Cas9 orthologs are also preferred. Particularly preferred are D10 and H840 in SpCas9. However, in other Cas9s, residues corresponding to SpCas9 D10 and H840 are also preferred. These are advantageous as when singly mutated they provide nickase activity and when both mutations are present the Cas9 is converted into a catalytically null mutant which is useful for generic DNA binding. Further mutations have been identified and characterized. In some forms, the mutated Cas 9 enzyme can be fused to or operably linked to domains which include but are not limited to a transcriptional activator, transcriptional repressor, a recombinase, a transposase, a histone remodeler, a DNA methyltransferase, a cryptochrome, a light inducible/controllable domain or a chemically inducible/controllable domain. In some forms, the mutated Cas9 enzyme may be fused to a protein domain, e.g., such as a transcriptional activation domain. In some forms, a transcriptional activation domain is VP64. In some forms, a transcription repression domains is KRAB. In some forms, a transcription repression domain is SID, or concatemers of SID (i.e. SID4X). In some forms, an epigenetic modifying enzyme is provided. In some forms, an activation domain is provided, which may be the P65 activation domain.

CRISPR enzyme advantageously is a nickase enzyme, optionally a Cas9 enzyme comprising at least one mutation in a catalytic domain. For instance, the at least one mutation can be in the RuvC domain and optionally is selected from the group consisting of D10A, E762A and D986A, or is in the HNH domain and optionally is selected from the group consisting of H840A, N854A and N863A. In some forms, the compositions disclosed herein may be used to deliver one or paired nickases. A paired nickase system may comprise a first CRISPR-Cas system comprising a first guide sequence and a second CRISPR-Cas system comprising a second guide sequence. The first guide sequence is hybridizable to a first target sequence and the second guide sequence is hybridizable to a separate target sequence. The first guide sequence directs cleavage of one strand of a DNA duplex near the first target sequence (first nick) and the second guide sequence directs cleavage of the opposite strand of the DNA duplex near the second target sequence (second nick), thereby inducing a break in the DNA. The first nick and the second nick in the DNA is offset relative to each other by at least one base pair of the duplex. The first nick and second nick may also be offset relative to each other by at least one base pair of the duplex, or such that the resulting DNA break has a 3′ overhang. In some forms, the first nick and the second nick are offset relative to each other so that the resulting DNA break has a 5′ overhang. In some forms, the first nick and the second nick are positioned relative to each other such that the overhang is at least 1 nt, at least 10 nt, at least 15 nt, at least 26 nt, at least 30 nt, at least 50 nt or more that at least 50 nt. Other forms of the compositions and methods where the resulting offset double nicked DNA strand can be appreciated by those skilled in the art, and exemplary uses of the double nick system are provided herein. In some forms, the offset between the 5′ ends of the first polynucleotide and the second polynucleotide can be greater than −8 bp or −278 to +58 bp or −200 to +200 bp or up to or over 100 bp or −4 to 20 bp or +23 bp or +16 or +20 or +16 to +20 bp or −3 to +18 bp; and it being understood that where appropriate one may use the term nucleotide or nt for bp. The cleavage of said first strand and of said opposite strand of the DNA duplex may occur 3′ to a PAM (Protospacer adjacent motif) on each strand, and wherein said PAM on said first strand is separated from said PAM on said opposite strand by from 30 to 150 base pairs. The overhang can be at most 200 bases, at most 100 bases, or at most 50 bases; e.g., the overhang can be at least 1 base, at least 10 bases, at least 15 bases, at least 26 bases or at least 30 bases.; or, the overhang can be between 34 and 50 bases or between 1 and 34 bases. Advantageously in inventive methods, any or all of the polynucleotide sequence encoding the CRISPR enzyme, the first and the second guide sequence, the first and the second tracr mate sequence or the first and the second tracr sequence, is/are RNA, and optionally wherein any or all are delivered via the compositions disclosed herein. Additional nickase and paired nickases are disclosed in International Patent Application Nos. WO/2014/204725 and WO/2014/093622.

In some example forms, the Cas9 protein may comprise an inducible dimer, or comprises or consists essentially of or consists of an inducible heterodimer. In some forms, the first half or a first portion or a first fragment of the inducible heterodimer is or comprises or consists of or consists essentially of an FKBP, optionally FKBP12. In some forms, of the inducible CRISPR-Cas system, the second half or a second portion or a second fragment of the inducible heterodimer is or comprises or consists of or consists essentially of FRB. The arrangement of the first CRISPR enzyme fusion construct may comprise or consist of or consist essentially of N′ terminal Cas9 part-FRB-NES. The arrangement of the first CRISPR enzyme fusion construct may also comprise or consists of or consists essentially of NES-N′ terminal Cas9 part-FRB-NES. The arrangement of the second CRISPR enzyme fusion construct may comprise, or consists essentially of, or consists of C′ terminal Cas9 part-FKBP-NLS. The arrangement of the second CRISPR enzyme fusion construct may comprise or consists of or consists essentially of NLS-C′ terminal Cas9 part-FKBP-NLS. There may be a linker that separates the Cas9 part from the half or portion or fragment of the inducible dimer. The inducer energy source may comprise, or consists essentially of, or consists of rapamycin. The inducible dimer may be an inducible homodimer. In some forms, in inducible CRISPR-Cas system, the CRISPR enzyme is Cas9, e.g., SpCas9 or SaCas9. In some forms of inducible CRISPR-Cas system, the Cas9 is split into two parts at any one of the following split points, according or with reference to SpCas9: a split position between 202A/203S; a split position between 255F/256D; a split position between 310E/311I; a split position between 534R/535K; a split position between 572E/573C; a split position between 713S/714G; a split position between 1003L/104E; a split position between 1054G/1055E; a split position between 1114N/1115S; a split position between 1152K/1153S; a split position between 1245K/1246G; or a split between 1098 and 1099. In some forms, in the inducible CRISPR-Cas system, one or more functional domains are associated with one or both parts of the Cas9 enzyme, e.g., the functional domains optionally including a transcriptional activator, a transcriptional or a nuclease such as a Fok1 nuclease. In some forms, in the inducible CRISPR-Cas system, the functional CRISPR-Cas system binds to the target sequence and the enzyme is a deadCas9, optionally having a diminished nuclease activity of at least 97%, or 100% (or no more than 3% and advantageously 0% nuclease activity) as compared with the CRISPR enzyme not having the at least one mutation. In some forms, in the inducible CRISPR-Cas system, the deadCas9 (CRISPR enzyme) comprises two or more mutations wherein two or more of D10, E762, H840, N854, N863, or D986 according to SpCas9 protein or any corresponding ortholog or N580 according to SaCas9 protein are mutated, or the CRISPR enzyme comprises at least one mutation, e.g., wherein at least H840 is mutated. Examples of split Cas9 enzymes are further disclosed and discussed in International Patent Application No. WO/2015/089427.

Some forms of the compositions and methods can use chimeric Cas9 proteins and methods of generating chimeric Cas9 proteins. Chimeric Cas9 proteins are proteins that comprise fragments that originate from different Cas9 orthologs. For instance, the N-terminal of a first Cas9 ortholog may be fused with the C-terminal of a second Cas9 ortholog to generate a resultant Cas9 chimeric protein. These chimeric Cas9 proteins may have a higher specificity or a higher efficiency than the original specificity or efficiency of either of the individual Cas9 enzymes from which the chimeric protein was generated. These chimeric proteins may also comprise one or more mutations or may be linked to one or more functional domains. Therefore, some forms of compositions and methods relate to a chimeric Cas enzyme wherein the enzyme comprises one or more fragments from a first Cas ortholog and one or more fragments from a second Cas ortholog. In some forms, the one or more fragments of the first or second Cas ortholog are from the C- or N-terminal of the first or second Cas ortholog. In some forms, the first or second Cas ortholog is selected from a genus belonging to the group consisting of Corynebacter, Sutterella, Legionella, Treponema, Filifactor, Eubacterium, Streptococcus, Lactobacillus, Mycoplasma, Bacteroides, Flaviivola, Flavobacterium, Sphaerochaeta, Azospirillum, Gluconacetobacter, Neisseria, Roseburia, Parvibaculum, Staphylococcus, Nitratifractor, Mycoplasma and Campylobacter.

b. Cas12

In some forms, the CRISPR effector is a class 2, type V CRISPR effector. In some forms, the CRISPR effector is a class 2, type V-A CRISPR effector. In some forms, the CRISPR effector is a class 2, type V-B CRISPR effector. In some forms, the CRISPR effector is a class 2, type V-C CRISPR effector. In some forms, the CRISPR effector is Cas12a (Cpf1). In some forms, the CRISPR effector is Cas12b (C2c1). In some forms, the CRISPR effector is Cas12c (C2c3). In some forms, the CRISPR effector is a class 2, type V-U CRISPR effector. In some forms, the CRISPR effector is a class 2, type V-U1 CRISPR effector (e.g. C2c4). In some forms, the CRISPR effector is a class 2, type V-U2 CRISPR effector (e.g. C2c8). In some forms, the CRISPR effector is a class 2, type V-U3 CRISPR effector (e.g. C2c10). In some forms, the CRISPR effector is a class 2, type V-U4 CRISPR effector (e.g. C2c9). In some forms, the CRISPR effector is a class 2, type V-U5 CRISPR effector (e.g. C2c5).

(A) Cas12a (Cpf1)

Cas12s effector proteins include effector proteins derived from an organism from a genus comprising Streptococcus, Campylobacter, Nitratifractor, Staphylococcus, Parvibaculum, Roseburia, Neisseria, Gluconacetobacter, Azospirillum, Sphaerochaeta, Lactobacillus, Eubacterium, Corynebacter, Carnobacterium, Rhodobacter, Listeria, Paludibacter, Clostridium, Lachnospiraceae, Clostridiaridium, Leptotrichia, Francisella, Legionella, Alicyclobacillus, Methanomethyophilus, Porphyromonas, Prevotella, Bacteroidetes, Helcococcus, Letospira, Desulfovibrio, Desulfonatronum, Opitutaceae, Tuberibacillus, Bacillus, Brevibacilus, Methylobacterium or Acidaminococcus.

In some forms, the effector protein (e.g., a Cpf1) comprises an effector protein (e.g., a Cpf1) from an organism from S. mutans, S. agalactiae, S. equisimilis, S. sanguinis, S. pneumonia; C. jejuni, C. coli; N. salsuginis, N. tergarcus; S. auricularis, S. carnosus; N. meningitides, N. gonorrhoeae; L. monocytogenes, L. ivanovii; C. botulinum, C. difficile, C. tetani, C. sordellii.

The effector protein may comprise a chimeric effector protein comprising a first fragment from a first effector protein (e.g., a Cpf1) ortholog and a second fragment from a second effector (e.g., a Cpf1) protein ortholog, and wherein the first and second effector protein orthologs are different. At least one of the first and second effector protein (e.g., a Cpf1) orthologs may comprise an effector protein (e.g., a Cpf1) from an organism comprising Streptococcus, Campylobacter, Nitratifractor, Staphylococcus, Parvibaculum, Roseburia, Neisseria, Gluconacetobacter, Azospirillum, Sphaerochaeta, Lactobacillus, Eubacterium, Corynebacter, Carnobacterium, Rhodobacter, Listeria, Paludibacter, Clostridium, Lachnospiraceae, Clostridiaridium, Leptotrichia, Francisella, Legionella, Alicyclobacillus, Methanomethyophilus, Porphyromonas, Prevotella, Bacteroidetes, Helcococcus, Letospira, Desulfovibrio, Desulfonatronum, Opitutaceae, Tuberibacillus, Bacillus, Brevibacilus, Methylobacterium or Acidaminococcus; e.g., a chimeric effector protein comprising a first fragment and a second fragment wherein each of the first and second fragments is selected from a Cpf1 of an organism comprising Streptococcus, Campylobacter, Nitratifractor, Staphylococcus, Parvibaculum, Roseburia, Neisseria, Gluconacetobacter, Azospirillum, Sphaerochaeta, Lactobacillus, Eubacterium, Corynebacter, Carnobacterium, Rhodobacter, Listeria, Paludibacter, Clostridium, Lachnospiraceae, Clostridiaridium, Leptotrichia, Francisella, Legionella, Alicyclobacillus, Methanomethyophilus, Porphyromonas, Prevotella, Bacteroidetes, Helcococcus, Letospira, Desulfovibrio, Desulfonatronum, Opitutaceae, Tuberibacillus, Bacillus, Brevibacilus, Methylobacterium or Acidaminococcus wherein the first and second fragments are not from the same bacteria; for instance a chimeric effector protein comprising a first fragment and a second fragment wherein each of the first and second fragments is selected from a Cpf1 of S. mutans, S. agalactiae, S. equisimilis, S. sanguinis, S. pneumonia; C. jejuni, C. coli; N. salsuginis, N. tergarcus; S. auricularis, S. carnosus; N. meningitides, N. gonorrhoeae; L. monocytogenes, L. ivanovii; C. botulinum, C. difficile, C. tetani, C. sordellii; Francisella tularensis 1, Prevotella albinisms, Lachnospiraceae bacterium MC2017 1, Butyrivibrio proteoclasticus, Peregrinibacteria bacterium GW2011_GWA2_33_10, Parcubacteria bacterium GW2011_GWC2_44_17, Smithella sp. SCADC, Acidaminococcus sp. BV3L6, Lachnospiraceae bacterium MA2020, Candidatus Methanoplasma termitum, Eubacterium eligens, Moraxella bovoculi 237, Leptospira inadai, Lachnospiraceae bacterium ND2006, Porphyromonas crevioricanis 3, Prevotella disiens and Porphyromonas macacae, wherein the first and second fragments are not from the same bacteria.

In some forms, the effector protein is derived from a Cpf1 locus (herein such effector proteins are also referred to as “Cpf1p”), e.g., a Cpf1 protein (and such effector protein or Cpf1 protein or protein derived from a Cpf1 locus is also called “CRISPR enzyme”). Cpf1 loci include but are not limited to the Cpf1 loci of bacterial species listed in FIG. 64. In more preferred forms, the Cpf1p is derived from a bacterial species selected from Francisella tularensis 1, Prevotella albensis, Lachnospiraceae bacterium MC2017 1, Butyrivibrio proteoclasticus, Peregrinibacteria bacterium GW2011_GWA2_33_10, Parcubacteria bacterium GW2011_GWC2_44_17, Smithella sp. SCADC, Acidaminococcus sp. BV3L6, Lachnospiraceae bacterium MA2020, Candidatus Methanoplasma termitum, Eubacterium eligens, Moraxella bovoculi 237, Leptospira inadai, Lachnospiraceae bacterium ND2006, Porphyromonas crevioricanis 3, Prevotella disiens and Porphyromonas macacae. In some forms, the Cpf1p is derived from a bacterial species selected from Acidaminococcus sp. BV3L6, Lachnospiraceae bacterium MA2020. In some forms, the effector protein is derived from a subspecies of Francisella tularensis 1, including but not limited to Francisella tularensis subsp. Novicida.

Cpf1 effector proteins may be modified, e.g., an engineered or non-naturally-occurring effector protein or Cpf1. In some forms, the modification may comprise mutation of one or more amino acid residues of the effector protein. The one or more mutations may be in one or more catalytically active domains of the effector protein. The effector protein may have reduced or abolished nuclease activity compared with an effector protein lacking said one or more mutations. The effector protein may not direct cleavage of one or other DNA or RNA strand at the target locus of interest. The effector protein may not direct cleavage of either DNA or RNA strand at the target locus of interest. In preferred forms, the one or more mutations may comprise two mutations. In preferred forms, the one or more amino acid residues are modified in a Cpf1 effector protein, e.g., an engineered or non-naturally-occurring effector protein or Cpf1. In preferred forms, the Cpf1 effector protein is an FnCpf1 effector protein. In preferred forms, the one or more modified or mutated amino acid residues are D917A, E1006A or D1255A with reference to the amino acid position numbering of the FnCpf1 effector protein. In further preferred forms, the one or more mutated amino acid residues are D908A, E993A, and D1263A with reference to the amino acid positions in AsCpf1 or LbD832A, E925A, D947A, and D1180A with reference to the amino acid positions in LbCpf1.

In some forms, one or more mutations of the two or more mutations can be in a catalytically active domain of the effector protein comprising a RuvC domain. In some forms, the RuvC domain may comprise a RuvCI, RuvCII or RuvCIII domain, or a catalytically active domain which is homologous to a RuvCI, RuvCII or RuvCIII domain etc. or to any relevant domain as described in any of the herein described methods. The effector protein may comprise one or more heterologous functional domains. The one or more heterologous functional domains may comprise one or more nuclear localization signal (NLS) domains. The one or more heterologous functional domains may comprise at least two or more NLS domains. The one or more NLS domain(s) may be positioned at or near or in proximity to a terminus of the effector protein (e.g., Cpf1) and if two or more NLSs, each of the two may be positioned at or near or in proximity to a terminus of the effector protein (e.g., Cpf1) The one or more heterologous functional domains may comprise one or more transcriptional activation domains. In preferred forms, the transcriptional activation domain may comprise VP64. The one or more heterologous functional domains may comprise one or more transcriptional repression domains. In preferred forms, the transcriptional repression domain comprises a KRAB domain or a SID domain (e.g. SID4X). The one or more heterologous functional domains may comprise one or more nuclease domains. In preferred forms, a nuclease domain comprises Fok1.

In some forms, the one or more heterologous functional domains can have one or more of the following activities: methylase activity, demethylase activity, transcription activation activity, transcription repression activity, transcription release factor activity, histone modification activity, nuclease activity, single-strand RNA cleavage activity, double-strand RNA cleavage activity, single-strand DNA cleavage activity, double-strand DNA cleavage activity and nucleic acid binding activity. At least one or more heterologous functional domains may be at or near the amino-terminus of the effector protein and/or wherein at least one or more heterologous functional domains is at or near the carboxy-terminus of the effector protein. The one or more heterologous functional domains may be fused to the effector protein. The one or more heterologous functional domains may be tethered to the effector protein. The one or more heterologous functional domains may be linked to the effector protein by a linker moiety.

In some forms, a protospacer adjacent motif (PAM) or PAM-like motif directs binding of the effector protein complex to the target locus of interest. In some forms, the PAM is 5′ TTN, where N is A/C/G or T and the effector protein is FnCpf1p. In some forms, the PAM is 5′ TTTV, where V is A/C or G and the effector protein is AsCpf1, LbCpf1 or PaCpf1p. In some forms, the PAM is 5′ TTN, where N is A/C/G or T, the effector protein is FnCpf1p, and the PAM is located upstream of the 5′ end of the protospacer. In some forms, the PAM is 5′ CTA, where the effector protein is FnCpf1p, and the PAM is located upstream of the 5′ end of the protospacer or the target locus. In some forms, an expanded targeting range for RNA guided genome editing nucleases can be used, where the T-rich PAMs of the Cpf1 family allow for targeting and editing of AT-rich genomes.

In some forms, the CRISPR enzyme is engineered and can comprise one or more mutations that reduce or eliminate a nuclease activity. The amino acid positions in the FnCpf1p RuvC domain include but are not limited to D917A, E1006A, E1028A, D1227A, D1255A, N1257A, D917A, E1006A, E1028A, D1227A, D1255A and N1257A. A putative second nuclease domain is known that is most similar to PD-(D/E)XK nuclease superfamily and HincII endonuclease like. The point mutations to be generated in this putative nuclease domain to substantially reduce nuclease activity include but are not limited to N580A, N584A, T587A, W609A, D610A, K613A, E614A, D616A, K624A, D625A, K627A and Y629A. In preferred forms, the mutation in the FnCpf1p RuvC domain is D917A or E1006A, wherein the D917A or E1006A mutation completely inactivates the DNA cleavage activity of the FnCpf1 effector protein. In other forms, the mutation in the FnCpf1p RuvC domain is D1255A, wherein the mutated FnCpf1 effector protein has significantly reduced nucleolytic activity.

The amino acid positions in the AsCpf1p RuvC domain include but are not limited to 908, 993, and 1263. In preferred forms, the mutation in the AsCpf1p RuvC domain is D908A, E993A, and D1263A, wherein the D908A, E993A, and D1263A mutations completely inactivates the DNA cleavage activity of the AsCpf1 effector protein. The amino acid positions in the LbCpf1p RuvC domain include but are not limited to 832, 947 or 1180. In preferred forms, the mutation in the LbCpf1p RuvC domain is LbD832A, E925A, D947A or D1180A, wherein the LbD832A E925A, D947A or D1180A mutations completely inactivates the DNA cleavage activity of the LbCpf1 effector protein.

Mutations can also be made at neighboring residues, e.g., at amino acids near those indicated above that participate in the nuclease activity. In some forms, only the RuvC domain is inactivated, and in other forms, another putative nuclease domain is inactivated, wherein the effector protein complex functions as a nickase and cleaves only one DNA strand. In preferred forms, the other putative nuclease domain is a HincII-like endonuclease domain. In some forms, two FnCpf1, AsCpf1 or LbCpf1 variants (each a different nickase) are used to increase specificity, two nickase variants are used to cleave DNA at a target (where both nickases cleave a DNA strand, while minimizing or eliminating off-target modifications where only one DNA strand is cleaved and subsequently repaired). In preferred forms, the Cpf1 effector protein cleaves sequences associated with or at a target locus of interest as a homodimer comprising two Cpf1 effector protein molecules. In preferred forms, the homodimer may comprise two Cpf1 effector protein molecules comprising a different mutation in their respective RuvC domains.

In some forms, two or more nickases can be used, in particular a dual or double nickase approach. In some forms, a single type FnCpf1, AsCpf1 or LbCpf1 nickase may be delivered, for example a modified FnCpf1, AsCpf1 or LbCpf1 or a modified FnCpf1, AsCpf1 or LbCpf1 nickase as described herein. This results in the target DNA being bound by two FnCpf1 nickases. In addition, it is also envisaged that different orthologs may be used, e.g., an FnCpf1, AsCpf1 or LbCpf1 nickase on one strand (e.g., the coding strand) of the DNA and an ortholog on the non-coding or opposite DNA strand. The ortholog can be, but is not limited to, a Cas9 nickase such as a SaCas9 nickase or a SpCas9 nickase. It may be advantageous to use two different orthologs that require different PAMs and may also have different guide requirements, thus allowing a greater deal of control for the user. In some forms, DNA cleavage will involve at least four types of nickases, wherein each type is guided to a different sequence of target DNA, wherein each pair introduces a first nick into one DNA strand and the second introduces a nick into the second DNA strand. In such methods, at least two pairs of single stranded breaks are introduced into the target DNA wherein upon introduction of first and second pairs of single-strand breaks, target sequences between the first and second pairs of single-strand breaks are excised. In some forms, one or both of the orthologs is controllable, i.e. inducible.

The Cas12a enzymes may further include dCpf1 fused to an adenosine or cytidine deaminase such as those disclosed in U.S. Provisional Application Nos. 62/508,293, 62/561,663, and 62/568,133, 62/609,949, and 62/610,065.

Additional Cas12a enzymes that may be delivered used the compositions disclosed herein are discussed in International Patent Application Nos. WO/2016/205711, WO/2017/106657, and WO/2017/172682.

(B) Cas12b (C2c1) and Cas12c (C2c3)

In some forms, the Cas protein may comprise a Cas12b or Cas12c effector protein. The Cas12b (C2c1) or Cas12c (C2c2) effector protein may be derived from an organism from a genus comprising Streptococcus, Campylobacter, Nitratifractor, Staphylococcus, Parvibaculum, Roseburia, Neisseria, Gluconacetobacter, Azospirillum, Sphaerochaeta, Lactobacillus, Eubacterium, Corynebacter, Carnobacterium, Rhodobacter, Listeria, Paludibacter, Clostridium, Lachnospiraceae, Clostridiaridium, Leptotrichia, Francisella, Legionella, Alicyclobacillus, Methanomethyophilus, Porphyromonas, Prevotella, Bacteroidetes, Helcococcus, Letospira, Desulfovibrio, Desulfonatronum, Opitutaceae, Tuberibacillus, Bacillus, Brevibacilus, Methylobacterium or Acidaminococcus. The effector protein may comprise a chimeric effector protein comprising a first fragment from a first effector protein ortholog and a second fragment from a second effector protein ortholog, and wherein the first and second effector protein orthologs are different. At least one of the first and second effector protein orthologs may comprise an effector protein from an organism comprising Streptococcus, Campylobacter, Nitratifractor, Staphylococcus, Parvibaculum, Roseburia, Neisseria, Gluconacetobacter, Azospirillum, Sphaerochaeta, Lactobacillus, Eubacterium, Corynebacter, Carnobacterium, Rhodobacter, Listeria, Paludibacter, Clostridium, Lachnospiraceae, Clostridiaridium, Leptotrichia, Francisella, Legionella, Alicyclobacillus, Methanomethyophilus, Porphyromonas, Prevotella, Bacteroidetes, Helcococcus, Letospira, Desulfovibrio, Desulfonatronum, Opitutaceae, Tuberibacillus, Bacillus, Brevibacilus, Methylobacterium or Acidaminococcus.

In some forms, the effector protein, particularly a Type V loci effector protein, more particularly a Type V-B loci effector protein, even more particularly a C2c1p, may originate from, may be isolated from or may be derived from a bacterial species belonging to the taxa Bacilli, Verrucomicrobia, alpha-proteobacteria or delta-proteobacteria. In some forms, the effector protein, particularly a Type V loci effector protein, more particularly a Type V-B loci effector protein, even more particularly a C2c1p, may originate from, may be isolated from or may be derived from a bacterial species belonging to a genus selected from the group consisting of Alicyclobacillus, Desulfovibrio, Desulfonatronum, Opitutaceae, Tuberibacillus, Bacillus, Brevibacillus, Desulfatirhabdium, Citrobacter, and Methylobacterium. In some forms, the effector protein, particularly a Type V loci effector protein, more particularly a Type V-B loci effector protein, even more particularly a C2c1p, may originate, may be isolated or may be derived from a bacterial species selected from the group consisting of Alicyclobacillus acidoterrestris (e.g., ATCC 49025), Alicyclobacillus contaminans (e.g., DSM 17975), Desulfovibrio inopinatus (e.g., DSM 10711), Desulfonatronum thiodismutans (e.g., strain MLF-1), Opitutaceae bacterium TAV5, Tuberibacillus calidus (e.g., DSM 17572), Bacillus thermoamylovorans (e.g., strain B4166), Brevibacillus sp. CF112, Bacillus sp. NSP2.1, Desulfatirhabdium butyrativorans (e.g., DSM 18734), Alicyclobacillus herbarius (e.g., DSM 13609), Citrobacter freundii (e.g., ATCC 8090), Brevibacillus agri (e.g., BAB-2500), Methylobacterium nodulans (e.g., ORS 2060).

Additional Cas12a and Cas12c orthologs are disclosed in International Patent Application Publication No. WO/2016/205749.

Cas12b and Cas12c effector protein may further comprise modifications wherein one or more amino acid residues of the effector protein may be modified e.g., an engineered or non-naturally-occurring effector protein or C2c1 or C2c3. In some forms, the modification may comprise mutation of one or more amino acid residues of the effector protein. The one or more mutations may be in one or more catalytically active domains of the effector protein. The effector protein may have reduced or abolished nuclease activity compared with an effector protein lacking said one or more mutations. The effector protein may not direct cleavage of one or other DNA or RNA strand at the target locus of interest. The effector protein may not direct cleavage of either DNA or RNA strand at the target locus of interest. In preferred forms, the one or more mutations may comprise two mutations. In preferred forms, the one or more amino acid residues are modified in a C2c1 or C2c3 effector protein, e.g., an engineered or non-naturally-occurring effector protein or C2c1 or C2c3.

In some forms, the one or more mutations of the two or more mutations to be in a catalytically active domain of the effector protein comprising a RuvC domain. In some forms, the RuvC domain may comprise a RuvCI, RuvCII or RuvCIII domain, or a catalytically active domain which is homologous to a RuvCI, RuvCII or RuvCIII domain etc. or to any relevant domain as described in any of the herein described methods. In some forms, the one or more mutations of the two or more mutations may be in a catalytically active domain of the effector protein comprising a HEPN domain, or a catalytically active domain which is homologous to a HEPN domain. The effector protein may comprise one or more heterologous functional domains. The one or more heterologous functional domains may comprise one or more nuclear localization signal (NLS) domains. The one or more heterologous functional domains may comprise at least two or more NLS domains. The one or more NLS domain(s) may be positioned at or near or in proximity to a terminus of the effector protein (e.g., C2c1 or C2c3) and if two or more NLSs, each of the two may be positioned at or near or in proximity to a terminus of the effector protein (e.g., C2c1 or C2c3). The one or more heterologous functional domains may comprise one or more transcriptional activation domains. In preferred forms, the transcriptional activation domain may comprise VP64. The one or more heterologous functional domains may comprise one or more transcriptional repression domains. In preferred forms, the transcriptional repression domain comprises a KRAB domain or a SID domain (e.g. SID4X). The one or more heterologous functional domains may comprise one or more nuclease domains. In preferred forms, a nuclease domain comprises Fok1.

In some forms, the one or more heterologous functional domains can have one or more of the following activities: methylase activity, demethylase activity, transcription activation activity, transcription repression activity, transcription release factor activity, histone modification activity, nuclease activity, single-strand RNA cleavage activity, double-strand RNA cleavage activity, single-strand DNA cleavage activity, double-strand DNA cleavage activity and nucleic acid binding activity. At least one or more heterologous functional domains may be at or near the amino-terminus of the effector protein and/or wherein at least one or more heterologous functional domains is at or near the carboxy-terminus of the effector protein. The one or more heterologous functional domains may be fused to the effector protein. The one or more heterologous functional domains may be tethered to the effector protein. The one or more heterologous functional domains may be linked to the effector protein by a linker moiety. In some forms, the heterologous functional domain may be an adenosine or cytidine deaminase such as disclosed in U.S. 62/610,041 and U.S. 62/610,005.

c. Cas13

In some forms, the CRISPR effector is a class 2, type VI CRISPR effector. In some forms, the CRISPR effector is a class 2, type VI-A CRISPR effector. In some forms, the CRISPR effector is a class 2, type VI-B CRISPR effector. In some forms, the CRISPR effector is a class 2, type VI-B1 CRISPR effector. In some forms, the CRISPR effector is a class 2, type VI-B2 CRISPR effector. In some forms, the CRISPR effector is a class 2, type VI-C CRISPR effector. In some forms, the CRISPR effector is Cas13a (C2c2). In some forms, the CRISPR effector is Cas13b (C2c6). In some forms, the CRISPR effector is Cas13c (C2c7).

In some forms, the Cas13 protein is a Cas13d protein. Yan et al. Molecular Cell, 70, 327-339 (2018).

In general, a CRISPR system as used herein and in documents, such as WO 2014/093622 (PCT/US2013/074667), refers collectively to transcripts and other elements involved in the expression of or directing the activity of CRISPR-associated (“Cas”) genes, including sequences encoding a Cas gene, and where applicable, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or “RNA(s)” as that term is herein used (e.g., RNA(s) to guide Cas, such as Cas9, e.g. CRISPR RNA and transactivating (tracr) RNA or a single guide RNA (sgRNA) (chimeric RNA)) or other sequences and transcripts from a CRISPR locus. In general, a CRISPR system is characterized by elements that promote the formation of a CRISPR complex at the site of a target sequence (also referred to as a protospacer in the context of an endogenous CRISPR system). See, e.g., Shmakov et al. (2015) “Discovery and Functional Characterization of Diverse Class 2 CRISPR-Cas Systems,” Molecular Cell, DOI: dx.doi.org/10.1016/j.molcel.2015.10.008.

In some forms, a protospacer adjacent motif (PAM) or PAM-like motif directs binding of the effector protein complex as disclosed herein to the target locus of interest. In some forms, the PAM may be a 5′ PAM (i.e., located upstream of the 5′ end of the protospacer). In other forms, the PAM may be a 3′ PAM (i.e., located downstream of the 5′ end of the protospacer). The term “PAM” may be used interchangeably with the term “PFS” or “protospacer flanking site” or “protospacer flanking sequence.”

In preferred forms, the CRISPR effector protein may recognize a 3′ PAM. In some forms, the CRISPR effector protein may recognize a 3′ PAM which is 5′H, wherein H is A, C or U.

In the context of formation of a CRISPR complex, “target sequence” refers to a sequence to which a guide sequence is designed to have complementarity, where hybridization between a target sequence and a guide sequence promotes the formation of a CRISPR complex. A target sequence can comprise RNA polynucleotides. The term “target RNA” refers to a RNA polynucleotide being or comprising the target sequence. In other words, the target RNA may be a RNA polynucleotide or a part of a RNA polynucleotide to which a part of the gRNA, i.e. the guide sequence, is designed to have complementarity and to which the effector function mediated by the complex comprising CRISPR effector protein and a gRNA is to be directed. In some forms, a target sequence is located in the nucleus or cytoplasm of a cell.

d. Dead Cas

In some forms, the CRISPR enzyme is a deadCas (dCas), which is a CRISPR enzyme having a diminished nuclease activity. For example, the nuclease activity can be diminished by at least 97% or 100% (i.e., no more than 3% and advantageously 0% nuclease activity) as compared with the CRISPR enzyme not having any mutations. In some forms, dCas can be a deadCas9 (dCas9). In some forms, the dCas9 can comprise at least one mutation or two or more mutations. In some forms, the at least one mutation can be at position H840 (or at the corresponding position in any corresponding ortholog). In some forms, the two or more mutations can comprise mutations at two or more of the positions D10, E762, H840, N854, N863, or D986 according to SpCas9 protein (or corresponding positions in any corresponding ortholog), at position N580 according to SaCas9 protein (or corresponding positions in any corresponding ortholog).

In some forms, dCas can be a deadCas13 (dCas13). In some forms, the dCas13 can comprise at least one mutation or two or more mutations. In some forms, the dead Cas13 is a dead Cas13a protein which comprises one or more mutations in the HEPN domain. In some forms, the dead Cas13a comprises a mutation corresponding to R474A and R1046A in Leptotrichia wadei (LwaCas13a). In some forms, the dead Cas13 is a dead Cas13b protein which comprises one or more of R116A, H121A, R1177A, and H1182A of a Cas13b protein originating from Bergeyella zoohelcum ATCC 43767 or amino acid positions corresponding thereto of a Cas13b ortholog.

e. CRISPR-Cas Systems

CRISPR-Cas and other DNA and RNA editing systems can be configured and used in multiple ways and for multiple purposes. Generally, particular or specialized CRISPR-Cas components are used in certain forms of CRISPR-Cas systems to achieve certain purposes. For example, dCas with a functional molecule attached can be used to bring the functional molecule to a particular target sequence (dCas system); paired Cas nickases can be used to produce a double strand cleavage at a target site with a desired overhang length and orientation (paired nickase system); two or more dCas proteins targeted to sites flanking a main target site can be used with an active Cas targeted to the main target in order to make the main target more accessible and the editing at the main target more efficiently (proxy-CRISPR system); and Cas can be used with separate and shortened gRNA and tracrRNA (rather than an sgRNA that combines a guide RNA and tracrRNA in a single molecule) to increase the efficiency of editing (Alt-R CRISPR system).

CRISPR-Cas systems can be used individually or in combinations. Use of combinations of CRISPR-Cas systems can be referred to as multiplex CRISPR-Cas systems. For example, two or more CRISR-Cas systems, each targeted to a different target sequence, can be used together to edit all of the targets in parallel; paired Cas nickases can be used together to produce a double strand cleavage at a target site; and two or more dCas proteins targeted to sites flanking a main target site can be used with an active Cas targeted to the main target in order to make the main target more accessible and the editing at the main target more efficiently. Combinations of these and any other CRISPR-Cas systems can be used together.

In some forms, the CRISPR-Cas system can be a Cas9 system, a Cas12 system, a Cas13 system, a dCas system, a nickase system, a paired nickase system, an Alt-R CRISPR system, a proxy-CRISPR system, an Alt-R dCas system, an Alt-R nickase system, an Alt-R paired nickase system, an Alt-R proxy-CRISPR system, a proxy-dCas system, a proxy-nickase system, a proxy-paired nickase system, an Alt-R proxy-dCas system, an Alt-R proxy-nickase system, or an Alt-R proxy-paired nickase system. Alt-R CRISPR features can be used with any suitable CRISPR-Cas systems, such as Cas systems, dCas systems, nickase systems, paired nickase systems, and proxy-CRISPR systems. Proxy-CRISPR features can be used with any suitable CRISPR-Cas systems, such as Cas systems, dCas systems, nickase systems, paired nickase systems, and Alt-R CRISPR systems. Alt-R CRISPR features and proxy-CRISPR features can be used together with any suitable CRISPR-Cas systems, such as Cas systems, dCas systems, nickase systems, and paired nickase systems.

CRISPR-Cas systems can use any suitable CRISPR-Cas effector protein or combinations of CRISPR-Cas effector proteins. In some forms, the CRISPR-Cas system can include SpCas9, dCas9, Cas nickase, Cas9 nickase, FnCas9, StCas9, SaCas9, LpCas9, FnCas12, Cas12 nickase, AsCas12, LbCas12, Cas12a, Cas12b, Cas12c, Cas13, Cas13d, or combinations thereof.

In some forms, the CRISPR-Cas system can comprises a paired Cas9 nickase system, a paired Cas9 nickase system, a dCas/Cas proxy-CRISPR system, a dCas9/Cas9 proxy-CRISPR system, or an SpdCas9/FnCas9 proxy-CRISPR system.

In some forms, the proxy-CRISPR system can comprise two first dCas ribonucleoproteins targeted to different sites flanking and on opposite sides of a main target site and a Cas ribonucleoprotein targeted to the main target site. In some forms, the proxy-CRISPR system can further comprise two additional dCas ribonucleoproteins targeted to different sites flanking and on opposite sides of the main target site, where the sites to which the additional dCas ribonucleoproteins are targeted are not the same as the target sites to which the first dCas ribonucleoproteins are targeted.

In some forms, the Alt-R CRISPR system can comprise a separate, shortened gRNA, a separate, shortened tracrRNA, a Cas9 protein. In some forms, the paired nickase system leaves 5′ overhangs. In some forms, the cargo comprises one or more components of two or more CRISPR-Cas systems.

f. Nucleotide Base Editors

The CRISPR system can also include a dCas-based nucleotide base editor (NBE). For example, nucleotide base editors can be formed of dCas coupled to nucleotide-specific enzyme evolved to efficiently act on a nucleotide at the target site for the dCas (based on the guide RNA). Examples of nucleotide base editors include adenine base editors (ABEs) that mediate the conversion of A.T to G.C in genomic DNA (Gaudelli et al., Nature 551:464-471 (2017)). Gaudelli adapted a transfer RNA adenosine deaminase to operate on DNA when fused to a catalytically impaired CRISPR-Cas9 mutant. Such ABEs introduce point mutations more efficiently and cleanly, and with less off-target genome modification, than other Cas9 nuclease-based methods. Thus, specific base editors allow the direct, programmable introduction of all four transition mutations without double-stranded DNA cleavage.

g. Further Aspects of CRISPR-Cas Systems

In some forms, the CRISPR enzyme is codon-optimized for expression in a eukaryotic cell. Cas optimization may be used to enhance function or to develop new functions, for example one can generate chimeric Cas9 proteins. Example chimeric Cas9 proteins are disclosed in International Patent Application Publication No. WO/2014/02475. Chimeric Cas9 proteins can be made by combining fragments from different Cas9 homologs. For example, the N-terminus of St1Cas9 can be combined with C-term of SpCas9.

In some forms, the Type II CRISPR enzyme is a Cas12 enzyme. In some forms, the Type II CRISPR enzyme is a Cas13 enzyme. In some forms, the Type II CRISPR enzyme is a dCas enzyme.

Additional effectors for use with the disclosed compositions and methods can be identified by their proximity to cast genes, for example, though not limited to, within the region 20 kb from the start of the cast gene and 20 kb from the end of the cast gene. In some forms, the effector protein comprises at least one HEPN domain and at least 500 amino acids, and wherein the C2c2 effector protein is naturally present in a prokaryotic genome within 20 kb upstream or downstream of a Cas gene or a CRISPR array. Non-limiting examples of Cas proteins include Cast, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologues thereof, or modified versions thereof. In some forms, the C2c2 effector protein is naturally present in a prokaryotic genome within 20 kb upstream or downstream of a Cas 1 gene. The terms “orthologue” (also referred to as “ortholog” herein) and “homologue” (also referred to as “homolog” herein) are well known in the art. By means of further guidance, a “homologue” of a protein as used herein is a protein of the same species which performs the same or a similar function as the protein it is a homologue of. Homologous proteins may but need not be structurally related, or are only partially structurally related. An “orthologue” of a protein as used herein is a protein of a different species which performs the same or a similar function as the protein it is an orthologue of. Orthologous proteins may but need not be structurally related, or are only partially structurally related.

In some forms, the CRISPR-Cas protein is a dead Cas13. In some forms, the dead Cas13 is a dead Cas13a protein which comprises one or more mutations in the HEPN domain. In some forms, the dead Cas13a comprises a mutation corresponding to R474A and R1046A in Leptotrichia wadei (LwaCas13a). In some forms, the dead Cas13 is a dead Cas13b protein which comprises one or more of R116A, H121A, R1177A, and H1182A of a Cas13b protein originating from Bergeyella zoohelcum ATCC 43767 or amino acid positions corresponding thereto of a Cas13b ortholog.

In some forms, the dCas13 is fused to an adenosine or cytidine deaminase protein or functional domain thereof, such as those disclosed in International Patent Application Nos. PCT/US2018/39616 and PCT/US2018/039618.

As used herein, the term “crRNA” or “guide RNA” or “single guide RNA” or “sgRNA” or “one or more nucleic acid components” of a Type V or Type VI CRISPR-Cas locus effector protein comprises any polynucleotide sequence having sufficient complementarity with a target nucleic acid sequence to hybridize with the target nucleic acid sequence and direct sequence-specific binding of a nucleic acid-targeting complex to the target nucleic acid sequence. In some forms, the degree of complementarity, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies; available at www.novocraft.com), ELAND (Illumina, San Diego, Calif.), SOAP (available at soap.genomics.org.cn), and Maq (available at maq.sourceforge.net). The ability of a guide sequence (within a nucleic acid-targeting guide RNA) to direct sequence-specific binding of a nucleic acid-targeting complex to a target nucleic acid sequence may be assessed by any suitable assay. For example, the components of a nucleic acid-targeting CRISPR system sufficient to form a nucleic acid-targeting complex, including the guide sequence to be tested, may be provided to a host cell having the corresponding target nucleic acid sequence, such as by transfection with vectors encoding the components of the nucleic acid-targeting complex, followed by an assessment of preferential targeting (e.g., cleavage) within the target nucleic acid sequence, such as by Surveyor assay as described herein. Similarly, cleavage of a target nucleic acid sequence may be evaluated in a test tube by providing the target nucleic acid sequence, components of a nucleic acid-targeting complex, including the guide sequence to be tested and a control guide sequence different from the test guide sequence, and comparing binding or rate of cleavage at the target sequence between the test and control guide sequence reactions. Other assays are possible, and will occur to those skilled in the art. A guide sequence, and hence a nucleic acid-targeting guide may be selected to target any target nucleic acid sequence. The target sequence may be DNA. The target sequence may be any RNA sequence. In some forms, the target sequence may be a sequence within a RNA molecule selected from the group consisting of messenger RNA (mRNA), pre-mRNA, ribosomal RNA (rRNA), transfer RNA (tRNA), micro-RNA (miRNA), small interfering RNA (siRNA), small nuclear RNA (snRNA), small nucleolar RNA (snoRNA), double stranded RNA (dsRNA), non-coding RNA (ncRNA), long non-coding RNA (lncRNA), and small cytoplasmic RNA (scRNA). In some preferred forms, the target sequence may be a sequence within a RNA molecule selected from the group consisting of mRNA, pre-mRNA, and rRNA. In some preferred forms, the target sequence may be a sequence within a RNA molecule selected from the group consisting of ncRNA, and lncRNA. In some more preferred forms, the target sequence may be a sequence within an mRNA molecule or a pre-mRNA molecule.

In some forms, a nucleic acid-targeting guide is selected to reduce the degree secondary structure within the nucleic acid-targeting guide. In some forms, about or less than about 75%, 50%, 40%, 30%, 25%, 20%, 15%, 10%, 5%, 1%, or fewer of the nucleotides of the nucleic acid-targeting guide participate in self-complementary base pairing when optimally folded. Optimal folding may be determined by any suitable polynucleotide folding algorithm. Some programs are based on calculating the minimal Gibbs free energy. An example of one such algorithm is mFold, as described by Zuker and Stiegler (Nucleic Acids Res. 9 (1981), 133-148). Another example folding algorithm is the online webserver RNAfold, developed at Institute for Theoretical Chemistry at the University of Vienna, using the centroid structure prediction algorithm (see e.g., A. R. Gruber et al., 2008, Cell 106(1): 23-24; and PA Carr and GM Church, 2009, Nature Biotechnology 27(12): 1151-62).

In some forms, a guide RNA or crRNA may comprise, consist essentially of, or consist of a direct repeat (DR) sequence and a guide sequence or spacer sequence. In some forms, the guide RNA or crRNA may comprise, consist essentially of, or consist of a direct repeat sequence fused or linked to a guide sequence or spacer sequence. In some forms, the direct repeat sequence may be located upstream (i.e., 5′) from the guide sequence or spacer sequence. In other forms, the direct repeat sequence may be located downstream (i.e., 3′) from the guide sequence or spacer sequence.

In some forms, the crRNA comprises a stem loop, preferably a single stem loop. In some forms, the direct repeat sequence forms a stem loop, preferably a single stem loop.

In some forms, the spacer length of the guide RNA is from 15 to 35 nt. In some forms, the spacer length of the guide RNA is at least 15 nucleotides. In some forms, the spacer length is from 15 to 17 nt, e.g., 15, 16, or 17 nt, from 17 to 20 nt, e.g., 17, 18, 19, or 20 nt, from 20 to 24 nt, e.g., 20, 21, 22, 23, or 24 nt, from 23 to 25 nt, e.g., 23, 24, or 25 nt, from 24 to 27 nt, e.g., 24, 25, 26, or 27 nt, from 27-30 nt, e.g., 27, 28, 29, or 30 nt, from 30-35 nt, e.g., 30, 31, 32, 33, 34, or 35 nt, or 35 nt or longer.

The “tracrRNA” sequence or analogous terms includes any polynucleotide sequence that has sufficient complementarity with a crRNA sequence to hybridize. In some forms, the degree of complementarity between the tracrRNA sequence and crRNA sequence along the length of the shorter of the two when optimally aligned is about or more than about 25%, 30%, 40%, 50%, 60%, 70%, 80%, 90%, 95%, 97.5%, 99%, or higher. In some forms, the tracr sequence is about or more than about 5, 6, 7, 8, 9, 10, 11, 12, 13, 14, 15, 16, 17, 18, 19, 20, 25, 30, 40, 50, or more nucleotides in length. In some forms, the tracr sequence and crRNA sequence are contained within a single transcript, such that hybridization between the two produces a transcript having a secondary structure, such as a hairpin. In some forms, the transcript or transcribed polynucleotide sequence has at least two or more hairpins. In preferred forms, the transcript has two, three, four or five hairpins. In some forms, the transcript has at most five hairpins. In a hairpin structure the portion of the sequence 5′ of the final “N” and upstream of the loop corresponds to the tracr mate sequence, and the portion of the sequence 3′ of the loop corresponds to the tracr sequence.

In general, degree of complementarity is with reference to the optimal alignment of the sca sequence and tracr sequence, along the length of the shorter of the two sequences. Optimal alignment may be determined by any suitable alignment algorithm, and may further account for secondary structures, such as self-complementarity within either the sca sequence or tracr sequence. In some forms, the degree of complementarity between the tracr sequence and sca sequence along the length of the shorter of the two when optimally aligned is about or more than about 25%, 30%, 40%, 50%, 60%, 70%, 80%, 90%, 95%, 97.5%, 99%, or higher.

In general, the CRISPR-Cas, CRISPR-Cas9 or CRISPR system may be as used in the foregoing documents, such as WO 2014/093622 (PCT/US2013/074667) and refers collectively to transcripts and other elements involved in the expression of or directing the activity of CRISPR-associated (“Cas”) genes, including sequences encoding a Cas gene, in particular a Cas9 gene in the case of CRISPR-Cas9, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or “RNA(s)” as that term is herein used (e.g., RNA(s) to guide Cas9, e.g. CRISPR RNA and transactivating (tracr) RNA or a single guide RNA (sgRNA) (chimeric RNA)) or other sequences and transcripts from a CRISPR locus. In general, a CRISPR system is characterized by elements that promote the formation of a CRISPR complex at the site of a target sequence (also referred to as a protospacer in the context of an endogenous CRISPR system). In the context of formation of a CRISPR complex, “target sequence” refers to a sequence to which a guide sequence is designed to have complementarity, where hybridization between a target sequence and a guide sequence promotes the formation of a CRISPR complex. The section of the guide sequence through which complementarity to the target sequence is important for cleavage activity is referred to herein as the seed sequence. A target sequence may comprise any polynucleotide, such as DNA or RNA polynucleotides. In some forms, a target sequence is located in the nucleus or cytoplasm of a cell, and may include nucleic acids in or from mitochondrial, organelles, vesicles, liposomes or particles present within the cell. In some forms, especially for non-nuclear uses, NLSs are not preferred. In some forms, a CRISPR system comprises one or more nuclear exports signals (NESs). In some forms, a CRISPR system comprises one or more NLSs and one or more NESs. In some forms, direct repeats may be identified in silico by searching for repetitive motifs that fulfill any or all of the following criteria: 1. found in a 2 Kb window of genomic sequence flanking the type II CRISPR locus; 2. span from 20 to 50 bp; and 3. interspaced by 20 to 50 bp. In some forms, 2 of these criteria may be used, for instance 1 and 2, 2 and 3, or 1 and 3. In some forms, all 3 criteria may be used.

In some forms, the terms guide sequence and guide RNA (i.e. RNA capable of guiding Cas to a target genomic locus) are used interchangeably as in foregoing cited documents such as WO 2014/093622 (PCT/US2013/074667). In general, a guide sequence is any polynucleotide sequence having sufficient complementarity with a target polynucleotide sequence to hybridize with the target sequence and direct sequence-specific binding of a CRISPR complex to the target sequence. In some forms, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), ClustalW, Clustal X, BLAT, Novoalign (Novocraft Technologies; available at www.novocraft.com), ELAND (Illumina, San Diego, Calif.), SOAP (available at soap.genomics.org.cn), and Maq (available at maq.sourceforge.net). In some forms, a guide sequence is about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some forms, a guide sequence is less than about 75, 50, 45, 40, 35, 30, 25, 20, 15, 12, or fewer nucleotides in length. Preferably the guide sequence is 10 to 30 nucleotides long. The ability of a guide sequence to direct sequence-specific binding of a CRISPR complex to a target sequence may be assessed by any suitable assay. For example, the components of a CRISPR system sufficient to form a CRISPR complex, including the guide sequence to be tested, may be provided to a host cell having the corresponding target sequence, such as by transfection with vectors encoding the components of the CRISPR sequence, followed by an assessment of preferential cleavage within the target sequence, such as by Surveyor assay as described herein. Similarly, cleavage of a target polynucleotide sequence may be evaluated in a test tube by providing the target sequence, components of a CRISPR complex, including the guide sequence to be tested and a control guide sequence different from the test guide sequence, and comparing binding or rate of cleavage at the target sequence between the test and control guide sequence reactions. Other assays are possible, and will occur to those skilled in the art.

In some forms of CRISPR-Cas systems, the degree of complementarity between a guide sequence and its corresponding target sequence can be about or more than about 50%, 60%, 75%, 80%, 85%, 90%, 95%, 97.5%, 99%, or 100%; a guide or RNA or sgRNA can be about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length; or guide or RNA or sgRNA can be less than about 75, 50, 45, 40, 35, 30, 25, 20, 15, 12, or fewer nucleotides in length; and advantageously tracr RNA is 30 or 50 nucleotides in length. However, in some forms, off-target interactions can be reduced, by, for example, reducing interaction of the guide with a target sequence having low complementarity. It has been demonstrated that mutations that result in the CRISPR-Cas system being able to distinguish between target and off-target sequences have greater than 80% to about 95% complementarity, e.g., 83%-84% or 88-89% or 94-95% complementarity (for instance, distinguishing between a target having 18 nucleotides from an off-target of 18 nucleotides having 1, 2 or 3 mismatches). Accordingly, in preferred forms, the degree of complementarity between a guide sequence and its corresponding target sequence can be greater than 94.5% or 95% or 95.5% or 96% or 96.5% or 97% or 97.5% or 98% or 98.5% or 99% or 99.5% or 99.9%, or 100%. Off target is less than 100% or 99.9% or 99.5% or 99% or 99% or 98.5% or 98% or 97.5% or 97% or 96.5% or 96% or 95.5% or 95% or 94.5% or 94% or 93% or 92% or 91% or 90% or 89% or 88% or 87% or 86% or 85% or 84% or 83% or 82% or 81% or 80% complementarity between the sequence and the guide, with it advantageous that off target is 100% or 99.9% or 99.5% or 99% or 99% or 98.5% or 98% or 97.5% or 97% or 96.5% or 96% or 95.5% or 95% or 94.5% complementarity between the sequence and the guide.

In some forms, the guide RNA (capable of guiding Cas to a target locus) may comprise (1) a guide sequence capable of hybridizing to a genomic target locus in the eukaryotic cell; (2) a tracr sequence; and (3) a tracr mate sequence. All (1) to (3) may reside in a single RNA, i.e. an sgRNA (arranged in a 5′ to 3′ orientation), or the tracr RNA may be a different RNA than the RNA containing the guide and tracr sequence. The tracr hybridizes to the tracr mate sequence and directs the CRISPR-Cas complex to the target sequence. Where the tracr RNA is on a different RNA than the RNA containing the guide and tracr sequence, the length of each RNA may be optimized to be shortened from their respective native lengths, and each may be independently chemically modified to protect from degradation by cellular RNase or otherwise increase stability.

In some forms, the disclosed compositions and methods can be used to induce one or more mutations in a eukaryotic cell (in vitro, i.e. in an isolated eukaryotic cell) as herein discussed comprising delivering to cell a vector as herein discussed. The mutation(s) can include the introduction, deletion, or substitution of one or more nucleotides at each target sequence of cell(s) via the guide(s) RNA(s) or sgRNA(s). The mutations can include the introduction, deletion, or substitution of 1-75 nucleotides at each target sequence of said cell(s) via the guide(s) RNA(s) or sgRNA(s). The mutations can include the introduction, deletion, or substitution of 1, 5, 10, 11, 12, 13, 14, 15, 16, 17, 18, 19, 20, 21, 22, 23, 24, 25, 26, 27, 28, 29, 30, 35, 40, 45, 50, or 75 nucleotides at each target sequence of said cell(s) via the guide(s) RNA(s) or sgRNA(s). The mutations can include the introduction, deletion, or substitution of 5, 10, 11, 12, 13, 14, 15, 16, 17, 18, 19, 20, 21, 22, 23, 24, 25, 26, 27, 28, 29, 30, 35, 40, 45, 50, or 75 nucleotides at each target sequence of said cell(s) via the guide(s) RNA(s) or sgRNA(s). The mutations include the introduction, deletion, or substitution of 10, 11, 12, 13, 14, 15, 16, 17, 18, 19, 20, 21, 22, 23, 24, 25, 26, 27, 28, 29, 30, 35, 40, 45, 50, or 75 nucleotides at each target sequence of said cell(s) via the guide(s) RNA(s) or sgRNA(s). The mutations can include the introduction, deletion, or substitution of 20, 21, 22, 23, 24, 25, 26, 27, 28, 29, 30, 35, 40, 45, 50, or 75 nucleotides at each target sequence of said cell(s) via the guide(s) RNA(s) or sgRNA(s). The mutations can include the introduction, deletion, or substitution of 40, 45, 50, 75, 100, 200, 300, 400 or 500 nucleotides at each target sequence of said cell(s) via the guide(s) RNA(s) or sgRNA(s).

For minimization of toxicity and off-target effect, it may be important to control the concentration of Cas mRNA and guide RNA delivered. Optimal concentrations of Cas mRNA and guide RNA can be determined by testing different concentrations in a cellular or non-human eukaryote animal model and using deep sequencing the analyze the extent of modification at potential off-target genomic loci. Alternatively, to minimize the level of toxicity and off-target effect, Cas nickase mRNA (for example S. pyogenes Cas9 with the D10A mutation) can be delivered with a pair of guide RNAs targeting a site of interest. Guide sequences and strategies to minimize toxicity and off-target effects can be as in WO 2014/093622 (PCT/US2013/074667); or, via mutation as herein.

Typically, in the context of an endogenous CRISPR system, formation of a CRISPR complex (comprising a guide sequence hybridized to a target sequence and complexed with one or more Cas proteins) results in cleavage of one or both strands in or near (e.g. within 1, 2, 3, 4, 5, 6, 7, 8, 9, 10, 20, 50, or more base pairs from) the target sequence. Without wishing to be bound by theory, the tracr sequence, which may comprise or consist of all or a portion of a wild-type tracr sequence (e.g. about or more than about 20, 26, 32, 45, 48, 54, 63, 67, 85, or more nucleotides of a wild-type tracr sequence), may also form part of a CRISPR complex, such as by hybridization along at least a portion of the tracr sequence to all or a portion of a tracr mate sequence that is operably linked to the guide sequence.

h. Guide Modifications

In some forms, the disclosed guides can comprise non-naturally occurring nucleic acids and/or non-naturally occurring nucleotides and/or nucleotide analogs, and/or chemically modifications. Non-naturally occurring nucleic acids can include, for example, mixtures of naturally and non-naturally occurring nucleotides. Non-naturally occurring nucleotides and/or nucleotide analogs may be modified at the ribose, phosphate, and/or base moiety. In some forms, a guide nucleic acid comprises ribonucleotides and non-ribonucleotides. In some such forms, a guide comprises one or more ribonucleotides and one or more deoxyribonucleotides. In some forms, the guide comprises one or more non-naturally occurring nucleotide or nucleotide analog such as a nucleotide with phosphorothioate linkage, boranophosphate linkage, a locked nucleic acid (LNA) nucleotides comprising a methylene bridge between the 2′ and 4′ carbons of the ribose ring, peptide nucleic acids (PNA), bridged nucleic acids (BNA), or morpholinos. Other examples of modified nucleotides include 2′-O-methyl analogs, 2′-deoxy analogs, 2-thiouridine analogs, N6-methyladenosine analogs, or 2′-fluoro analogs. Further examples of modified nucleotides include linkage of chemical moieties at the 2′ position, including but not limited to peptides, nuclear localization sequence (NLS), peptide nucleic acid (PNA), morpholinos, polyethylene glycol (PEG), triethylene glycol, or tetraethyleneglycol (TEG). Further examples of modified bases include, but are not limited to, 2-aminopurine, 5-bromo-uridine, pseudouridine (Ψ), N¹-methylpseudouridine (me¹Ψ), 5-methoxyuridine (5moU), inosine, 7-methylguanosine. Examples of guide RNA chemical modifications include, without limitation, incorporation of 2′-O-methyl (M), 2′-O-methyl-3′-phosphorothioate (MS), phosphorothioate (PS), S-constrained ethyl(cEt), 2′-O-methyl-3′-thioPACE (MSP), or 2′-O-methyl-3′-phosphonoacetate (MP) at one or more terminal nucleotides. Such chemically modified guides can comprise increased stability and increased activity as compared to unmodified guides, though on-target vs. off-target specificity is not predictable. (See, Hendel, 2015, Nat Biotechnol. 33(9):985-9, doi: 10.1038/nbt.3290, published online 29 Jun. 2015; Ragdarm et al., 0215, PNAS, E7110-E7111; Allerson et al., J. Med. Chem. 2005, 48:901-904; Bramsen et al., Front. Genet., 2012, 3:154; Deng et al., PNAS, 2015, 112:11870-11875; Sharma et al., Med Chem Comm., 2014, 5:1454-1471; Hendel et al., Nat. Biotechnol. (2015) 33(9): 985-989; Li et al., Nature Biomedical Engineering, 2017, 1, 0066 DOI:10.1038/s41551-017-0066; Ryan et al., Nucleic Acids Res. (2018) 46(2): 792-803). In some forms, the 5′ and/or 3′ end of a guide RNA is modified by a variety of functional moieties including fluorescent dyes, polyethylene glycol, cholesterol, proteins, or detection tags. (See Kelly et al., 2016, J. Biotech. 233:74-83). In some forms, a guide comprises ribonucleotides in a region that binds to a target DNA and one or more deoxyribonucleotides and/or nucleotide analogs in a region that binds to Cas9, Cpf1, or C2c1. In some forms, deoxyribonucleotides and/or nucleotide analogs are incorporated in engineered guide structures, such as, without limitation, 5′ and/or 3′ end, stem-loop regions, and the seed region. In some forms, the modification is not in the 5′-handle of the stem-loop regions. Chemical modification in the 5′-handle of the stem-loop region of a guide may abolish its function (see Li, et al., Nature Biomedical Engineering, 2017, 1:0066). In some forms, at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, or 75 nucleotides of a guide is chemically modified. In some forms, 3-5 nucleotides at either the 3′ or the 5′ end of a guide is chemically modified. In some forms, only minor modifications are introduced in the seed region, such as 2′-F modifications. In some forms, 2′-F modification is introduced at the 3′ end of a guide. In some forms, three to five nucleotides at the 5′ and/or the 3′ end of the guide are chemically modified with 2′-O-methyl (M), 2′-O-methyl-3′-phosphorothioate (MS), S-constrained ethyl(cEt), 2′-O-methyl-3′-thioPACE (MSP), or 2′-O-methyl-3′-phosphonoacetate (MP). Such modification can enhance genome editing efficiency (see Hendel et al., Nat. Biotechnol. (2015) 33(9): 985-989; Ryan et al., Nucleic Acids Res. (2018) 46(2): 792-803). In some forms, all of the phosphodiester bonds of a guide are substituted with phosphorothioates (PS) for enhancing levels of gene disruption. In some forms, more than five nucleotides at the 5′ and/or the 3′ end of the guide are chemically modified with 2′-O-Me, 2′-F or S-constrained ethyl (cEt). Such chemically modified guide can mediate enhanced levels of gene disruption (see Ragdarm et al., 0215, PNAS, E7110-E7111). In some forms, a guide is modified to comprise a chemical moiety at its 3′ and/or 5′ end. Such moieties include, but are not limited to amine, azide, alkyne, thio, dibenzocyclooctyne (DBCO), Rhodamine, peptides, nuclear localization sequence (NLS), peptide nucleic acid (PNA), morpholinos, polyethylene glycol (PEG), triethylene glycol, or tetraethyleneglycol (TEG). In some forms, the chemical moiety is conjugated to the guide by a linker, such as an alkyl chain. In some forms, the chemical moiety of the modified guide can be used to attach the guide to another molecule, such as DNA, RNA, protein, nanoparticle, or nucleic acid assembly. Such chemically modified guide can be used to identify or enrich cells generically edited by a CRISPR system (see Lee et al., eLife, 2017, 6:e25312, DOI:10.7554). In some forms, 3 nucleotides at each of the 3′ and 5′ ends are chemically modified. In specific forms, the modifications comprise 2′-O-methyl or phosphorothioate analogs. In specific forms, 12 nucleotides in the tetraloop and 16 nucleotides in the stem-loop region are replaced with 2′-O-methyl analogs. Such chemical modifications improve in vivo editing and stability (see Finn et al., Cell Reports (2018), 22: 2227-2235). In some forms, more than 60 or 70 nucleotides of the guide are chemically modified. In some forms, this modification comprises replacement of nucleotides with 2′-O-methyl or 2′-fluoro nucleotide analogs or phosphorothioate (PS) modification of phosphodiester bonds. In some forms, the chemical modification comprises 2′-O-methyl or 2′-fluoro modification of guide nucleotides extending outside of the nuclease protein when the CRISPR complex is formed or PS modification of 20 to 30 or more nucleotides of the 3′-terminus of the guide. In particular forms, the chemical modification further comprises 2′-O-methyl analogs at the 5′ end of the guide or 2′-fluoro analogs in the seed and tail regions. Such chemical modifications improve stability to nuclease degradation and maintain or enhance genome-editing activity or efficiency, but modification of all nucleotides may abolish the function of the guide (see Yin et al., Nat. Biotech. (2018), 35(12): 1179-1187). Such chemical modifications may be guided by knowledge of the structure of the CRISPR complex, including knowledge of the limited number of nuclease and RNA 2′-OH interactions (see Yin et al., Nat. Biotech. (2018), 35(12): 1179-1187). In some forms, one or more guide RNA nucleotides may be replaced with DNA nucleotides. In some forms, up to 2, 4, 6, 8, 10, or 12 RNA nucleotides of the 5′-end tail/seed guide region are replaced with DNA nucleotides. In some forms, the majority of guide RNA nucleotides at the 3′ end are replaced with DNA nucleotides. In particular forms, 16 guide RNA nucleotides at the 3′ end are replaced with DNA nucleotides. In particular forms, 8 guide RNA nucleotides of the 5′-end tail/seed region and 16 RNA nucleotides at the 3′ end are replaced with DNA nucleotides. In particular forms, guide RNA nucleotides that extend outside of the nuclease protein when the CRISPR complex is formed are replaced with DNA nucleotides. Such replacement of multiple RNA nucleotides with DNA nucleotides leads to decreased off-target activity but similar on-target activity compared to an unmodified guide; however, replacement of all RNA nucleotides at the 3′ end may abolish the function of the guide (see Yin et al., Nat. Chem. Biol. (2018) 14, 311-316). Such modifications may be guided by knowledge of the structure of the CRISPR complex, including knowledge of the limited number of nuclease and RNA 2′-OH interactions (see Yin et al., Nat. Chem. Biol. (2018) 14, 311-316).

In some forms, the guide comprises a modified crRNA for Cpf1, having a 5′-handle and a guide segment further comprising a seed region and a 3′-terminus. In some forms, the modified guide can be used with a Cpf1 of any one of Acidaminococcus sp. BV3L6 Cpf1 (AsCpf1); Francisella tularensis subsp. Novicida U112 Cpf1 (FnCpf1); L. bacterium MC2017 Cpf1 (Lb3Cpf1); Butyrivibrio proteoclasticus Cpf1 (BpCpf1); Parcubacteria bacterium GWC2011_GWC2_44_17 Cpf1 (PbCpf1); Peregrinibacteria bacterium GW2011_GWA_33_10 Cpf1 (PeCpf1); Leptospira inadai Cpf1 (LiCpf1); Smithella sp. SC_K08D17 Cpf1 (SsCpf1); L. bacterium MA2020 Cpf1 (Lb2Cpf1); Porphyromonas crevioricanis Cpf1 (PcCpf1); Porphyromonas macacae Cpf1 (PmCpf1); Candidatus Methanoplasma termitum Cpf1 (CMtCpf1); Eubacterium eligens Cpf1 (EeCpf1); Moraxella bovoculi 237 Cpf1 (MbCpf1); Prevotella disiens Cpf1 (PdCpf1); or L. bacterium ND2006 Cpf1 (LbCpf1).

In some forms, the modification to the guide is a chemical modification, an insertion, a deletion or a split. In some forms, the chemical modification includes, but is not limited to, incorporation of 2′-O-methyl (M) analogs, 2′-deoxy analogs, 2-thiouridine analogs, N6-methyladenosine analogs, 2′-fluoro analogs, 2-aminopurine, 5-bromo-uridine, pseudouridine N′-methylpseudouridine (me¹Ψ), 5-methoxyuridine (5moU), inosine, 7-methylguanosine, 2′-O-methyl-3′-phosphorothioate (MS), S-constrained ethyl(cEt), phosphorothioate (PS), 2′-O-methyl-3′-thioPACE (MSP), or 2′-O-methyl-3′-phosphonoacetate (MP). In some forms, the guide comprises one or more of phosphorothioate modifications. In some forms, at least 1, 2, 3, 4, 5, 6, 7, 8, 9, 10, 11, 12, 13, 14, 15, 16, 17, 18, 19, 20, or 25 nucleotides of the guide are chemically modified. In some forms, all nucleotides are chemically modified. In some forms, one or more nucleotides in the seed region are chemically modified. In some forms, one or more nucleotides in the 3′-terminus are chemically modified. In some forms, none of the nucleotides in the 5′-handle is chemically modified. In some forms, the chemical modification in the seed region is a minor modification, such as incorporation of a 2′-fluoro analog. In specific forms, one nucleotide of the seed region is replaced with a 2′-fluoro analog. In some forms, 5 or 10 nucleotides in the 3′-terminus are chemically modified. Such chemical modifications at the 3′-terminus of the Cpf1 CrRNA improve gene cutting efficiency (see Li, et al., Nature Biomedical Engineering, 2017, 1:0066). In specific forms, 5 nucleotides in the 3′-terminus are replaced with 2′-fluoro analogues. In specific forms, 10 nucleotides in the 3′-terminus are replaced with 2′-fluoro analogues. In specific forms, 5 nucleotides in the 3′-terminus are replaced with 2′-O-methyl (M) analogs. In some forms, 3 nucleotides at each of the 3′ and 5′ ends are chemically modified. In specific forms, the modifications comprise 2′-O-methyl or phosphorothioate analogs. In specific forms, 12 nucleotides in the tetraloop and 16 nucleotides in the stem-loop region are replaced with 2′-O-methyl analogs. Such chemical modifications improve in vivo editing and stability (see Finn et al., Cell Reports (2018), 22: 2227-2235).

In some forms, the loop of the 5′-handle of the guide is modified. In some forms, the loop of the 5′-handle of the guide is modified to have a deletion, an insertion, a split, or chemical modifications. In some forms, the loop comprises 3, 4, or 5 nucleotides. In some forms, the loop comprises the sequence of UCUU, UUUU, UAUU, or UGUU. In some forms, the guide molecule forms a stemloop with a separate non-covalently linked sequence, which can be DNA or RNA.

i. Synthetically Linked Guide

In some forms, the guide comprises a tracr sequence and a tracr mate sequence that are chemically linked or conjugated via a non-phosphodiester bond. In some forms, the guide comprises a tracr sequence and a tracr mate sequence that are chemically linked or conjugated via a non-nucleotide loop. In some forms, the tracr and tracr mate sequences are joined via a non-phosphodiester covalent linker. Examples of the covalent linker include but are not limited to a chemical moiety selected from the group consisting of carbamates, ethers, esters, amides, imines, amidines, aminotrizines, hydrozone, disulfides, thioethers, thioesters, phosphorothioates, phosphorodithioates, sulfonamides, sulfonates, sulfones, sulfoxides, ureas, thioureas, hydrazide, oxime, triazole, photolabile linkages, C—C bond forming groups such as Diels-Alder cyclo-addition pairs or ring-closing metathesis pairs, and Michael reaction pairs.

In some forms, the tracr and tracr mate sequences are first synthesized using the standard phosphoramidite synthetic protocol (Herdewijn, P., ed., Methods in Molecular Biology Col 288, Oligonucleotide Synthesis: Methods and Applications, Humana Press, New Jersey (2012)). In some forms, the tracr or tracr mate sequences can be functionalized to contain an appropriate functional group for ligation using the standard protocol known in the art (Hermanson, G. T., Bioconjugate Techniques, Academic Press (2013)). Examples of functional groups include, but are not limited to, hydroxyl, amine, carboxylic acid, carboxylic acid halide, carboxylic acid active ester, aldehyde, carbonyl, chlorocarbonyl, imidazolylcarbonyl, hydrozide, semicarbazide, thio semicarbazide, thiol, maleimide, haloalkyl, sufonyl, ally, propargyl, diene, alkyne, and azide. Once the tracr and the tracr mate sequences are functionalized, a covalent chemical bond or linkage can be formed between the two oligonucleotides. Examples of chemical bonds include, but are not limited to, those based on carbamates, ethers, esters, amides, imines, amidines, aminotrizines, hydrozone, disulfides, thioethers, thioesters, phosphorothioates, phosphorodithioates, sulfonamides, sulfonates, sulfones, sulfoxides, ureas, thioureas, hydrazide, oxime, triazole, photolabile linkages, C—C bond forming groups such as Diels-Alder cyclo-addition pairs or ring-closing metathesis pairs, and Michael reaction pairs.

In some forms, the tracr and tracr mate sequences can be chemically synthesized. In some forms, the chemical synthesis uses automated, solid-phase oligonucleotide synthesis machines with 2′-acetoxyethyl orthoester (2′-ACE) (Scaringe et al., J. Am. Chem. Soc. (1998) 120: 11820-11821; Scaringe, Methods Enzymol. (2000) 317: 3-18) or 2′-thionocarbamate (2′-TC) chemistry (Dellinger et al., J. Am. Chem. Soc. (2011) 133: 11540-11546; Hendel et al., Nat. Biotechnol. (2015) 33:985-989).

In some forms, the tracr and tracr mate sequences can be covalently linked using various bioconjugation reactions, loops, bridges, and non-nucleotide links via modifications of sugar, internucleotide phosphodiester bonds, and purine and pyrimidine residues. Sletten et al., Angew. Chem. Int. Ed. (2009) 48:6974-6998; Manoharan, M. Curr. Opin. Chem. Biol. (2004) 8: 570-9; Behlke et al., Oligonucleotides (2008) 18: 305-19; Watts, et al., Drug. Discov. Today (2008) 13: 842-55; Shukla, et al., Chem Med Chem (2010) 5: 328-49.

In some forms, the tracr and tracr mate sequences can be covalently linked using click chemistry. In some forms, the tracr and tracr mate sequences can be covalently linked using a triazole linker. In some forms, the tracr and tracr mate sequences can be covalently linked using Huisgen 1,3-dipolar cycloaddition reaction involving an alkyne and azide to yield a highly stable triazole linker (He et al., Chem Bio Chem (2015) 17: 1809-1812; WO 2016/186745). In some forms, the tracr and tracr mate sequences are covalently linked by ligating a 5′-hexyne tracrRNA and a 3′-azide crRNA. In some forms, either or both of the 5′-hexyne tracrRNA and a 3′-azide crRNA can be protected with 2′-acetoxyethl orthoester (2′-ACE) group, which can be subsequently removed using Dharmacon protocol (Scaringe et al., J. Am. Chem. Soc. (1998) 120: 11820-11821; Scaringe, Methods Enzymol. (2000) 317: 3-18).

In some forms, the tracr and tracr mate sequences can be covalently linked via a linker (e.g., a non-nucleotide loop) that comprises a moiety such as spacers, attachments, bioconjugates, chromophores, reporter groups, dye labeled RNAs, and non-naturally occurring nucleotide analogues. More specifically, suitable spacers include, but are not limited to, polyethers (e.g., polyethylene glycols, polyalcohols, polypropylene glycol or mixtures of ethylene and propylene glycols), polyamines group (e.g., spennine, spermidine and polymeric derivatives thereof), polyesters (e.g., poly(ethyl acrylate)), polyphosphodiesters, alkylenes, and combinations thereof. Suitable attachments include any moiety that can be added to the linker to add additional properties to the linker, such as but not limited to, fluorescent labels. Suitable bioconjugates include, but are not limited to, peptides, glycosides, lipids, cholesterol, phospholipids, diacyl glycerols and dialkyl glycerols, fatty acids, hydrocarbons, enzyme substrates, steroids, biotin, digoxigenin, carbohydrates, polysaccharides. Suitable chromophores, reporter groups, and dye-labeled RNAs include, but are not limited to, fluorescent dyes such as fluorescein and rhodamine, chemiluminescent, electrochemiluminescent, and bioluminescent marker compounds. The design of example linkers conjugating two RNA components are also described in WO 2004/015075.

The linker (e.g., a non-nucleotide loop) can be of any length. In some forms, the linker has a length equivalent to about 0-16 nucleotides. In some forms, the linker has a length equivalent to about 0-8 nucleotides. In some forms, the linker has a length equivalent to about 0-4 nucleotides. In some forms, the linker has a length equivalent to about 2 nucleotides. Example linker design is also described in WO2011/008730.

A typical Type II Cas9 sgRNA comprises (in 5′ to 3′ direction): a guide sequence, a poly U tract, a first complimentary stretch (the “repeat”), a loop (tetraloop), a second complimentary stretch (the “anti-repeat” being complimentary to the repeat), a stem, and further stem loops and stems and a poly A (often poly U in RNA) tail (terminator). In preferred forms, some forms of guide architecture are retained, some forms of guide architecture cam be modified, for example, by addition, subtraction, or substitution of features, and some forms of guide architecture are maintained. Preferred locations for engineered sgRNA modifications, including but not limited to insertions, deletions, and substitutions include guide termini and regions of the sgRNA that are exposed when complexed with CRISPR protein and/or target, for example the tetraloop and/or loop2.

In some forms, the guides comprise specific binding sites (e.g. aptamers) for adapter proteins, which may comprise one or more functional domains (e.g. via fusion protein). When such a guides forms a CRISPR complex (i.e. CRISPR enzyme binding to guide and target) the adapter proteins bind and, the functional domain associated with the adapter protein is positioned in a spatial orientation which is advantageous for the attributed function to be effective. For example, if the functional domain is a transcription activator (e.g. VP64 or p65), the transcription activator is placed in a spatial orientation which allows it to affect the transcription of the target. Likewise, a transcription repressor will be advantageously positioned to affect the transcription of the target and a nuclease (e.g. Fok1) will be advantageously positioned to cleave or partially cleave the target.

The skilled person will understand that modifications to the guide which allow for binding of the adapter+functional domain but not proper positioning of the adapter+functional domain (e.g. due to steric hindrance within the three dimensional structure of the CRISPR complex) are modifications which are not intended. The one or more modified guide may be modified at the tetra loop, the stem loop 1, stem loop 2, or stem loop 3, as described herein, preferably at either the tetra loop or stem loop 2, and most preferably at both the tetra loop and stem loop 2.

The repeat:anti repeat duplex will be apparent from the secondary structure of the sgRNA. It may be typically a first complimentary stretch after (in 5′ to 3′ direction) the poly U tract and before the tetraloop; and a second complimentary stretch after (in 5′ to 3′ direction) the tetraloop and before the poly A tract. The first complimentary stretch (the “repeat”) is complimentary to the second complimentary stretch (the “anti-repeat”). As such, they Watson-Crick base pair to form a duplex of dsRNA when folded back on one another. As such, the anti-repeat sequence is the complimentary sequence of the repeat and in terms to A-U or C-G base pairing, but also in terms of the fact that the anti-repeat is in the reverse orientation due to the tetraloop.

In some forms, modification of guide architecture comprises replacing bases in stemloop 2. For example, in some forms, “actt” (“acuu” in RNA) and “aagt” (“aagu” in RNA) bases in stemloop2 are replaced with “cgcc” and “gcgg.” In some forms, “actt” and “aagt” bases in stemloop2 are replaced with complimentary GC-rich regions of 4 nucleotides. In some forms, the complimentary GC-rich regions of 4 nucleotides are “cgcc” and “gcgg” (both in 5′ to 3′ direction). In some forms, the complimentary GC-rich regions of 4 nucleotides are “gcgg” and “cgcc” (both in 5′ to 3′ direction). Other combination of C and G in the complimentary GC-rich regions of 4 nucleotides will be apparent including CCCC and GGGG.

In some forms, the stemloop 2, e.g., “ACTTgtttAAGT” (SEQ ID NO:170) can be replaced by any “XXXXgtttYYYY,” e.g., where XXXX and YYYY represent any complementary sets of nucleotides that together will base pair to each other to create a stem.

In some forms, the stem comprises at least about 4 bp comprising complementary X and Y sequences, although stems of more, e.g., 5, 6, 7, 8, 9, 10, 11 or 12 or fewer, e.g., 3, 2, base pairs are also contemplated. Thus, for example X2-12 and Y2-12 (wherein X and Y represent any complementary set of nucleotides) may be contemplated. In some forms, the stem made of the X and Y nucleotides, together with the “gttt,” will form a complete hairpin in the overall secondary structure; and, this may be advantageous and the amount of base pairs can be any amount that forms a complete hairpin. In some forms, any complementary X:Y basepairing sequence (e.g., as to length) is tolerated, so long as the secondary structure of the entire sgRNA is preserved. In some forms, the stem can be a form of X:Y basepairing that does not disrupt the secondary structure of the whole sgRNA in that it has a DR:tracr duplex, and 3 stemloops. In some forms, the “gttt” tetraloop that connects ACTT and AAGT (or any alternative stem made of X:Y basepairs) can be any sequence of the same length (e.g., 4 basepair) or longer that does not interrupt the overall secondary structure of the sgRNA. In some forms, the stemloop can be something that further lengthens stemloop2, e.g. can be MS2 aptamer. In some forms, the stemloop3 “GGCACCGagtCGGTGC” (SEQ ID NO:171) can likewise take on a “XXXXXXXagtYYYYYYY” form, e.g., wherein X7 and Y7 represent any complementary sets of nucleotides that together will base pair to each other to create a stem. In some forms, the stem comprises about 7 bp comprising complementary X and Y sequences, although stems of more or fewer basepairs are also contemplated. In some forms, the stem made of the X and Y nucleotides, together with the “agt,” will form a complete hairpin in the overall secondary structure. In some forms, any complementary X:Y basepairing sequence is tolerated, so long as the secondary structure of the entire sgRNA is preserved. In some forms, the stem can be a form of X:Y basepairing that doesn't disrupt the secondary structure of the whole sgRNA in that it has a DR:tracr duplex, and 3 stemloops. In some forms, the “agt” sequence of the stemloop 3 can be extended or be replaced by an aptamer, e.g., a MS2 aptamer or sequence that otherwise generally preserves the architecture of stemloop3. In some forms of alternative Stemloops 2 and/or 3, each X and Y pair can refer to any basepair. In some forms, non-Watson Crick basepairing is contemplated, where such pairing otherwise generally preserves the architecture of the stemloop at that position.

In some forms, the DR:tracrRNA duplex can be replaced with the form: gYYYYag(N)NNNNxxxxNNNN(AAN)uuRRRRu (using standard IUPAC nomenclature for nucleotides), wherein (N) and (AAN) represent part of the bulge in the duplex, and “xxxx” represents a linker sequence. NNNN on the direct repeat can be anything so long as it basepairs with the corresponding NNNN portion of the tracrRNA. In some forms, the DR:tracrRNA duplex can be connected by a linker of any length (xxxx . . . ), any base composition, as long as it doesn't alter the overall structure.

In some forms, the sgRNA structural requirement is to have a duplex and 3 stemloops. In most forms, the actual sequence requirement for many of the particular base requirements are lax, in that the architecture of the DR:tracrRNA duplex should be preserved, but the sequence that creates the architecture, i.e., the stems, loops, bulges, etc., may be altered.

j. Aptamers

One guide with a first aptamer/RNA-binding protein pair can be linked or fused to an activator, while a second guide with a second aptamer/RNA-binding protein pair can be linked or fused to a repressor. The guides are for different targets (loci), so this allows one gene to be activated and one repressed. For example, the following schematic shows such an approach:

Guide 1—MS2 aptamer - - - MS2 RNA-binding protein - - - VP64 activator; and
Guide 2—PP7 aptamer - - - PP7 RNA-binding protein - - - SID4x repressor.

In some forms, the CRISPR-Cas system can involve orthogonal PP7/MS2 gene targeting. In these forms, sgRNA targeting different loci are modified with distinct RNA loops in order to recruit MS2-VP64 or PP7-SID4X, which activate and repress their target loci, respectively. PP7 is the RNA-binding coat protein of the bacteriophage Pseudomonas. Like MS2, it binds a specific RNA sequence and secondary structure. The PP7 RNA-recognition motif is distinct from that of MS2. Consequently, PP7 and MS2 can be multiplexed to mediate distinct effects at different genomic loci simultaneously. For example, an sgRNA targeting locus A can be modified with MS2 loops, recruiting MS2-VP64 activators, while another sgRNA targeting locus B can be modified with PP7 loops, recruiting PP7-SID4X repressor domains. In the same cell, dCas9 can thus mediate orthogonal, locus-specific modifications. This principle can be extended to incorporate other orthogonal RNA-binding proteins such as Q-beta.

An alternative option for orthogonal repression includes incorporating non-coding RNA loops with transactive repressive function into the guide (either at similar positions to the MS2/PP7 loops integrated into the guide or at the 3′ terminus of the guide). For instance, guides were designed with non-coding (but known to be repressive) RNA loops (e.g. using the Alu repressor (in RNA) that interferes with RNA polymerase II in mammalian cells). The Alu RNA sequence was located: in place of the MS2 RNA sequences as used herein (e.g. at tetraloop and/or stem loop 2); and/or at 3′ terminus of the guide. This gives possible combinations of MS2, PP7 or Alu at the tetraloop and/or stemloop 2 positions, as well as, optionally, addition of Alu at the 3′ end of the guide (with or without a linker).

The use of two different aptamers (distinct RNA) allows an activator-adaptor protein fusion and a repressor-adaptor protein fusion to be used, with different guides, to activate expression of one gene, whilst repressing another. They, along with their different guides can be administered together, or substantially together, in a multiplexed approach. A large number of such modified guides can be used all at the same time, for example 10 or 20 or 30 and so forth, whilst only one (or at least a minimal number) of Cas9s to be delivered, as a comparatively small number of Cas9s can be used with a large number modified guides. The adaptor protein may be associated (preferably linked or fused to) one or more activators or one or more repressors. For example, the adaptor protein may be associated with a first activator and a second activator. The first and second activators may be the same, but they are preferably different activators. For example, one might be VP64, whilst the other might be p65, although these are just examples and other transcriptional activators are envisaged. Three or more or even four or more activators (or repressors) may be used, but package size may limit the number being higher than 5 different functional domains. Linkers are preferably used, over a direct fusion to the adaptor protein, where two or more functional domains are associated with the adaptor protein. Suitable linkers might include the GlySer linker.

It is also envisaged that the enzyme-guide complex as a whole may be associated with two or more functional domains. For example, there may be two or more functional domains associated with the enzyme, or there may be two or more functional domains associated with the guide (via one or more adaptor proteins), or there may be one or more functional domains associated with the enzyme and one or more functional domains associated with the guide (via one or more adaptor proteins).

The fusion between the adaptor protein and the activator or repressor may include a linker. For example, GlySer linkers GGGS (SEQ ID NO:172) can be used. They can be used in repeats of 3 ((GGGS)₃ (SEQ ID NO:173) or 6, 9 or even 12 or more, to provide suitable lengths, as required. Linkers can be used between the RNA-binding protein and the functional domain (activator or repressor), or between the CRISPR Enzyme (Cas9) and the functional domain (activator or repressor). The linkers the user to engineer appropriate amounts of “mechanical flexibility.”

k. Dead Guides

In some forms, the guide sequences are modified in a manner which allows for formation of the CRISPR complex and successful binding to the target, while at the same time, not allowing for successful nuclease activity (i.e. without nuclease activity/without indel activity). For matters of explanation such modified guide sequences are referred to as “dead guides” or “dead guide sequences.” These dead guides or dead guide sequences can be thought of as catalytically inactive or conformationally inactive with regard to nuclease activity. Nuclease activity may be measured using surveyor analysis or deep sequencing as commonly used in the art, preferably surveyor analysis. Similarly, dead guide sequences may not sufficiently engage in productive base pairing with respect to the ability to promote catalytic activity or to distinguish on-target and off-target binding activity. Briefly, the surveyor assay involves purifying and amplifying a CRISPR target site for a gene and forming heteroduplexes with primers amplifying the CRISPR target site. After re-anneal, the products are treated with SURVEYOR nuclease and SURVEYOR enhancer S (Transgenomics) following the manufacturer's recommended protocols, analyzed on gels, and quantified based upon relative band intensities.

Hence, in related forms, a non-naturally occurring or engineered composition Cas9 CRISPR-Cas system comprising a functional Cas9 as described herein and guide RNA (gRNA) can be used, where the gRNA comprises a dead guide sequence whereby the gRNA is capable of hybridizing to a target sequence such that the Cas9 CRISPR-Cas system is directed to a genomic locus of interest in a cell without detectable indel activity resultant from nuclease activity of a non-mutant Cas9 enzyme of the system as detected by a SURVEYOR assay. For shorthand purposes, a gRNA comprising a dead guide sequence whereby the gRNA is capable of hybridizing to a target sequence such that the Cas9 CRISPR-Cas system is directed to a genomic locus of interest in a cell without detectable indel activity resultant from nuclease activity of a non-mutant Cas9 enzyme of the system as detected by a SURVEYOR assay is herein termed a “dead gRNA.” It is to be understood that any of gRNAs as described herein or elsewhere may be used as dead gRNAs/gRNAs comprising a dead guide sequence as described herein. Any of the methods, products, compositions and uses as described herein elsewhere is equally applicable with the dead gRNAs/gRNAs comprising a dead guide sequence as further detailed below.

The ability of a dead guide sequence to direct sequence-specific binding of a CRISPR complex to a target sequence may be assessed by any suitable assay. For example, the components of a CRISPR system sufficient to form a CRISPR complex, including the dead guide sequence to be tested, may be provided to a host cell having the corresponding target sequence, such as by transfection with vectors encoding the components of the CRISPR sequence, followed by an assessment of preferential cleavage within the target sequence, such as by Surveyor assay as described herein. Similarly, cleavage of a target polynucleotide sequence may be evaluated in a test tube by providing the target sequence, components of a CRISPR complex, including the dead guide sequence to be tested and a control guide sequence different from the test dead guide sequence, and comparing binding or rate of cleavage at the target sequence between the test and control guide sequence reactions. Other assays are possible, and will occur to those skilled in the art. A dead guide sequence may be selected to target any target sequence. In some forms, the target sequence is a sequence within a genome of a cell.

As explained further herein, several structural parameters allow for a proper framework to arrive at such dead guides. Dead guide sequences are shorter than respective guide sequences which result in active Cas9-specific indel formation. Dead guides are 5%, 10%, 20%, 30%, 40%, 50%, shorter than respective guides directed to the same Cas9 leading to active Cas9-specific indel formation.

As explained below and known in the art, some forms of gRNA—Cas9 specificity is the direct repeat sequence, which is to be appropriately linked to such guides. In particular, this implies that the direct repeat sequences are designed dependent on the origin of the Cas9. Thus, structural data available for validated dead guide sequences may be used for designing Cas9 specific equivalents. Structural similarity between, e.g., the orthologous nuclease domains RuvC of two or more Cas9 effector proteins may be used to transfer design equivalent dead guides. Thus, the dead guide herein may be appropriately modified in length and sequence to reflect such Cas9 specific equivalents, allowing for formation of the CRISPR complex and successful binding to the target, while at the same time, not allowing for successful nuclease activity.

The use of dead guides in the context herein as well as the state of the art provides a surprising and unexpected platform for network biology and/or systems biology in both in vitro, ex vivo, and in vivo applications, allowing for multiplex gene targeting, and in particular bidirectional multiplex gene targeting. Prior to the use of dead guides, addressing multiple targets, for example for activation, repression and/or silencing of gene activity, has been challenging and in some cases not possible. With the use of dead guides, multiple targets, and thus multiple activities, may be addressed, for example, in the same cell, in the same animal, or in the same patient. Such multiplexing may occur at the same time or staggered for a desired timeframe.

For example, the dead guides now allow for the first time to use gRNA as a means for gene targeting, without the consequence of nuclease activity, while at the same time providing directed means for activation or repression. Guide RNA comprising a dead guide may be modified to further include elements in a manner which allow for activation or repression of gene activity, in particular protein adaptors (e.g. aptamers) as described herein elsewhere allowing for functional placement of gene effectors (e.g. activators or repressors of gene activity). One example is the incorporation of aptamers, as explained herein and in the state of the art. By engineering the gRNA comprising a dead guide to incorporate protein-interacting aptamers (Konermann et al., “Genome-scale transcription activation by an engineered CRISPR-Cas9 complex,” doi:10.1038/nature14136, incorporated herein by reference), one may assemble a synthetic transcription activation complex consisting of multiple distinct effector domains. Such may be modeled after natural transcription activation processes. For example, an aptamer, which selectively binds an effector (e.g. an activator or repressor; dimerized MS2 bacteriophage coat proteins as fusion proteins with an activator or repressor), or a protein which itself binds an effector (e.g. activator or repressor) may be appended to a dead gRNA tetraloop and/or a stem-loop 2. In the case of MS2, the fusion protein MS2-VP64 binds to the tetraloop and/or stem-loop 2 and in turn mediates transcriptional up-regulation, for example for Neurog2. Other transcriptional activators are, for example, VP64. P65, HSF1, and MyoD1. By mere example of this concept, replacement of the MS2 stem-loops with PP7-interacting stem-loops may be used to recruit repressive elements.

Thus, in some forms, a gRNA that comprises a dead guide can be used, where the gRNA further comprises modifications which provide for gene activation or repression, as described herein. The dead gRNA may comprise one or more aptamers. The aptamers may be specific to gene effectors, gene activators or gene repressors. Alternatively, the aptamers may be specific to a protein which in turn is specific to and recruits/binds a specific gene effector, gene activator or gene repressor. If there are multiple sites for activator or repressor recruitment, it is preferred that the sites are specific to either activators or repressors. If there are multiple sites for activator or repressor binding, the sites may be specific to the same activators or same repressors. The sites may also be specific to different activators or different repressors. The gene effectors, gene activators, gene repressors may be present in the form of fusion proteins.

In some forms, the dead gRNA as described herein or the Cas9 CRISPR-Cas complex as described herein includes a non-naturally occurring or engineered composition comprising two or more adaptor proteins, wherein each protein is associated with one or more functional domains and wherein the adaptor protein binds to the distinct RNA sequence(s) inserted into the at least one loop of the dead gRNA.

Hence, some forms provide a non-naturally occurring or engineered composition comprising a guide RNA (gRNA) comprising a dead guide sequence capable of hybridizing to a target sequence in a genomic locus of interest in a cell, wherein the dead guide sequence is as defined herein, a Cas9 comprising at least one or more nuclear localization sequences, wherein the Cas9 optionally comprises at least one mutation wherein at least one loop of the dead gRNA is modified by the insertion of distinct RNA sequence(s) that bind to one or more adaptor proteins, and wherein the adaptor protein is associated with one or more functional domains; or, wherein the dead gRNA is modified to have at least one non-coding functional loop, and wherein the composition comprises two or more adaptor proteins, wherein the each protein is associated with one or more functional domains.

In some forms, the adaptor protein is a fusion protein comprising the functional domain, the fusion protein optionally comprising a linker between the adaptor protein and the functional domain, the linker optionally including a GlySer linker.

In some forms, the at least one loop of the dead gRNA is not modified by the insertion of distinct RNA sequence(s) that bind to the two or more adaptor proteins. In some forms, the one or more functional domains associated with the adaptor protein is a transcriptional activation domain.

In some forms, the one or more functional domains associated with the adaptor protein is a transcriptional activation domain comprising VP64, p65, MyoD1, HSF1, RTA or SETT/9.

In some forms, the one or more functional domains associated with the adaptor protein is a transcriptional repressor domain.

In some forms, the transcriptional repressor domain is a KRAB domain.

In some forms, the transcriptional repressor domain is a NuE domain, NcoR domain, SID domain or a SID4X domain.

In some forms, at least one of the one or more functional domains associated with the adaptor protein have one or more activities comprising methylase activity, demethylase activity, transcription activation activity, transcription repression activity, transcription release factor activity, histone modification activity, DNA integration activity RNA cleavage activity, DNA cleavage activity or nucleic acid binding activity.

In some forms, the DNA cleavage activity is due to a Fok1 nuclease.

In some forms, the dead gRNA is modified so that, after dead gRNA binds the adaptor protein and further binds to the Cas9 and target, the functional domain is in a spatial orientation allowing for the functional domain to function in its attributed function.

In some forms, the at least one loop of the dead gRNA is tetra loop and/or loop2. In some forms, the tetra loop and loop 2 of the dead gRNA are modified by the insertion of the distinct RNA sequence(s).

In some forms, the insertion of distinct RNA sequence(s) that bind to one or more adaptor proteins is an aptamer sequence. In some forms, the aptamer sequence is two or more aptamer sequences specific to the same adaptor protein. In some forms, the aptamer sequence is two or more aptamer sequences specific to different adaptor protein.

In some forms, the adaptor protein comprises MS2, PP7, F2, GA, fr, JP501, M12, R17, BZ13, JP34, JP500, KU1, M11, MX1, TW18, VK, SP, FI, ID2, NL95, TW19, AP205, ϕCb5, ϕCb8r, ϕCb12r, ϕCb23r, 7s, PRR1.

In some forms, the cell is a eukaryotic cell. In some forms, the eukaryotic cell is a mammalian cell, optionally a mouse cell. In some forms, the mammalian cell is a human cell.

In some forms, a first adaptor protein is associated with a p65 domain and a second adaptor protein is associated with a HSF1 domain.

In some forms, the composition comprises a Cas9 CRISPR-Cas complex having at least three functional domains, at least one of which is associated with the Cas9 and at least two of which are associated with dead gRNA.

In some forms, the composition further comprises a second gRNA, wherein the second gRNA is a live gRNA capable of hybridizing to a second target sequence such that a second Cas9 CRISPR-Cas system is directed to a second genomic locus of interest in a cell with detectable indel activity at the second genomic locus resultant from nuclease activity of the Cas9 enzyme of the system.

In some forms, the composition further comprises a plurality of dead gRNAs and/or a plurality of live gRNAs.

In some forms, the CRISPR-Cas systems can take advantage of the modularity and customizability of the gRNA scaffold to establish a series of gRNA scaffolds with different binding sites (in particular aptamers) for recruiting distinct types of effectors in an orthogonal manner. Again, for matters of example and illustration of the broader concept, replacement of the MS2 stem-loops with PP7-interacting stem-loops may be used to bind/recruit repressive elements, enabling multiplexed bidirectional transcriptional control. Thus, in general, gRNA comprising a dead guide may be employed to provide for multiplex transcriptional control and preferred bidirectional transcriptional control. This transcriptional control is most preferred of genes. For example, one or more gRNA comprising dead guide(s) may be employed in targeting the activation of one or more target genes. At the same time, one or more gRNA comprising dead guide(s) may be employed in targeting the repression of one or more target genes. Such a sequence may be applied in a variety of different combinations, for example the target genes are first repressed and then at an appropriate period other targets are activated, or select genes are repressed at the same time as select genes are activated, followed by further activation and/or repression. As a result, multiple components of one or more biological systems may advantageously be addressed together.

In some forms, nucleic acid molecule(s) encoding dead gRNA or the Cas9 CRISPR-Cas complex or the composition can be used.

In some forms, a vector system can be used, where the vector system comprises a nucleic acid molecule encoding dead guide RNA as defined herein. In some forms, the vector system further comprises a nucleic acid molecule(s) encoding Cas9. In some forms, the vector system further comprises a nucleic acid molecule(s) encoding (live) gRNA. In some forms, the nucleic acid molecule or the vector further comprises regulatory element(s) operable in a eukaryotic cell operably linked to the nucleic acid molecule encoding the guide sequence (gRNA) and/or the nucleic acid molecule encoding Cas9 and/or the optional nuclear localization sequence(s).

In other forms, structural analysis may also be used to study interactions between the dead guide and the active Cas9 nuclease that enable DNA binding, but no DNA cutting. In this way amino acids important for nuclease activity of Cas9 are determined. Modification of such amino acids allows for improved Cas9 enzymes used for gene editing.

Some forms combine the use of dead guides as explained herein with other applications of CRISPR, as explained herein as well as known in the art. For example, gRNA comprising dead guide(s) for targeted multiplex gene activation or repression or targeted multiplex bidirectional gene activation/repression may be combined with gRNA comprising guides which maintain nuclease activity, as explained herein. Such gRNA comprising guides which maintain nuclease activity may or may not further include modifications which allow for repression of gene activity (e.g. aptamers). Such gRNA comprising guides which maintain nuclease activity may or may not further include modifications which allow for activation of gene activity (e.g. aptamers). In such a manner, a further means for multiplex gene control is introduced (e.g. multiplex gene targeted activation without nuclease activity/without indel activity may be provided at the same time or in combination with gene targeted repression with nuclease activity).

For example, 1) using one or more gRNA (e.g. 1-50, 1-40, 1-30, 1-20, preferably 1-10, more preferably 1-5) comprising dead guide(s) targeted to one or more genes and further modified with appropriate aptamers for the recruitment of gene activators; 2) may be combined with one or more gRNA (e.g. 1-50, 1-40, 1-30, 1-20, preferably 1-10, more preferably 1-5) comprising dead guide(s) targeted to one or more genes and further modified with appropriate aptamers for the recruitment of gene repressors. 1) and/or 2) may then be combined with 3) one or more gRNA (e.g. 1-50, 1-40, 1-30, 1-20, preferably 1-10, more preferably 1-5) targeted to one or more genes. This combination can then be carried out in turn with 1)+2)+3) with 4) one or more gRNA (e.g. 1-50, 1-40, 1-30, 1-20, preferably 1-10, more preferably 1-5) targeted to one or more genes and further modified with appropriate aptamers for the recruitment of gene activators. This combination can then be carried in turn with 1)+2)+3)+4) with 5) one or more gRNA (e.g. 1-50, 1-40, 1-30, 1-20, preferably 1-10, more preferably 1-5) targeted to one or more genes and further modified with appropriate aptamers for the recruitment of gene repressors. As a result various uses and combinations can be used in and with the disclosed compositions and methods. For example, combination 1)+2); combination 1)+3); combination 2)+3); combination 1)+2)+3); combination 1)+2)+3)+4); combination 1)+3)+4); combination 2)+3)+4); combination 1)+2)+4); combination 1)+2)+3)+4)+5); combination 1)+3)+4)+5); combination 2)+3)+4)+5); combination 1)+2)+4)+5); combination 1)+2)+3)+5); combination 1)+3)+5); combination 2)+3)+5); combination 1)+2)+5).

In some forms, an algorithm for designing, evaluating, or selecting a dead guide RNA targeting sequence (dead guide sequence) for guiding a Cas9 CRISPR-Cas system to a target gene locus can be used. In particular, it has been determined that dead guide RNA specificity relates to and can be optimized by varying (i) GC content and (ii) targeting sequence length. In some forms, an algorithm for designing or evaluating a dead guide RNA targeting sequence that minimizes off-target binding or interaction of the dead guide RNA can be used. In some forms, the algorithm for selecting a dead guide RNA targeting sequence for directing a CRISPR system to a gene locus in an organism comprises (a) locating one or more CRISPR motifs in the gene locus, (b) analyzing the 20 nt sequence downstream of each CRISPR motif by (i) determining the GC content of the sequence; and (ii) determining whether there are off-target matches of the 15 downstream nucleotides nearest to the CRISPR motif in the genome of the organism, and (c) selecting the 15 nucleotide sequence for use in a dead guide RNA if the GC content of the sequence is 70% or less and no off-target matches are identified. In some forms, the sequence is selected for a targeting sequence if the GC content is 60% or less. In some forms, the sequence is selected for a targeting sequence if the GC content is 55% or less, 50% or less, 45% or less, 40% or less, 35% or less or 30% or less. In some forms, two or more sequences of the gene locus are analyzed and the sequence having the lowest GC content, or the next lowest GC content, or the next lowest GC content is selected. In some forms, the sequence is selected for a targeting sequence if no off-target matches are identified in the genome of the organism. In some forms, the targeting sequence is selected if no off-target matches are identified in regulatory sequences of the genome.

In some forms, a dead guide RNA targeting sequence can be selected for directing a functionalized CRISPR system to a gene locus in an organism. This can be accomplished by, for example, (a) locating one or more CRISPR motifs in the gene locus; (b) analyzing the 20 nt sequence downstream of each CRISPR motif by: (i) determining the GC content of the sequence; and (ii) determining whether there are off-target matches of the first 15 nt of the sequence in the genome of the organism; and (c) selecting the sequence for use in a guide RNA if the GC content of the sequence is 70% or less and no off-target matches are identified. In some forms, the sequence is selected if the GC content is 50% or less. In some forms, the sequence is selected if the GC content is 40% or less. In some forms, the sequence is selected if the GC content is 30% or less. In some forms, two or more sequences are analyzed and the sequence having the lowest GC content is selected. In some forms, off-target matches are determined in regulatory sequences of the organism. In some forms, the gene locus is a regulatory region. In some forms, a dead guide RNA is provided that comprises the targeting sequence selected according to the aforementioned methods.

In some forms, a dead guide RNA for targeting a functionalized CRISPR system to a gene locus in an organism can be used. In some forms, the dead guide RNA comprises a targeting sequence wherein the CG content of the target sequence is 70% or less, and the first 15 nt of the targeting sequence does not match an off-target sequence downstream from a CRISPR motif in the regulatory sequence of another gene locus in the organism. In some forms, the GC content of the targeting sequence 60% or less, 55% or less, 50% or less, 45% or less, 40% or less, 35% or less or 30% or less. In some forms, the GC content of the targeting sequence is from 70% to 60% or from 60% to 50% or from 50% to 40% or from 40% to 30%. In some forms, the targeting sequence has the lowest CG content among potential targeting sequences of the locus.

In some forms, the first 15 nt of the dead guide match the target sequence. In other forms, first 14 nt of the dead guide match the target sequence. In other forms, the first 13 nt of the dead guide match the target sequence. In other forms, first 12 nt of the dead guide match the target sequence. In other forms, first 11 nt of the dead guide match the target sequence. In other forms, the first 10 nt of the dead guide match the target sequence. In some forms, the first 15 nt of the dead guide do not match an off-target sequence downstream from a CRISPR motif in the regulatory region of another gene locus. In other forms, the first 14 nt, or the first 13 nt of the dead guide, or the first 12 nt of the guide, or the first 11 nt of the dead guide, or the first 10 nt of the dead guide, does not match an off-target sequence downstream from a CRISPR motif in the regulatory region of another gene locus. In other forms, the first 15 nt, or 14 nt, or 13 nt, or 12 nt, or 11 nt of the dead guide do not match an off-target sequence downstream from a CRISPR motif in the genome.

In some forms, the dead guide RNA includes additional nucleotides at the 3′-end that do not match the target sequence. Thus, a dead guide RNA that includes the first 15 nt, or 14 nt, or 13 nt, or 12 nt, or 11 nt downstream of a CRISPR motif can be extended in length at the 3′ end to 12 nt, 13 nt, 14 nt, 15 nt, 16 nt, 17 nt, 18 nt, 19 nt, 20 nt, or longer.

In some forms, the disclosed compositions and methods can be used for directing a Cas9 CRISPR-Cas system, including but not limited to a dead Cas9 (dCas9) or functionalized Cas9 system (which may comprise a functionalized Cas9 or functionalized guide), to a gene locus. In some forms, the disclosed compositions and methods can use a dead guide RNA targeting sequence to direct a functionalized CRISPR system to a gene locus in an organism. In some forms, the disclosed compositions and methods can use a dead guide RNA targeting sequence to effect gene regulation of a target gene locus by a functionalized Cas9 CRISPR-Cas system. In some forms, the disclosed compositions and methods can be used to effect target gene regulation while minimizing off-target effects. In some forms, the disclosed compositions and methods can use two or more dead guide RNA targeting sequences to effect gene regulation of two or more target gene loci by a functionalized Cas9 CRISPR-Cas system. In some forms, the disclosed compositions and methods can be used to effect regulation of two or more target gene loci while minimizing off-target effects.

In some forms, the disclosed compositions and methods can use a dead guide RNA targeting sequence for directing a functionalized Cas9 to a gene locus in an organism. This can comprise, for example, (a) locating one or more CRISPR motifs in the gene locus; (b) analyzing the sequence downstream of each CRISPR motif by (i) selecting 10 to 15 nt adjacent to the CRISPR motif, (ii) determining the GC content of the sequence; and (c) selecting the 10 to 15 nt sequence as a targeting sequence for use in a guide RNA if the GC content of the sequence is 40% or more. In some forms, the sequence is selected if the GC content is 50% or more. In some forms, the sequence is selected if the GC content is 60% or more. In some forms, the sequence is selected if the GC content is 70% or more. In some forms, two or more sequences are analyzed and the sequence having the highest GC content is selected. In some forms, the method further comprises adding nucleotides to the 3′ end of the selected sequence which do not match the sequence downstream of the CRISPR motif. In some forms, a dead guide RNA is provided that comprises the targeting sequence selected according to the aforementioned methods.

In some forms, a dead guide RNA can be used for directing a functionalized CRISPR system to a gene locus in an organism wherein the targeting sequence of the dead guide RNA consists of 10 to 15 nucleotides adjacent to the CRISPR motif of the gene locus, wherein the CG content of the target sequence is 50% or more. In some forms, the dead guide RNA further comprises nucleotides added to the 3′ end of the targeting sequence which do not match the sequence downstream of the CRISPR motif of the gene locus.

In some forms, a single effector can be directed to one or more, or two or more gene loci. In some forms, the effector is associated with a Cas9, and one or more, or two or more selected dead guide RNAs are used to direct the Cas9-associated effector to one or more, or two or more selected target gene loci. In some forms, the effector is associated with one or more, or two or more selected dead guide RNAs, each selected dead guide RNA, when complexed with a Cas9 enzyme, causing its associated effector to localize to the dead guide RNA target. One non-limiting example of such CRISPR systems modulates activity of one or more, or two or more gene loci subject to regulation by the same transcription factor.

In some forms, two or more effectors can be directed to one or more gene loci. In some forms, two or more dead guide RNAs are employed, each of the two or more effectors being associated with a selected dead guide RNA, with each of the two or more effectors being localized to the selected target of its dead guide RNA. One non-limiting example of such CRISPR systems modulates activity of one or more, or two or more gene loci subject to regulation by different transcription factors. Thus, in some non-limiting forms, two or more transcription factors are localized to different regulatory sequences of a single gene. In other non-limiting forms, two or more transcription factors are localized to different regulatory sequences of different genes. In some forms, one transcription factor is an activator. In some forms, one transcription factor is an inhibitor. In some forms, one transcription factor is an activator and another transcription factor is an inhibitor. In some forms, gene loci expressing different components of the same regulatory pathway are regulated. In some forms, gene loci expressing components of different regulatory pathways are regulated.

In some forms, dead guide RNAs that are specific for target DNA cleavage or target binding and gene regulation mediated by an active Cas9 CRISPR-Cas system can be designed and selected. In some forms, the Cas9 CRISPR-Cas system provides orthogonal gene control using an active Cas9 which cleaves target DNA at one gene locus while at the same time binds to and promotes regulation of another gene locus.

In some forms, a dead guide RNA targeting sequence can be used for directing a functionalized Cas9 to a gene locus in an organism, without cleavage. Such a dead guide RNA can be selected by, for example, (a) locating one or more CRISPR motifs in the gene locus, (b) analyzing the sequence downstream of each CRISPR motif by (i) selecting 10 to 15 nt adjacent to the CRISPR motif, (ii) determining the GC content of the sequence, and (c) selecting the 10 to 15 nt sequence as a targeting sequence for use in a dead guide RNA if the GC content of the sequence is 30% more, 40% or more. In some forms, the GC content of the targeting sequence is 35% or more, 40% or more, 45% or more, 50% or more, 55% or more, 60% or more, 65% or more, or 70% or more. In some forms, the GC content of the targeting sequence is from 30% to 40% or from 40% to 50% or from 50% to 60% or from 60% to 70%. In some forms, two or more sequences in a gene locus are analyzed and the sequence having the highest GC content is selected.

In some forms, the portion of the targeting sequence in which GC content is evaluated is 10 to 15 contiguous nucleotides of the 15 target nucleotides nearest to the PAM. In some forms, the portion of the guide in which GC content is considered is the 10 to 11 nucleotides or 11 to 12 nucleotides or 12 to 13 nucleotides or 13, or 14, or 15 contiguous nucleotides of the 15 nucleotides nearest to the PAM.

In some forms, dead guide RNAs that promote CRISPR system gene locus cleavage while avoiding functional activation or inhibition can be used. It is observed that increased GC content in dead guide RNAs of 16 to 20 nucleotides coincides with increased DNA cleavage and reduced functional activation.

It is also demonstrated herein that efficiency of functionalized Cas9 can be increased by addition of nucleotides to the 3′ end of a guide RNA which do not match a target sequence downstream of the CRISPR motif. For example, of dead guide RNA 11 to 15 nt in length, shorter guides may be less likely to promote target cleavage, but are also less efficient at promoting CRISPR system binding and functional control. In some forms, addition of nucleotides that don't match the target sequence to the 3′ end of the dead guide RNA increase activation efficiency while not increasing undesired target cleavage. In some forms, dead guide RNAs that effectively promote CRISPRP system function in DNA binding and gene regulation while not promoting DNA cleavage can be used. Thus, in some forms, the dead guide RNA can include the first 15 nt, or 14 nt, or 13 nt, or 12 nt, or 11 nt downstream of a CRISPR motif and be extended in length at the 3′ end by nucleotides that mismatch the target to 12 nt, 13 nt, 14 nt, 15 nt, 16 nt, 17 nt, 18 nt, 19 nt, 20 nt, or longer.

In some forms, the disclosed method can effect selective orthogonal gene control. Dead guide selection, taking into account guide length and GC content, provides effective and selective transcription control by a functional Cas9 CRISPR-Cas system, for example to regulate transcription of a gene locus by activation or inhibition and minimize off-target effects. Accordingly, by providing effective regulation of individual target loci, the compositions and methods also provide effective orthogonal regulation of two or more target loci.

In some forms, orthogonal gene control is by activation or inhibition of two or more target loci. In some forms, orthogonal gene control is by activation or inhibition of one or more target locus and cleavage of one or more target locus.

In some forms, a cell can be made that comprises a non-naturally occurring Cas9 CRISPR-Cas system comprising one or more dead guide RNAs disclosed or made according to a method or algorithm described herein wherein the expression of one or more gene products has been altered. In some forms, the disclosed compositions and methods can be used to alter the expression in the cell of two or more gene products. In some forms, a cell line can be produced from such a cell.

In some forms, the disclosed compositions and methods can be used to produce a multicellular organism comprising one or more cells comprising a non-naturally occurring Cas9 CRISPR-Cas system comprising one or more dead guide RNAs. In some forms, a product can be produced from a cell, cell line, or multicellular organism comprising a non-naturally occurring Cas9 CRISPR-Cas system comprising one or more dead guide RNAs disclosed or made using the disclosed compositions and methods.

In some forms, the disclosed compositions and methods can use a gRNA comprising dead guide(s) as described herein, optionally in combination with gRNA comprising guide(s) as described herein or in the art, in combination with systems e.g. cells, transgenic animals, transgenic mice, inducible transgenic animals, inducible transgenic mice) which are engineered for either overexpression of Cas9 or preferably knock in Cas9. As a result a single system (e.g. transgenic animal, cell) can serve as a basis for multiplex gene modifications in systems/network biology. On account of the dead guides, this is now possible in both in vitro, ex vivo, and in vivo.

For example, once the Cas9 is provided for, one or more dead gRNAs may be provided to direct multiplex gene regulation, and preferably multiplex bidirectional gene regulation. The one or more dead gRNAs may be provided in a spatially and temporally appropriate manner if necessary or desired (for example tissue specific induction of Cas9 expression). On account that the transgenic/inducible Cas9 is provided for (e.g. expressed) in the cell, tissue, animal of interest, both gRNAs comprising dead guides or gRNAs comprising guides are equally effective. In the same manner, the disclosed compositions and methods can use a gRNA comprising dead guide(s) as described herein, optionally in combination with gRNA comprising guide(s) as described herein or in the state of the art, in combination with systems (e.g. cells, transgenic animals, transgenic mice, inducible transgenic animals, inducible transgenic mice) which are engineered for knockout Cas9 CRISPR-Cas.

As a result, the combination of dead guides as described herein with CRISPR applications described herein and CRISPR applications known in the art results in a highly efficient and accurate means for multiplex screening of systems (e.g. network biology). Such screening allows, for example, identification of specific combinations of gene activities for identifying genes responsible for diseases (e.g. on/off combinations), in particular gene related diseases. A preferred application of such screening is cancer. In the same manner, screening for treatment for such diseases can be performed using the disclosed compositions and methods. Cells or animals may be exposed to aberrant conditions resulting in disease or disease like effects. Candidate compositions may be provided and screened for an effect in the desired multiplex environment. For example a patient's cancer cells may be screened for which gene combinations will cause them to die, and then use this information to establish appropriate therapies.

The structural information provided herein allows for interrogation of dead gRNA interaction with the target DNA and the Cas9 permitting engineering or alteration of dead gRNA structure to optimize functionality of the entire Cas9 CRISPR-Cas system. For example, loops of the dead gRNA may be extended, without colliding with the Cas9 protein by the insertion of adaptor proteins that can bind to RNA. These adaptor proteins can further recruit effector proteins or fusions which comprise one or more functional domains.

In some preferred forms, the functional domain is a transcriptional activation domain, preferably VP64. In some forms, the functional domain is a transcription repression domain, preferably KRAB. In some forms, the transcription repression domain is SID, or concatemers of SID (e.g. SID4X). In some forms, the functional domain is an epigenetic modifying domain, such that an epigenetic modifying enzyme is provided. In some forms, the functional domain is an activation domain, which may be the P65 activation domain.

In some forms, the above elements are comprised in a single composition or comprised in individual compositions. These compositions may advantageously be applied to a host to elicit a functional effect on the genomic level.

In general, the dead gRNA are modified in a manner that provides specific binding sites (e.g. aptamers) for adapter proteins comprising one or more functional domains (e.g. via fusion protein) to bind to. The modified dead gRNA are modified such that once the dead gRNA forms a CRISPR complex (i.e. Cas9 binding to dead gRNA and target) the adapter proteins bind and, the functional domain on the adapter protein is positioned in a spatial orientation which is advantageous for the attributed function to be effective. For example, if the functional domain is a transcription activator (e.g. VP64 or p65), the transcription activator is placed in a spatial orientation which allows it to affect the transcription of the target. Likewise, a transcription repressor will be advantageously positioned to affect the transcription of the target and a nuclease (e.g. Fok1) will be advantageously positioned to cleave or partially cleave the target.

The skilled person will understand that modifications to the dead gRNA which allow for binding of the adapter+functional domain but not proper positioning of the adapter+functional domain (e.g. due to steric hindrance within the three dimensional structure of the CRISPR complex) are modifications which are not intended. The one or more modified dead gRNA may be modified at the tetra loop, the stem loop 1, stem loop 2, or stem loop 3, as described herein, preferably at either the tetra loop or stem loop 2, and most preferably at both the tetra loop and stem loop 2.

As explained herein the functional domains may be, for example, one or more domains from the group consisting of methylase activity, demethylase activity, transcription activation activity, transcription repression activity, transcription release factor activity, histone modification activity, RNA cleavage activity, DNA cleavage activity, nucleic acid binding activity, and molecular switches (e.g. light inducible). In some cases it is advantageous that additionally at least one NLS is provided. In some instances, it is advantageous to position the NLS at the N terminus. When more than one functional domain is included, the functional domains may be the same or different.

The dead gRNA may be designed to include multiple binding recognition sites (e.g. aptamers) specific to the same or different adapter protein. The dead gRNA may be designed to bind to the promoter region −1000-+1 nucleic acids upstream of the transcription start site (i.e. TSS), preferably −200 nucleic acids. This positioning improves functional domains which affect gene activation (e.g. transcription activators) or gene inhibition (e.g. transcription repressors). The modified dead gRNA may be one or more modified dead gRNAs targeted to one or more target loci (e.g. at least 1 gRNA, at least 2 gRNA, at least 5 gRNA, at least 10 gRNA, at least 20 gRNA, at least 30 gRNA, at least 50 gRNA) comprised in a composition.

The adaptor protein may be any number of proteins that binds to an aptamer or recognition site introduced into the modified dead gRNA and which allows proper positioning of one or more functional domains, once the dead gRNA has been incorporated into the CRISPR complex, to affect the target with the attributed function. As explained in detail in this application such may be coat proteins, preferably bacteriophage coat proteins. The functional domains associated with such adaptor proteins (e.g. in the form of fusion protein) may include, for example, one or more domains from the group consisting of methylase activity, demethylase activity, transcription activation activity, transcription repression activity, transcription release factor activity, histone modification activity, RNA cleavage activity, DNA cleavage activity, nucleic acid binding activity, and molecular switches (e.g. light inducible). Preferred domains are Fok1, VP64, P65, HSF1, and MyoD1. In the event that the functional domain is a transcription activator or transcription repressor it is advantageous that additionally at least an NLS is provided and preferably at the N terminus. When more than one functional domain is included, the functional domains may be the same or different. The adaptor protein may utilize known linkers to attach such functional domains.

Thus, the modified dead gRNA, the (inactivated) Cas9 (with or without functional domains), and the binding protein with one or more functional domains, may each individually be comprised in a composition and administered to a host individually or collectively. Alternatively, these components may be provided in a single composition for administration to a host. Administration to a host may be performed via viral vectors known to the skilled person or described herein for delivery to a host (e.g. lentiviral vector, adenoviral vector, AAV vector). As explained herein, use of different selection markers (e.g. for lentiviral gRNA selection) and concentration of gRNA (e.g. dependent on whether multiple gRNAs are used) may be advantageous for eliciting an improved effect.

On the basis of this concept, several variations are appropriate to elicit a genomic locus event, including DNA cleavage, gene activation, or gene deactivation. Using the provided compositions, the person skilled in the art can advantageously and specifically target single or multiple loci with the same or different functional domains to elicit one or more genomic locus events. The compositions may be applied in a wide variety of methods for screening in libraries in cells and functional modeling in vivo (e.g. gene activation of lincRNA and identification of function; gain-of-function modeling; loss-of-function modeling; the use the disclosed compositions and methods to establish cell lines and transgenic animals for optimization and screening purposes).

The disclosed compositions and methods can be used to establish and utilize conditional or inducible CRISPR transgenic cell/animals. For example, the target cell comprises Cas9 conditionally or inducibly (e.g. in the form of Cre dependent constructs) and/or the adapter protein conditionally or inducibly and, on expression of a vector introduced into the target cell, the vector expresses that which induces or gives rise to the condition of Cas9 expression and/or adaptor expression in the target cell. In some forms, inducible genomic events affected by functional domains can be used. One example of this is the creation of a CRISPR knock-in/conditional transgenic animal (e.g. mouse comprising e.g. a Lox-Stop-polyA-Lox(LSL) cassette) and subsequent delivery of one or more compositions providing one or more modified dead gRNA (e.g. −200 nucleotides to TSS of a target gene of interest for gene activation purposes) as described herein (e.g. modified dead gRNA with one or more aptamers recognized by coat proteins, e.g. MS2), one or more adapter proteins as described herein (MS2 binding protein linked to one or more VP64) and means for inducing the conditional animal (e.g. Cre recombinase for rendering Cas9 expression inducible). Alternatively, the adaptor protein may be provided as a conditional or inducible element with a conditional or inducible Cas9 to provide an effective model for screening purposes, which advantageously only requires minimal design and administration of specific dead gRNAs for a broad number of applications.

In other forms, the dead guides are further modified to improve specificity. Protected dead guides may be synthesized, whereby secondary structure is introduced into the 3′ end of the dead guide to improve its specificity. A protected guide RNA (pgRNA) comprises a guide sequence capable of hybridizing to a target sequence in a genomic locus of interest in a cell and a protector strand, wherein the protector strand is optionally complementary to the guide sequence and wherein the guide sequence may in part be hybridizable to the protector strand. The pgRNA optionally includes an extension sequence. The thermodynamics of the pgRNA-target DNA hybridization is determined by the number of bases complementary between the guide RNA and target DNA. By employing ‘thermodynamic protection’, specificity of dead gRNA can be improved by adding a protector sequence. For example, one method adds a complementary protector strand of varying lengths to the 3′ end of the guide sequence within the dead gRNA. As a result, the protector strand is bound to at least a portion of the dead gRNA and provides for a protected gRNA (pgRNA). In turn, the dead gRNA references herein may be easily protected using the described forms, resulting in pgRNA. The protector strand can be either a separate RNA transcript or strand or a chimeric version joined to the 3′ end of the dead gRNA guide sequence.

1. Tandem Guides

CRISPR enzymes can employ more than one RNA guide without losing activity. This enables the use of the CRISPR enzymes, systems or complexes as defined herein for targeting multiple DNA targets, genes or gene loci, with a single enzyme, system or complex as defined herein. The guide RNAs may be tandemly arranged, optionally separated by a nucleotide sequence such as a direct repeat as defined herein. The position of the different guide RNAs in the tandem does not influence the activity. In preferred forms, said CRISPR enzyme, CRISPR-Cas enzyme or Cas enzyme is Cas9, or any one of the modified or mutated variants thereof described herein elsewhere.

In some forms, the disclosed compositions and methods can use a non-naturally occurring or engineered CRISPR enzyme, preferably a class 2 CRISPR enzyme, preferably a Type V or VI CRISPR enzyme as described herein, such as without limitation Cas9 as described herein elsewhere, used for tandem or multiplex targeting. It is to be understood that any of the CRISPR (or CRISPR-Cas or Cas) enzymes, complexes, or systems as described herein elsewhere may be used in such an approach. Any of the methods, products, compositions and uses as described herein elsewhere are equally applicable with the multiplex or tandem targeting approach further detailed below.

In some forms, the disclosed compositions and methods can use a Cas9 enzyme, complex, or system as defined herein for targeting multiple gene loci. In some forms, this can be established by using multiple (tandem or multiplex) guide RNA (gRNA) sequences.

In some forms, the disclosed compositions and methods can use one or more elements of a Cas9 enzyme, complex or system as defined herein for tandem or multiplex targeting, wherein said CRISP system comprises multiple guide RNA sequences. Preferably, said gRNA sequences are separated by a nucleotide sequence, such as a direct repeat as defined herein elsewhere.

The Cas9 enzyme, system or complex as defined herein provides an effective means for modifying multiple target polynucleotides. The Cas9 enzyme, system or complex as defined herein has a wide variety of utility including modifying (e.g., deleting, inserting, translocating, inactivating, activating) one or more target polynucleotides in a multiplicity of cell types. As such the Cas9 enzyme, system or complex as defined herein has a broad spectrum of applications in, e.g., gene therapy, drug screening, disease diagnosis, and prognosis, including targeting multiple gene loci within a single CRISPR system.

In some forms, the disclosed compositions and methods can use a Cas9 enzyme, system or complex as defined herein, i.e. a Cas9 CRISPR-Cas complex having a Cas9 protein having at least one destabilization domain associated therewith, and multiple guide RNAs that target multiple nucleic acid molecules such as DNA molecules, whereby each of said multiple guide RNAs specifically targets its corresponding nucleic acid molecule, e.g., DNA molecule. Each nucleic acid molecule target, e.g., DNA molecule can encode a gene product or encompass a gene locus. Using multiple guide RNAs hence enables the targeting of multiple gene loci or multiple genes. In some forms, the Cas9 enzyme may cleave the DNA molecule encoding the gene product. In some forms, expression of the gene product is altered. The Cas9 protein and the guide RNAs do not naturally occur together. In some forms, the guide RNAs can comprise tandemly arranged guide sequences. The disclosed compositions and methods can use coding sequences for the Cas9 protein being codon optimized for expression in a eukaryotic cell. In preferred forms, the eukaryotic cell is a mammalian cell, a plant cell or a yeast cell and in more preferred forms, the mammalian cell is a human cell. Expression of the gene product may be decreased. The Cas9 enzyme may form part of a CRISPR system or complex, which further comprises tandemly arranged guide RNAs (gRNAs) comprising a series of 2, 3, 4, 5, 6, 7, 8, 9, 10, 15, 25, 25, 30, or more than 30 guide sequences, each capable of specifically hybridizing to a target sequence in a genomic locus of interest in a cell. In some forms, the functional Cas9 CRISPR system or complex binds to the multiple target sequences. In some forms, the functional CRISPR system or complex may edit the multiple target sequences, e.g., the target sequences may comprise a genomic locus, and in some forms, there may be an alteration of gene expression. In some forms, the functional CRISPR system or complex may comprise further functional domains. In some forms, the disclosed compositions and methods can be used for altering or modifying expression of multiple gene products. The method may comprise introducing into a cell containing said target nucleic acids, e.g., DNA molecules, or containing and expressing target nucleic acid, e.g., DNA molecules; for instance, the target nucleic acids may encode gene products or provide for expression of gene products (e.g., regulatory sequences).

In preferred forms, the CRISPR enzyme used for multiplex targeting is Cas9, or the CRISPR system or complex comprises Cas9. In some forms, the CRISPR enzyme used for multiplex targeting is AsCas9, or the CRISPR system or complex used for multiplex targeting comprises an AsCas9. In some forms, the CRISPR enzyme is an LbCas9, or the CRISPR system or complex comprises LbCas9. In some forms, the Cas9 enzyme used for multiplex targeting cleaves both strands of DNA to produce a double strand break (DSB). In some forms, the CRISPR enzyme used for multiplex targeting is a nickase. In some forms, the Cas9 enzyme used for multiplex targeting is a dual nickase. In some forms, the Cas9 enzyme used for multiplex targeting is a Cas9 enzyme such as a DD Cas9 enzyme as defined herein elsewhere.

In some general forms, the Cas9 enzyme used for multiplex targeting is associated with one or more functional domains. In some more specific forms, the CRISPR enzyme used for multiplex targeting is a deadCas9 as defined herein elsewhere.

In some forms, the disclosed compositions and methods can be used for delivering the Cas9 enzyme, system, or complex for use in multiple targeting as defined herein or the polynucleotides defined herein. Non-limiting examples of such delivery include, for example, particle(s) delivering component(s) of the complex, vector(s) comprising the polynucleotide(s) discussed herein (e.g., encoding the CRISPR enzyme, providing the nucleotides encoding the CRISPR complex). In some forms, the vector may be a plasmid or a viral vector such as AAV, or lentivirus. Transient transfection with plasmids, e.g., into HEK cells may be advantageous, especially given the size limitations of AAV and that while Cas9 fits into AAV, one may reach an upper limit with additional guide RNAs.

Also provided is a model that constitutively expresses the Cas9 enzyme, complex or system as used herein for use in multiplex targeting. The organism may be transgenic and may have been transfected with the present vectors or may be the offspring of an organism so transfected. In some forms, the compositions can comprise the CRISPR enzyme, system and complex as defined herein or the polynucleotides or vectors described herein. Also provided are CRISPR-Cas systems or complexes (e.g., CRISPR-Cas9) comprising multiple guide RNAs, preferably in a tandemly arranged format. Said different guide RNAs may be separated by nucleotide sequences such as direct repeats.

Also provided is a method of treating a subject, e.g., a subject in need thereof, comprising inducing gene editing by transforming the subject with the polynucleotide encoding the CRISPR-Cas system or complex (e.g., CRISPR-Cas9) or any of polynucleotides or vectors described herein and administering them to the subject. A suitable repair template may also be provided, for example delivered by a vector comprising said repair template. Also provided is a method of treating a subject, e.g., a subject in need thereof, comprising inducing transcriptional activation or repression of multiple target gene loci by transforming the subject with the polynucleotides or vectors described herein, wherein said polynucleotide or vector encodes or comprises the effector enzyme (e.g., Cas9), complex or system comprising multiple guide RNAs, preferably tandemly arranged. Where any treatment is occurring ex vivo, for example in a cell culture, then it will be appreciated that the term ‘subject’ may be replaced by the phrase “cell or cell culture.”

Compositions comprising a Cas enzyme (e.g., Cas9), complex or system comprising multiple guide RNAs, preferably tandemly arranged, or the polynucleotide or vector encoding or comprising said Cas enzyme (e.g., Cas9), complex or system comprising multiple guide RNAs, preferably tandemly arranged, for use in the methods of treatment as defined herein elsewhere are also provided. A kit of parts may be provided including such compositions. Use of said composition in the manufacture of a medicament for such methods of treatment are also provided. A CRISPR-Cas9 system can be used in the disclosed compositions and methods for screening, e.g., gain of function screens. Cells which are artificially forced to overexpress a gene are be able to down regulate the gene over time (re-establishing equilibrium) e.g. by negative feedback loops. By the time the screen starts the unregulated gene might be reduced again. Using an inducible Cas9 activator allows one to induce transcription right before the screen and therefore minimizes the chance of false negative hits. Accordingly, by use of the disclosed compositions and methods in screening, e.g., gain of function screens, the chance of false negative results may be minimized.

In some forms, the disclosed compositions and methods can use an engineered, non-naturally occurring CRISPR system comprising a Cas9 protein and multiple guide RNAs that each specifically target a DNA molecule encoding a gene product in a cell, whereby the multiple guide RNAs each target their specific DNA molecule encoding the gene product and the Cas9 protein cleaves the target DNA molecule encoding the gene product, whereby expression of the gene product is altered; and, wherein the CRISPR protein and the guide RNAs do not naturally occur together. The disclosed compositions and methods can use multiple guide RNAs comprising multiple guide sequences, preferably separated by a nucleotide sequence such as a direct repeat and optionally fused to a tracr sequence. In some forms, the CRISPR protein is a type V or VI CRISPR-Cas protein and in more preferred forms, the CRISPR protein is a Cas9 protein. The disclosed compositions and methods can also use a Cas9 protein that is codon optimized for expression in a eukaryotic cell. In preferred forms, the eukaryotic cell is a mammalian cell and in more preferred forms, the mammalian cell is a human cell. In some forms, the expression of the gene product is decreased.

In other forms, the disclosed compositions and methods can use an engineered, non-naturally occurring vector system comprising one or more vectors comprising a first regulatory element operably linked to the multiple CRISPR-Cas system guide RNAs that each specifically target a DNA molecule encoding a gene product and a second regulatory element operably linked coding for a CRISPR protein. Both regulatory elements may be located on the same vector or on different vectors of the system. The multiple guide RNAs target the multiple DNA molecules encoding the multiple gene products in a cell and the CRISPR protein may cleave the multiple DNA molecules encoding the gene products (it may cleave one or both strands or have substantially no nuclease activity), whereby expression of the multiple gene products is altered; and, wherein the CRISPR protein and the multiple guide RNAs do not naturally occur together. In preferred forms, the CRISPR protein is Cas9 protein, optionally codon optimized for expression in a eukaryotic cell. In preferred forms, the eukaryotic cell is a mammalian cell, a plant cell or a yeast cell and in more preferred forms, the mammalian cell is a human cell. In some forms, the expression of each of the multiple gene products is altered, preferably decreased.

In some forms, the disclosed compositions and methods can use a vector system comprising one or more vectors. In some forms, the system comprises: (a) a first regulatory element operably linked to a direct repeat sequence and one or more insertion sites for inserting one or more guide sequences up- or downstream (whichever applicable) of the direct repeat sequence, wherein when expressed, the one or more guide sequence(s) direct(s) sequence-specific binding of the CRISPR complex to the one or more target sequence(s) in a eukaryotic cell, wherein the CRISPR complex comprises a Cas9 enzyme complexed with the one or more guide sequence(s) that is hybridized to the one or more target sequence(s); and (b) a second regulatory element operably linked to an enzyme-coding sequence encoding said Cas9 enzyme, preferably comprising at least one nuclear localization sequence and/or at least one NES; wherein components (a) and (b) are located on the same or different vectors of the system. Where applicable, a tracr sequence may also be provided. In some forms, component (a) further comprises two or more guide sequences operably linked to the first regulatory element, wherein when expressed, each of the two or more guide sequences direct sequence specific binding of a CRISPR-Cas complex to a different target sequence in a eukaryotic cell. In some forms, the CRISPR complex comprises one or more nuclear localization sequences and/or one or more NES of sufficient strength to drive accumulation of said CRISPR-Cas complex in a detectable amount in or out of the nucleus of a eukaryotic cell. In some forms, the first regulatory element is a polymerase III promoter. In some forms, the second regulatory element is a polymerase II promoter. In some forms, each of the guide sequences is at least 16, 17, 18, 19, 20, 25 nucleotides, or between 16-30, or between 16-25, or between 16-20 nucleotides in length.

Recombinant expression vectors can comprise the polynucleotides encoding the Cas9 enzyme, system or complex for use in multiple targeting as defined herein in a form suitable for expression of the nucleic acid in a host cell, which means that the recombinant expression vectors include one or more regulatory elements, which may be selected on the basis of the host cells to be used for expression, that is operatively-linked to the nucleic acid sequence to be expressed. Within a recombinant expression vector, “operably linked” is intended to mean that the nucleotide sequence of interest is linked to the regulatory element(s) in a manner that allows for expression of the nucleotide sequence (e.g., in an in vitro transcription/translation system or in a host cell when the vector is introduced into the host cell).

In some forms, a host cell is transiently or non-transiently transfected with one or more vectors comprising the polynucleotides encoding the Cas9 enzyme, system or complex for use in multiple targeting as defined herein. In some forms, a cell is transfected as it naturally occurs in a subject. In some forms, a cell that is transfected is taken from a subject. In some forms, the cell is derived from cells taken from a subject, such as a cell line. A wide variety of cell lines for tissue culture are known in the art and exemplified herein elsewhere. Cell lines are available from a variety of sources known to those with skill in the art (see, e.g., the American Type Culture Collection (ATCC) (Manassas, Va.)). In some forms, a cell transfected with one or more vectors comprising the polynucleotides encoding the Cas9 enzyme, system or complex for use in multiple targeting as defined herein is used to establish a new cell line comprising one or more vector-derived sequences. In some forms, a cell transiently transfected with the components of a CRISPR-Cas system or complex (e.g., CRISPR-Cas9) for use in multiple targeting as described herein (such as by transient transfection of one or more vectors, or transfection with RNA), and modified through the activity of a CRISPR-Cas system or complex, is used to establish a new cell line comprising cells containing the modification but lacking any other exogenous sequence. In some forms, cells transiently or non-transiently transfected with one or more vectors comprising the polynucleotides encoding the Cas9 enzyme, system or complex for use in multiple targeting as defined herein, or cell lines derived from such cells are used in assessing one or more test compounds.

The term “regulatory element” is as defined herein elsewhere.

Advantageous vectors include lentiviruses and adeno-associated viruses, and types of such vectors can also be selected for targeting particular types of cells.

In some forms, the disclosed compositions and methods can be used to produce a eukaryotic host cell comprising (a) a first regulatory element operably linked to a direct repeat sequence and one or more insertion sites for inserting one or more guide RNA sequences up- or downstream (whichever applicable) of the direct repeat sequence, wherein when expressed, the guide sequence(s) direct(s) sequence-specific binding of the CRISPR-Cas complex to the respective target sequence(s) in a eukaryotic cell, wherein the CRISPR-Cas complex comprises a Cas9 enzyme complexed with the one or more guide sequence(s) that is hybridized to the respective target sequence(s); and/or (b) a second regulatory element operably linked to an enzyme-coding sequence encoding said Cas9 enzyme comprising preferably at least one nuclear localization sequence and/or NES. In some forms, the host cell comprises components (a) and (b). Where applicable, a tracr sequence may also be provided. In some forms, component (a), component (b), or components (a) and (b) are stably integrated into a genome of the host eukaryotic cell. In some forms, component (a) further comprises two or more guide sequences operably linked to the first regulatory element, and optionally separated by a direct repeat, wherein when expressed, each of the two or more guide sequences direct sequence specific binding of a CRISPR-Cas complex to a different target sequence in a eukaryotic cell. In some forms, the Cas9 enzyme comprises one or more nuclear localization sequences and/or nuclear export sequences or NES of sufficient strength to drive accumulation of said CRISPR enzyme in a detectable amount in and/or out of the nucleus of a eukaryotic cell.

In some forms, the Cas9 enzyme is a type V or VI CRISPR system enzyme. In some forms, the Cas9 enzyme is a Cas9 enzyme. In some forms, the Cas9 enzyme is derived from Francisella tularensis 1, Francisella tularensis subsp. novicida, Prevotella albensis, Lachnospiraceae bacterium MC2017 1, Butyrivibrio proteoclasticus, Peregrinibacteria bacterium GW2011_GWA2_33_10, Parcubacteria bacterium GW2011_GWC2_44_17, Smithella sp. SCADC, Acidaminococcus sp. BV3L6, Lachnospiraceae bacterium MA2020, Candidatus Methanoplasma termitum, Eubacterium eligens, Moraxella bovoculi 237, Leptospira inadai, Lachnospiraceae bacterium ND2006, Porphyromonas crevioricanis 3, Prevotella disiens, or Porphyromonas macacae Cas9, and may include further alterations or mutations of the Cas9 as defined herein elsewhere, and can be a chimeric Cas9. In some forms, the Cas9 enzyme is codon-optimized for expression in a eukaryotic cell. In some forms, the CRISPR enzyme directs cleavage of one or two strands at the location of the target sequence. In some forms, the first regulatory element is a polymerase III promoter. In some forms, the second regulatory element is a polymerase II promoter. In some forms, the one or more guide sequence(s) is (are each) at least 16, 17, 18, 19, 20, 25 nucleotides, or between 16-30, or between 16-25, or between 16-20 nucleotides in length. When multiple guide RNAs are used, they are preferably separated by a direct repeat sequence. In some forms, the disclosed compositions and methods can be used to produce a non-human eukaryotic organism; preferably a multicellular eukaryotic organism, comprising a eukaryotic host cell according to any of the described forms. In other forms, the disclosed compositions and methods can be used to produce a eukaryotic organism; preferably a multicellular eukaryotic organism, comprising a eukaryotic host cell according to any of the described forms. The organism in some of these forms may be an animal; for example a mammal. Also, the organism may be an arthropod such as an insect. The organism also may be a plant. Further, the organism may be a fungus.

Also disclosed are kits comprising one or more of the components described herein. In some forms, the kit comprises a vector system and instructions for using the kit. In some forms, the vector system comprises (a) a first regulatory element operably linked to a direct repeat sequence and one or more insertion sites for inserting one or more guide sequences up- or downstream (whichever applicable) of the direct repeat sequence, wherein when expressed, the guide sequence directs sequence-specific binding of a CRISPR-Cas complex to a target sequence in a eukaryotic cell, wherein the CRISPR-Cas complex comprises a Cas9 enzyme complexed with the guide sequence that is hybridized to the target sequence; and/or (b) a second regulatory element operably linked to an enzyme-coding sequence encoding said Cas9 enzyme comprising a nuclear localization sequence. Where applicable, a tracr sequence may also be provided. In some forms, the kit comprises components (a) and (b) located on the same or different vectors of the system. In some forms, component (a) further comprises two or more guide sequences operably linked to the first regulatory element, wherein when expressed, each of the two or more guide sequences direct sequence specific binding of a CRISPR complex to a different target sequence in a eukaryotic cell. In some forms, the Cas9 enzyme comprises one or more nuclear localization sequences of sufficient strength to drive accumulation of said CRISPR enzyme in a detectable amount in the nucleus of a eukaryotic cell. In some forms, the CRISPR enzyme is a type V or VI CRISPR system enzyme. In some forms, the CRISPR enzyme is a Cas9 enzyme. In some forms, the Cas9 enzyme is derived from Francisella tularensis 1, Francisella tularensis subsp. novicida, Prevotella albensis, Lachnospiraceae bacterium MC2017 1, Butyrivibrio proteoclasticus, Peregrinibacteria bacterium GW2011_GWA2_33_10, Parcubacteria bacterium GW2011_GWC2_44_17, Smithella sp. SCADC, Acidaminococcus sp. BV3L6, Lachnospiraceae bacterium MA2020, Candidatus Methanoplasma termitum, Eubacterium eligens, Moraxella bovoculi 237, Leptospira inadai, Lachnospiraceae bacterium ND2006, Porphyromonas crevioricanis 3, Prevotella disiens, or Porphyromonas macacae Cas9 (e.g., modified to have or be associated with at least one DD), and may include further alteration or mutation of the Cas9, and can be a chimeric Cas9. In some forms, the DD-CRISPR enzyme is codon-optimized for expression in a eukaryotic cell. In some forms, the DD-CRISPR enzyme directs cleavage of one or two strands at the location of the target sequence. In some forms, the DD-CRISPR enzyme lacks or substantially DNA strand cleavage activity (e.g., no more than 5% nuclease activity as compared with a wild type enzyme or enzyme not having the mutation or alteration that decreases nuclease activity). In some forms, the first regulatory element is a polymerase III promoter. In some forms, the second regulatory element is a polymerase II promoter. In some forms, the guide sequence is at least 16, 17, 18, 19, 20, 25 nucleotides, or between 16-30, or between 16-25, or between 16-20 nucleotides in length.

In some forms, the disclosed compositions and methods can be used to modifying multiple target polynucleotides in a host cell such as a eukaryotic cell. In some forms, the method comprises allowing a Cas9CRISPR complex to bind to multiple target polynucleotides, e.g., to effect cleavage of said multiple target polynucleotides, thereby modifying multiple target polynucleotides, wherein the Cas9CRISPR complex comprises a Cas9 enzyme complexed with multiple guide sequences each of the being hybridized to a specific target sequence within said target polynucleotide, wherein said multiple guide sequences are linked to a direct repeat sequence. Where applicable, a tracr sequence may also be provided (e.g. to provide a single guide RNA, sgRNA). In some forms, said cleavage comprises cleaving one or two strands at the location of each of the target sequence by said Cas9 enzyme. In some forms, said cleavage results in decreased transcription of the multiple target genes. In some forms, the method further comprises repairing one or more of said cleaved target polynucleotide by homologous recombination with an exogenous template polynucleotide, wherein said repair results in a mutation comprising an insertion, deletion, or substitution of one or more nucleotides of one or more of said target polynucleotides. In some forms, said mutation results in one or more amino acid changes in a protein expressed from a gene comprising one or more of the target sequence(s). In some forms, the method further comprises delivering one or more vectors to said eukaryotic cell, wherein the one or more vectors drive expression of one or more of: the Cas9 enzyme and the multiple guide RNA sequence linked to a direct repeat sequence. Where applicable, a tracr sequence may also be provided. In some forms, said vectors are delivered to the eukaryotic cell in a subject. In some forms, said modifying takes place in said eukaryotic cell in a cell culture. In some forms, the method further comprises isolating said eukaryotic cell from a subject prior to said modifying. In some forms, the method further comprises returning said eukaryotic cell and/or cells derived therefrom to said subject.

In some forms, the disclosed compositions and methods can be used to modify expression of multiple polynucleotides in a eukaryotic cell. In some forms, the method comprises allowing a CRISPR-Cas complex to bind to multiple polynucleotides such that said binding results in increased or decreased expression of said polynucleotides; wherein the CRISPR-Cas complex comprises a Cas9 enzyme complexed with multiple guide sequences each specifically hybridized to its own target sequence within said polynucleotide, wherein said guide sequences are linked to a direct repeat sequence. Where applicable, a tracr sequence may also be provided. In some forms, the method further comprises delivering one or more vectors to said eukaryotic cells, wherein the one or more vectors drive expression of one or more of: the Cas9 enzyme and the multiple guide sequences linked to the direct repeat sequences. Where applicable, a tracr sequence may also be provided.

In some forms, the disclosed compositions and methods can use a recombinant polynucleotide comprising multiple guide RNA sequences up- or downstream (whichever applicable) of a direct repeat sequence, wherein each of the guide sequences when expressed directs sequence-specific binding of a Cas9CRISPR complex to its corresponding target sequence present in a eukaryotic cell. In some forms, the target sequence is a viral sequence present in a eukaryotic cell. Where applicable, a tracr sequence may also be provided. In some forms, the target sequence is a proto-oncogene or an oncogene.

In some forms, the disclosed compositions and methods can use a non-naturally occurring or engineered composition that may comprise a guide RNA (gRNA) comprising a guide sequence capable of hybridizing to a target sequence in a genomic locus of interest in a cell and a Cas9 enzyme as defined herein that may comprise at least one or more nuclear localization sequences.

In some forms, methods of modifying a genomic locus of interest can be used to change gene expression in a cell by introducing into the cell any of the compositions described herein.

In some forms, the above elements are comprised in a single composition or comprised in individual compositions. These compositions may advantageously be applied to a host to elicit a functional effect on the genomic level.

As used herein, the term “guide RNA” or “gRNA” has the leaning as used herein elsewhere and comprises any polynucleotide sequence having sufficient complementarity with a target nucleic acid sequence to hybridize with the target nucleic acid sequence and direct sequence-specific binding of a nucleic acid-targeting complex to the target nucleic acid sequence. Each gRNA may be designed to include multiple binding recognition sites (e.g., aptamers) specific to the same or different adapter protein. Each gRNA may be designed to bind to the promoter region −1000-+1 nucleic acids upstream of the transcription start site (i.e. TSS), preferably −200 nucleic acids. This positioning improves functional domains which affect gene activation (e.g., transcription activators) or gene inhibition (e.g., transcription repressors). The modified gRNA may be one or more modified gRNAs targeted to one or more target loci (e.g., at least 1 gRNA, at least 2 gRNA, at least 5 gRNA, at least 10 gRNA, at least 20 gRNA, at least 30 g RNA, at least 50 gRNA) comprised in a composition. Said multiple gRNA sequences can be tandemly arranged and are preferably separated by a direct repeat.

Thus, gRNA, the CRISPR enzyme as defined herein may each individually be comprised in a composition and administered to a host individually or collectively. Alternatively, these components may be provided in a single composition for administration to a host. Administration to a host may be performed via viral vectors known to the skilled person or described herein for delivery to a host (e.g., lentiviral vector, adenoviral vector, AAV vector). As explained herein, use of different selection markers (e.g., for lentiviral sgRNA selection) and concentration of gRNA (e.g., dependent on whether multiple gRNAs are used) may be advantageous for eliciting an improved effect. On the basis of this concept, several variations are appropriate to elicit a genomic locus event, including DNA cleavage, gene activation, or gene deactivation. Using the provided compositions, the person skilled in the art can advantageously and specifically target single or multiple loci with the same or different functional domains to elicit one or more genomic locus events. The compositions may be applied in a wide variety of methods for screening in libraries in cells and functional modeling in vivo (e.g., gene activation of lincRNA and identification of function; gain-of-function modeling; loss-of-function modeling; the use the disclosed compositions to establish cell lines and transgenic animals for optimization and screening purposes).

The disclosed compositions and methods can be used to establish and utilize conditional or inducible CRISPR transgenic cell/animals; see, e.g., Platt et al., Cell (2014), 159(2): 440-455, or PCT patent publications cited herein, such as WO 2014/093622 (PCT/US2013/074667). For example, cells or animals such as non-human animals, e.g., vertebrates or mammals, such as rodents, e.g., mice, rats, or other laboratory or field animals, e.g., cats, dogs, sheep, etc., may be ‘knock-in’ whereby the animal conditionally or inducibly expresses Cas9 akin to Platt et al. The target cell or animal thus comprises the CRISPR enzyme (e.g., Cas9) conditionally or inducibly (e.g., in the form of Cre dependent constructs), on expression of a vector introduced into the target cell, the vector expresses that which induces or gives rise to the condition of the CRISPR enzyme (e.g., Cas9) expression in the target cell.

In some forms, phenotypic alteration is preferably the result of genome modification when a genetic disease is targeted, especially in methods of therapy and preferably where a repair template is provided to correct or alter the phenotype.

In some forms, diseases that may be targeted include those concerned with disease-causing splice defects.

In some forms, cellular targets include Hemopoietic Stem/Progenitor Cells (CD34+); Human T cells; and Eye (retinal cells)—for example photoreceptor precursor cells.

In some forms, Gene targets include: Human Beta Globin—HBB (for treating Sickle Cell Anemia, including by stimulating gene-conversion (using closely related HBD gene as an endogenous template)); CD3 (T-Cells); and CEP920—retina (eye).

In some forms, disease targets also include: cancer; Sickle Cell Anemia (based on a point mutation); HBV, HIV; Beta-Thalassemia; and ophthalmic or ocular disease—for example Leber Congenital Amaurosis (LCA)-causing Splice Defect.

In some forms, delivery methods include: Cationic Lipid Mediated “direct” delivery of Enzyme-Guide complex (RiboNucleoProtein) and electroporation of plasmid DNA.

Methods, products and uses described herein may be used for non-therapeutic purposes. Furthermore, any of the methods described herein may be applied in vitro and ex vivo.

In some forms, provided is a non-naturally occurring or engineered composition comprising: (I) two or more CRISPR-Cas system polynucleotide sequences comprising (a) a first guide sequence capable of hybridizing to a first target sequence in a polynucleotide locus, (b) a second guide sequence capable of hybridizing to a second target sequence in a polynucleotide locus, and (c) a direct repeat sequence; and (II) a Cas9 enzyme or a second polynucleotide sequence encoding it, where when transcribed, the first and the second guide sequences direct sequence-specific binding of a first and a second CRISPR-Cas complex to the first and second target sequences respectively. In some forms, the first CRISPR complex comprises the Cas9 enzyme complexed with the first guide sequence that is hybridizable to the first target sequence, the second CRISPR complex comprises the Cas9 enzyme complexed with the second guide sequence that is hybridizable to the second target sequence, and the first guide sequence directs cleavage of one strand of the DNA duplex near the first target sequence and the second guide sequence directs cleavage of the other strand near the second target sequence inducing a double strand break, thereby modifying the organism or the non-human or non-animal organism. Similarly, compositions comprising more than two guide RNAs can be envisaged e.g. each specific for one target, and arranged tandemly in the composition or CRISPR system or complex as described herein.

In other forms, the Cas9 is delivered into the cell as a protein. In particularly preferred forms, the Cas9 is delivered into the cell as a protein or as a nucleotide sequence encoding it. Delivery to the cell as a protein may include delivery of a Ribonucleoprotein (RNP) complex, where the protein is complexed with the multiple guides.

In some forms, host cells and cell lines can be modified by or can comprise the disclosed compositions, systems, or modified enzymes, including stem cells, and progeny thereof.

In some forms, methods of cellular therapy are provided, where, for example, a single cell or a population of cells is sampled or cultured, wherein that cell or cells is or has been modified ex vivo as described herein, and is then re-introduced (sampled cells) or introduced (cultured cells) into the organism. Stem cells, whether embryonic or induce pluripotent or totipotent stem cells, are also particularly preferred in this regard. But, of course, in vivo forms are also envisaged.

Inventive methods can further comprise delivery of templates, such as repair templates, which may be dsODN or ssODN, see below. Delivery of templates may be via the cotemporaneous or separate from delivery of any or all the CRISPR enzyme or guide RNAs and via the same delivery mechanism or different. In some forms, it is preferred that the template is delivered together with the guide RNAs and, preferably, also the CRISPR enzyme. An example may be an AAV vector where the CRISPR enzyme is AsCas9 or LbCas9.

Inventive methods can further comprise: (a) delivering to the cell a double-stranded oligodeoxynucleotide (dsODN) comprising overhangs complimentary to the overhangs created by said double strand break, wherein said dsODN is integrated into the locus of interest; or—(b) delivering to the cell a single-stranded oligodeoxynucleotide (ssODN), wherein said ssODN acts as a template for homology directed repair of said double strand break. Inventive methods can be for the prevention or treatment of disease in an individual, optionally wherein said disease is caused by a defect in said locus of interest. Inventive methods can be conducted in vivo in the individual or ex vivo on a cell taken from the individual, optionally wherein said cell is returned to the individual.

The disclosed compositions and methods can also use CRISPR enzyme or Cas enzyme or Cas9 enzyme or CRISPR-CRISPR enzyme or CRISPR-Cas system or CRISPR-Cas9 system in tandem or multiple targeting as defined herein.

m. Escorted Guides

In some forms, the disclosed compositions and methods can use escorted Cas9 CRISPR-Cas systems or complexes, especially such a system involving an escorted Cas9 CRISPR-Cas system guide. By “escorted” is meant that the Cas9 CRISPR-Cas system or complex or guide is delivered to a selected time or place within a cell, so that activity of the Cas9 CRISPR-Cas system or complex or guide is spatially or temporally controlled. For example, the activity and destination of the Cas9 CRISPR-Cas system or complex or guide may be controlled by an escort RNA aptamer sequence that has binding affinity for an aptamer ligand, such as a cell surface protein or other localized cellular component. Alternatively, the escort aptamer may for example be responsive to an aptamer effector on or in the cell, such as a transient effector, such as an external energy source that is applied to the cell at a particular time.

The escorted Cas9 CRISPR-Cas systems or complexes have a gRNA with a functional structure designed to improve gRNA structure, architecture, stability, genetic expression, or any combination thereof. Such a structure can include an aptamer.

Aptamers are biomolecules that can be designed or selected to bind tightly to other ligands, for example using a technique called systematic evolution of ligands by exponential enrichment (SELEX; Tuerk C, Gold L: “Systematic evolution of ligands by exponential enrichment: RNA ligands to bacteriophage T4 DNA polymerase.” Science 1990, 249:505-510). Nucleic acid aptamers can for example be selected from pools of random-sequence oligonucleotides, with high binding affinities and specificities for a wide range of biomedically relevant targets, suggesting a wide range of therapeutic utilities for aptamers (Keefe, Anthony D., Supriya Pai, and Andrew Ellington. “Aptamers as therapeutics.” Nature Reviews Drug Discovery 9.7 (2010): 537-550). These characteristics also suggest a wide range of uses for aptamers as drug delivery vehicles (Levy-Nissenbaum, Etgar, et al. “Nanotechnology and aptamers: applications in drug delivery.” Trends in biotechnology 26.8 (2008): 442-449; and, Hicke B J, Stephens A W. “Escort aptamers: a delivery service for diagnosis and therapy.” J Clin Invest 2000, 106:923-928.). Aptamers may also be constructed that function as molecular switches, responding to a que by changing properties, such as RNA aptamers that bind fluorophores to mimic the activity of green fluorescent protein (Paige, Jeremy S., Karen Y. Wu, and Samie R. Jaffrey. “RNA mimics of green fluorescent protein.” Science 333.6042 (2011): 642-646). It has also been suggested that aptamers may be used as components of targeted siRNA therapeutic delivery systems, for example targeting cell surface proteins (Zhou, Jiehua, and John J. Rossi. “Aptamer-targeted cell-specific RNA interference.” Silence 1.1 (2010): 4).

Accordingly, provided herein is a gRNA modified, e.g., by one or more aptamer(s) designed to improve gRNA delivery, including delivery across the cellular membrane, to intracellular compartments, or into the nucleus. Such a structure can include, either in addition to the one or more aptamer(s) or without such one or more aptamer(s), moiety(ies) so as to render the guide deliverable, inducible or responsive to a selected effector. An gRNA that responds to normal or pathological physiological conditions, including without limitation pH, hypoxia, O₂ concentration, temperature, protein concentration, enzymatic concentration, lipid structure, light exposure, mechanical disruption (e.g. ultrasound waves), magnetic fields, electric fields, or electromagnetic radiation is contemplated.

In some forms, non-naturally occurring or engineered compositions can be used, where the composition comprises, for example, an escorted guide RNA (egRNA) comprising: an RNA guide sequence capable of hybridizing to a target sequence in a genomic locus of interest in a cell; and an escort RNA aptamer sequence, wherein the escort aptamer has binding affinity for an aptamer ligand on or in the cell, or the escort aptamer is responsive to a localized aptamer effector on or in the cell, wherein the presence of the aptamer ligand or effector on or in the cell is spatially or temporally restricted.

The escort aptamer may, for example, change conformation in response to an interaction with the aptamer ligand or effector in the cell.

The escort aptamer may have specific binding affinity for the aptamer ligand.

The aptamer ligand may be localized in a location or compartment of the cell, for example on or in a membrane of the cell. Binding of the escort aptamer to the aptamer ligand may accordingly direct the egRNA to a location of interest in the cell, such as the interior of the cell by way of binding to an aptamer ligand that is a cell surface ligand. In this way, a variety of spatially restricted locations within the cell may be targeted, such as the cell nucleus or mitochondria.

The egRNA may include an RNA aptamer linking sequence, operably linking the escort RNA sequence to the RNA guide sequence.

In forms, the egRNA may include one or more photolabile bonds or non-naturally occurring residues.

In some forms, the escort RNA aptamer sequence may be complementary to a target miRNA, which may or may not be present within a cell, so that only when the target miRNA is present is there binding of the escort RNA aptamer sequence to the target miRNA which results in cleavage of the egRNA by an RNA-induced silencing complex (RISC) within the cell.

In forms, the escort RNA aptamer sequence may for example be from 10 to 200 nucleotides in length, and the egRNA may include more than one escort RNA aptamer sequence.

It is to be understood that any of the RNA guide sequences as described herein elsewhere can be used in the egRNA described herein. In some forms, the guide RNA or mature crRNA comprises, consists essentially of, or consists of a direct repeat sequence and a guide sequence or spacer sequence. In some forms, the guide RNA or mature crRNA comprises, consists essentially of, or consists of a direct repeat sequence linked to a guide sequence or spacer sequence. In some forms, the guide RNA or mature crRNA comprises 19 nt of partial direct repeat followed by 23-25 nt of guide sequence or spacer sequence. In some forms, the effector protein is an FnCas9 effector protein and requires at least 16 nt of guide sequence to achieve detectable DNA cleavage and a minimum of 17 nt of guide sequence to achieve efficient DNA cleavage in vitro. In some forms, the direct repeat sequence is located upstream (i.e., 5′) from the guide sequence or spacer sequence. In preferred forms, the seed sequence (i.e. the sequence essential critical for recognition and/or hybridization to the sequence at the target locus) of the FnCas9 guide RNA is approximately within the first 5 nt on the 5′ end of the guide sequence or spacer sequence.

The egRNA may be included in a non-naturally occurring or engineered Cas9 CRISPR-Cas complex composition, together with a Cas9 which may include at least one mutation, for example a mutation so that the Cas9 has no more than 5% of the nuclease activity of a Cas9 not having the at least one mutation, for example having a diminished nuclease activity of at least 97%, or 100% as compared with the Cas9 not having the at least one mutation. The Cas9 may also include one or more nuclear localization sequences. Mutated Cas9 enzymes having modulated activity such as diminished nuclease activity are described herein elsewhere.

The engineered Cas9 CRISPR-Cas composition may be provided in a cell, such as a eukaryotic cell, a mammalian cell, or a human cell.

In forms, the compositions described herein comprise a Cas9 CRISPR-Cas complex having at least three functional domains, at least one of which is associated with Cas9 and at least two of which are associated with egRNA.

The compositions described herein may be used to introduce a genomic locus event in a host cell, such as a eukaryotic cell, in particular a mammalian cell, or a non-human eukaryote, in particular a non-human mammal such as a mouse, in vivo. The genomic locus event may comprise affecting gene activation, gene inhibition, or cleavage in a locus. The compositions described herein may also be used to modify a genomic locus of interest to change gene expression in a cell.

Disclosed are compositions and methods by which gRNA-mediated gene editing activity can be adapted. In some forms, the disclosed compositions and methods can use secondary structures that improve cutting efficiency by increasing gRNA and/or increasing the amount of RNA delivered into the cell. The gRNA may include light labile or inducible nucleotides.

Light responsiveness of an inducible system may be achieved via the activation and binding of cryptochrome-2 and CIB1. Blue light stimulation induces an activating conformational change in cryptochrome-2, resulting in recruitment of its binding partner CIB1. This binding is fast and reversible, achieving saturation in <15 sec following pulsed stimulation and returning to baseline<15 min after the end of stimulation. These rapid binding kinetics result in a system temporally bound only by the speed of transcription/translation and transcript/protein degradation, rather than uptake and clearance of inducing agents. Crytochrome-2 activation is also highly sensitive, allowing for the use of low light intensity stimulation and mitigating the risks of phototoxicity. Further, in a context such as the intact mammalian brain, variable light intensity may be used to control the size of a stimulated region, allowing for greater precision than vector delivery alone may offer.

Energy sources such as electromagnetic radiation, sound energy or thermal energy can be used to induce the guide. Advantageously, the electromagnetic radiation is a component of visible light. In preferred forms, the light is a blue light with a wavelength of about 450 to about 495 nm. In especially preferred forms, the wavelength is about 488 nm. In other preferred forms, the light stimulation is via pulses. The light power may range from about 0-9 mW/cm². In preferred forms, a stimulation paradigm of as low as 0.25 sec every 15 sec should result in maximal activation.

Cells used with the disclosed compositions and methods can be a prokaryotic cell or a eukaryotic cell, advantageously an animal cell a plant cell or a yeast cell, more advantageously a mammalian cell.

The chemical or energy sensitive guide may undergo a conformational change upon induction by the binding of a chemical source or by the energy allowing it act as a guide and have the Cas9 CRISPR-Cas system or complex function. The disclosed compositions and methods can involve applying the chemical source or energy so as to have the guide function and the Cas9 CRISPR-Cas system or complex function; and optionally further determining that the expression of the genomic locus is altered.

There are several different designs of this chemical inducible system: 1. ABI-PYL based system inducible by Abscisic Acid (ABA) (see, e.g., http://stke.sciencemag.org/cgi/content/abstract/sigtrans;4/164/rs2), 2. FKBP-FRB based system inducible by rapamycin (or related chemicals based on rapamycin) (see, e.g., http://www.nature.com/nmeth/journal/v2/n6/full/nmeth763.html), 3. GID1-GAI based system inducible by Gibberellin (GA) (see, e.g., http://www.nature.com/nchembio/journal/v8/n5/full/nchembio.922.html).

A chemical inducible system based on change in sub-cellular localization can also be used. In some forms, the disclosed compositions and methods can use a system in which the polypeptide includes a DNA binding domain comprising at least five or more Transcription activator-like effector (TALE) monomers and at least one or more half-monomers specifically ordered to target the genomic locus of interest linked to at least one or more effector domains are further linker to a chemical or energy sensitive protein. This protein will lead to a change in the sub-cellular localization of the entire polypeptide (i.e. transportation of the entire polypeptide from cytoplasm into the nucleus of the cells) upon the binding of a chemical or energy transfer to the chemical or energy sensitive protein. This transportation of the entire polypeptide from one sub-cellular compartments or organelles, in which its activity is sequestered due to lack of substrate for the effector domain, into another one in which the substrate is present would allow the entire polypeptide to come in contact with its desired substrate (i.e. genomic DNA in the mammalian nucleus) and result in activation or repression of target gene expression.

This type of system could also be used to induce the cleavage of a genomic locus of interest in a cell when the effector domain is a nuclease.

A chemical inducible system can be an estrogen receptor (ER) based system inducible by 4-hydroxytamoxifen (4OHT) (see, e.g., http://www.pnas.org/content/104/3/1027.abstract). A mutated ligand-binding domain of the estrogen receptor called ERT2 translocates into the nucleus of cells upon binding of 4-hydroxytamoxifen. In some forms, any naturally occurring or engineered derivative of any nuclear receptor, thyroid hormone receptor, retinoic acid receptor, estrogen receptor, estrogen-related receptor, glucocorticoid receptor, progesterone receptor, androgen receptor may be used in inducible systems analogous to the ER based inducible system.

Another inducible system is based on the design using Transient receptor potential (TRP) ion channel based system inducible by energy, heat or radio-wave (see, e.g., http://www.sciencemag.org/content/336/6081/604). These TRP family proteins respond to different stimuli, including light and heat. When this protein is activated by light or heat, the ion channel will open and allow the entering of ions such as calcium into the plasma membrane. This influx of ions will bind to intracellular ion interacting partners linked to a polypeptide including the guide and the other components of the Cas9 CRISPR-Cas complex or system, and the binding will induce the change of sub-cellular localization of the polypeptide, leading to the entire polypeptide entering the nucleus of cells. Once inside the nucleus, the guide protein and the other components of the Cas9 CRISPR-Cas complex will be active and modulating target gene expression in cells.

This type of system could also be used to induce the cleavage of a genomic locus of interest in a cell; and, in this regard, it is noted that the Cas9 enzyme is a nuclease. The light could be generated with a laser or other forms of energy sources. The heat could be generated by raise of temperature results from an energy source, or from nano-particles that release heat after absorbing energy from an energy source delivered in the form of radio-wave.

While light activation may be an advantageous form, sometimes it may be disadvantageous especially for in vivo applications in which the light may not penetrate the skin or other organs. In this instance, other methods of energy activation are contemplated, in particular, electric field energy and/or ultrasound which have a similar effect.

Electric field energy is preferably administered substantially as described in the art, using one or more electric pulses of from about 1 Volt/cm to about 10 kVolts/cm under in vivo conditions. Instead of or in addition to the pulses, the electric field may be delivered in a continuous manner. The electric pulse may be applied for between 1 μs and 500 milliseconds, preferably between 1 μs and 100 milliseconds. The electric field may be applied continuously or in a pulsed manner for 5 about minutes.

As used herein, ‘electric field energy’ is the electrical energy to which a cell is exposed. Preferably the electric field has a strength of from about 1 Volt/cm to about 10 kVolts/cm or more under in vivo conditions (see WO97/49450).

As used herein, the term “electric field” includes one or more pulses at variable capacitance and voltage and including exponential and/or square wave and/or modulated wave and/or modulated square wave forms. References to electric fields and electricity should be taken to include reference the presence of an electric potential difference in the environment of a cell. Such an environment may be set up by way of static electricity, alternating current (AC), direct current (DC), etc., as known in the art. The electric field may be uniform, non-uniform or otherwise, and may vary in strength and/or direction in a time dependent manner.

Single or multiple applications of electric field, as well as single or multiple applications of ultrasound are also possible, in any order and in any combination. The ultrasound and/or the electric field may be delivered as single or multiple continuous applications, or as pulses (pulsatile delivery).

n. Self-Inactivating CRISPR-Cas

Once intended alterations have been introduced, such as by editing intended copies of a gene in the genome of a cell, continued CRISPR-Cas9 expression in that cell is no longer necessary. Indeed, sustained expression would be undesirable in certain cases of off-target effects at unintended genomic sites, etc. Thus time-limited expression would be useful. Inducible expression offers one approach, but in addition a self-inactivating Cas9 CRISPR-Cas system that relies on the use of a non-coding guide target sequence within the CRISPR vector itself can be used. Thus, after expression begins, the CRISPR system will lead to its own destruction, but before destruction is complete it will have time to edit the genomic copies of the target gene (which, with a normal point mutation in a diploid cell, requires at most two edits). Simply, the self-inactivating Cas9 CRISPR-Cas system includes additional RNA (i.e., guide RNA) that targets the coding sequence for the CRISPR enzyme itself or that targets one or more non-coding guide target sequences complementary to unique sequences present in one or more of the following: (a) within the promoter driving expression of the non-coding RNA elements, (b) within the promoter driving expression of the Cas9 gene, (c) within 100 bp of the ATG translational start codon in the Cas9 coding sequence, (d) within the inverted terminal repeat (iTR) of a viral delivery vector, e.g., in an AAV genome.

o. Proximal CRISPR

Proximal CRISPR (proxy-CRISPR) is a system that can increase the efficiency, effectiveness, and specificity of editing at a target. In proxy-CRISPR, inactive Cas proteins (e.g., dCas) targeted to sequences flanking a main target site are used with an active Cas nuclease targeted to the main target site. The inactive Cas make the main target more accessible to the active Cas, thus increasing efficiency of targeting and editing. This increased access at the main target can also increase the specificity of targeting since other sites in the genome that might be targeted by active Cas will not have the flanking sequences that the inactive Cas target. These and other features of proxy-CRISPR are described in Chen et al., Nature Communications doi: 10.1038/ncomms14958 (2017). Proxy-CRISPR concepts can be used with any CRISPR-Cas system.

p. Alt-R CRISPR

Alt-R CRISPR involves principles for configuring a CRISPR-Cas system to increase efficiency and effectiveness. Generally, Alt-R CRISPR involves use of a Cas effector protein itself (rather than a mRNA encoding the Cas effector protein or a vector that can express the Cas effector protein) with separate guide RNA and tracrRNA (rather than a single guide RNA that combines gRNA and tracrRNA in a single molecule). The maximum efficiency is generally obtained when the gRNA, tracrRNA, or both are shortened (to about 36 nt and about 67 nt, respectively). An example of Alt-R CRISPR was used in Ohtsuka et al., Genome Biology 19:25 (2018) (doi: 10.1186/s13059-018-1400-x).

q. Protected Guides

In some forms, the disclosed compositions and methods can be used with CRISP systems and components that enhance the specificity of Cas9 given individual guide RNAs through thermodynamic tuning of the binding specificity of the guide RNA to target DNA. This is a general approach of introducing mismatches, elongation or truncation of the guide sequence to increase/decrease the number of complimentary bases vs. mismatched bases shared between a genomic target and its potential off-target loci, in order to give thermodynamic advantage to targeted genomic loci over genomic off-targets.

In some forms, the guide sequence can be modified by secondary structure to increase the specificity of the Cas9 CRISPR-Cas system, whereby the secondary structure can protect against exonuclease activity and allow for 3′ additions to the guide sequence.

In some forms, a “protector RNA” can be hybridized to a guide sequence, wherein the “protector RNA” is an RNA strand complementary to the 5′ end of the guide RNA (gRNA), to thereby generate a partially double-stranded gRNA. In some forms, protecting the mismatched bases with a perfectly complementary protector sequence decreases the likelihood of target DNA binding to the mismatched base pairs at the 3′ end. In some forms, additional sequences comprising an extended length may also be present.

Guide RNA (gRNA) extensions matching the genomic target provide gRNA protection and enhance specificity. Extension of the gRNA with matching sequence distal to the end of the spacer seed for individual genomic targets is envisaged to provide enhanced specificity. Matching gRNA extensions that enhance specificity have been observed in cells without truncation. Prediction of gRNA structure accompanying these stable length extensions has shown that stable forms arise from protective states, where the extension forms a closed loop with the gRNA seed due to complimentary sequences in the spacer extension and the spacer seed. These results demonstrate that the protected guide concept also includes sequences matching the genomic target sequence distal of the 20mer spacer-binding region. Thermodynamic prediction can be used to predict completely matching or partially matching guide extensions that result in protected gRNA states. This extends the concept of protected gRNAs to interaction between X and Z, where X will generally be of length 17-20 nt and Z is of length 1-30 nt. Thermodynamic prediction can be used to determine the optimal extension state for Z, potentially introducing small numbers of mismatches in Z to promote the formation of protected conformations between X and Z. Throughout the present application, the terms “X” and seed length (SL) are used interchangeably with the term exposed length (EpL) which denotes the number of nucleotides available for target DNA to bind; the terms “Y” and protector length (PL) are used interchangeably to represent the length of the protector; and the terms “Z,” “E,” “E′” and “EL” are used interchangeably to correspond to the term extended length (ExL) which represents the number of nucleotides by which the target sequence is extended.

An extension sequence which corresponds to the extended length (ExL) may optionally be attached directly to the guide sequence at the 3′ end of the protected guide sequence. The extension sequence may be 2 to 12 nucleotides in length. Preferably ExL may be denoted as 0, 2, 4, 6, 8, 10 or 12 nucleotides in length. In preferred forms, the ExL is denoted as 0 or 4 nucleotides in length. In more preferred forms, the ExL is 4 nucleotides in length. The extension sequence may or may not be complementary to the target sequence.

An extension sequence may further optionally be attached directly to the guide sequence at the 5′ end of the protected guide sequence as well as to the 3′ end of a protecting sequence. As a result, the extension sequence serves as a linking sequence between the protected sequence and the protecting sequence. Without wishing to be bound by theory, such a link may position the protecting sequence near the protected sequence for improved binding of the protecting sequence to the protected sequence. It will be understood that the above-described relationship of seed, protector, and extension applies where the distal end (i.e., the targeting end) of the guide is the 5′ end, e.g. a guide that functions is a Cas9 system. In some forms where the distal end of the guide is the 3′ end, the relationship will be the reverse. In such some forms, a “protector RNA” can be hybridized to a guide sequence, wherein the “protector RNA” is an RNA strand complementary to the 3′ end of the guide RNA (gRNA), to thereby generate a partially double-stranded gRNA.

Addition of gRNA mismatches to the distal end of the gRNA can demonstrate enhanced specificity. The introduction of unprotected distal mismatches in Y or extension of the gRNA with distal mismatches (Z) can demonstrate enhanced specificity. This concept as mentioned is tied to X, Y, and Z components used in protected gRNAs. The unprotected mismatch concept may be further generalized to the concepts of X, Y, and Z described for protected guide RNAs.

In some forms, the disclosed compositions and methods can use enhanced Cas9 specificity wherein the double stranded 3′ end of the protected guide RNA (pgRNA) allows for two possible outcomes: (1) the guide RNA-protector RNA to guide RNA-target DNA strand exchange will occur and the guide will fully bind the target, or (2) the guide RNA will fail to fully bind the target and because Cas9 target cleavage is a multiple step kinetic reaction that requires guide RNA:target DNA binding to activate Cas9-catalyzed DSBs, wherein Cas9 cleavage does not occur if the guide RNA does not properly bind. According to particular forms, the protected guide RNA improves specificity of target binding as compared to a naturally occurring CRISPR-Cas system. According to particular forms, the protected modified guide RNA improves stability as compared to a naturally occurring CRISPR-Cas. According to particular forms, the protector sequence has a length between 3 and 120 nucleotides and comprises 3 or more contiguous nucleotides complementary to another sequence of guide or protector. According to particular forms, the protector sequence forms a hairpin. According to particular forms, the guide RNA further comprises a protected sequence and an exposed sequence. According to particular forms, the exposed sequence is 1 to 19 nucleotides. More particularly, the exposed sequence is at least 75%, at least 90% or about 100% complementary to the target sequence. According to particular forms, the guide sequence is at least 90% or about 100% complementary to the protector strand. According to particular forms, the guide sequence is at least 75%, at least 90% or about 100% complementary to the target sequence. According to particular forms, the guide RNA further comprises an extension sequence. More particularly, when the distal end of the guide is the 3′ end, the extension sequence is operably linked to the 3′ end of the protected guide sequence, and optionally directly linked to the 3′ end of the protected guide sequence. According to particular forms, the extension sequence is 1-12 nucleotides. According to particular forms, the extension sequence is operably linked to the guide sequence at the 3′ end of the protected guide sequence and the 5′ end of the protector strand and optionally directly linked to the 3′ end of the protected guide sequence and the 5′ end of the protector strand, wherein the extension sequence is a linking sequence between the protected sequence and the protector strand. According to particular forms, the extension sequence is 100% not complementary to the protector strand, optionally at least 95%, at least 90%, at least 80%, at least 70%, at least 60%, or at least 50% not complementary to the protector strand. According to particular forms, the guide sequence further comprises mismatches appended to the end of the guide sequence, wherein the mismatches thermodynamically optimize specificity.

Design options include, without limitation, i) adjusting the length of protector strand that binds to the protected strand, ii) adjusting the length of the portion of the protected strand that is exposed, iii) extending the protected strand with a stem-loop located external (distal) to the protected strand (i.e. designed so that the stem loop is external to the protected strand at the distal end), iv) extending the protected strand by addition of a protector strand to form a stem-loop with all or part of the protected strand, v) adjusting binding of the protector strand to the protected strand by designing in one or more base mismatches and/or one or more non-canonical base pairings, vi) adjusting the location of the stem formed by hybridization of the protector strand to the protected strand, and vii) addition of a non-structured protector to the end of the protected strand.

In some forms, the disclosed compositions and methods can use an engineered, non-naturally occurring CRISPR-Cas system comprising a Cas9 protein and a protected guide RNA that targets a DNA molecule encoding a gene product in a cell, whereby the protected guide RNA targets the DNA molecule encoding the gene product and the Cas9 protein cleaves the DNA molecule encoding the gene product, whereby expression of the gene product is altered; and, wherein the Cas9 protein and the protected guide RNA do not naturally occur together. The protected guide RNA can comprise a guide sequence fused to a direct repeat sequence. In some forms, the CRISPR protein can be codon optimized for expression in a eukaryotic cell. In preferred forms, the eukaryotic cell is a mammalian cell, a plant cell or a yeast cell and in more preferred forms, the mammalian cell is a human cell. In some forms, the expression of the gene product is decreased. In some forms, the CRISPR protein is Cas9. In some forms, the CRISPR protein is Cas12a. In some forms, the Cas12a protein is Acidaminococcus sp. BV3L6, Lachnospiraceae bacterium or Francisella Novicida Cas12a, and may include mutated Cas12a derived from these organisms. The protein may be a further Cas9 or Cas12a homolog or ortholog. In some forms, the nucleotide sequence encoding the Csa9 or Cas12a protein is codon-optimized for expression in a eukaryotic cell. In some forms, the Cas9 or Cas12a protein directs cleavage of one or two strands at the location of the target sequence. In some forms, the first regulatory element is a polymerase III promoter. In some forms, the second regulatory element is a polymerase II promoter. In general, and throughout this specification, the term “vector” refers to a nucleic acid molecule capable of transporting another nucleic acid to which it has been linked. Vectors include, but are not limited to, nucleic acid molecules that are single-stranded, double-stranded, or partially double-stranded; nucleic acid molecules that comprise one or more free ends, no free ends (e.g., circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g., retroviruses, replication defective retroviruses, adenoviruses, replication defective adenoviruses, and adeno-associated viruses). Viral vectors also include polynucleotides carried by a virus for transfection into a host cell. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors.” Common expression vectors of utility in recombinant DNA techniques are often in.

Cas13 is a type II nuclease that does not make use of tracrRNA. Orthologs of Cas13 have been identified in different bacterial species as described herein. Further type II nucleases with similar properties can be identified using methods described in the art (Shmakov et al. 2015, 60:385-397; Abudayeh et al. 2016, Science, 5; 353(6299)). In particular forms, such methods for identifying novel CRISPR effector proteins may comprise the steps of selecting sequences from the database encoding a seed which identifies the presence of a CRISPR Cas locus, identifying loci located within 10 kb of the seed comprising Open Reading Frames (ORFs) in the selected sequences, selecting therefrom loci comprising ORFs of which only a single ORF encodes a novel CRISPR effector having greater than 700 amino acids and no more than 90% homology to a known CRISPR effector. In particular forms, the seed is a protein that is common to the CRISPR-Cas system, such as Cas1. In some forms, the CRISPR array is used as a seed to identify new effector proteins.

Preassembled recombinant CRISPR-Cas13 complexes comprising Cas13 and crRNA be delivered and result in high mutation rates and absence of detectable off-target mutations. Hur et al, Nat Biotechnol. 2016 Jun. 6. doi: 10.1038/nbt.3596. Genome-wide analyses shows that Cas13 is highly specific. By one measure, in vitro cleavage sites determined for Cas13 in human HEK293T cells were significantly fewer that for SpCas9. Kim et al., Nat Biotechnol. 2016 Jun. 6. doi: 10.1038/nbt.3609. An efficient multiplexed system employing Cas13 has been demonstrated in Drosophila employing gRNAs processed from an array containing inventing tRNAs. Port, F. et al, Expansion of the CRISPR toolbox in an animal with tRNA-flanked Cas9 and Cas13 gRNAs. doi:dx.doi.org/10.1101/046417. Also, Tsai et al., Nature Biotechnology 32(6): 569-77 (2014), relates to dimeric RNA-guided FokI Nucleases that recognize extended sequences and can edit endogenous genes with high efficiencies in human cells.

With respect to general information on CRISPR-Cas Systems, components thereof, and delivery of such components, including methods, materials, delivery vehicles, vectors, particles, and making and using thereof, including as to amounts and formulations, as well as CRISPR-Cas-expressing eukaryotic cells, CRISPR-Cas expressing eukaryotes, such as a mouse, reference is made to: U.S. Pat. Nos. 8,999,641, 8,993,233, 8,697,359, 8,771,945, 8,795,965, 8,865,406, 8,871,445, 8,889,356, 8,889,418, 8,895,308, 8,906,616, 8,932,814, and 8,945,839; US Patent Publications US 2014-0310830 (U.S. application Ser. No. 14/105,031), US 2014-0287938 A1 (U.S. application Ser. No. 14/213,991), US 2014-0273234 A1 (U.S. application Ser. No. 14/293,674), US2014-0273232 A1 (U.S. application Ser. No. 14/290,575), US 2014-0273231 (U.S. application Ser. No. 14/259,420), US 2014-0256046 A1 (U.S. application Ser. No. 14/226,274), US 2014-0248702 A1 (U.S. application Ser. No. 14/258,458), US 2014-0242700 A1 (U.S. application Ser. No. 14/222,930), US 2014-0242699 A1 (U.S. application Ser. No. 14/183,512), US 2014-0242664 A1 (U.S. application Ser. No. 14/104,990), US 2014-0234972 A1 (U.S. application Ser. No. 14/183,471), US 2014-0227787 A1 (U.S. application Ser. No. 14/256,912), US 2014-0189896 A1 (U.S. application Ser. No. 14/105,035), US 2014-0186958 (U.S. application Ser. No. 14/105,017), US 2014-0186919 A1 (U.S. application Ser. No. 14/104,977), US 2014-0186843 A1 (U.S. application Ser. No. 14/104,900), US 2014-0179770 A1 (U.S. application Ser. No. 14/104,837) and US 2014-0179006 A1 (U.S. application Ser. No. 14/183,486), US 2014-0170753 (U.S. application Ser. No. 14/183,429); US 2015-0184139 (U.S. application Ser. No. 14/324,960); Ser. No. 14/054,414 European Patent Applications EP 2 771 468 (EP13818570.7), EP 2 764 103 (EP13824232.6), and EP 2 784 162 (EP14170383.5); and PCT Patent Publications WO2014/093661 (PCT/US2013/074743), WO2014/093694 (PCT/US2013/074790), WO2014/093595 (PCT/US2013/074611), WO2014/093718 (PCT/US2013/074825), WO2014/093709 (PCT/US2013/074812), WO2014/093622 (PCT/US2013/074667), WO2014/093635 (PCT/US2013/074691), WO2014/093655 (PCT/US2013/074736), WO2014/093712 (PCT/US2013/074819), WO2014/093701 (PCT/US2013/074800), WO2014/018423 (PCT/US2013/051418), WO2014/204723 (PCT/US2014/041790), WO2014/204724 (PCT/US2014/041800), WO2014/204725 (PCT/US2014/041803), WO2014/204726 (PCT/US2014/041804), WO2014/204727 (PCT/US2014/041806), WO2014/204728 (PCT/US2014/041808), WO2014/204729 (PCT/US2014/041809), WO2015/089351 (PCT/US2014/069897), WO2015/089354 (PCT/US2014/069902), WO2015/089364 (PCT/US2014/069925), WO2015/089427 (PCT/US2014/070068), WO2015/089462 (PCT/US2014/070127), WO2015/089419 (PCT/US2014/070057), WO2015/089465 (PCT/US2014/070135), WO2015/089486 (PCT/US2014/070175), WO2015/058052 (PCT/US2014/061077), WO2015/070083 (PCT/US2014/064663), WO2015/089354 (PCT/US2014/069902), WO2015/089351 (PCT/US2014/069897), WO2015/089364 (PCT/US2014/069925), WO2015/089427 (PCT/US2014/070068), WO2015/089473 (PCT/US2014/070152), WO2015/089486 (PCT/US2014/070175), WO2016/049258 (PCT/US2015/051830), WO2016/094867 (PCT/US2015/065385), WO2016/094872 (PCT/US2015/065393), WO2016/094874 (PCT/US2015/065396), WO2016/106244 (PCT/US2015/067177).

Mention is also made of U.S. application 62/180,709, 17 Jun. 2015, PROTECTED GUIDE RNAS (PGRNAS); U.S. application 62/091,455, filed, 12 Dec. 2014, PROTECTED GUIDE RNAS (PGRNAS); U.S. application 62/096,708, 24 Dec. 2014, PROTECTED GUIDE RNAS (PGRNAS); U.S. applications 62/091,462, 12 Dec. 2014, Ser. No. 62/096,324, 23 Dec. 2014, 62/180,681, 17 Jun. 2015, and 62/237,496, 5 Oct. 2015, DEAD GUIDES FOR CRISPR TRANSCRIPTION FACTORS; U.S. application 62/091,456, 12 Dec. 2014 and 62/180,692, 17 Jun. 2015, ESCORTED AND FUNCTIONALIZED GUIDES FOR CRISPR-CAS SYSTEMS; U.S. application 62/091,461, 12 Dec. 2014, DELIVERY, USE AND THERAPEUTIC APPLICATIONS OF THE CRISPR-CAS SYSTEMS AND COMPOSITIONS FOR GENOME EDITING AS TO HEMATOPOETIC STEM CELLS (HSCs); U.S. application 62/094,903, 19 Dec. 2014, UNBIASED IDENTIFICATION OF DOUBLE-STRAND BREAKS AND GENOMIC REARRANGEMENT BY GENOME-WISE INSERT CAPTURE SEQUENCING; U.S. application 62/096,761, 24 Dec. 2014, ENGINEERING OF SYSTEMS, METHODS AND OPTIMIZED ENZYME AND GUIDE SCAFFOLDS FOR SEQUENCE MANIPULATION; U.S. application 62/098,059, 30 Dec. 2014, 62/181,641, 18 Jun. 2015, and 62/181,667, 18 Jun. 2015, RNA-TARGETING SYSTEM; U.S. application 62/096,656, 24 Dec. 2014 and 62/181,151, 17 Jun. 2015, CRISPR HAVING OR ASSOCIATED WITH DESTABILIZATION DOMAINS; U.S. application 62/096,697, 24 Dec. 2014, CRISPR HAVING OR ASSOCIATED WITH AAV; U.S. application 62/098,158, 30 Dec. 2014, ENGINEERED CRISPR COMPLEX INSERTIONAL TARGETING SYSTEMS; U.S. application 62/151,052, 22 Apr. 2015, CELLULAR TARGETING FOR EXTRACELLULAR EXOSOMAL REPORTING; U.S. application 62/054,490, 24 Sep. 2014, DELIVERY, USE AND THERAPEUTIC APPLICATIONS OF THE CRISPR-CAS SYSTEMS AND COMPOSITIONS FOR TARGETING DISORDERS AND DISEASES USING PARTICLE DELIVERY COMPONENTS; U.S. application 61/939,154, 12 Feb. 2014, SYSTEMS, METHODS AND COMPOSITIONS FOR SEQUENCE MANIPULATION WITH OPTIMIZED FUNCTIONAL CRISPR-CAS SYSTEMS; U.S. application 62/055,484, 25 Sep. 2014, SYSTEMS, METHODS AND COMPOSITIONS FOR SEQUENCE MANIPULATION WITH OPTIMIZED FUNCTIONAL CRISPR-CAS SYSTEMS; U.S. application 62/087,537, 4 Dec. 2014, SYSTEMS, METHODS AND COMPOSITIONS FOR SEQUENCE MANIPULATION WITH OPTIMIZED FUNCTIONAL CRISPR-CAS SYSTEMS; U.S. application 62/054,651, 24 Sep. 2014, DELIVERY, USE AND THERAPEUTIC APPLICATIONS OF THE CRISPR-CAS SYSTEMS AND COMPOSITIONS FOR MODELING COMPETITION OF MULTIPLE CANCER MUTATIONS IN VIVO; U.S. application 62/067,886, 23 Oct. 2014, DELIVERY, USE AND THERAPEUTIC APPLICATIONS OF THE CRISPR-CAS SYSTEMS AND COMPOSITIONS FOR MODELING COMPETITION OF MULTIPLE CANCER MUTATIONS IN VIVO; U.S. applications 62/054,675, 24 Sep. 2014 and 62/181,002, 17 Jun. 2015, DELIVERY, USE AND THERAPEUTIC APPLICATIONS OF THE CRISPR-CAS SYSTEMS AND COMPOSITIONS IN NEURONAL CELLS/TISSUES; U.S. application 62/054,528, 24 Sep. 2014, DELIVERY, USE AND THERAPEUTIC APPLICATIONS OF THE CRISPR-CAS SYSTEMS AND COMPOSITIONS IN IMMUNE DISEASES OR DISORDERS; U.S. application 62/055,454, 25 Sep. 2014, DELIVERY, USE AND THERAPEUTIC APPLICATIONS OF THE CRISPR-CAS SYSTEMS AND COMPOSITIONS FOR TARGETING DISORDERS AND DISEASES USING CELL PENETRATION PEPTIDES (CPP); U.S. application 62/055,460, 25 Sep. 2014, MULTIFUNCTIONAL-CRISPR COMPLEXES AND/OR OPTIMIZED ENZYME LINKED FUNCTIONAL-CRISPR COMPLEXES; U.S. application 62/087,475, 4 Dec. 2014 and 62/181,690, 18 Jun. 2015, FUNCTIONAL SCREENING WITH OPTIMIZED FUNCTIONAL CRISPR-CAS SYSTEMS; U.S. application 62/055,487, 25 Sep. 2014, FUNCTIONAL SCREENING WITH OPTIMIZED FUNCTIONAL CRISPR-CAS SYSTEMS; U.S. application 62/087,546, 4 Dec. 2014 and 62/181,687, 18 Jun. 2015, MULTIFUNCTIONAL CRISPR COMPLEXES AND/OR OPTIMIZED ENZYME LINKED FUNCTIONAL-CRISPR COMPLEXES; and U.S. application 62/098,285, 30 Dec. 2014, CRISPR MEDIATED IN VIVO MODELING AND GENETIC SCREENING OF TUMOR GROWTH AND METASTASIS.

Mention is made of U.S. applications 62/181,659, 18 Jun. 2015 and 62/207,318, 19 Aug. 2015, ENGINEERING AND OPTIMIZATION OF SYSTEMS, METHODS, ENZYME AND GUIDE SCAFFOLDS OF CAS9 ORTHOLOGS AND VARIANTS FOR SEQUENCE MANIPULATION. Mention is made of U.S. applications 62/181,663, 18 Jun. 2015 and 62/245,264, 22 Oct. 2015, NOVEL CRISPR ENZYMES AND SYSTEMS, U.S. applications 62/181,675, 18 Jun. 2015, 62/285,349, 22 Oct. 2015, 62/296,522, 17 Feb. 2016, and 62/320,231, 8 Apr. 2016, NOVEL CRISPR ENZYMES AND SYSTEMS, U.S. application 62/232,067, 24 Sep. 2015, U.S. application Ser. No. 14/975,085, 18 Dec. 2015, European application No. 16150428.7, U.S. application 62/205,733, 16 Aug. 2015, U.S. application 62/201,542, 5 Aug. 2015, U.S. application 62/193,507, 16 Jul. 2015, and U.S. application 62/181,739, 18 Jun. 2015, each entitled NOVEL CRISPR ENZYMES AND SYSTEMS and of U.S. application 62/245,270, 22 Oct. 2015, NOVEL CRISPR ENZYMES AND SYSTEMS. Mention is also made of U.S. application 61/939,256, 12 Feb. 2014, and WO 2015/089473 (PCT/US2014/070152), 12 Dec. 2014, each entitled ENGINEERING OF SYSTEMS, METHODS AND OPTIMIZED GUIDE COMPOSITIONS WITH NEW ARCHITECTURES FOR SEQUENCE MANIPULATION. Mention is also made of PCT/US2015/045504, 15 Aug. 2015, U.S. application 62/180,699, 17 Jun. 2015, and U.S. application 62/038,358, 17 Aug. 2014, each entitled GENOME EDITING USING CAS9 NICKASES.

ii. Functional Nucleic Acids

In some forms, the cargo comprises a functional nucleic acid (e.g., antisense nucleic acid, mRNA, miRNA, piRNA, siRNA or combination thereof). Functional nucleic acids that inhibit the transcription, translation or function of a target gene are described.

Functional nucleic acids are nucleic acid molecules that have a specific function, such as binding a target molecule or catalyzing a specific reaction. Functional nucleic acid molecules can be divided into the following non-limiting categories: antisense molecules, RNAi (siRNA, miRNA, piRNA), aptamers, ribozymes, triplex forming molecules, and external guide sequences. The functional nucleic acid molecules can act as effectors, inhibitors, modulators, and stimulators of a specific activity possessed by a target molecule, or the functional nucleic acid molecules can possess a de novo activity independent of any other molecules.

a. Antisense RNA

Functional nucleic acid molecules can interact with any macromolecule, such as DNA, RNA, polypeptides, or carbohydrate chains. Thus, functional nucleic acids can interact with the mRNA or the genomic DNA of a target polypeptide or they can interact with the target polypeptide itself. Functional nucleic acids are often designed to interact with other nucleic acids based on sequence homology between the target molecule and the functional nucleic acid molecule. In other situations, the specific recognition between the functional nucleic acid molecule and the target molecule is not based on sequence homology between the functional nucleic acid molecule and the target molecule, but rather is based on the formation of tertiary structure that allows specific recognition to take place. Therefore the compositions can include one or more functional nucleic acids designed to reduce expression or function of a target protein.

Methods of making and using vectors for in vivo expression of the described functional nucleic acids such as antisense oligonucleotides, siRNA, shRNA, miRNA, EGSs, ribozymes, and aptamers are known in the art.

(A) Antisense Molecules

The functional nucleic acids can be antisense molecules. Antisense molecules are designed to interact with a target nucleic acid molecule through either canonical or non-canonical base pairing. The interaction of the antisense molecule and the target molecule is designed to promote the destruction of the target molecule through, for example, RNAse H mediated RNA-DNA hybrid degradation. Alternatively the antisense molecule is designed to interrupt a processing function that normally would take place on the target molecule, such as transcription or replication. Antisense molecules can be designed based on the sequence of the target molecule. There are numerous methods for optimization of antisense efficiency by finding the most accessible regions of the target molecule. Exemplary methods include in vitro selection experiments and DNA modification studies using DMS and DEPC. It is preferred that antisense molecules bind the target molecule with a dissociation constant (Kd) less than or equal to 10⁻⁶, 10⁻⁸, 10⁻¹⁰, or 10⁻¹².

b. siRNA

(A) RNA Interference

In some forms, the functional nucleic acids induce gene silencing through RNA interference (siRNA). Expression of a target gene can be effectively silenced in a highly specific manner through RNA interference.

Gene silencing was originally observed with the addition of double stranded RNA (dsRNA) (Fire, et al. (1998) Nature, 391:806-11; Napoli, et al. (1990) Plant Cell 2:279-89; Hannon, (2002) Nature, 418:244-51). Once dsRNA enters a cell, it is cleaved by an RNase III-like enzyme called Dicer, into double stranded small interfering RNAs (siRNA) 21-23 nucleotides in length that contain 2 nucleotide overhangs on the 3′ ends (Elbashir, et al., Genes Dev., 15:188-200 (2001); Bernstein, et al., Nature, 409:363-6 (2001); Hammond, et al., Nature, 404:293-6 (2000); Nykanen, et al., Cell, 107:309-21 (2001); Martinez, et al., Cell, 110:563-74 (2002)). The effect of iRNA or siRNA or their use is not limited to any type of mechanism.

In some forms, a siRNA triggers the specific degradation of homologous RNA molecules, such as mRNAs, within the region of sequence identity between both the siRNA and the target RNA. Sequence specific gene silencing can be achieved in mammalian cells using synthetic, short double-stranded RNAs that mimic the siRNAs produced by the enzyme dicer (Elbashir, et al., Nature, 411:494-498 (2001)) (Ui-Tei, et al., FEBS Lett, 479:79-82 (2000)). siRNA can be chemically or in vitro-synthesized or can be the result of short double-stranded hairpin-like RNAs (shRNAs) that are processed into siRNAs inside the cell. In some forms, a long double stranded RNA molecule (dsRNA) that is at least 24 nucleotides in length is processed into a biologically active siRNA of 21-23 nucleotides by the activity of the endogenous cellular enzymes, for example the enzyme Dicer and Dicer-like enzymes within the target organism. The dsRNA contains a nucleotide sequence that is complimentary to one or more genes that are to be targeted for down-regulation. WO 02/44321 describes siRNAs capable of sequence-specific degradation of target mRNAs when base-paired with 3′ overhanging ends, herein incorporated by reference for the method of making these siRNAs. Synthetic siRNAs are generally designed using algorithms and a conventional DNA/RNA synthesizer. Suppliers include Ambion (Austin, Tex.), ChemGenes (Ashland, Mass.), Dharmacon (Lafayette, Colo.), Glen Research (Sterling, Va.), MWB Biotech (Esbersberg, Germany), Proligo (Boulder, Colo.), and Qiagen (Vento, The Netherlands). siRNA can also be synthesized in vitro using kits such as Ambion's SILENCER® siRNA Construction Kit.

Therefore, in some forms, the composition includes a vector expressing the siRNA. The production of siRNA from a vector is more commonly done through the transcription of a short hairpin RNAse (shRNAs). Kits for the production of vectors including shRNA are available, such as, for example, Imgenex's GENESUPPRESSOR™ Construction Kits and Invitrogen's BLOCK-IT™ inducible RNAi plasmid and lentivirus vectors. In some forms, the functional nucleic acid is siRNA, shRNA, or miRNA.

c. miRNA

A miRNA is a small RNA that adopts a hairpin conformation. The miRNA can be cleaved into biologically active dsRNA within the target cell by the activity of the endogenous cellular enzymes, for example the enzyme Dicer and Dicer-like enzymes.

The one or more target genes can be of any desired sequence. In some forms, the sequence of the RNA is 100% complementary to the sequence of the target gene. In other forms, the RNA is less than 100% complementary to the target gene. In some forms, the RNA is at least 95%, at least 90%, at least 85% or at least 80% complementary to the nucleotide sequence of the target gene, so that sequence variations that can occur, for example due to genetic mutation, evolutionary divergence and strain polymorphism can be tolerated.

d. piRNA

The biogenesis of miRNAs and siRNAs typically depends on RNase III type enzymes that convert their double-stranded RNA precursors into functional small RNAs. By contrast, piRNAs derive from single-stranded RNAs and, consequently, require alternative processing machinery.

Synthetic piRNAs can be used to block the synthesis of target proteins by binding to mRNAs, as has been attempted with miRNAs, might have the advantage of not requiring processing by enzymes such as Dicer, which is required by miRNAs. Additional speculative advantages of piRNAs over miRNAs include the possibility of targets with better specificity because each miRNA regulates several mRNAs and there is the potential to access undesirable long non-coding RNAs with possible implications in disease processes (Assumpcao, et al., Epigenomics, 7(6):975-984 (2015)). piRNAs can be the therapeutic agent or can be target sequences for post-transcriptional silencing. For example, synthetic piRNAs designed to couple to PIWI proteins and exert genomic silencing on PIWI genes at a transcriptional level is a possible strategy.

In some forms, the functional nucleic acid is siRNA, shRNA, miRNA, or piRNA. In some forms, the composition includes a vector expressing the functional nucleic acid. Methods of making and using vectors for in vivo expression of functional nucleic acids such as antisense oligonucleotides, siRNA, shRNA, miRNA, piRNA, EGSs, ribozymes, and aptamers are known in the art.

e. Aptamers

The functional nucleic acids can be aptamers. Aptamers are molecules that interact with a target molecule, preferably in a specific way. Typically aptamers are small nucleic acids ranging from 15-50 bases in length that fold into defined secondary and tertiary structures, such as stem-loops or G-quartets. Aptamers can bind small molecules, such as ATP and theophiline, as well as large molecules, such as reverse transcriptase and thrombin. Aptamers can bind very tightly with Kds from the target molecule of less than 10-12 M. It is preferred that the aptamers bind the target molecule with a Kd less than 10-6, 10-8, 10-10, or 10-12. Aptamers can bind the target molecule with a very high degree of specificity. For example, aptamers have been isolated that have greater than a 10,000 fold difference in binding affinities between the target molecule and another molecule that differ at only a single position on the molecule. It is preferred that the aptamer have a Kd with the target molecule at least 10, 100, 1000, 10,000, or 100,000 fold lower than the Kd with a background binding molecule. It is preferred when doing the comparison for a molecule such as a polypeptide, that the background molecule be a different polypeptide.

f. Ribozymes

The functional nucleic acids can be ribozymes. Ribozymes are nucleic acid molecules that are capable of catalyzing a chemical reaction, either intra-molecularly or inter-molecularly. It is preferred that the ribozymes catalyze intermolecular reactions. Different types of ribozymes that catalyze nuclease or nucleic acid polymerase-type reactions which are based on ribozymes found in natural systems, such as hammerhead ribozymes are described. Ribozymes that are not found in natural systems, but which have been engineered to catalyze specific reactions de novo are also described. Preferred ribozymes cleave RNA or DNA substrates, and more preferably cleave RNA substrates. Ribozymes typically cleave nucleic acid substrates through recognition and binding of the target substrate with subsequent cleavage. This recognition is often based mostly on canonical or non-canonical base pair interactions. This property makes ribozymes particularly good candidates for targeting specific cleavage of nucleic acids because recognition of the target substrate is based on the target substrates sequence.

g. Triplex Forming Oligonucleotides

The functional nucleic acids can be triplex forming oligonucleotide molecules. Triplex forming functional nucleic acid molecules are molecules that can interact with either double-stranded or single-stranded nucleic acid. When triplex molecules interact with a target region, a structure called a triplex is formed in which there are three strands of DNA forming a complex dependent on both Watson-Crick and Hoogsteen base-pairing. Triplex molecules are preferred because they can bind target regions with high affinity and specificity. It is preferred that the triplex forming molecules bind the target molecule with a Kd less than 10-6, 10-8, 10-10, or 10-12.

h. External Guide Sequences

The functional nucleic acids can be external guide sequences. External guide sequences (EGSs) are molecules that bind a target nucleic acid molecule forming a complex, which is recognized by RNase P, which then cleaves the target molecule. EGSs can be designed to specifically target a RNA molecule of choice. RNAse P aids in processing transfer RNA (tRNA) within a cell. Bacterial RNAse P can be recruited to cleave virtually any RNA sequence by using an EGS that causes the target RNA:EGS complex to mimic the natural tRNA substrate. Similarly, eukaryotic EGS/RNAse P-directed cleavage of RNA can be utilized to cleave desired targets within eukaryotic cells. Representative examples of how to make and use EGS molecules to facilitate cleavage of a variety of different target molecules are known in the art.

iii. Therapeutic Compounds

In some forms, the cargo comprises therapeutic and/or prophylactic agents, such as biologic agent(s), which may be physically entrapped, encapsulated, and/or non-covalently associated with the assemblies.

Suitable biologic agents include monoclonal antibodies (mAbs), polyclonal antibodies, immunoglobulin, and antigen binding fragments thereof, growth factors (e.g., recombinant human growth factors), antigens, interferons, cytokines, hormones, and other proteins, amino acids, and peptides such as insulin, and combinations thereof. In some instances, the biologic agents are monoclonal antibodies (mAb) selected from infliximab (REMICADE®), adalimumab (HUMIRA®), or combinations thereof.

Other antibodies known in the art include, but are not limited to, those discussed in Kaplon H et al., MAbs. 2018 February/March;10(2):183-203, which is specifically incorporated by reference in entirety. Exemplary antibodies include lanadelumab, crizanlizumab, ravulizumab, eptinezumab, risankizumab, satralizumab, brolucizumab, PRO140, sacituzumab govitecan, moxetumomab pasudotox, cemiplimab, ublituximab, lampalizumab, roledumab, emapalumab, fasinumab, tanezumab, etrolizumab, NEOD001, gantenerumab, anifrolumab, tremelimumab, isatuximab, BCD-100, carotuximab, camrelizumab, IBI308, glembatumumab vedotin, mirvetuximab soravtansine, oportuzumab monatox, L19IL2/L19TNF.

Other antibodies are disclosed in International Publication No. WO2017186928, WO2018007327, WO2018031954, WO2018039247, WO2018015539, and U.S. Patent Publication No. US20180037634, US20180000935, each of which is specifically incorporated by reference in entirety.

Exemplary biologic agents can also be FDA approved therapeutic monoclonal antibodies which include, but are not limited to, ACTEMRA® (tocilizumab, GENENTECH), ADCETRIS® (brentuximab vedotin, SEATTLE GENETICS), AMJEVITA® (adalimumab-atto, AMGEN INC), ANTHIM® (obiltoxaximab, ELUSYS THERAPEUTICS INC), ARZERRA® (ofatumumab, GLAXO GRP LTD), AVASTIN® (bevacizumab, GENENTECH), BAVENCIO® (avelumab, EMD SERONO INC), BENLYSTA® (belimumab, HUMAN GENOME SCIENCES INC.), BESPONSA® (inotuzumab ozogamicin, WYETH PHARMS INC), BLINCYTO® (blinatumomab, AMGEN), CAMPATH® (alemtuzumab, GENZYME), CIMZIA® (certolizumab pegol, UCB INC), CINQAIR® (reslizumab, TEVA RESPIRATORY LLC), COSENTYX® (secukinumab, NOVARTIS PHARMS CORP), CYLTEZO® (adalimumab-adbm, BOEHRINGER INGELHEIM), CYRAMZA® (ramucirumab, ELI LILLY AND CO), DARZALEX® (daratumumab, JANSSEN), DERMABET® (betamethasone valerate, TARO), DUPIXENT® (dupilumab, REGENERON PHARMACEUTICALS), EMPLICITI® (elotuzumab, BRISTOL MYERS SQUIBB), ENTYVIO® (vedolizumab, TAKEDA PHARMS USA), ERBITUX® (cetuximab, IMCLONE), FASENRA® (benralizumab, ASTRAZENECA AB), GAZYVA® (obinutuzumab, GENENTECH), HEMLIBRA® (emicizumab, GENENTECH INC), HERCEPTIN® (trastuzumab, GENENTECH), HUMIRA® (adalimumab, ABBVIE INC), ILARIS® (canakinumab, NOVARTIS PHARMS), ILUMYA® (tildrakizumab-asmn, MERCK SHARP DOHME), IMFINZI® (durvalumab, ASTRAZENECA UK LTD), INFLECTRA® (infliximab-dyyb, CELLTRION INC), IXIFI® (infliximab-qbtx, PFIZER INC), KADCYLA® (ado-trastuzumab emtansine, GENENTECH), KEVZARA® (sarilumab, SANOFI SYNTHELABO), KEYTRUDA® (pembrolizumab, MERCK SHARP DOHME), LARTRUVO® (olaratumab, ELI LILLY AND CO), LEMTRADA® (alemtuzumab, GENZYME), LUCENTIS® (ranibizumab, GENENTECH), MVASI® (bevacizumab-awwb, AMGEN INC), MYLOTARG® (gemtuzumab ozogamicin, WYETH PHARMS INC), MYOSCINT® (imciromab pentetate, CENTOCOR INC), NUCALA® (mepolizumab, GLAXOSMITHKLINE LLC), OCREVUS® (ocrelizumab, GENENTECH INC), OGIVRI® (trastuzumab-dkst, MYLAN GMBH), OPDIVO® (nivolumab, BRISTOL MYERS SQUIBB), PERJETA® (pertuzumab, GENENTECH), PORTRAZZA® (necitumumab, ELI LILLY CO), PRALUENT® (alirocumab, SANOFI AVENTIS), PRAXBIND® (idarucizumab, BOEHRINGER INGELHEIM), PROLIA® (denosumab, AMGEN), PROSTASCINT® (capromab pendetide, CYTOGEN), RAXIBACUMAB® (raxibacumab, HUMAN GENOME SCIENCES INC.), REMICADE® (infliximab, CENTOCOR INC), RENFLEXIS® (infliximab-abda, SAMSUNG BIOEPSIS CO LTD), REOPRO® (abciximab, CENTOCOR INC), REPATHA® (evolocumab, AMGEN INC), RITUXAN® (rituximab, GENENTECH), SILIQ® (brodalumab, VALEANT LUXEMBOURG), SIMPONI ARIA® (golimumab, JANSSEN BIOTECH), SIMULECT® (basiliximab, NOVARTIS), SOLIRIS® (eculizumab, ALEXION PHARM), STELARA® (ustekinumab, CENTOCOR ORTHO BIOTECH INC), STELARA® (ustekinumab, JANSSEN BIOTECH), SYLVANT® (siltuximab, JANSSEN BIOTECH), SYNAGIS® (palivizumab, MEDIMMUNE), TALTZ® (ixekizumab, ELI LILLY AND CO), TECENTRIQ® (atezolizumab, GENENTECH INC), TREMFYA® (guselkumab, JANSSEN BIOTECH), TROGARZO® (ibalizumab-uiyk, TAIMED BIOLOGICS USA), TYSABRI® (natalizumab, BIOGEN IDEC), UNITUXIN® (dinutuximab, UNITED THERAP), VECTIBIX® (panitumumab, AMGEN), XGEVA® (denosumab, AMGEN), XOLAIR® (omalizumab, GENENTECH), YERVOY® (ipilimumab, BRISTOL MYERS SQUIBB), ZEVALIN® (ibritumomab tiuxetan, SPECTRUM PHARMS), ZINBRYTA® (daclizumab, BIOGEN), ZINPLAVA® (bezlotoxumab, MERCK SHARP DOHME).

In some forms, two or more agents, as described above, may be physically entrapped, encapsulated, and/or non-covalently associated with the assemblies. One agent may potentiate the efficacy of another encapsulated agent.

In yet another form, the compositions include a mixture of agents (e.g., a cocktail of proteins) for continuous delivery to a tissue or a cell in need thereof.

Other types of therapeutic or prophylactic agents may be selected from proteins, anti-inflammatory drugs, non-anti-inflammatory agents, steroids, anesthetics (such as lidocaine), analgesics, anti-pyretic agents, anti-infectious agents such as antibacterial, antifungal agents, contraceptives, immunosuppressants, chemotherapeutics, growth factors, cytokines, immunomodulatory molecules. These may be small molecules, proteins, peptides, sugars and polysaccharides, lipids and lipoproteins or lipopolysaccharides, and nucleic acids such as small interfering RNA, microRNA, PiRNA, ribozymes, and nucleotides encoding proteins or peptides. In some cases, cells can be delivered.

3. Bridging Molecules

Bridging molecules are molecules or parts of molecules that provide for attachment of cargo to a nucleic acid assembly. Preferably, bridging molecules provide for self assembly and for achieving desired stoichiometric ratios of cargo molecules. Bridging molecules can provide for attachment in any suitable form. For example, bridging molecules can be directly or indirectly attached, specifically attached, attached by covalent bond, non-covalent bond, nucleic acid hybridization, click chemistry reaction, specific binding molecules, or specific binding molecule pairs, or can be part of, or attached to, pairs of bridging molecules, the other bridging molecule in the pair of bridging molecules, or corresponding cargo molecules. Bridging molecules can also be embodied in or as part of any of the other components of the disclosed compositions, such a nucleic acids forming the nucleic acid assembly, cargo molecules, and effector molecules.

In some forms, each bridging molecule is part of or directly or indirectly attached to either or both the nucleic acid assembly and one or more of the cargo molecules. Cargo molecules and bridging molecules that are part of or attach to each other are said to correspond to each other. These relationships are preferably selected and designed so that the bridging molecules collectively attach cargo molecules to the nucleic acid assembly in the defined stoichiometric ratio for the cargo molecules having a defined stoichiometric ratio.

Generally, bridging molecules have components or regions that facilitate attachment to nucleic acid assemblies, cargo, and effector molecules, which can be referred to as “attachment points.” Attachment points can have or be functionalized to have desired properties such as a specific binding to target components or regions of, for example, other bridging molecules, nucleic acid assemblies, nucleic acid components of nucleic acid assemblies, cargo molecules, and effector molecules. In some forms, the attachment point can be biotinylated for capturing a streptavidin-functionalized cargo molecule or effector molecule. In some forms, the attachment point of a bridging molecule can be modified with chemical moieties. Non-limiting examples include Click-chemistry groups (e.g., azide group, alkyne group, DIBO/DBCO), amine groups, and thiol groups. In some forms, cargo components, such as DNA-binding proteins and guide RNAs, can be used as bridging molecules or attachment points of bridging molecules to attach other components to the nucleic acid assembly.

Bridging molecules can be, for example, short single strands of nucleic acids (e.g., DNA) to direct the attachment of cargo and effector molecules. Such nucleic acid bridging molecules can have, for example, sequence that is complementary to sequence on a nucleic acid molecule of a nucleic acid assembly (such as on a scaffold strand or a stable overhang of a staple strand). Such features can serve as the basis for attachment of the bridging molecule to nucleic acid assemblies. Nucleic acid bridging molecules can also have additional nucleotides that facilitate attachment of cargo and effector molecules, which can be referred to as “attachment points.” Attachment points can be functionalized to have desired properties such as a specific sequence to hybridize to a target nucleic acid sequence or a targeting element (such as on a cargo molecule). In some forms, the attachment point can be biotinylated for capturing a streptavidin-functionalized cargo molecule or effector molecule. In some forms, the attachment point of the bridging molecule can be modified with chemical moieties. Non-limiting examples include Click-chemistry groups (e.g., azide group, alkyne group, DIBO/DBCO), amine groups, and thiol groups. In some forms, some bases located inside the nucleic acid bridging molecule can be modified using base analogs (e.g., 2-Aminopurine, locked nucleic acids, such as those modified with an extra bridge connecting the 2′ oxygen and 4′ carbon), which can serve as a linker to attach functional moieties (e.g., lipids, proteins). In some forms, cargo components, such as DNA-binding proteins and guide RNAs, can be used as bridging molecules or attachment points of bridging molecules to attach other components to the nucleic acid assembly.

Bridging molecules can attach components (e.g., cargo molecules, effector molecules) to nucleic acid assemblies in any suitable way. In some forms, a bridging molecule is directly attached to the nucleic acid assembly and directly attached to the component. In some forms, a bridging molecule is indirectly attached to the nucleic acid assembly and directly attached to the component. In some forms, a bridging molecule is directly attached to the nucleic acid assembly and indirectly attached to the component. In some forms, a bridging molecule is indirectly attached to the nucleic acid assembly and indirectly attached to the component.

In some forms, direct attachments of bridging molecules to the nucleic acid assembly and/or cargo molecules each comprise a covalent bond, a non-covalent bond, or both a covalent bond and a non-covalent bond. In some forms, a plurality of the non-covalent bonds are involved in nucleic acid hybridization, whereby specificity of the attachment of the cargo molecule to the nucleic acid assembly is provided by the specificity of the nucleic acid hybridization. In some forms, at least one of the covalent bonds is formed by a click chemistry reaction, whereby specificity of the attachment of the cargo molecule to the nucleic acid assembly is provided by the specificity of the click chemistry reaction. In some forms, at least one of the non-covalent bonds is formed by specific binding molecules in a specific binding molecule pair, whereby specificity of the attachment of the cargo molecule to the nucleic acid assembly is provided by the specificity of the specific binding molecule pair.

Bridging molecules can also be used pairs, where the bridging molecules in the pair specifically bind or attach to each other. Generally, this binding or attachment is direct, but it can also be indirect. Such pairs of bridging molecules generally have components or regions that facilitate binding or attachment to the other bridging molecule in the pair, which can be referred to as “bridging points.” Bridging points can have or be functionalized to have desired properties such as a specific binding to target components or regions of the other bridging molecule in the pair. In some forms, the bridging point can be biotinylated for capturing streptavidin on the other bridging molecule in the pair. In some forms, the bridging point of a bridging molecule can be modified with chemical moieties. Non-limiting examples include Click-chemistry groups (e.g., azide group, alkyne group, DIBO/DBCO), amine groups, and thiol groups.

Where the bridging molecules in a pair are short single strands of nucleic acids (e.g., DNA), the bridging points of such nucleic acid bridging molecules can each have, for example, sequence that is complementary to sequence on the other bridging molecule in the pair. Such features can serve as the basis for attachment of the pair of bridging molecules to each other. Bridging points can be functionalized to have desired properties such as a specific sequence to hybridize to the bridging point on the other bridging molecule in the pair.

In some forms, a bridging molecule can be part of the nucleic acid assembly, where the bridging molecule that is part of the nucleic acid assembly attaches directly or indirectly to a corresponding cargo molecule. Based on this, the bridging molecule that is part of the nucleic acid assembly specifically attaches the corresponding cargo molecule to the nucleic acid assembly.

In some forms, a bridging molecule can be part of a cargo molecule, where the bridging molecule that is part of the cargo molecule attaches directly or indirectly to the nucleic acid assembly. Based on this, bridging molecule that is part of the cargo molecule specifically attaches the cargo molecule to the nucleic acid assembly.

In some forms, a bridging molecule can be part of a cargo molecule, where the bridging molecule that is part of the cargo molecule is part of the nucleic acid assembly. Based on this, the bridging molecule that is part of the cargo molecule specifically attaches the cargo molecule to the nucleic acid assembly.

Pairs of bridging molecules can bind or attach to each other indirectly. In some forms, indirect binding or attachment of bridging molecules in a pair can be via a connector molecule, where the connector molecule binds to or attaches to both of the bridging molecules in the pair. Generally, this binding or attachment is direct, but it can also be indirect. Such pairs of bridging molecules generally have components or regions that facilitate binding or attachment to the connector molecule. Similarly, the connector molecule corresponding to the pair of bridging molecules has one component or region that facilitates binding or attachment to one of the bridging molecules of the pair and another component or region that facilitates binding or attachment to the other bridging molecules of the pair. In some forms, both of the components or regions on the connector molecule that facilitate binding or attachment to the bridging molecules can be the same, and the components or regions on the bridging molecules that facilitate binding or attachment to the connector molecule can be the same, such that the connector molecule can bind or attach to the bridging molecules in either orientation. The components or regions on the connector molecule that facilitate binding or attachment to the bridging molecules can be referred to as “connector points.” Connector points can have or be functionalized to have desired properties such as a specific binding to target components or regions of the bridging molecules in a pair. In some forms, the connector point can be biotinylated for capturing streptavidin on a bridging molecule. In some forms, the connector point of a connector molecule can be modified with chemical moieties. Non-limiting examples include Click-chemistry groups (e.g., azide group, alkyne group, DIBO/DBCO), amine groups, and thiol groups.

Generally for each pair of bridging molecules, one bridging molecule of the pair is part of or is directly or indirectly attached to the nucleic acid assembly and the other bridging molecule of the pair is part of or directly or indirectly attached to a corresponding cargo molecule. Based on this, specific binding of the pair of bridging molecules specifically attaches the corresponding cargo molecule to the nucleic acid assembly.

In some forms, one of the bridging molecules of a pair can be part of the nucleic acid assembly, where the other bridging molecule of the pair attaches directly or indirectly to a corresponding cargo molecule. Based on this, the bridging molecule pair specifically attaches the corresponding cargo molecule to the nucleic acid assembly.

In some forms, one of the bridging molecules of a pair can be part of a cargo molecule, where the other bridging molecule of the pair attaches directly or indirectly to the nucleic acid assembly. Based on this, bridging molecule pair specifically attaches the cargo molecule to the nucleic acid assembly.

In some forms, one of the bridging molecules of a pair can be part of a cargo molecule, where the other bridging molecule of the pair is part of the nucleic acid assembly. Based on this, the bridging molecule pair specifically attaches the cargo molecule to the nucleic acid assembly.

In some forms, the attachments of bridging molecules to nucleic acid assemblies and to components can be, independently, covalent or non-covalent. In some forms, the attachments of bridging molecules to nucleic acid assemblies and to components can be, independently, formed prior to, during, or following assembly of the nucleic acid assembly. In some forms, the attachments of bridging molecules to nucleic acid assemblies and to components can be, independently, via attachment points on, in, part of, or attached to the bridging molecule. In some forms, the attachment points of bridging molecules can be, independently, covalent or non-covalent.

In some forms, direct attachment of one bridging molecule of a pair to the nucleic acid assembly and/or direct attachment of the other bridging molecule of the pair to a cargo molecule each comprise a covalent bond, a non-covalent bond, or both a covalent bond and a non-covalent bond. In some forms, a plurality of such non-covalent bonds are involved in nucleic acid hybridization, whereby specificity of the attachment of the cargo molecule to the nucleic acid assembly is provided by the specificity of the nucleic acid hybridization. In some forms, at least one of the covalent bonds is formed by a click chemistry reaction, whereby specificity of the attachment of the cargo molecule to the nucleic acid assembly is provided by the specificity of the click chemistry reaction. In some forms, at least one of the non-covalent bonds is formed by specific binding molecules in a specific binding molecule pair, whereby specificity of the attachment of the cargo molecule to the nucleic acid assembly is provided by the specificity of the specific binding molecule pair.

Bridging molecules can facilitate compositions having defined stoichiometric ratios of cargo molecules. In some forms, stoichiometric ratios can be established by a one-to-one correspondence of bridging molecules and their corresponding cargo molecules such that the number of a particular form of bridging molecule (and/or the number of attachment points on the nucleic acid assembly for this particular form of bridging molecule) match the number of the corresponding cargo molecule desired to be attached to the nucleic acid assembly. In this way, the ratio of different forms of bridging molecules (and/or the attachment points on the nucleic acid assembly for the different forms of bridging molecules) can establish the stoichiometric ratio of the different corresponding cargo molecules.

Self assembly of cargo in and on nucleic acid assemblies can be facilitated by, for example, selection of specific binding and/or attachment of bridging molecules, corresponding cargo molecules, nucleic acid assemblies. Generally, using the same corresponding attachment points, bridging points, and/or connector points for the same forms of bridging molecules and their corresponding cargo molecules and using the different corresponding attachment points, bridging points, and/or connector points for different forms of bridging molecules and their corresponding cargo molecules can allow association and attachment of the different forms of cargo molecules at the same time in the same mixture, relying on the specificity of the different attachment points, bridging points, and/or connector points to facilitate assembly of all of the different forms of cargo molecules in the desired ratios.

Bridging molecules can also facilitate release of cargo. As just one example, the use of RNA/DNA hybrids as part of the bridging molecules—in particular via the attachment, bridging, and/or connector points—can facilitate release of cargo, break-up of the nucleic acid assembly, or both (through the action of RNA/DNA specific nuclease, for example). Other labile points in bridging molecules and/or in their attachments to nucleic acid assemblies and/or cargo can be sued to facilitate release cargo and other components of the compositions.

4. Targeting Molecules

In some forms, the compositions comprise one or more targeting molecules that specifically targets the nucleic acid assembly to one or more types of cells, tissues, organs, or microenvironments relative to other types of cells, tissues, organs, or microenvironments in vivo, or to mediate specific binding to a protein, lipid, polysaccharide, nucleic acid, etc.

Exemplary targeting molecules include proteins, peptides, nucleic acids, lipids, saccharides, or polysaccharides that bind to one or more targets associated with an organ, tissue, cell, or extracellular matrix, or specific type of tumor or infected cell. The degree of specificity with which the nucleic acid assemblies are targeted can be modulated through the selection of a targeting molecule with the appropriate affinity and specificity. For example, antibodies, or antigen-binding fragments thereof are very specific.

Typically, the targeting moieties exploit the surface-markers specific to a biologically functional class of cells, such as antigen presenting cells. Dendritic cells express a number of cell surface receptors that can mediate endocytosis. In some forms, overhang sequences include nucleotide sequences that are complementary to nucleotide sequences of interest, for example HIV-1 RNA viral genome.

In some forms, the disclosed compositions can be targeted via lectin-mediated endocytosis of primary hepatocytes and other biotechnologically relevant cell lines. For example, most hybridoma cells express either CD22 or CD44, two lectins with biochemical profiles comparable to ASGPR (32, 48). The availability of glycomimetic ligands for both receptors enables the nucleic acid assembly-based delivery of, for example, CRISPR RNPs to facilitate antibody engineering and production (49-51). Furthermore, the disclosed compositions and methods will produce translational impacts for the industrial production of biomolecules where the screening of a large number of genetic constructs is required to improve yields (2). Here, the use of lectin-expressing HEK293 cell lines such as HKB11 cells allows for high-fidelity and -efficiency gene editing with the disclosed nucleic acid assembly-based delivery platform (52, 53).

Additional functional groups can be introduced on the staple strand for example by incorporating biotinylated nucleotides into the staple strand. Any streptavidin-coated targeting molecules are therefore introduced via biotin-streptavidin interaction. In other forms, non-naturally occurring nucleotides are included for desired functional groups for further modification. Exemplary functional groups include targeting elements, immunomodulatory elements, chemical groups, biological macromolecules, and combinations thereof.

i. Antibodies

In some forms, nucleic acid assemblies are modified to include one or more antibodies. Antibodies that function by binding directly to one or more epitopes, other ligands, or accessory molecules at the surface of cells can be coupled directly or indirectly to the assemblies. In some forms, the antibody or antigen binding fragment thereof has affinity for a receptor at the surface of a specific cell type, such as a receptor expressed at the surface of macrophage cells, dendritic cells, or epithelial lining cells. In some forms, the antibody binds one or more target receptors at the surface of a cell that enables, enhances or otherwise mediates cellular uptake of the antibody-bound assembly, or intracellular translocation of the antibody-bound assembly, or both.

Any specific antibody can be used to modify the nucleic acid assemblies. For example, antibodies can include an antigen binding site that binds to an epitope on the target cell. Binding of an antibody to a “target” cell can enhance or induce uptake of the associated nucleic acid assemblies by the target cell protein via one or more distinct mechanisms.

In some forms, the antibody or antigen binding fragment binds specifically to an epitope. The epitope can be a linear epitope. The epitope can be specific to one cell type or can be expressed by multiple different cell types. In other forms, the antibody or antigen binding fragment thereof can bind a conformational epitope that includes a 3-D surface feature, shape, or tertiary structure at the surface of the target cell.

In some forms, the antibody or antigen binding fragment that binds specifically to an epitope on the target cell can only bind if the protein epitope is not bound by a ligand or small molecule.

Various types of antibodies and antibody fragments can be used to modify nucleic acid assemblies, including whole immunoglobulin of any class, fragments thereof, and synthetic proteins containing at least the antigen binding variable domain of an antibody. The antibody can be an IgG antibody, such as IgG1, IgG2, IgG3, or IgG4 subtypes. An antibody can be in the form of an antigen binding fragment including a Fab fragment, F(ab′)₂ fragment, a single chain variable region, and the like. Antibodies can be polyclonal, or monoclonal (mAb). Monoclonal antibodies include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they specifically bind the target antigen and/or exhibit the desired biological activity (U.S. Pat. No. 4,816,567; and Morrison, et al., Proc. Natl. Acad. Sci. USA, 81: 6851-6855 (1984)). The antibodies can also be modified by recombinant means, for example by deletions, additions or substitutions of amino acids, to increase efficacy of the antibody in mediating the desired function. Substitutions can be conservative substitutions. For example, at least one amino acid in the constant region of the antibody can be replaced with a different residue (see, e.g., U.S. Pat. Nos. 5,624,821; 6,194,551; WO 9958572; and Angal, et al., Mol. Immunol. 30:105-08 (1993)). In some cases changes are made to reduce undesired activities, e.g., complement-dependent cytotoxicity. The antibody can be a bi-specific antibody having binding specificities for at least two different antigenic epitopes. In some forms, the epitopes are from the same antigen. In other forms, the epitopes are from two different antigens. Bi-specific antibodies can include bi-specific antibody fragments (see, e.g., Hollinger, et al., Proc. Natl. Acad. Sci. U.S.A., 90:6444-48 (1993); Gruber, et al., J. Immunol., 152:5368 (1994)).

Antibodies that target the nucleic acid assemblies to a specific epitope can be generated by any means known in the art. Exemplary descriptions of techniques for antibody generation and production include Delves, Antibody Production: Essential Techniques (Wiley, 1997); Shephard, et al., Monoclonal Antibodies (Oxford University Press, 2000); Goding, Monoclonal Antibodies: Principles And Practice (Academic Press, 1993); and Current Protocols In Immunology (John Wiley & Sons, most recent edition). Fragments of intact Ig molecules can be generated using methods well known in the art, including enzymatic digestion and recombinant means.

ii. Capture Tags

In some forms, assemblies include one or more sequences of nucleic acids that act as capture tags, or “bait” sequences to specifically bind one or more targeted molecules. For example, in some forms, overhang sequences include nucleotide “bait” sequences that are complementary to any target nucleotide sequence, for example HIV-1 RNA viral genome. In some forms, functional groups are present on one or more staple strands to act as capture tags. For example, in some forms, one or more biotinylated nucleotides are incorporated into the staple strand. Streptavidin-coated molecules are therefore introduced via biotin-streptavidin interaction.

Typically, targeting moieties exploit the surface-markers specific to a group of cells to be targeted. Exemplary targeting elements include proteins, peptides, nucleic acids, lipids, saccharides, or polysaccharides that bind to one or more targets associated with cell, or extracellular matrix, or specific type of tumor or infected cell. Targeting molecules can be selected based on the desired physical properties, such as the appropriate affinity and specificity for the target. Exemplary targeting molecules having high specificity and affinity include antibodies, or antigen-binding fragments thereof. Therefore, in some forms, nucleic acid assemblies include one or more antibodies or antigen binding fragments specific to an epitope. The epitope can be a linear epitope. The epitope can be specific to one cell type or can be expressed by multiple different cell types. In other forms, the antibody or antigen binding fragment thereof can bind a conformational epitope that includes a 3-D surface feature, shape, or tertiary structure at the surface of the target cell.

iii. Lectins

Lectins that can be covalently attached to microparticles, nanoparticles, and nucleic acid assemblies to render them target specific to the mucin and mucosal cell layer include lectins isolated from Abrus precatroius, Agaricus bisporus, Anguilla anguilla, Arachis hypogaea, Pandeiraea simplicifolia, Bauhinia purpurea, Caragan arobrescens, Cicer arietinum, Codium fragile, Datura stramonium, Dolichos biflorus, Erythrina corallodendron, Erythrina cristagalli, Euonymus europaeus, Glycine max, Helix aspersa, Helix pomatia, Lathyrus odoratus, Lens culinaris, Limulus polyphemus, Lysopersicon esculentum, Maclura pomifera, Momordica charantia, Mycoplasma gallisepticum, Naja mocambique, as well as the lectins Concanavalin A, Succinyl-Concanavalin A, Triticum vulgaris, Ulex europaeus I, II and III, Sambucus nigra, Maackia amurensis, Limax fluvus, Homarus americanus, Cancer antennarius, and Lotus tetragonolobus.

The choice of targeting molecule will depend on the method of administration of the assembly composition and the cells or tissues to be targeted. The targeting molecule may generally increase the binding affinity of the particles for cell or tissues or may target the assembly to a particular tissue in an organ or a particular cell type in a tissue.

iv. Aptamers

In some forms, compositions described herein are conjugated with nucleic acid-based aptamers which contribute to their preferential targeting to one or more types of cells, tissues, organs, or microenvironments. In some forms, the aptamer specifically binds to surface or transmembrane proteins, such as, for example, integrin αvβ3, VEGF receptor, EGF receptor, HER2, HER3, MUC1, PSMA, and receptor tyrosine kinase RET. The aptamer may comprise modified or unmodified DNA or RNA. In some forms, the aptamers are nuclease resistant. In some forms, the aptamer is an RNA aptamer that is 2′-modified (e.g., 2′-fluro and 2′-O-methyl). Non-limiting examples of aptamers contemplated for use in the disclosed compositions and methods that have been recently used to target assemblies are provided in Friedman A D, et al. (see Table 7).

5. Intracellular Trafficking Molecules

Bioactive macromolecular peptides and oligonucleotides have significant therapeutic potential. However, due to their size, they have no ability to enter the cytoplasm of cells. Peptide/Protein transduction domains (PTDs), also called cell-penetrating peptides (CPPs), can promote uptake of macromolecules via endocytosis. Other CPPs facilitate entry other than via endosomes. Where cargo is internalized via an endosome, endosomal escape into the cytoplasm is important. Hydrophobic amino acid R groups are known to play a vital role in viral escape from endosomes, and so-called endosome escape elements generally embody this feature. Finally, for cargo that acts in the cell nucleus, transport or passage of the cargo into the nucleus is important. Nuclear localization signals can be used to facilitate nuclear trafficking of cargo.

i. Cell Penetrating Peptides

Cell penetrating peptides (CPPs), which can also be referred to as a “protein transduction domains” (PTDs), a “membrane translocating sequences,” and a “Trojan peptides,” are commonly short peptides (e.g., from 4 to about 40 amino acids) that have the ability to translocate across a cellular membrane to gain access to the interior of a cell and to carry into the cells a variety of covalently and noncovalently conjugated cargoes, including proteins, oligonucleotides, and liposomes. They are typically highly cationic and rich in arginine and lysine amino acids.

Endocytosis is the primary mode of CPP cellular entry, although some CPPs appear to directly pass through the cell membrane. Endocytosis is a broad term encompassing a variety of pathways utilized by the cell to bring outside molecules in for a multitude of purposes—receptor & lipid recycling, inhibition of signal transduction, uptake of solutes/nutrients, and destruction of foreign/unwanted materials. The unifying property of endocytic pathways is, simply put, engagement of the plasma membrane for the formation of an intracellular membrane-bound organelle which will then transport the molecules to some destination within the cell. It is a tightly coordinated, energy-dependent process requiring extensive cellular adaptors and cytoskeletal rearrangements. Following organelle formation, an endocytic vesicle undergoes a pH-dependent maturation process as it transitions from the early sorting endosome (pH˜6.5-6) into the late endosome or multivesicular body (MVB; pH˜5). The acidification process is dependent on ionic gradients, requiring calcium, sodium and potassium efflux as well as hydrogen and chloride influx.

Endocytosis can further be broken down into phagocytosis and pinocytosis depending on whether entry is fluid-phase or not. Pinocytic pathways are most commonly engaged by CPPs—macropinocytosis, clathrin-dependent, and caveolin-dependent endocytosis, and lipid-raft endocytosis.

Macropinocytosis is defined as a receptor-independent and coat-independent process. Macropinocytosis is classically thought to be a non-specific mechanism to bring solutes/nutrients into the cell via large endocytic vesicles. However, in some cases the plasma membrane needs to be stimulated via the presence of extracellular growth factors. Further, different cell types may utilize this process in different ways given their particular needs. For example, some cells use this process to obtain nutrients or to recycle portions of their membranes while immune cells utilize macropinocytosis for the regulation of antigen presentation.

Clathrin-dependent endocytosis is a receptor-driven process that results in the formation of a ‘coated’ vesicle. During endocytosis, three-footed triskelion subunits assemble via adaptor proteins at cholesterol-deficient regions of the plasma membrane, forming into a lattice work to create a highly-ordered ‘caged’ structure which is internalized. This pathway is considered ubiquitous across all cell types and is utilized in a variety of ways; transferrins, low-density lipoproteins, hormones and neurotransmitters (during reuptake) are a few examples of molecules taken up by this pathway.

Caveolin-mediated entry shares both similarities and distinctions from clathrin-mediated entry. Caveolins are also coat proteins that form tight associations with cholesterol present in the plasma membrane. Unlike the trimeric clathrins, following recruitment to the plasma membrane caveolins form into a distinct “U” shape, with both N- and C-termini pointing towards the cytoplasm. The resulting invaginations resemble cave-like structures called caveolae. Not surprisingly, caveolins are found localized to cholesterol-rich lipid rafts. Many growth factors utilize this pathway, as do some pathogens. Further, caveolin-dependent endocytosis is important in transendothelial transport. Unlike the more ubiquitous mechanism of clathrin-dependent endocytosis, caveolae formation is impacted by many cellular factors such as cell type as well as cell cycle progression. Further, some cells express caveolins at low levels or not at all.

Lipid-raft endocytosis is a non receptor mediated, concentration-dependent form of endocytosis occurring at cholesterol-enriched lipid rafts in the plasma membrane but does not rely on caveolin coat formation. In this form of endocytosis, glycosylphosphatidylinositol-anchored proteins (GPI-AP) group into distinct microdomains, invaginate and form into GPI-enriched intracellular vesicles. This pathway is primarily used as a constitutive means of bringing in extracellular fluids through lipid-raft mediated pathways based in membrane-molecule interactions and have been described for SV40-virions, vitamins, GPI-binding proteins, MHC-class I, IL-2, and IgE. Even though these pathways show significant variability for the requirement of local mediators and in cargo fate (recycling, degradation, intracellular release), they share commonalities in that they are receptor mediated and proceed via an absorptive fluid phase mechanism.

Individual CPPs may engage one or more of the forms of pinocytosis defined above. For example, TAT appears use all of the endocytic mechanisms. Further, some CPPs such as the well-studied arginine-rich, 16-residue peptide corresponding to the third helix of the Drosophila melanogaster transcription factor Antennapedia homeodomain may enter via direct penetration.

Examples of hydrophilic/cationic CPPs include Penetratin or Antenapedia PTD (RQIKWFQNRRMKWKK (SEQ ID NO:174)), TAT (YGRKKRRQRRR (SEQ ID NO:175)), SynB1 (RGGRLSYSRRRFSTSTGR (SEQ ID NO:176)), SynB3 (RRLSYSRRRF (SEQ ID NO:177)), PTD-4 (PIRRRKKLRRLK (SEQ ID NO:178)), PTD-5 (RRQRRTSKLMKR (SEQ ID NO:179)), FHV Coat-(35-49) (RRRRNRTRRNRRRVR (SEQ ID NO:180)), BMV Gag-(7-25) (KMTRAQRRAAARRNRWTAR (SEQ ID NO:181)), HTLV-II Rex-(4-16) (TRRQRTRRARRNR (SEQ ID NO:182)), D-Tat (GRKKRRQRRRPPQ (SEQ ID NO:183)), and R9-Tat (GRRRRRRRRRPPQ (SEQ ID NO:184)).

Examples of amphipathic CPPs include Transportan (GWTLNSAGYLLGKINLKALAALAKKIL (SEQ ID NO:185)), MAP (KLALKLALKLALALKLA (SEQ ID NO:186)), SBP (MGLGLHLLVLAAALQGAWSQPKKKRKV (SEQ ID NO:187)), FBP (GALFLGWLGAAGSTMGAWSQPKKKRKV (SEQ ID NO:188)), MPG (ac-GALFLGFLGAAGSTMGAWSQPKKKRKV-cya (SEQ ID NO:189)), MPG^{(ΔNLS)} (ac-GALFLGFLGAAGSTMGAWSQPKSKRKV-cya (SEQ ID NO:190)), Pep-1 (ac-KETWWETWWTEWSQPKKKRKV-cya (SEQ ID NO:191)), and Pep-2 (ac-KETWFETWFTEWSQPKKKRKV-cya (SEQ ID NO:192)).

Examples of CPPs with periodic sequences include Polyarginines R×N (4<N<17), Polylysines K×N (4<N<17), (RAca)6R, (RAbu)6R, (RG)6R, (RM)6R, (RT)6R, (RS)6R, R10, (RA)6R, and R7.

ii. Endosomal Escape Elements

The cargo and other components of the disclosed compositions can include or be associated with one or more endosomal escape elements. An endosome has three potential fates: it may be targeted to the lysosome from the MVB for destruction of its intraluminal contents or its contents may be recycled back to the plasma membrane either directly from the sorting endosome or after routing from the MVB to the trans Golgi network. Alternatively, some endosomal contents such as nutrients are needed by the cell and thus must be released into the cytosol. This later process is mediated by the formation of intraluminal vesicles (ILVs) inside the endosome, essentially the formation of an exosome within the endosome. The ILV then undergoes a process known as ‘backfusion’ where it fuses with the endosomal membrane and its contents are released into the cytosol. Because of this, endosomes represent a potential trap preventing cargo from reaching the cytoplasm or nucleus of the cell.

Examples of endosomal escape elements include chloroquine, TAT peptide, melittin, mellitin-like peptides, fusogenic lipid, and fusogenic protein. A suitable fusogenic lipid may be dioleoylphosphatidyl-ethanolamine (DOPE). Fucosogenic peptides include HSWYG peptide (GLFHAIAHFIHGGWHGLIHGWYGGC (SEQ ID NO:193)) and 8 mer polyarginine.

Viruses have evolutionarily addressed the endosomal escape problem by destabilizing the endosomal lipid bilayer membrane by insertion of motifs containing hydrophobic amino acid R groups. For example, the hemagglutinin (HA2) endosomal escape domain from influenza virus has been used to enhance cargo delivery into cells. Likewise, addition of hydrophobic aromatic ring containing amino acids, Phe (F) or Trp (W), can enhance effective cargo delivery. Other endosomal escape elements have been developed based on hydrophobic amino acids (U.S. Application Publication No. 2017/0275650).

Endosomal escape can also be aided by promoting endosomolysis using endosomolytic agents (Erazo-Oliveras et al., Nat Methods. 11(8):861-867 (2014); Najjar et al., J Vis Exp. 103 (2015)). Endosomal escape can also be aided by separating or releasing the cargo form the cell penetrating peptide. If cellular entry is receptor-mediated, it could well be that high affinity of the CPP for its receptor is in part due to low off rates and hence trapping of its linked cargo in the endosomes may essentially be a kinetic problem. Separating or releasing the cargo from the CPP can be facilitated by attaching the CPP to the nucleic acid assembly and releasing the cargo from the nucleic acid assembly. Cargo released from association with the CPP can then escape by, for example endosomal leakage.

iii. Nuclear Localization Sequences

The cargo and other components of the disclosed compositions can include or be associated with one or more (e.g., two or more, three or more, or four or more) nuclear localization sequences (NLSs). Any convenient NLS can be used. Examples include Class 1 and Class 2 “monopartite NLSs,” as well as NLSs of Classes 3-5 (Kosugi et al., J Biol Chem. 284(1):478-485 (2009)). In some cases, an NLS has the formula: (K/R)(K/R)X₁₀₋₁₂(K/R)₃₋₅. In some cases, an NLS has the formula: K(K/R)X(K/R) (SEQ ID NO:194).

Examples of NLSs that can be used as an NLS-containing peptide (or conjugated to any convenient cationic polypeptide such as an HTP or cationic polymer or cationic amino acid polymer or anionic amino acid polymer) include: T-ag NLS (PKKKRKV (SEQ ID NO:195)), T-Ag-derived NLS (PKKKRKVEDPYC-SV40 (SEQ ID NO:196)), NLS SV40 (PKKKRKVGPKKKRKVGPKKKRKVGPKKKRKVGC (SEQ ID NO:197)), CYGRKKRRQRRR-N-terminal cysteine of cysteine-TAT (SEQ ID NO:198), CSIPPEVKFNKPFVYLI (SEQ ID NO:199), DRQIKIWFQNRRMKVVKK (SEQ ID NO:200), PKKKRKVEDPYC-C-term cysteine of an SV40 T-Ag-derived NLS (SEQ ID NO:196), and cMyc NLS (PAAKRVKLD (SEQ ID NO:165)). Other useful NLSs are described in Kosugi et al., J Biol Chem. 284(1):478-485 (2009).

iv. Mitochondrial Localization Sequences

The cargo and other components of the disclosed compositions can include or be associated with one or more (e.g., two or more, three or more, or four or more) mitochondrial localization sequences (MLSs). Any convenient mitochondrial localization sequence can be used. Examples of mitochondrial localization sequences include: PEDEIWLPEPESVDVPAKPISTSSMMM (SEQ ID NO:166), a mitochondrial localization sequence of SDHB, mono/di/triphenylphosphonium or other phosphoniums, VAMP 1A, VAMP 1B, the 67 N-terminal amino acids of DGAT2, and the 20 N-terminal amino acids of Bax.

6. Nucleic Acid Modifications

The disclosed nucleic acids can, in some forms, comprise non-naturally occurring nucleic acids and/or non-naturally occurring nucleotides and/or nucleotide analogs, and/or chemically modifications. For example, nucleic acids that are components of, or are used with, nucleic acid assemblies (such as scaffolds, staples, bridging molecules, etc.), nucleic acids that are cargo (such as guide sequences, HCR templates, mRNA, functional RNA, etc.), and targeting and effector molecules (such as aptamers). Non-naturally occurring nucleic acids can include, for example, mixtures of naturally and non-naturally occurring nucleotides. Non-naturally occurring nucleotides and/or nucleotide analogs may be modified at the ribose, phosphate, and/or base moiety. In some forms, nucleic acids can comprise ribonucleotides and non-ribonucleotides. In some such forms, nucleic acids can comprise one or more ribonucleotides and one or more deoxyribonucleotides. In some forms, nucleic acids can comprise one or more non-naturally occurring nucleotide or nucleotide analog such as a nucleotide with phosphorothioate linkage, boranophosphate linkage, a locked nucleic acid (LNA) nucleotides comprising a methylene bridge between the 2′ and 4′ carbons of the ribose ring, peptide nucleic acids (PNA), bridged nucleic acids (BNA), or morpholinos. Other examples of modified nucleotides include 2′-O-methyl analogs, 2′-deoxy analogs, 2-thiouridine analogs, N6-methyladenosine analogs, or 2′-fluoro analogs. Further examples of modified nucleotides include linkage of chemical moieties at the 2′ position, including but not limited to peptides, nuclear localization sequence (NLS), peptide nucleic acid (PNA), morpholinos, polyethylene glycol (PEG), triethylene glycol, or tetraethyleneglycol (TEG). Further examples of modified bases include, but are not limited to, 2-aminopurine, 5-bromo-uridine, pseudouridine (Ψ), N¹-methylpseudouridine (me¹Ψ), 5-methoxyuridine (5moU), inosine, 7-methylguanosine. Examples of nucleic acid chemical modifications include, without limitation, incorporation of 2′-O-methyl (M), 2′-O-methyl-3′-phosphorothioate (MS), phosphorothioate (PS), S-constrained ethyl(cEt), 2′-O-methyl-3′-thioPACE (MSP), or 2′-O-methyl-3′-phosphonoacetate (MP) at one or more terminal nucleotides. Such chemically modified nucleic acids can comprise increased stability and increased activity (for active nucleic acids) as compared to unmodified nucleic acids.

In some forms, the 5′ and/or 3′ end of a nucleic acids can be modified by a variety of functional moieties including fluorescent dyes, polyethylene glycol, cholesterol, proteins, or detection tags. In some forms, at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 75, 100, 150, 200, 250, 300, 400, 500, 600, 700, 800, 900, 1,000 nucleotides of a nucleic acid can be chemically modified. In some forms, 3-5 nucleotides at either the 3′ or the 5′ end of a nucleic acid can be chemically modified. In some forms, 2′-F modification is introduced at the 3′ end of a nucleic acid. In some forms, three to five nucleotides at the 5′ and/or the 3′ end of the nucleic acid are chemically modified with 2′-O-methyl (M), 2′-O-methyl-3′-phosphorothioate (MS), S-constrained ethyl(cEt), 2′-O-methyl-3′-thioPACE (MSP), or 2′-O-methyl-3′-phosphonoacetate (MP). In some forms, some or all of the phosphodiester bonds of a nucleic acid are substituted with phosphorothioates (PS) for enhancing levels of gene disruption. In some forms, more than five nucleotides at the 5′ and/or the 3′ end of a nucleic acid can be chemically modified with 2′-O-Me, 2′-F, or S-constrained ethyl (cEt). In some forms, a nucleic acid can be modified to comprise a chemical moiety at its 3′ and/or 5′ end. Such moieties include, but are not limited to amine, azide, alkyne, thio, dibenzocyclooctyne (DBCO), Rhodamine, peptides, nuclear localization sequence (NLS), peptide nucleic acid (PNA), morpholinos, polyethylene glycol (PEG), triethylene glycol, or tetraethyleneglycol (TEG). In some forms, the chemical moiety can be conjugated to the nucleic acid by a linker, such as an alkyl chain. In some forms, the chemical moiety can be used to attach the nucleic acid to another molecule, such as DNA, RNA, protein, nanoparticle, or nucleic acid assembly.

Examples of modified nucleotides (such as non-naturally occurring nucleotides) include, but are not limited to, diaminopurine, S²T, 5-fluorouracil, 5-bromouracil, 5-chlorouracil, 5-iodouracil, hypoxanthine, xantine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-D46-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Nucleic acid molecules may also be modified at the base moiety (e.g., at one or more atoms that typically are available to form a hydrogen bond with a complementary nucleotide and/or at one or more atoms that are not typically capable of forming a hydrogen bond with a complementary nucleotide), sugar moiety or phosphate backbone. Nucleic acid molecules may also contain amine-modified groups, such as aminoallyl-dUTP (aa-dUTP) and aminohexhylacrylamide-dCTP (aha-dCTP) to allow covalent attachment of amine reactive moieties, such as N-hydroxy succinimide esters (NHS).

In some forms, phosphorothioate modified backbone on the nucleic acid can be used to improve stability of the nucleic acids to degradation by exonuclease. For example, in some forms, the nucleic acids include modified nucleic acids that protect one or more regions of the nucleic acid from enzymic degradation or disruption in vivo. In some forms, nucleic acids can include modified nucleic acids at specific locations within the nucleic acid that direct the timing of the enzymic degradation of specific parts of the nucleic acid.

Locked nucleic acid (LNA) is a family of conformationally locked nucleotide analogues which, amongst other benefits, imposes truly unprecedented affinity and very high nuclease resistance to DNA and RNA oligonucleotides (Wahlestedt C, et al., Proc. Natl Acad. Sci. USA, 975633-5638 (2000); Braasch, D A, et al., Chem. Biol. 81-7 (2001); Kurreck J, et al., Nucleic Acids Res. 301911-1918 (2002)). In some forms, the nucleic acids are synthetic RNA-like high affinity nucleotide analogue, locked nucleic acids. In some forms, nucleic acids can be locked nucleic acids.

Peptide nucleic acid (PNA) is a nucleic acid analog in which the sugar phosphate backbone of natural nucleic acid has been replaced by a synthetic peptide backbone usually formed from N-(2-amino-ethyl)-glycine units, resulting in an achiral and uncharged mimic (Nielsen P E et al., Science 254, 1497-1500 (1991)). It is chemically stable and resistant to hydrolytic (enzymatic) cleavage. In some forms, nucleic acids can be PNAs. In other forms, the staple strands are PNAs.

In some forms, nucleic acid can comprise morpholino oligonucleotides. Morpholino oligonucleotides are typically composed of two more morpholino monomers containing purine or pyrimidine base-pairing moieties effective to bind, by base-specific hydrogen bonding, to a base in a polynucleotide, which are linked together by phosphorus-containing linkages, one to three atoms long, joining the morpholino nitrogen of one monomer to the 5′ exocyclic carbon of an adjacent monomer. The purine or pyrimidine base-pairing moiety is typically adenine, cytosine, guanine, uracil or thymine. The synthesis, structures, and binding characteristics of morpholino oligomers are detailed in U.S. Pat. Nos. 5,698,685, 5,217,866, 5,142,047, 5,034,506, 5,166,315, 5,521,063, and 5,506,337.

Important properties of the morpholino-based subunits typically include: the ability to be linked in a oligomeric form by stable, uncharged backbone linkages; the ability to support a nucleotide base (e.g. adenine, cytosine, guanine, thymidine, uracil or inosine) such that the polymer formed can hybridize with a complementary-base target nucleic acid, including target RNA, with high Tₘ, even with oligomers as short as 10-14 bases; the ability of the oligomer to be actively transported into mammalian cells; and the ability of an oligomer:RNA heteroduplex to resist RNAse degradation.

Every compound, molecule, and composition described herein is intended to be and should be considered to be specifically disclosed herein. Further, every subgroup that can be identified within the above definition is intended to be and should be considered to be specifically disclosed herein. As a result, it is specifically contemplated that any nucleic acid assembly, or subgroup of nucleic acid assemblies can be either specifically included for or excluded from use or included in or excluded from a list of nucleic acid assemblies. For example, as one option, a group of nucleic acid assemblies is contemplated where each nucleic acid assembly is as described herein but is not icosahedral or tetrahedral. As another example, a group of nucleic acid molecules is contemplated where each nucleic acid molecule is as described herein and is able to assemble together to form a nucleic acid assembly.

### B. Kits

The materials described above as well as other materials can be packaged together in any suitable combination as a kit useful for performing, or aiding in the performance of, the disclosed method. It is useful if the kit components in a given kit are designed and adapted for use together in the disclosed method.

### C. Mixtures

Disclosed are mixtures formed by performing or preparing to perform the disclosed method. For example, disclosed are mixtures comprising nucleic acid molecules and cargo molecules.

Whenever the method involves mixing or bringing into contact compositions or components or reagents, performing the method creates a number of different mixtures. For example, if the method includes 3 mixing steps, after each one of these steps a unique mixture is formed if the steps are performed separately. In addition, a mixture is formed at the completion of all of the steps regardless of how the steps were performed. The present disclosure contemplates these mixtures, obtained by the performance of the disclosed methods as well as mixtures containing any disclosed reagent, composition, or component, for example, disclosed herein.

### D. Systems

Disclosed are systems useful for performing, or aiding in the performance of, the disclosed method. Systems generally comprise combinations of articles of manufacture such as structures, machines, devices, and the like, and compositions, compounds, materials, and the like. Such combinations that are disclosed or that are apparent from the disclosure are contemplated.

### Methods

Disclosed herein are various methods related to the disclosed nucleic acid assemblies and their use. For example, disclosed are methods of assembly of nucleic acid assemblies, attachment of cargo to nucleic acid assemblies, attachment of targeting moieties, effector molecules, and intracellular trafficking molecules to nucleic acid assemblies, production of nucleic acid molecules, production of crispr-cas effector proteins, production of bridging molecules, and administration of nucleic acid assembly compositions.

### A. Administration of Nucleic Acid Assembly Compositions

The disclosed nucleic acid assembly compositions can be administered to any cell, tissue, or subject in need thereof. Generally, the nucleic acid assembly compositions can be administered to cells, tissues, and subjects based on the cargo of the nucleic acid assembly and the need for the cargo of the cells, tissues, and subjects.

The term “modulate” as used herein refers to the ability of a compound to change an activity in some measurable way as compared to an appropriate control. As a result of the presence of compounds in the assays, activities can increase or decrease as compared to controls in the absence of these compounds. Preferably, an increase in activity is at least 25%, more preferably at least 50%, most preferably at least 100% compared to the level of activity in the absence of the compound. Similarly, a decrease in activity is preferably at least 25%, more preferably at least 50%, most preferably at least 100% compared to the level of activity in the absence of the compound. A compound that increases a known activity is an “agonist.” One that decreases, or prevents, a known activity is an “antagonist.”

The term “inhibit” means to reduce or decrease in activity or expression. This can be a complete inhibition of activity or expression, or a partial inhibition. Inhibition can be compared to a control or to a standard level. Inhibition can be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%.

The term “monitoring” as used herein refers to any method in the art by which an activity can be measured.

The term “providing” as used herein refers to any means of adding a compound or molecule to something known in the art. Examples of providing can include the use of pipettes, pipettemen, syringes, needles, tubing, guns, etc. This can be manual or automated. It can include transfection by any mean or any other means of providing nucleic acids to dishes, cells, tissue, cell-free systems and can be in vitro or in vivo.

The term “preventing” as used herein refers to administering a compound prior to the onset of clinical symptoms of a disease or conditions so as to prevent a physical manifestation of aberrations associated with the disease or condition.

The term “in need of treatment” as used herein refers to a judgment made by a caregiver (e.g. physician, nurse, nurse practitioner, or individual in the case of humans; veterinarian in the case of animals, including non-human mammals) that a subject requires or will benefit from treatment. This judgment is made based on a variety of factors that are in the realm of a care giver's expertise, but that include the knowledge that the subject is ill, or will be ill, as the result of a condition that is treatable by the compositions and methods.

As used herein, “subject” includes, but is not limited to, animals, plants, bacteria, viruses, parasites and any other organism or entity. The subject can be a vertebrate, more specifically a mammal (e.g., a human, horse, pig, rabbit, dog, sheep, goat, non-human primate, cow, cat, guinea pig or rodent), a fish, a bird or a reptile or an amphibian. The subject can be an invertebrate, more specifically an arthropod (e.g., insects and crustaceans). The term does not denote a particular age or sex. Thus, adult and newborn subjects, as well as fetuses, whether male or female, are intended to be covered. A patient refers to a subject afflicted with a disease or disorder. The term “patient” includes human and veterinary subjects.

By “treatment” and “treating” is meant the medical management of a subject with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. It is understood that treatment, while intended to cure, ameliorate, stabilize, or prevent a disease, pathological condition, or disorder, need not actually result in the cure, amelioration, stabilization or prevention. The effects of treatment can be measured or assessed as described herein and as known in the art as is suitable for the disease, pathological condition, or disorder involved. Such measurements and assessments can be made in qualitative and/or quantitative terms. Thus, for example, characteristics or features of a disease, pathological condition, or disorder and/or symptoms of a disease, pathological condition, or disorder can be reduced to any effect or to any amount.

A cell can be in vitro. Alternatively, a cell can be in vivo and can be found in a subject. A “cell” can be a cell from any organism including, but not limited to, a bacterium.

In some forms, the compounds described herein can be administered to a subject comprising a human or an animal including, but not limited to, a mouse, dog, cat, horse, bovine or ovine and the like, that is in need of alleviation or amelioration from a recognized medical condition.

By the term “effective amount” of a compound as provided herein is meant a nontoxic but sufficient amount of the compound to provide the desired result. As will be pointed out below, the exact amount required will vary from subject to subject, depending on the species, age, and general condition of the subject, the severity of the disease that is being treated, the particular compound used, its mode of administration, and the like. Thus, it is not possible to specify an exact “effective amount.” However, an appropriate effective amount can be determined by one of ordinary skill in the art using only routine experimentation.

The dosages or amounts of the compounds described herein are large enough to produce the desired effect in the method by which delivery occurs. The dosage should not be so large as to cause adverse side effects, such as unwanted cross-reactions, anaphylactic reactions, and the like. Generally, the dosage will vary with the age, condition, sex and extent of the disease in the subject and can be determined by one of skill in the art. The dosage can be adjusted by the individual physician based on the clinical condition of the subject involved. The dose, schedule of doses and route of administration can be varied.

The efficacy of administration of a particular dose of the compounds or compositions according to the methods described herein can be determined by evaluating the particular forms of the medical history, signs, symptoms, and objective laboratory tests that are known to be useful in evaluating the status of a subject in need treatment of a given diseases and/or conditions. These signs, symptoms, and objective laboratory tests will vary, depending upon the particular disease or condition being treated or prevented, as will be known to any clinician who treats such patients or a researcher conducting experimentation in this field. For example, if, based on a comparison with an appropriate control group and/or knowledge of the normal progression of the disease in the general population or the particular individual: (1) a subject's physical condition is shown to be improved (e.g., a tumor has partially or fully regressed), (2) the progression of the disease or condition is shown to be stabilized, or slowed, or reversed, or (3) the need for other medications for treating the disease or condition is lessened or obviated, then a particular treatment regimen will be considered efficacious.

By “pharmaceutically acceptable” is meant a material that is not biologically or otherwise undesirable, i.e., the material can be administered to a subject along with the selected compound without causing any undesirable biological effects or interacting in a deleterious manner with any of the other components of the pharmaceutical composition in which it is contained.

Any of the compounds having the formula I can be used therapeutically in combination with a pharmaceutically acceptable carrier. The compounds described herein can be conveniently formulated into pharmaceutical compositions composed of one or more of the compounds in association with a pharmaceutically acceptable carrier. See, e.g., Remington's Pharmaceutical Sciences, latest edition, by E.W. Martin Mack Pub. Co., Easton, Pa., which discloses typical carriers and conventional methods of preparing pharmaceutical compositions that can be used in conjunction with the preparation of formulations of the compounds described herein and which is incorporated by reference herein. These most typically would be standard carriers for administration of compositions to humans. In some forms, humans and non-humans, including solutions such as sterile water, saline, and buffered solutions at physiological pH. Other compounds will be administered according to standard procedures used by those skilled in the art.

The pharmaceutical compositions described herein can include, but are not limited to, carriers, thickeners, diluents, buffers, preservatives, surface active agents and the like in addition to the molecule of choice. Pharmaceutical compositions can also include one or more active ingredients such as antimicrobial agents, antiinflammatory agents, anesthetics, and the like.

The compounds and pharmaceutical compositions described herein can be administered to the subject in a number of ways depending on whether local or systemic treatment is desired, and on the area to be treated. Thus, for example, a compound or pharmaceutical composition described herein can be administered as an ophthalmic solution and/or ointment to the surface of the eye. Moreover, a compound or pharmaceutical composition can be administered to a subject vaginally, rectally, intranasally, orally, by inhalation, or parenterally, for example, by intradermal, subcutaneous, intramuscular, intraperitoneal, intrarectal, intraarterial, intralymphatic, intravenous, intrathecal and intratracheal routes. Parenteral administration, if used, is generally characterized by injection. Injectables can be prepared in conventional forms, either as liquid solutions or suspensions, solid forms suitable for solution or suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

Preparations for parenteral administration include sterile aqueous or non-aqueous solutions, suspensions, and emulsions which can also contain buffers, diluents and other suitable additives. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives can also be present such as, for example, antimicrobials, anti-oxidants, chelating agents, and inert gases and the like.

Formulations for topical administration can include ointments, lotions, creams, gels, drops, suppositories, sprays, liquids and powders. Conventional pharmaceutical carriers, aqueous, powder or oily bases, thickeners and the like can be necessary or desirable.

Compositions for oral administration can include powders or granules, suspensions or solutions in water or non-aqueous media, capsules, sachets, or tablets. Thickeners, flavorings, diluents, emulsifiers, dispersing aids or binders can be desirable.

The term “hit” refers to a test compound that shows desired properties in an assay. The term “test compound” refers to a chemical to be tested by one or more screening method(s) as a putative modulator. A test compound can be any chemical, such as an inorganic chemical, an organic chemical, a protein, a peptide, a carbohydrate, a lipid, or a combination thereof. Usually, various predetermined concentrations of test compounds are used for screening, such as 0.01 micromolar, 1 micromolar and 10 micromolar. Test compound controls can include the measurement of a signal in the absence of the test compound or comparison to a compound known to modulate the target.

The terms “high,” “higher,” “increases,” “elevates,” or “elevation” refer to increases above basal levels, e.g., as compared to a control. The terms “low,” “lower,” “reduces,” or “reduction” refer to decreases below basal levels, e.g., as compared to a control.

As used herein, the term “pharmacological activity” refers to the inherent physical properties of a composition, assembly, or component thereof. These properties include but are not limited to half-life, solubility, and stability and other pharmacokinetic properties.

A residue of a monomer unit or moiety refers to the portion of the monomer or moiety that is the resulting product of the monomer unit or moiety in a particular reaction scheme or subsequent formulation or chemical product, regardless of whether the portion of the monomer or moiety is actually obtained from the monomer unit or moiety. Thus, an ethylene glycol residue in a polyester refers to one or more —OCH₂CH₂O— units in the polyester, regardless of whether ethylene glycol was used to prepare the polyester. Similarly, an amino acid residue in a peptide refers to one or more —CO—CHR—NH— moieties in the polyester, regardless of whether the residue is obtained by reacting the amino acid to obtain the peptide.

As used herein, the term “activity” refers to a biological activity.

### Illustrations

Illustration 1: Formulation of Nucleic Acid Assemblies with Complex Cargo

The use of viral and viral-like particles to co-deliver sgRNA and DNA encoding for Cas proteins such as Cas9 or Cpf1 has been explored extensively. However, viral vectors suffer from off-target editing and genome instability due to Cas protein overexpression (Ran, F. A. et al., Nature 520, 186-91 (2015); Yin, H. et al., Nat Biotechnol 34, 328-33 (2016); Fu, Y. et al., Nat Biotechnol 31, 822-6 (2013)). Furthermore, viral genome size limitations do not allow for multiplexed delivery of CRISPR RNPs in addition to HDR template DNA. Liposomal delivery platforms provide only approximate control over RNP stoichiometry and encapsulation requires highly charged RNPs, limiting their utility for uncharged Cas proteins such as Cpf1. Finally, the used cationic lipids and polymers are toxic and thereby reduce cell viability, an important aspect for in vitro gene editing in difficult-to-transfect cells (Han, X. et al., Sci Adv 1, e1500454 (2015)).

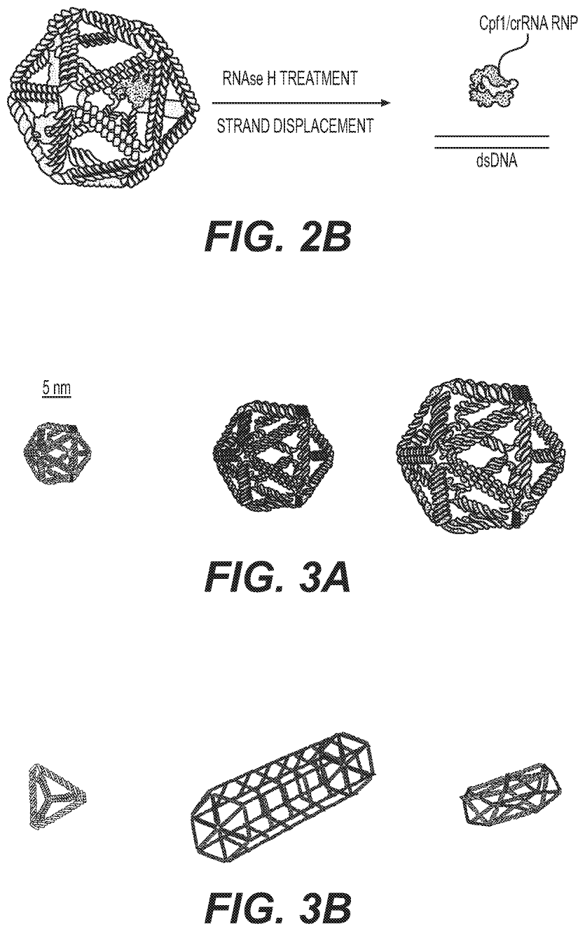

Alternatively, nucleic acid assemblies can act as delivery platforms for intact CRISPR RNPs. Watson-Crick base pairing offers unprecedented control over the stoichiometry and spatial organization of various targeting ligands and cargo (Sacca, B. et al., Angew Chem Int Ed Engl 49, 9378-83 (2010); Sacca, B. & Niemeyer, C. M., Chemical Society Reviews 40, 5910-5921 (2011); Meyer, R., et al., Angew Chem Int Ed Engl 54, 12039-43 (2015)). Nucleic acid assemblies are typically composed of double-stranded helices having 20 to 60 nick positions, enabling functionalization at any and all of these positions. Functionalization can be achieved via, for example, 10 to 20 nucleotide single-stranded overhangs or the chemical modification of staple strands. Overhangs can be oriented outward for functionalization with targeting ligands or inward to protect cargo from enzymatic degradation. For example, the Cas protein Cpf1 bound to the corresponding CRISPR RNA (crRNA) can be packaged in the interior of nucleic acid assemblies. A 3′ barcode on the crRNA that hybridizes with ssDNA overhangs on the nucleic acid assembly can be used to generate multiplexed delivery platform containing one to four CRISPR RNPs (FIG. 2A). At the same time, an HDR template can be analogously packaged in the interior of the nucleic acid assemblies. Thus, the delivery platform allows for multi-site cleavage along the genome and the co-delivery of HDR template.

After assembly, the nucleic acid assembly-CRISPR/Cpf1-crRNA complexes can be characterized, assessed, and validated using biochemical assays. Packaged HDR template can be similarly validated. In some forms, functionalization can be achieved via, for example, RNA-DNA hybridization, which allows at least three different methods for the controlled release of the cargo. The cargo can be protected from proteases and nuclease by functionalization of the nucleic acid assemblies with shielding molecules such as polyethylene glycol (PEG). Moreover, modified nucleotides can be used to increase the enzymatic stability of the crRNA and nucleic acid assemblies (30, 57). The resulting nucleic acid assembly compositions thus provide a customized delivery platform representing a new method of CRISPR RNP delivery, solving an important outstanding problem in the field of genome editing.

The nucleic acid assembly compositions can be designed, assessed, and optimized using one of more of the following assessments. This will be illustrated using an example of the disclosed compositions involving CRISPR-Cas as the cargo. In this example, Cas protein Cpf1 is used to illustrate RNP complexation with nucleic acid assemblies. Cpf1 benefits from less charge compared to Cas9, as well as being a staggered cleaving enzyme as opposed to the blunt cleaving Cas9, leading to an increased HDR likelihood. The 3′ end of the Cpf1 crRNA can be extended without loss of activity, and, unlike 5′ extensions, it is not removed by the enzyme but is processed in the cell (Zetsche, B. et al., Cell 163, 759-71 (2015); Zetsche, B. et al., Nat Biotechnol 35, 31-34 (2017)). These characteristics allow a native release method. To implement this, the crRNA is extended in 3′ direction to include a 25 nucleotide RNA barcode orthogonal to the human genome and complementary to single-stranded overhangs on the nucleic acid assembly. Thus, CRISPR/Cpf1 RNPs will bind the nucleic acid assemblies through these barcode sequences for packaging and delivery to cells (FIG. 2A). Complexation can be tested by, for example, gel shift assays or high-resolution analytical gel filtration, and complemented by imaging with transmission electron microscopy. Functionalized nucleic acid assembly monodispersity can be analyzed by, for example, dynamic light scattering (DLS) and surface charge can be analyzed by, for example, monitoring the zeta potential. Additionally, the functionalization with CRISPR/Cpf1 RNPs can be detected using, for example, antibodies binding the enzyme.

Loading and stoichiometry can be controlled by the number of matching overhangs (all corresponding to the same cargo molecule) and the number of different overhangs (each corresponding to a different cargo molecule). Each different overhang can be initially tested alone for complexation with the corresponding cargo molecule and the kinetics of release can be evaluated by, for example, varying the number of complementary of nucleotides on the barcode or in the presence of RNase H, which specifically cleaves RNA at RNA/DNA duplexes (FIG. 2B). Release of the Cpf1 can be assayed by, for example, surface plasmon resonance (SPR), total internal reflection fluorescence (TIRF) microscopy. Activity before and after release can be compared using, for example, plasmid cutting assays. Multiple different single-stranded overhang sequences can be added to the nucleic acid assemblies to allow for multiplexed delivery of Cpf1 RNP per nucleic acid assembly, allowing for multiple cleavage targets per single nucleic acid assembly. Numerous different stoichiometries and levels of multiplexed complexations can be used.

Known and validated crRNA sequences against targets in EMX1, DNMT1, and VEGFA can be used to assess crRNA sequences against other targets using the nucleic acid assembly compositions. Nuclease protection of the crRNA, DNA template, scaffold, and staples can be accomplished by, for example, incorporation of chemically modified nucleotides such as α-phosphorothiorates, 5-metylcytosine, and pseudouridine (Yin, H. et al., Nat Biotechnol (2017); Conway, J. W., et al., Chem Commun (Camb) 49, 1172-4 (2013)). Non-modified DNA staples can be protected from exonucleases on the 5′ and 3′ ends by, for example, conjugation to PEG, while phosphorothiorates along the phosphate backbone and base methylation can be used to protect from endonucleases. Additionally, branched PEG can be used to shield nucleic acid assemblies and cargo (Ponnuswamy, N. et al., Nat Commun 8, 15654 (2017)). The influence of shielding and other chemical modification on degradation kinetics in presence of nucleases and proteases can be monitored using, for example, gel shift assays (Veneziano, R. et al., bioRxiv (2017)). The influence of the low pH values found in the endosomal compartment on the release and nuclease activity of Cpf1 can be evaluated using, for example, plasmid cutting and gel shift assays.

It has been demonstrated that 3′ extended CRISPR/Cpf1 RNPs are capable of stably binding to nucleic acid assemblies with tetrahedral geometry. Furthermore, Cpf1 has be used in a dsDNA cleaving assay, showing maintained activity. One or more CRISPRCpf1-crRNA complexes can be stably packaged within nucleic acid assemblies shielded by PEG. Complexation and shielding can be assessed by, for example, gel shift assays and TIRF microscopy. Purified nucleic acid assembly-CRISPR/Cpf1-crRNA complexes can subsequently be tested for release in biochemical assays, such as the cleavage of plasmid DNA in vitro. The experiments will evaluate Release kinetics in presence of RNase H and by strand displacement can be optimized and assessed by, for example, using different numbers of complementary barcode nucleotides. Simultaneous packaging and controlled release of multiple CRISPR-Cas effector proteins can be used to optimize for HDR. CRISPR RNPs can be loaded during assembly of nucleic acid assemblies or on pre-assembled nucleic acid assemblies. Although this illustration describes particular CRISPR systems, alternative CRISPR systems such as Staphylococcus aureus Cas9 or zinc finger nucleases and TAL effector nucleases (TALENs) (Bedell, V. M. et al., Nature 491, 114-8 (2012); Urnov, F. D., et al., Nat Rev Genet 11, 636-46 (2010)), are also compatible with the platform, and may exhibit more optimal binding and release properties. Similarly, numerous other cargos can be used with the disclosed nucleic acid assemblies.

### Illustration 2: Targeting Molecules to Promote Nucleic Acid Assembly Endocytosis

The internalization of large and multicomponent cargo, such as CRISPR RNPs, represents an integral step towards treating and affecting difficult-to-transfect cells. While various methods were developed to facilitate this process, many rely on the disruption of the plasma membrane and thereby critically reduce cell viability (Kim, S, et al., Genome Res 24, 1012-9 (2014); Han, X. et al., Sci Adv 1, e1500454 (2015); Ramakrishna, S., et al., Genome Res 24, 1020-7 (2014)). Alternatively, the individual components of multicomponent cargo have been complexed with cationic and anionic lipids (Zuris, J. A. et al., Nat Biotechnol 33, 73-80 (2015)). The prepared liposomes with CRISPR RNP components were internalized by endocytosis to promote gene editing in vitro. However, this approach is limited by long incubation times and the cytotoxicity of the used lipids. By comparison, receptor-mediated endocytosis of nucleic acid assemblies is considerably more efficient and has been leveraged for the targeted in vivo delivery of various cargos (Angata, T., et al., Trends Pharmacol Sci 36, 645-60 (2015); Sanhueza, C. A., et al., J Am Chem Soc 139, 3528-3536 (2017)). Capitalizing on the discoveries describes here, large and multicomponent cargo (such as CRISPR RNPs) can be complexed with nucleic acid assemblies thereby avoiding the use of toxic lipids and achieving stoichiometric control for the cargo.

Effective delivery of large and complex cargo to difficult-to-transfect cells can be facilitated via the receptor-mediated endocytosis of these nucleic acid assemblies. As indicated above, primary hepatocytes are useful both for assessing the function of nucleic acid assemblies but also because they are biotechnologically relevant, for example, for the development of ‘liver-on-a-chip’ devices to study the metabolism of toxins and drugs (Bhatia, S. N., et al., Sci Transl Med 6, 245sr2 (2014); Griffith, L. G., et al., Hepatology 60, 1426-34 (2014)). In contrast to transformed cell lines, they display in vitro phenotypes closely resembling that of the human liver (Nyberg, S. L. et al., Ann Surg 220, 59-67 (1994)). However, primary hepatocytes display limited proliferation and viability in vitro. Prior efforts to address these limitations include genetic engineering or differentiation from induced pluripotent stem cells (iPSCs; Guye, P., et al., Nat Commun 7, 10243 (2016); Dong, J., et al., Cell 130, 1120-33 (2007)). As primary hepatocytes and iPSCs are difficult to transfect, both approaches will benefit from efficient delivery systems, which can produce, for example, efficient CRISPR-Cas-mediated gene editing on short timescales (Han, X., et al., Sci Adv 1, e1500454 (2015); Gresch, O., et al., Methods Mol Biol 801, 65-74 (2012)). The feasibility of targeted in vivo delivery of siRNA and other cargo to hepatocytes has also been explored extensively (Prakash, T. P. et al., J Med Chem 59, 2718-33 (2016); Sanhueza, C. A., et al., J Am Chem Soc 139, 3528-3536 (2017)). Hepatocytes express the endocytic lectin ASGPR, a trimeric receptor recognizing GalNAc and other glycans (Renz, M., et al., Proc Natl Acad Sci USA 109, E2989-97 (2012)).

ASGPR has been observed to promote the endocytosis of targeted nanoparticles based on multiple mechanisms. Interestingly, and helpfully, Ca²⁺-dependent interactions with glycans or glycomimetics are abrogated in the early endosome resulting in the release of the nanoparticles and the avoidance of lysosomal cargo degradation (Onizuka, T., et al., FEBS J 279, 2645-56 (2012)). Notably, the unbound receptor has been demonstrated to recycle back to the plasma membrane to mediate the internalization of additional nanoparticles (Schwartz, A. L., et al., J Cell Biol 98, 732-8 (1984)). Thus, ASGPR-mediated endocytosis is a good choice for effective delivery of the disclosed nucleic acid assemblies and their cargo. The specificity of ASGPR-mediated endocytosis is also helpful in targeting delivery to cells of interest while avoiding affecting non-target cells, an important feature for gene editing in hepatocyte co-cultures.

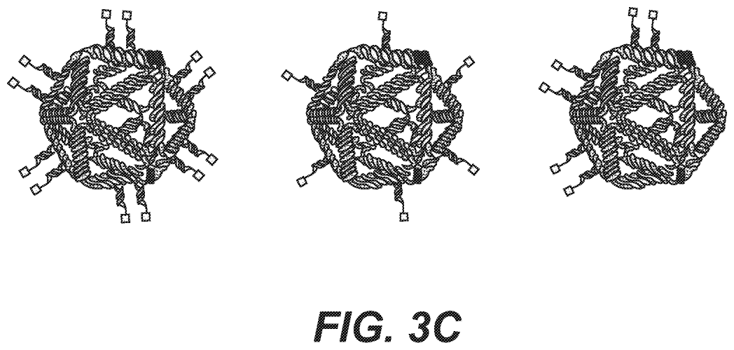

Several synthetic strategies have been developed to control the organization of GalNAc on the 1 to 10 nm scale (Huang, X., et al., Bioconjug Chem 28, 283-295 (2017)). Prominently, the divalent organization on dsDNA and the design of trivalent glycoclusters has resulted in substantially increased avidities for the trimeric ASGPR (Sanhueza, C. A., et al., J Am Chem Soc 139, 3528-3536 (2017); Schmidt, K., et al., Nucleic Acids Res 45, 2294-2306 (2017)). Additionally, the discovery of potent glycomimetic ligands has been reported (Sanhueza, C. A., et al., J Am Chem Soc 139, 3528-3536 (2017)). These synthetic strategies are adapted here to functionalize nucleic acid assemblies on single-overhangs. Generalizing this allows for the controlled organization of any ligands or other target molecules on the 10 to 100 nm scale (FIG. 3C). The design of nucleic acid assemblies of different sizes and geometries can be combined with systematic variations in ligand densities and functionalization symmetry to optimize effective delivery.

Overall, the disclosed compositions and methods enables the control of molecular and mechanical parameters on different scales ranging from 1 to 100 nm. The synthesis of a carefully designed library of nucleic acid assemblies will serve to supply a variety of different platforms for effective delivery of different cargo. This can be especially important in obtaining efficient delivery via receptor-mediated endocytosis involving, for example, clustered receptors or membrane deformation (Renz, M., et al., Proc Natl Acad Sci USA 109, E2989-97 (2012); Boulant, S., et al., Nat Cell Biol 13, 1124-31 (2011)).

In some forms, design and implementation of different nucleic acid assemblies can take account of both synthetic feasibility and the use of molecular and mechanic parameters of target molecules and target cells. Nucleic acid assemblies can be designed and synthesized based on a variety of geometries (FIG. 3B), including simple geometries such as tetrahedra or icosahedra (FIGS. 3B and 3A, respectively). Nucleic acid assemblies can be functionalized with targeting molecules, such as monovalent GalNAc ligands, on, for example, single-stranded overhangs (FIG. 3C). The size, ligand density, and functionalization symmetry can be varied (FIGS. 3A and 3C). More elaborate designs and synthetic strategies can be used, such as the use of ligand mimetics (e.g., glycomimetics) or the multivalent organization of ligands on the 1 to 10 nm scale to increase the avidity for the targeting molecules. Divalent organization of targeting molecules can use, for example, overhangs that are both attached to a targeting molecule and that are hybridized with an additional ssDNA attached to another targeting molecule (Schmidt, K., et al., Nucleic Acids Res 45, 2294-2306 (2017)). Useful ways to couple targeting molecules to bridging molecules include, for example, maleimide-thiol conjugations and azide-alkyne cycloaddition to modify the bridging molecules. Other, more non-canonical nucleic acid assembly geometries can be used to determine optimum shapes and sizes for the target and cargo being used (FIG. 3B; Agarwal, R. et al., Proc Natl Acad Sci USA 110, 17247-52 (2013)). Other physiochemical properties of nucleic acid assemblies can be provided by attachment of other effector molecules, such as extended PEG linkers, for ligand organization or the mechanical properties of the nucleic acid assemblies. The software CanDo and DAEDALUS can be used to assess these properties computationally (Veneziano, R. et al., Science 352, 1534 (2016); Castro, C. E., et al., Nat Methods 8, 221-9 (2011); Pan, K., et al., Nucleic Acids Res 45, 6284-6298 (2017)).

Nucleic acid assemblies designed on the above principles can be used in test setups, such as in transformed hepatocytes cell lines Hep G2 and SK Hep, to assess the function of the nucleic acid assemblies. For this purpose, for example, a plate-based colorimetric internalization assay can be used. For example, the nucleic acid assemblies can be functionalized with biotin on single-stranded overhangs and can thus be detected using a streptavidin-horseradish peroxidase (Strep-HRP) conjugate. Importantly, endocytosis can be distinguished from binding by washing the cells with EDTA prior to permeabilization to abrogate Ca²⁺-dependent interactions at the plasma membrane. While the Strep-HRP assay is characterized by excellent sensitivity, other approaches such as fluorescence-based flow cytometry experiments can be used to provide improved throughput and quantifiable read-out.

Both endocytosis and intracellular trafficking of the nucleic acid assemblies shown to be efficiently endocytosed can be analyzed in more detail. For example, diffraction-limited confocal microscopy can be used to assay for colocalization with endosomal and lysosomal markers. As another example, pH-sensitive fluorophores can be employed to evaluate the chemical environment of the nucleic acid assemblies. Notably, the tool DNA-PRISM can be used for barcoding nucleic acid assemblies in localization assays (Guo, S.-M. et al., bioRxiv (2017); Wang, Y. et al. Nano Lett 17, 6131-6139 (2017)). This approach can substantially improve analysis of intracellular trafficking of nucleic acid assemblies and cargo. Finally, super-resolution microscopy (e.g. DNA-PAINT) can be used to infer the underlying mechanism of nucleic acid assembly-mediated target molecule clustering and endocytosis (Guo, S.-M. et al., bioRxiv (2017); Jungmann, R. et al., Nat Methods 11, 313-8 (2014)).

The plasma membrane represents the first cellular barrier for the nuclear delivery of cargo, such as CRISPR RNPs. The adaptability of the size and shape of nucleic acid assemblies, as well as the ability to control the placement and density of targeting molecules provide the tools for optimizing target-mediated endocytosis for any endocytosing target molecule. For example, an avidity threshold for promoting internalization as well as an optimal nucleic acid assembly size can be determined giving the wide design space for nucleic acid assemblies and their complexation with targeting molecules. This design space includes the scale and symmetry at which the targeting molecules are organized, geometry of the nucleic acid assemblies, and the mechanical properties of the nucleic acid assemblies.

### Illustration 3: Control of Endosomal Escape, Cargo Release, and Cytotoxicity

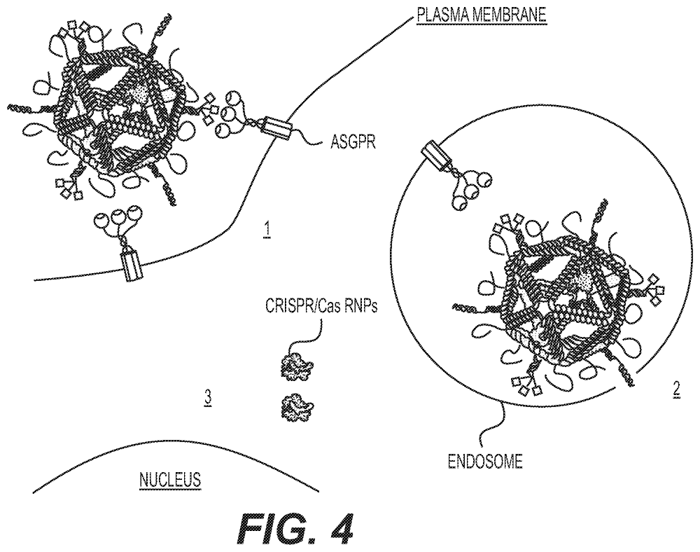

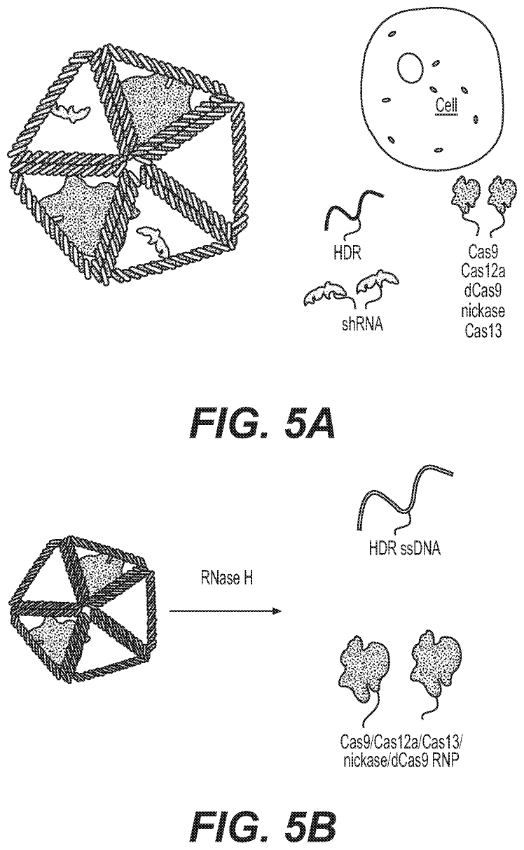

Endocytosis is the most common route of cell-entry for nanoparticles, and typically results in routing to the lysosome for degradation and scavenging (Canton, I. & Battaglia, G., Chem Soc Rev 41, 2718-39 (2012)). Thus, as well as efficient internalization, nucleic acid assemblies with cargo preferably will also efficiently escape the endosome prior to lysosomal delivery and degradation (FIG. 4). The fundamental aspects of this pathway are well defined and highly conserved, thus the developed strategies are applicable to a wide range of cell types and model systems (Grant, B. D. & Donaldson, J. G., Nat Rev Mol Cell Biol 10, 597-608 (2009)). Further, escape from the endosome is minimally perturbative compared to plasma membrane disruption exerted by traditional transfection methods and will thus result in improved cell viability and cargo effect (Liu, B. R. et al., PLoS One 8, e67100 (2013); Lonn, P., et al., Sci Rep 6, 32301 (2016)). Traditional methods involve electroporation, mechanical membrane deformation, cationic liposomes, and gold nanoparticles and typically exert stress on cells that potentially leads to apoptosis (Kim, S., et al., Genome Res 24, 1012-9 (2014); Han, X., et al., Sci Adv 1, e1500454 (2015); Frohlich, E., Int J Nanomedicine 7, 5577-91 (2012); Alkilany, A. M. & Murphy, C. J., J Nanopart Res 12, 2313-2333 (2010)).

Effective endosomal escape and intracellular trafficking are also aided by using the disclosed minimally perturbative transfection technique that is compatible with a wide range of cargos including CRISPR RNPs. Preferred design of endosome escape involves functionalization of nucleic acid assemblies with CPPs or endosome escape elements that avoid (e.g., TAT) or can escape endosomes or the release of cargo so that it can leave the endosome while the CPP remains bound. For transfer of cargo to the nucleus, nuclear localization signal peptides (NLSs) can be included in or attached to cargo. These peptides disrupt membranes and their activity can be tuned by controlling their spatial organization and density on nucleic acid assemblies or the cargo (Bechara, C. & Sagan, S., FEBS Lett 587, 1693-702 (2013)). Many CPPs, including the TAT peptide from HIV, also function as an NLS and mediate nuclear translocation (Vives, E., et al., J Biol Chem 272, 16010-7 (1997)). In addition, some CPPs are selectively active at low pH values found in the early endosome and show negligible activity at the plasma membrane. Such CPPs can be considered to include endosome escape elements. Endosomal escape elements can be attached to or incorporated onto other CPPs and/or the cargo. These peptides have proven useful for a variety of applications, and are generally less cytotoxic and more selective than their non-acid-activated counterparts (Vives, E., et al., J Biol Chem 272, 16010-7 (1997)). Thus, the functionalization of nucleic acid assemblies and cargo with CPPs and NLSs represents a tunable method to cytoplasmic and nuclear delivery of large and complex cargo, such as CRISPR RNPs.

For intracellular trafficking, synthetic strategies can be used to extend the scope of control release of cargo from the nucleic acid assemblies. For example, programmable linkers can be used between the RNPs and the nucleic acid assembly. These linkers can be sensitive to the biochemical environments within either the early endosome or the cytoplasm such that release happens after internalization of the RNP. Once RNPs enter the cytoplasm, NLSs on the RNP can be used to drive nuclear translocation (Staahl, B. T. et al., Nat Biotechnol 35, 431-434 (2017)). Optimal nucleic acid assembly designs for nuclear RNP delivery and gene editing can be selected by, for example, assaying for controlled release in vitro. Finally, maintaining cell viability during and following transfection is a challenging but essential goal, particularly when targeting primary hepatocytes. A loss in cell viability may occur due to cellular stress following excessive endosomal disruption or TLR9 signaling in response to the presence of DNA in endosomes (Lee, B. L. & Barton, G. M., Trends Cell Biol 24, 360-9 (2014)). The cellular signaling response, immunotoxicity, and cell viability of transfected cells in response to nucleic acid assembly internalization and nuclear CRISPR RNP delivery can be examined. These analyses can aid in producing nucleic acid assembly designs that minimize cellular immunotoxicity and optimize cell viability and gene editing efficiency.

The number and type of CPPs can also be tuned to achieve optimal endosomal escape of nucleic acid assemblies. For example, endosomal escape of the nucleic acid assemblies and CRISPR RNPs can be assessed, for example, in Hep G2 and SK Hep cells using fluorescently labeled nucleic acid assemblies by incorporating dyes within the scaffold DNA of the nucleic acid assemblies. Such highly fluorescent nucleic acid assemblies can be observed using multiple modes of microscopy and are compatible with high throughput imaging such as diffraction-limited confocal microscopy and epifluorescence microscopy in the far field. Fluorescent counterstains against nuclear DNA (DAPI), the glycocalyx covering plasma membrane (WGA), the lysosome (LAMP1), and early endosome (EEA1) can be used in order to follow the intracellular trafficking nucleic acid assemblies. A fluorescence microcopy technique recently described can be used, preferable concurrently, to quantify the magnitude of endosomal escape (Wittrup, A. et al., Nat Biotechnol 33, 870-6 (2015)). Libraries of nucleic acid assemblies with systematically varying densities and spatial organizations of different CPPs can be assessed using, for example, high-throughput imaging to determine optimal nucleic acid assembly compositions for nuclear CRISPR RNP delivery. CPPs bearing terminal peptide nucleic acid (PNA) strands can be hybridized to single-stranded overhangs on nucleic acid assemblies. The formation of PNA/DNA duplexes is robust and allows for stoichiometric control over different CPPs (Nielsen, P. E., et al., Bioconjug Chem 5, 3-7 (1994)). CPPs used will include the canonical TAT sequence, as well as the acid-activated TH peptide (Ziegler, A., et al., Biochemistry 44, 138-48 (2005); Zhang, W., et al., Bioconjug Chem 22, 1410-5 (2011)).

The controlled release of CRISPR RNPs and HDR template DNA and the ability to detect this process are preferred features of the disclosed nucleic acid assembly-based delivery platform. Preferred nucleic acid assemblies leverage the biochemical environment in the early endosome or the cytoplasm to facilitate release of cargo. In addition to native release and RNase H-mediated cleavage, which will act in the cytoplasm, programmable linkers can also be used (Cazenave, C., et al., J Biol Chem 269, 25185-92 (1994)). For example, two additional linker classes with nucleic acid assemblies: cleavable peptides and redox-sensitive disulfides, can be used. Furin cleavage sites can be used to initiate cargo release in the early endosome and will continue in the cytoplasm. By contrast, disulfide linkers can be used for cytoplasmic cargo release. The function of these options can be assessed, for example, in Hep G2 or SK Hep by confocal microscopy to observe colocalization of nucleic acid assemblies and cargo.

The gene editing efficiency achieved through the nucleic acid assembly-based delivery platform can be evaluated using standard readouts of genomic editing, including the Surveyor method to assay for site specific insertion and deletion events, cleavage of engineered cut sites in donor DNA following genomic incorporation, and whole genome sequencing to assess off-target editing. Gene editing efficiency can be correlated, preferably in parallel, with the intracellular trafficking of nucleic acid assemblies, CRISPR RNPs, and HDR template DNA assayed by confocal microscopy. Importantly, both data sets can be generated using the same cell sample. The effect of incubation of nucleic acid assemblies on cell viability can also be assessed.

These assays and assessments can be used to select optimal forms for nucleic acid assemblies to promote cytoplasmic and nuclear delivery of CRISPR RNPs following internalization into endosomes. The specific formulation can be optimized to cytotoxicity, while retaining their advantageous characteristics for receptor-mediated endocytosis, engineered intracellular trafficking, and high editing efficiency. Because of these selectable features of nucleic acid assemblies, they can produce improved gene editing for difficult-to-transfect cells, such as primary hepatocytes. Furthermore, these assessments allow identification and selection of specific nucleic acid assembly formulations that are generally suited for cytoplasmic or nuclear delivery of nucleases and other complex protein-based cargo. These formulations will facilitate the delivery of CRISPR RNPs into cellular environments of interest for both genetic editing and other challenging delivery applications.

The disclosed compositions and methods can be further understood through the following numbered paragraphs.

1. A composition comprising:

(a) a nucleic acid assembly comprising one or more nucleic acid molecules, the nucleic acid assembly comprising physiochemical properties that:
(i) enhance trafficking and/or targeting of the composition to one or more types of cells, tissues, organs, or microenvironments relative to other types of cells, tissues, organs, or microenvironments in vivo;
(ii) enhance stability and/or half-life of the composition in vivo; and/or
(iii) reduce immunogenicity of the composition; and

(b) cargo comprising two or more cargo molecules enclosed and/or protected by the nucleic acid assembly in a defined stoichiometric ratio.

2. The composition of paragraph 1, wherein the one or more nucleic acid molecules comprising the nucleic acid assembly hybridize together.

3. The composition of paragraph 1 or 2, wherein the one or more nucleic acid molecules comprising the nucleic acid assembly comprise RNA.

4. The composition of any one of paragraphs 1 to 3, further comprising a plurality of bridging molecules, wherein each bridging molecule is part of or directly or indirectly attached to either or both the nucleic acid assembly and one or more of the cargo molecules, wherein the cargo molecules and bridging molecules that are part of or attach to each other are said to correspond to each other,

whereby the bridging molecules collectively attach the cargo molecules to the nucleic acid assembly in the defined stoichiometric ratio.

5. The composition of paragraph 4, wherein two or more of the bridging molecules constitute one or more pairs of bridging molecules that specifically bind to the other bridging molecule in the pair,

wherein, for each pair, one bridging molecule of the pair is part of or is directly or indirectly attached to the nucleic acid assembly and the other bridging molecule of the pair is part of or directly or indirectly attached to a corresponding cargo molecule,

whereby specific binding of the pair of bridging molecules specifically attaches the corresponding cargo molecule to the nucleic acid assembly.

6. The composition of paragraph 4 or 5, wherein at least one of the bridging molecules is part of the nucleic acid assembly, wherein the bridging molecule that is part of the nucleic acid assembly attaches directly or indirectly to a corresponding cargo molecule,

whereby the bridging molecule that is part of the nucleic acid assembly specifically attaches the corresponding cargo molecule to the nucleic acid assembly.

7. The composition of any one of paragraphs 4 to 6, wherein at least one of the bridging molecules is part of a cargo molecule, wherein the bridging molecule that is part of the cargo molecule attaches directly or indirectly to the nucleic acid assembly,

whereby the bridging molecule that is part of the cargo molecule specifically attaches the cargo molecule to the nucleic acid assembly.

8. The composition of any one of paragraphs 4 to 7, wherein at least one of the bridging molecules is part of a cargo molecule, wherein the bridging molecule that is part of the cargo molecule is part of the nucleic acid assembly,

whereby the bridging molecule that is part of the cargo molecule specifically attaches the cargo molecule to the nucleic acid assembly.

9. The composition of any one of paragraphs 4 to 8, wherein direct attachments of bridging molecules to the nucleic acid assembly and/or cargo molecules each comprise a covalent bond, a non-covalent bond, or both a covalent bond and a non-covalent bond.

10. The composition of paragraph 9, wherein a plurality of the non-covalent bonds are involved in nucleic acid hybridization, whereby specificity of the attachment of the cargo molecule to the nucleic acid assembly is provided by the specificity of the nucleic acid hybridization.

11. The composition of paragraph 9 or 10, wherein at least one of the covalent bonds is formed by a click chemistry reaction, whereby specificity of the attachment of the cargo molecule to the nucleic acid assembly is provided by the specificity of the click chemistry reaction.

12. The composition of any one of paragraphs 9 to 11, wherein at least one of the non-covalent bonds is formed by specific binding molecules in a specific binding molecule pair, whereby specificity of the attachment of the cargo molecule to the nucleic acid assembly is provided by the specificity of the specific binding molecule pair.

13. The composition of any one of paragraphs 1 to 12, wherein the defined stoichiometric ratio of the cargo molecules is based on the stoichiometric ratio at which the cargo molecules function together.

14. The composition of any one of paragraphs 1 to 12, wherein the defined stoichiometric ratio of the cargo molecules is based on a desired relative effect of the cargo molecules.

15. The composition of any one of paragraphs 1 to 14, wherein the physiochemical properties are selected from structural properties, electric properties, biological properties, or a combination thereof.

16. The composition of any one of paragraphs 1 to 15, wherein the cargo comprises one or more components of one or more CRISPR-Cas systems.

17. The composition of paragraph 16, wherein the cargo comprises one or more CRISPR-Cas effector proteins.

18. The composition of paragraph 17, wherein the cargo further comprises one or more guide molecules and/or one or more template oligonucleotides.

19. The composition of paragraph 18, wherein one or more of the guide molecules is part of the nucleic acid assembly.

20. The composition of paragraph 18 or 19, wherein one or more of the template oligonucleotides is part of the nucleic acid assembly.

21. The composition of any one of paragraphs 18 to 20, wherein one or more of the template oligonucleotides is an HDR template.

22. The composition of any one of paragraphs 18 to 20, wherein one or more of the template oligonucleotides is an mRNA.

23. The composition of any one of paragraphs 18 to 22, wherein the cargo comprises two or more CRISPR-Cas effector proteins, two or more guide molecules, two or more template oligonucleotides, or a combination thereof.

24. The composition of any one of paragraphs 16 to 23, wherein at least one of the one or more CRISPR-Cas systems is a Cas9 system, a Cas12 system, a Cas13 system, a dCas system, a nickase system, a paired nickase system, an Alt-R CRISPR system, a proxy-CRISPR system, an Alt-R dCas system, an Alt-R nickase system, an Alt-R paired nickase system, an Alt-R proxy-CRISPR system, a proxy-dCas system, a proxy-nickase system, a proxy-paired nickase system, an Alt-R proxy-dCas system, an Alt-R proxy-nickase system, or an Alt-R proxy-paired nickase system.

25. The composition of any one of paragraphs 16 to 24, wherein at least one of the one or more CRISPR-Cas systems comprises one or more CRISPR-Cas effector proteins, wherein at least one of the CRISPR-Cas effector proteins is SpCas9, dCas9, Cas nickase, Cas9 nickase, FnCas9, StCas9, SaCas9, LpCas9, FnCas12, Cas12 nickase, AsCas12, LbCas12, Cas12a, Cas12b, Cas12c, Cas13, or Cas13d.

26. The composition of any one of paragraphs 16 to 25, wherein at least one of the one or more CRISPR-Cas systems comprises a paired Cas9 nickase system, a paired Cas9 nickase system, a dCas/Cas proxy-CRISPR system, a dCas9/Cas9 proxy-CRISPR system, or an SpdCas9/FnCas9 proxy-CRISPR system.

27. The composition of any one of paragraphs 24 to 26, wherein the proxy-CRISPR system comprises two first dCas ribonucleoproteins targeted to different sites flanking and on opposite sides of a main target site and a Cas ribonucleoprotein targeted to the main target site.

28. The composition of paragraph 27, wherein the proxy-CRISPR system further comprises two additional dCas ribonucleoproteins targeted to different sites flanking and on opposite sides of the main target site, wherein the sites to which the additional dCas ribonucleoproteins are targeted are not the same as the target sites to which the first dCas ribonucleoproteins are targeted.

29. The composition of any one of paragraphs 24 to 28, wherein the Alt-R CRISPR system comprises a separate, shortened gRNA, a separate, shortened tracrRNA, a Cas9 protein.

30. The composition of any one of paragraphs 24 to 29, wherein the paired nickase system leaves 5′ overhangs.

31. The composition of any one of paragraphs 16 to 30, wherein the cargo comprises one or more components of two or more CRISPR-Cas systems.

32. The composition of paragraph 23, wherein the cargo comprises three or more CRISPR-Cas effector proteins, three or more guide molecules, three or more template oligonucleotides, or a combination thereof.

33. The composition of any one of paragraphs 1 to 15, wherein the cargo does not comprise a CRISPR-Cas effector protein, guide molecule, or HDR template.

34. The composition of any one of paragraphs 1 to 33, wherein the cargo comprises an anti-sense nucleic acid, mRNA, miRNA, piRNA, siRNA, or a combination thereof.

35. The composition of any one of paragraphs 1 to 34, further comprising one or more targeting molecules that specifically targets the nucleic acid assembly to one or more types of cells, tissues, organs, or microenvironments relative to other types of cells, tissues, organs, or microenvironments in vivo.

36. The composition of paragraph 35, wherein the targeting molecules are selected from the group consisting of DNA aptamers, RNA aptamers, antibodies, nanobodies, lectins, small molecule binding compounds, protein binding domains, protein toxin subunits, peptides, and viral coat proteins.

37. The composition of any one of paragraphs 1 to 36, wherein the nucleic acid assembly forms a container.

38. The composition of paragraph 37, wherein the cargo is inside the container.

39. The composition of any one of paragraphs 1 to 38, wherein the nucleic acid assembly comprises one or more RNA/DNA hybrid regions.

40. The composition of any one of paragraphs 4 to 39, wherein one or more of the bridging molecules or bound bridging molecule pairs comprises an RNA/DNA hybrid region.

41. The composition of paragraph 39 or 40, wherein one or more of the RNA/DNA hybrid regions facilitates release of one or more cargo molecules in the presence of an RNA/DNA hybrid specific nuclease.

42. The composition of any one of paragraphs 1 to 41, wherein the nucleic acid assembly comprises a plurality of effector molecules, wherein the effector molecules produce or contribute to the physiochemical properties.

43. The composition of paragraph 42, wherein the effector molecules comprise polyethylene glycol molecules, lipids, polar groups, charged groups, amphipathic groups, albumin binding molecules, zwitterions, polyamines, RNA intercollators, DNA intercollators, backbone-modified nucleic acids, base-modified nucleic acids, or combinations thereof.

44. A method of delivering cargo to a cell, the method comprising bringing into contact a cell of interest and a composition of any one of paragraphs 1 to 43.

45. The method of paragraph 44, wherein bringing into contact is accomplished by administering the composition to a subject, wherein the subject harbors the cell.

46. The method of paragraph 45, wherein the cell is in a tissue of interest, organ of interest, or microenvironment of interest.

47. A method of producing a composition of any one of paragraphs 1 to 43, the method comprising bringing into contact the one or more nucleic acid molecules and the cargo under conditions that facilitate assembly of the nucleic acid molecules and the cargo to form the composition.

### EXAMPLES

### Example 1: Nucleic Acid Assembly Formed of RNA/DNA Hybrids that Include an HDR ssDNA

1. Design of RNA/DNA Hybrid Assemblies.

The mRNA sequence of enhanced green fluorescent protein with an additional 3′-untranslated region with the first 3 codons unstructured was used to generate a total RNA sequence 821 nucleotides in length. The sequence of the mRNA was:

(SEQ ID NO: 1)
GGUAGCUAAGGAGGUAAAUAAUGGUGAGCAAGGGCGAGGAG
CUGUUCACCGGGGUGGUGCCCAUCCUGGUCGAGCUGGACGGC
GACGUAAACGGCCACAAGUUCAGCGUGUCCGGCGAGGGCGAG
GGCGAUGCCACCUACGGCAAGCUGACCCUGAAGUUCAUCUGC
ACCACCGGCAAGCUGCCCGUGCCCUGGCCCACCCUCGUGACC
ACCCUGACCUACGGCGUGCAGUGCUUCAGCCGCUACCCCGAC
CACAUGAAGCAGCACGACUUCUUCAAGUCCGCCAUGCCCGAA
GGCUACGUCCAGGAGCGCACCAUCUUCUUCAAGGACGACGGC
AACUACAAGACCCGCGCCGAGGUGAAGUUCGAGGGCGACACC
CUGGUGAACCGCAUCGAGCUGAAGGGCAUCGACUUCAAGGAG
GACGGCAACAUCCUGGGGCACAAGCUGGAGUACAACUACAAC
AGCCACAACGUCUAUAUCAUGGCCGACAAGCAGAAGAACGGC
AUCAAGGUGAACUUCAAGAUCCGCCACAACAUCGAGGACGGC
AGCGUGCAGCUCGCCGACCACUACCAGCAGAACACCCCCAUC
GGCGACGGCCCCGUGCUGCUGCCCGACAACCACUACCUGAGC
ACCCAGUCCGCCCUGAGCAAAGACCCCAACGAGAAGCGCGAU
CACAUGGUCCUGCUGGAGUUCGUGACCGCCGCCGGGAUCACU
CUCGGCAUGGACGAGCUGUACAAGUAACUGCAGGCAUGCAAG
CUUGGCGUAAUCAUGGUCAUAGCUGUUUCCUGUGUGAAAUU
GUUAUCCGCUCACAAUUCCACACAA.

The scaffold RNA sequence used to make the nucleic acid assembly was:

(SEQ ID NO: 2)
AAGGGCGAGGAGCUGUUCACCGGGGUGGUGCCCAUCCUGGUC
GAGCUGGACGGCGACGUAAACGGCCACAAGUUCAGCGUGUCC
GGCGAGGGCGAGGGCGAUGCCACCUACGGCAAGCUGACCCUG
AAGUUCAUCUGCACCACCGGCAAGCUGCCCGUGCCCUGGCCC
ACCCUCGUGACCACCCUGACCUACGGCGUGCAGUGCUUCAGC
CGCUACCCCGACCACAUGAAGCAGCACGACUUCUUCAAGUCC
GCCAUGCCCGAAGGCUACGUCCAGGAGCGCACCAUCUUCUUC
AAGGACGACGGCAACUACAAGACCCGCGCCGAGGUGAAGUUC
GAGGGCGACACCCUGGUGAACCGCAUCGAGCUGAAGGGCAUC
GACUUCAAGGAGGACGGCAACAUCCUGGGGCACAAGCUGGAG
UACAACUACAACAGCCACAACGUCUAUAUCAUGGCCGACAAG
CAGAAGAACGGCAUCAAGGUGAACUUCAAGAUCCGCCACAAC
AUCGAGGACGGCAGCGUGCAGCUCGCCGACCACUACCAGCAG
AACACCCCCAUCGGCGACGGCCCCGUGCUGCUGCCCGACAAC
CACUACCUGAGCACCCAGUCCGCCCUGAGCAAAGACCCCAAC
GAGAAGCGCGAUCACAUGGUCCUGCUGGAGUUCGUGACCGCC
GCCGGGAUCACUCUCGGCAUGGACGAGCUGUACAAGUAACUG
CAGGCAUGCAAGCUUGGCGUAAUCAUGGUCAUAGCUGUUUCC
UGUGUGAAAUUGUUAUCCGCUCACAAUUCCACACAA.

This sequence was used as input along with a PLY design file describing a perfect tetrahedron with a minimal edge length of 66 basepairs into a modified form of the software DAEDALUS that creates crossovers based on A-form helix geometries and outputs a set of DNA staple sequences that will fold the RNA scaffold into the desired geometry (e.g., here a tetrahedron). The resulting 19 staple sequences were:

| | |
|---|---|
| 01_tetrahedron_66_0-222-E | GGTCGGGGTAGAGCTCCTCGCCCTTTTGTGTGGCTGCTTCATGT |
| | (SEQ ID NO: 3) |
| 01_tetrahedron_66_0-200-E | CTGCACGCCGTGATGGGCACCACCCCGGTGAACCGGCTGAAGCA |
| | (SEQ ID NO: 4) |
| 01_tetrahedron_66_1-541-V | TGGTAGTGGTCTTTTTGGCGAGCTGCATGGCGGACTTGTTTTTAA |
| | GAAGTCGTGAATTGTGAGCGTTTTTGATAACAATTT (SEQ ID |
| | NO: 5) |
| 01_tetrahedron_66_1-750-E | CAGCTATGACCGTCGCCGATGGGGGTGTTCTGCCACACAGGAAA |
| | (SEQ ID NO: 6) |
| 01_tetrahedron_66_1-728-E | AAGCTTGCATGTGTCGGGCAGCAGCACGGGGCCATGATTACGCC |
| | (SEQ ID NO: 7) |
| 01_tetrahedron_66_2-417-E | CCAGCTTGACGTTGTGGCTGTTGTAGTCTTTGC (SEQ ID NO: 8) |
| 01_tetrahedron_66_2-615-E | TCAGGGCGTCGCGCTTCTCGTTGGGGTTGTACT (SEQ ID NO: 9) |
| 01_tetrahedron_66_2-409-E | TGCCCCAGGATCCATGATATAG (SEQ ID NO: 10) |
| 01_tetrahedron_66_3-475-V | ATGCCGTTCTTTTTTTCTGCTTGTCGGGTTGCCGTCCTTTTTTCCT |
| | TGAAGTCGCGCGGGTCTTGTTTTTTAGTTGCCGTC (SEQ ID NO: 11) |
| 01_tetrahedron_66_3-508-E | TCGATGTTGTGTCCTGGACGTAGCCTTCGGGCACGCTGCCGTCC |
| | (SEQ ID NO: 12) |
| 01_tetrahedron_66_3-486-E | AGTTCACCTTGGTCCTTGAAGAAGATGGTGCGCGCGGATCTTGA |
| | (SEQ ID NO: 13) |
| 01_tetrahedron_66_4-662-V | GAACTCCAGCATTTTTGGACCATGTGAGACTGGGTGCTTTTTTCA |
| | GGTAGTGGTCCTGCAGTTACTTTTTTTGTACAGCTC (SEQ ID |
| | NO: 14) |
| 01_tetrahedron_66_4-76-E | CTGAACTTATCGCCCTCGCCCTCGCCGCCGGCG (SEQ ID NO: 15) |
| 01_tetrahedron_66_4-670-E | GCGGTCACGTCCATGCCGAGAGTGATCGACACG (SEQ ID NO: 16) |
| 01_tetrahedron_66_4-68-E | GTGGCCGTTTACCGTAGGTGGC (SEQ ID NO: 17) |
| 01_tetrahedron_66_5-134-V | GATGAACTTCATTTTTGGGTCAGCTTGCGTCGCCGTCCTTTTTAG |
| | CTCGACCAGAGGTCAGGGTGTTTTTGTCACGAGGGT (SEQ ID |
| | NO: 18) |
| 01_tetrahedron_66_5-340-E | CCCTCGAACTCGATGCGGTTCACCAGGTTGCCG (SEQ ID NO: 19) |
| 01_tetrahedron_66_5442-E | GTGGTGCAGGGCCAGGGCACGGGCAGCGTGTCG (SEQ ID NO: 20) |
| 01_tetrahedron_66_5-332-E | CTTCACCTCGGATGCCCTTCAG (SEQ ID NO: 21) |

2. Folding RNA/DNA Hybrid Nanoparticles

RNA was transcribed using the New England Biolabs HiScribe T7-based polymerase system with the DNA sequence encoding the mRNA containing a T7 RNA polymerase promoter (TAATACGACTCACTATAG; SEQ ID NO:22) with RNA initiating on the final 3′ G, using 2 μg of dsDNA template in a 20 μl transcription reaction. The RNA was purified using ThermoFisher RNeasy silica-column purification and eluted in water. The DNA staples were synthesized by Integrated DNA Technologies (IDT, Coralville, Iowa) and pooled in equimolar concentrations.

20 nM of purified RNA scaffold was added in a 50 μl folding reaction containing 30 mM HEPES-KOH pH 7.4, 200 mM-1M KCl, NaCl, K-acetate, or K-glutamate, and a 30-fold or higher molar excess of DNA staples. All buffers and salts and staples were prepared using RNase-free conditions. Presence of magnesium in the folding buffer was shown to negatively affect particle formation. The particles were folded by incubating at 95° C. for 30 sec, then 85° C. to 70° C. incrementing per degree for 30 sec, then from 70° C. to 25° C. decrementing per degree for 15 min.

100 kDa molecular weight cutoff spin filters (Amicon) were washed with RNase free water and pre-spun for 10 min at 4,000 RPMs. Water was then discarded and folded mixture was added to the upper chamber and diluted with purification buffer (30 mM HEPES-KOH pH 7.4, 300 mM KCl) and re-concentrated 10× by spinning at 3,000 RPMs for 30 min. This procedure was repeated for 5 rounds to purify from the staples. The folded RNA/DNA hybrid assembly was then run on a gel to visualize gel shift from pure scaffold or was characterized by cryo-electron microscopy. The tetrahedron shape was able to be reconstructed from the cryo-electron microscopy imaged sample, showing a tetrahedron folded with mRNA scaffold.

The RNA/DNA hybrid assembly was then incubated with RNase H and separately with RNase A/T1 and separately with DNase I. RNase H and RNase A/T1 showed clear degradation of the RNA scaffold by disappearance of the band on a gel, while digestion with DNase I showed a downward shift of the band on the gel, showing release of the RNA scaffold.

Any geometry and degradation described here with an RNA-scaffold and DNA-staples would additionally apply to DNA-scaffold and RNA-staples, as the form allows for fungible hybrid type.

3. Design and Folding of DNA Nanoparticles Containing an Internal Crossbeam

A tetrahedron composed entirely of DNA was designed to be 84 bp in edge length using the DAEDALUS software specific for fully DNA objects using the following customized DNA scaffold (sequence #1) for this geometry, which is 1,008 bases in length:

(SEQ ID NO: 23)
CGACTCACTATAGGTCTCGCTGGTGAAAAGAAAAACCACCCTGGCGCCCAA
TACGCAAACCGCCTCTCCCCGCGCGTTGGCCGATTCATTAATGCAGCTGGC
ACGACAGGTTTCCCGACTGGAAAGCGGGCAGTGAGCGCAACGCAATTAATG
TGAGTTAGCTCACTCATTAGGCACCCCAGGCTTTACACTTTATGCTTCCGG
CTCGTATGTTGTGTGGAATTGTGAGCGGATAACAATTTCACACAGGAAACA
GCTATGACCATGATTACGAATTCGAGCTCGGTACCCGGGGATCCTCTAGAG
TCGACCTGCAGGCATGCAAGCTTGGCACTGGCCGTCGTTTTACAACGTCGT
GACTGGGAAAACCCTGGCGTTACCCAACTTAATCGCCTTGCAGCACATCCC
CCTTTCGCCAGCTGGCGTAATAGCGAAGAGGCCCGCACCGATCGCCCTTCC
CAACAGTTGCGCAGCCTGAATGGCGAATGGCGCTTTGCCTGGTTTCCGGCA
CCAGAAGCGGTGCCGGAAAGCTGGCTGGAGTGCGATCTTCCTGAGGCCGAT
ACGGTCGTCGTCCCCTCAAACTGGCAGATGCACGGTTACGATGCGCCCATC
TACACCAACGTAACCTATCCCATTACGGTCAATCCGCCGTTTGTTCCCACG
GAGAATCCGACGGGTTGTTACTCGCTCACATTTAATGTTGATGAAAGCTGG
CTACAGGAAGGCCAGACGCGAATTATTTTTGATGGCGTTCCTATTGGTTAA
AAAATGAGCTGATTTAACAAAAATTTAACGCGAATTTTAACAAAATATTAA
CGTTTACAATTTAAATATTTGCTTATACAATCTTCCTGTTTTTGGGGCTTT
TCTGATTATCAACCGGGGTACATATGATTGACATGCTAGTTTTACGATTAC
CGTTCATCGATTCTCTTGTTTGCTCCAGACTCTCAGGCAATGACCTGATAG
CCTTTGTAGATCTCTCAAAAATAGCTACCCTCTCCGGCA

The ssDNA scaffold was generated using asymmetric PCR while the ssDNA staples were synthesized by IDT.

Edges of the tetrahedron most distal from each other were modified such that the nick position is 8 nucleotides away from the crossover, thus allowing for an internal-facing nick position with an extension of sequence complementary to a bridging sequence that crosses between the two distal edges. The bridging sequence that spans the edge overhangs included a target sequence complementary to a guide sequence. Staple sequences are, starting from sequence #2 (where sequence #1 is the scaffold sequence):

2:
(SEQ ID NO: 24)
GATGAACGGTCCATCAAAAATAATTCGCGTCACAAGAGAATC
3:
(SEQ ID NO: 25)
TATCAGGTCATCATCAACATTAAATGTGAGCTCTACAAAGGC
4:
(SEQ ID NO: 26)
GAGTAACAACCTTTTTCGTCGGATTCAGCTTGCATGCTTTTTCTGCAGGTC
GAGGGTAGCTATTTTTTTTTTGAGAGA
5:
(SEQ ID NO: 27)
TATAAGCAAATTTTTTATTTAAATTGATTTTTTAACCTTTTTAATAGGAAC
GAATCGTAAAACTTTTTTAGCATGTCA
6:
(SEQ ID NO: 28)
AAACGGCGGAGTAAAACGACGGCCAGTGCCATCCGTGGGAAC
7:
(SEQ ID NO: 29)
GATGGGCGCAGATTAAGTTGGGTAACGCCAGACGTTGGTGTA
8:
(SEQ ID NO: 30)
TCCCCGGGTACTATAGTGAGTCGTGCCGGAGACTCTAGAGGA
9:
(SEQ ID NO: 31)
GTTTCCTGTGCGTATTGGGCGCCAGGGTGGTTGGTCATAGCT
10:
(SEQ ID NO: 32)
AATGAGTGAGCTTTTTTAACTCACATCGCGCGGGGAGTTTTTAGGCGGTTT
GTGAAATTGTTATTTTTTCCGCTCACA
11:
(SEQ ID NO: 33)
AATCGGCCAATAATTGCGTTGCGCTCACTGCCTGCATTAATG
12:
(SEQ ID NO: 34)
GTAAAGCCACATACGAGCCGGAAGTCGGTGCG
13:
(SEQ ID NO: 35)
GGCCTCTTCTGTTGGGAAGGGCGACATAAAGT
14:
(SEQ ID NO: 36)
CAGCTGGCGATTCGCCATTCAGGCTGCGCAACGCTATTACGC
15:
(SEQ ID NO: 37)
TGCTAGGATGGCCGTTGCAGTCGGGGAAAAGCCCCAAAAACCCCCGGTT
16:
(SEQ ID NO: 38)
AGGAAGATTGATCATATGTA
17:
(SEQ ID NO: 39)
GATAATCAAAACCTGTCGTGCCAGCCGCTTTC
18:
(SEQ ID NO: 40)
TTTTTGTTTATTTTGTTAAAATTCCGCACTCC
19:
(SEQ ID NO: 41)
CTTCTGGTGCTTTGAGGGGACGACGACCGTATTCCGGCACCG
20:
(SEQ ID NO: 42)
AGCCAGCTTCGGCCTCAGGAAGATGCGTTAAA
21:
(SEQ ID NO: 43)
TCGTAACCGTGTTTTTCATCTGCCAGCGGAAACCAGGTTTTTCAAAGCGCC
AAAGGGGGATGTTTTTTGCTGCAAGGC
22:
(SEQ ID NO: 44)
AAATCAGCTCTAAACGTTAA
23:
(SEQ ID NO: 45)
TGGGGTGCCTATTCCACACA
24:
(SEQ ID NO: 46)
TTCGTAATCATTTTCTTTTCACCAGCGAGACCCGAGCTCGAA
25:
(SEQ ID NO: 47)
TCTGGAGCAATGGCCTTCCTGTAGCCAGCTTTTGCCTGAGAG
26:
(SEQ ID NO: 48)
CAGTCACGACGTTTTGACCGTAATGGGATAGGTTGGTTTTCCGGGCCCGGA
CCGGGGTCTTTG
27:
(SEQ ID NO: 49)
ACGGCCATCCTAGCATCCTGAGCACCCAGTCCGCCCTGAGCAAAGAC
CCCGGTCCGGGC
28:
(SEQ ID NO: 50)
CTCAGGGCGGACTGGGTGCTCAGG

4. Design and Folding of DNA Assemblies Containing Internally-Facing and Outwardly-Facing Overhang Single-Strand Sequences for Functionalization.

The same tetrahedron sequence as above was used to fold nanoparticles containing overhang ssDNA sequences that are internally facing. Extensions off of the staple sequence in the inwardly-facing nick position were choses from subsets of 240,000 unique orthogonal DNA sequences. Barcodes 50, 3000, and 3001 from the list of barcodes were chosen and bases were deleted from the 5′ end to get complementary sequences with melting temperatures of 50° C. in the buffer conditions to 15-19 nucleotides in length (e.g. TCTACCTTGCAGTGGCTAC; SEQ ID NO:51). Additional lengths of DNA that enter inside of the guide sequence of the CRISPR enzymes were added, thus extending 29 nucleotides and 36 nucleotides away from the nick position. Additionally, these overhang sequences designed for use as bridging molecules for attaching cargo. For example, the overhang sequences were chosen based on the orthogonality to the staple and scaffold sequences using BLASTN with a word size of 9. Any overhangs that were identified with a length of 9 or more complementary nucleotides were excluded as being an address. These 15 to 16 nucleotide addresses were added to the 3′ end of the staple sequence with a single T added between the nick and the address sequence. These addresses were used as baits for RNAs by transcribing crRNA and sgRNAs with a 3′ end that were complementary to the overhang sequence. For example, RNA sequence for VEGFA-targeting crRNA for Mb3Cpf1 were:

(SEQ ID NO: 52)
GAAUUUCUACUGUUUGUAGAUCAAAGCCCAUUCCCUCUUUAG
C [symbol] (capture address in
bold italic).

Another example being an address to capture the sgRNA of a Cas9 that targets EGFP for editing and includes a broccoli aptamer in the sgRNA loop:

(SEQ ID NO: 53)
GAGCACGGGGCCGUCGCCGAUGUUUUAGAGCUAUGCUGUUG
AGACGGUCGGGUCCAGAUAUUCGUAUCUGUCGAGUAGAGUG
UGGGCUCAACAGCAUAGCAAGUUAAAAUAAGGCUAGUCCGU
UAUCAACUUGAAAAAGUGGCACCGAGUCGGUGCUUCGACUU [symbol] [symbol]
(capture address in bold italic).

Alternatively, outwardly facing nick positions were generated by placing the nick position 13 bases away from the possible crossover position, and extending the sequences either 5′ or 3′ for a ssDNA overhang that is outwardly facing. This outwardly facing overhang can contain sequence or complementary sequences to aptamers, antibodies, or other modifying moieties. For example, one of the edges of the nanoparticle was edited to have an outwardly facing overhang that included a DNA aptamer sequence targeting HER2 receptors and the same staple was also made to have an Estrogen Receptor DNA aptamer sequence:

HER2 aptamer staple:
(SEQ ID NO: 54)
TCCTAGATGAATAAGTAAATTACATGCGAATCTTCCCTATATttttt
GCAGCGGTGTGGGGGCAGCGGTGTGGGGGCAGCGGTGTGGGG.
ER aptamer staple:
(SEQ ID NO: 55)
TCCTAGATGAATAAGTAAATTACATGCGAATCTTCCCTATATttttt
CCCGGCATGGTTGCGGAGCAGGAGTATAACACTACCATTG.

Staple mixes were pooled to equimolar concentrations 1 pool for the HER2, 1 pool for the ER, with inwardly facing bait sequences on the modified edge:

(SEQ ID NO: 56)
AAGTGGCTAACGCGCGTCGACTAGTACAACCTTAAAGATGAGt [symbol]
.

These were used as baits for capturing EMX1-targeting crRNA. The nucleic acid assembly was folded with 20 nM scaffold, 1×TAE buffer, 12 mM MgCl₂ in the folding protocol known in the art. Gel shift showed a folded assembly was feasible in these conditions with overhangs. The nucleic acid assembly was purified by buffer exchange to purify from additional staples.

5. Functionalization of Nanoparticles by Attachment to Overhangs

Folded and purified nanoparticles were purified in 30 mM HEPES-KOH pH 7.4 with 200 mM NaCl, 8 mM MgCl₂ with 5 mM dithiolthreotol and incubated with 2-10 molar excess of sgRNA and Cas9 protein or Cpf1/Cas12a protein. The sgRNA and CRISPR protein were incubated for 1 minute at 42° C. followed by 1 h at 37° C. and then annealed to room temperature. Coincubation using either the crossbeam approach for Cas9 or the address overhang sequences for Cpf1/Cas12a show clear gel shifts in the presence of protein and cr/sgRNAs. In the case of Cas9 protein interaction with crossbeam or address overhangs, the bound protein was additionally visualized by Coomassie blue staining of the protein in a 2% high-resolution agarose gel ran at 4° C. with 1×TAE and 12 mM MgCl₂.

In the case of the sgRNA targeting EGFP using Cas9, the broccoli aptamer in presence of dye was fluorescent both with and without the presence of Cas9, and the sgRNA-brocolli-Cas9 was capable of cutting an EGFP plasmid to completion. The sgRNA-broccoli aptamer was able to colocalizing to the staple containing the ssDNA bait after annealing from 42° C. to 25° C., and was released from the staple upon digestion with RNase H, as proposed, as visualized by staining a 6% PAGE gel.

Finally, crRNAs modified with fluorescent cy5-CTP at 10% nucleotide labeling were transcribed and incubated with nanoparticles folded with cy3-dCTP labeled scaffolds. Co-localization of the fluorescence signal further indicated capture of the RNA on the overhang. Bait sequence labeled with a FAM fluorescence group against the cy5-crRNA targeting VEGFA was shown to co-localize after annealing, and subsequent digestion with RNase H for 5 minutes at 37° C. showed the sequences no longer co-localized on a PAGE gel when visualized with a Typhoon imager, further showing the functionality of the release mechanism using tuned degradation of some but not all of the components of the system.

6. Design of DNA/RNA Assemblies Containing an HDR Template for Gene Correction and Aptamers for Targeting.

Wireframe origami is composed of DX-tile based edges that contain staple and scaffold crossovers. Such tiles can be used as an edge of a wireframe origami (e.g., DNA/RNA hybrid origami). Nucleic acid assemblies containing such tiles allow for delivery of various cargo (e.g., CRISPR, PET imager, fluorescent imager). As an example, the assemblies can be used for targeted delivery of HDR templates and Cas9 simultaneously using aptamers.

In this example, a DX tile composed of a DNA scaffold with RNA staples was generated. The tile contains a long DNA scaffold encoding an HDR template that mediates GFP to BFP conversion (Glaser A, et al., Mol Ther Nucleic Acids. 5(7):e334 (2016)), two flanking/end staples composed of RNA and having overhangs encoding an aptamer, and a central/internal RNA staple whose 5′ end extends into an spyCas9 single-guide RNA that cuts at a site to be repaired.

The aptamers used were the Gint4.T (Camorani S, et al., Mol Ther. 22(4):828-41 (2014)), Gint21.T (Catuogno S, et al., J Control Release., 210:147-59 (2015)) and Broccoli fluorescent RNA (Filonov G S, et al., J Am Chem Soc., 136(46):16299-308 (2014)). The following sequences were used:

| | |
|---|---|
| EGFPtoBFPhdr | GTCGTGCTGCTTCATGTGGTCGGGGTAGCGGCTGAAGCACTGCA |
| | CGCCGTGGCTCAGGGTGGTCACGAGGGTGGGCCAGGGCACGGG |
| | CAGCTTGCCGGT (SEQ ID NO: 57) |
| St2 | ACCGGCAAGCTAGCAGCACGAC (SEQ ID NO: 58) |
| st4 | GCGTGCAGTGCTTCGTGACCACCCTGAGCCACG (SEQ ID NO: 59) |
| st2broc_for | CGTAATACGACTCACTATAGGACCGGCAAGC (SEQ ID NO: 60) |
| st4broc_for | CGTAATACGACTCACTATAGGCGTGCAGTGC (SEQ ID NO: 61) |
| st2Gint4T_rev | TGTCGAATTGCATTTACTCGATGCCCCACGACAGTCGTGCTGCTA |
| | GCTTGCCGGTCCTATAGTGAGTCGTATTACG (SEQ ID NO: 62) |
| st4Gint21T_rev | GTGAGCCTCCTGTCGAATTGAGGCGATTGATCATCGTGGCTCAG |
| | GGTGGTCACGAAGCACTGCACGCCTATAGTGAGTCGTATTACG |
| | (SEQ ID NO: 63) |
| broc_for | GCTGTTGAGCCCACACTCTACTCGACAGATACGAATATCTGGAC |
| | CCGACCGTCTCAACAG (SEQ ID NO: 64) |
| st2broc_rev | CGTAATACGACTCACTATAGGACCGGCAAGCTAGCAGCACGACct |
| | gttgagacggtcggg (SEQ ID NO: 65) |
| st4broc_rev | CGTAATACGACTCACTATAGGCGTGCAGTGCTTCGTGACCACCC |
| | TGAGCCACGctgttgagacggtcggg (SEQ ID NO: 66) |
| spCas9EGFPsgRNA_st3 | CGTAATACGACTCACTATAGCTGAAGCACTGCACGCCGTgttttagag |
| | ctatgctgttgaaaaacagcatagcaagttaaaataaggctagtccgt |
| | tatcaacttgaaaaagtggcaccgagtcggtgctttttCTGGCCCACC |
| | CTCAGCCGCTACCCCGACCACATGAGCCCGTGCC (SEQ ID NO: 67) |
| st2Gint4T* | CGTAATACGACTCACTATAGGACCGGCAAGCTAGCAGCACGACT |
| | GTCGTGGGGCATCGAGTAAATGCAATTCGACA (SEQ ID NO: 167) |
| st4Gint21T* | CGTAATACGACTCACTATAGGCGTGCAGTGCTTCGTGACCACCC |
| | TGAGCCACGATGATCAATCGCCTCAATTCGACAGGAGGCTCAC |
| | (SEQ ID NO: 168) |
| st2broc* | CGTAATACGACTCACTATAGGACCGGCAAGCTAGCAGCACGACct |
| | gttgagacggtcgggtccagatattcgtatctgtcgagtagagtg |
| | tgggctcaacagc (SEQ ID NO: 169) |
| st4broc* | CGTAATACGACTCACTATAGGCGTGCAGTGCTTCGTGACCACCC |
| | TGAGCCACGctgttgagacggtcgggtccagatattcgtatctgt |
| | cgagtagagtgtgggctcaacagc (SEQ ID NO: 68) |

Sequences marked with an astericks (*) contain the T7 promoter (bold), the specific staple (italicized), and the aptamer sequences (underlined) respectively.

Each staple sequence was completed using PCR to double-stranded DNA (dsDNA) using Phusion polymerase, and column purified on a Qiagen column using standard cleanup (600 ul addition of buffer PB applied to the column, followed by washing with 750 ul PE, drying the column, and elution with water). The dsDNA template was then used as a template for transcription with an NEB HiScribe T7 transcription kit modified for short templates (using ¾ of the reagents as per manufacturer's recommendation). Transcribed RNA was cleaned by Qiagen cleanup, followed by denaturing PAGE purification on an 8% PAGE gel with 6M urea, extraction in 300 mM sodium acetate pH 5.2, and ethanol precipitation in 2.5 volumes of ice-cold ethanol at −20C for overnight. The precipitated RNA was pelleted at 14,000 RPM for 30 minutes, followed by ethanol removal, drying, and resuspension in water. The same procedure was applied to purify the HDR template.

Dx-tiles were made either containing the Gint or broccoli aptamers. To prepare the folded DX-tiles, the components (e.g., DNA scaffold, two flanking/end staples encoding either the Gint (i.e., Gint4.T and Gint21.T) or broccoli aptamers, and central staple) were mixed in equimolar concentrations and allowed to anneal over 2 hours from 95 C to 25 C. The resulting structures were visualized by gel electrophoresis.

Several bands were originally visualized, showing impurities in the production. However, visualization of fluorescence in the Broccoli channel (green fluorescent channel) showed an ability of the aptamer to maintain binding and function while on the DX tile. Given that staple 2 spans across the region targeted by the sgRNA and could therefore contribute to misfolding, in a repeat experiment, staple 2 was left out of the folding mixture. This yielded higher folding. Upon addition and annealing with Cas9, the DX tile band was closer to the well on the gel. Addition of RNase H both with and without Cas9 released the HDR template. The RNase treated, Cas9-bound DX tile showed evidence that the sgRNA was maintained.

7. Nucleotide Modifications to DNA/RNA Assemblies Allows for Control of Degradation.

An alternative assembly to that of Example 6 was generated. In this assembly, a tetrahedron was built composed of a single strand of RNA (enhanced green fluorescent protein mRNA) that routes through the assembly and DNA sequences are used for staple and scaffold crossovers. The sequences used to generate this structure are as follows:

RNA eGFP scaffold
(SEQ ID NO: 2)
AAGGGCGAGGAGCUGUUCACCGGGGUGGUGCCCAUCCUGGUCGAGCUGGAC
GGCGACGUAAACGGCCACAAGUUCAGCGUGUCCGGCGAGGGCGAGGGCGAU
GCCACCUACGGCAAGCUGACCCUGAAGUUCAUCUGCACCACCGGCAAGCUG
CCCGUGCCCUGGCCCACCCUCGUGACCACCCUGACCUACGGCGUGCAGUGC
UUCAGCCGCUACCCCGACCACAUGAAGCAGCACGACUUCUUCAAGUCCGCC
AUGCCCGAAGGCUACGUCCAGGAGCGCACCAUCUUCUUCAAGGACGACGGC
AACUACAAGACCCGCGCCGAGGUGAAGUUCGAGGGCGACACCCUGGUGAAC
CGCAUCGAGCUGAAGGGCAUCGACUUCAAGGAGGACGGCAACAUCCUGGGG
CACAAGCUGGAGUACAACUACAACAGCCACAACGUCUAUAUCAUGGCCGAC
AAGCAGAAGAACGGCAUCAAGGUGAACUUCAAGAUCCGCCACAACAUCGAG
GACGGCAGCGUGCAGCUCGCCGACCACUACCAGCAGAACACCCCCAUCGGC
GACGGCCCCGUGCUGCUGCCCGACAACCACUACCUGAGCACCCAGUCCGCC
CUGAGCAAAGACCCCAACGAGAAGCGCGAUCACAUGGUCCUGCUGGAGUUC
GUGACCGCCGCCGGGAUCACUCUCGGCAUGGACGAGCUGUACAAGUAACUG
CAGGCAUGCAAGCUUGGCGUAAUCAUGGUCAUAGCUGUUUCCUGUGUGAAA
UUGUUAUCCGCUCACAAUUCCACACAA
EGFP_Aform_st2
(SEQ ID NO: 69)
CGGCTGAAGCGCTCCTCGCCCTTGGAAACAGGGTCGGGGTAG
EGFP_Aform_st3
(SEQ ID NO: 70)
GGTGAACAACTGCACGCCGTAGGTCAGGGTGGGCACCACCCC
EGFP_Aform_st4
(SEQ ID NO: 71)
TTCAGGGTCAGTTTTTTTCTTGCCGTAGCGCCGTCCAGCTTTTTTTTCGAC
CAGGATGGTCACGAGGTTTTTTTGTGGGCCAGG
EGFP_Aform_st5
(SEQ ID NO: 72)
CCGTTTACGTGTGGCATCGC
EGFP_Aform_st6
(SEQ ID NO: 73)
ACTTGTGGCCTCGCCCTCGCCGGACATGTGAT
EGFP_Aform_st7
(SEQ ID NO: 74)
CGCGCTTCCGAACTCCAGCAGGACCACGCTGA
EGFP_Aform_st8
(SEQ ID NO: 75)
AGATGAACGCACGGGCAGCTTGCCCTTCACCT
EGFP_Aform_st9
(SEQ ID NO: 76)
CGGCGCGGGGGTGTCGCCCTCGAAGGTGGTGC
EGFP_Aform_st10
(SEQ ID NO: 77)
GCCGTCCTCGATTTTTTTTGTTGTGGCGAGAAGTCGTGCTTTTTTTTGCTT
CATGTCTATGACCATGTTTTTTTATTACGCCAA
EGFP_Aform_st11
(SEQ ID NO: 78)
TCACCTTGATCTTCGGGCATGGCGGACTTGAGATCTTGAAGT
EGFP_Aform_st12
(SEQ ID NO: 79)
GACGTAGCGCCGTTCTTCTGCTTGTCGGCTGGTGCGCTCCTG
EGFP_Aform_st13
(SEQ ID NO: 80)
CATGATATAGATTTTTTTCGTTGTGGCTTCGATGCCCTTTTTTTTTCAGCT
CGATGTGCCGTCGTCCTTTTTTTTTGAAGAAGA
EGFP_Aform_st14
(SEQ ID NO: 81)
GTCTTGTAGTCGGTTCACCA
EGFP_Aform_st15
(SEQ ID NO: 82)
CTCCTTGAAGGTTGTAGTTG
EGFP_Aform_st16
(SEQ ID NO: 83)
TTGCCGTCTACTCCAGCTTGTGCCGGTAGTGG
EGFP_Aform_st17
(SEQ ID NO: 84)
TTGTCGGGGCGGACTGGGTGCTCACCAGGATG
EGFP_Aform_st18
(SEQ ID NO: 85)
TGCAGTTACTGTGGTCGGCGAGCTGCACGCTGCTTGCATGCC
EGFP_Aform_st19
(SEQ ID NO: 86)
TGCTGGTATGTACAGCTCGTCCATGCCGAGATGGGGGTGTTC
EGFP_Aform_st20
(SEQ ID NO: 87)
TCGTTGGGGTCTTTTTTTTTTGCTCAGGCAGCAGCACGGTTTTTTTGGCCG
TCGCCGAGTGATCCCGTTTTTTTGCGGCGGTCA

The EGFP sequence was generated as a gBlock and cloned with a T7 promoter and Shine-Dalgarno (SD) sequence 5′ of the coding sequence into a pUC19 vector using restriction cloning (EcoRI, PstI). RNA was transcribed from a Phusion PCR-generated double-stranded DNA (dsDNA) template containing a 5′ T7 promoter and amplified and gel purified. Using the gel-purified dsDNA template, RNA was transcribed using the manufacturer's protocol for HiScribe T7 (NEB) for canonical base RNAs or DuraScribe T7 (Lucagen) for 2′-fluoro-modified base RNAs. Urea polyacrylamide gel (PAGE) or HPLC was used to purify the RNAs, and PAGE was used to validate the purity. PAGE purification was performed on a 6% gel containing 8M urea, with the RNA pre-cleaned on a ZymoClean RNA cleanup kit (RNA Clean-and-concentrator 5), followed by addition of 1×RNA denaturing buffer (NEB) and heating for 5 minutes at 70° C. RNA was sliced from the gel after visualization with SybrSafe (ThermoFisher) and eluted in 300 mM sodium acetate pH 5.2, precipitated in 70% ethanol at −20° C. for more than 2 hours, and then pelleted at 14,000 RPM for 30 minutes at 4° C.

Using RNase-free buffers and conditions, 20 nM of purified scaffold was mixed with 400 nM individual staples and buffer and salt and brought to a volume of 50 μl aliquots for temperature ramping. RNA/DNA hybrid nanoparticles were folded in 10 mM HEPES-KOH and concentrations of KCl and NaCl of >200 mM. Folding was performed using a modification of the previously published wireframe origami thermal annealing protocol (Veneziano, et al., Science, V. 352, (6293), pp. 1534 (2016)) but with reduced incubation time at high temperatures. Briefly, the folding protocol was 90° C. for 45 s; ramp 85° C. to 70° C. at 45s/° C.; ramp 70° C. to 29° C. at 15 m/° C.; ramp 29° C. to 25° C. at 10 m/° C.; 10 m at 37° C.; hold at 4° C. until purification. Folded particles were purified away from excess staples using Amicon Ultra 100 kDa 0.5 ml filter columns and buffer exchanged into the same buffer that was used for folding.

Biochemical stability in the presence of RNases A and H was tested. 50 nM RNA-scaffolded tetrahedron with 66-bp edge length was incubated for 5 minutes at 37° C. in the presence of buffer alone, 25 units of RNase H or 3.5 units of RNase A. RNA transcribed with 100% 2′-fluoro-deoxy-uridine and cytosine was folded to a tetrahedron with 66-bp edge lengths using the same staples, and also incubated for 5 minutes at 37° C. in the presence of 3.5 units of RNase A. Reactions were quenched at 4° C. and run at 65V for 180 minutes on a high-resolution 2.5% agarose gel in TBE with 2.5 mM Mg(OAc)₂, maintained at 4° C. on ice.

Cryo-EM validated the resulting structure to be a tetrahedron as expected from the input geometry. Incubation with RNase A or RNase H degraded the RNA in the structure within 5 minutes. However, the same RNA scaffold transcribed with 2′-fluorinated cytosine and uridine (100% replacement of C and U with 2′F-C and 2′F-U) did not show RNase A degradation in the same length of time. Use of modified nucleotides protected the nanoparticle from degradation in the same time scale as unmodified RNA. This indicates that degradation rates of DNA/RNA hybrid assemblies can be tuned by the presence of modified nucleotides, and this can allow for timed release of sets of DNA oligos (e.g., modified oligos, morphalinos, modified RNAs).

8. Generation of a DNA Pentagonal Bipyramid Assembly.

A staple set was generated for a DNA-scaffolded pentagonal bipyramid where the scaffold contained bacterially produced circular DNA. The staples were DNA except for 2 staples that were replaced by an RNA extension off of a sgRNA. An sgRNA targeting PCSK9 was extended 3′ to include the sequence that would be a replacement for the sequence of DNA staples being replaced, contained the following sequence: GAATTCTAATACGACTCACTATA_GGCTGATGAGGCCGCACATG_gttttagagctat gctgttCUUCGGaacagcatagcaagttaaaataaggctagtccgttatcaacttgaaaaagtggcaccgagtcggtgc_AAAAA_TCACCCAGAAACGCTGGTGAAAGTGTATGAGTATTCAACATTTCCG TGTCGCCCTTGTTTTTGCTGCAG (SEQ ID NO:88), where the sequences separated by underscores are T7promoter_MusPCSK9target_sgRNA for SpyCas9_Poly(A) linker_PB84 inside staples 71 and 72.

Staples for the Pentagonal Bipyramid:

| Staple # | Sequence |
|---|---|
| 2 | GCGAAATTAGACAAAGCTTTAAACAAAATCTG (SEQ ID NO: 89) |
| 3 | AAACTAAGCTTATATGTGTAAAAGGAACTGC (SEQ ID NO: 90) |
| 4 | TATGCAAATTGCACTCTGAATCTGTCTATAC (SEQ ID NO: 91) |
| 5 | CAGGTACGAGCTTTTTGAACGAAACCATAGAATCGGTTTTTTATCCAAAGA |
| | TCCGTGTATCATTTTTTAATTGTCCGA (SEQ ID NO: 92) |
| 6 | GGGAAAACTCAAATCCTATCTGTGTTTCTTA (SEQ ID NO: 93) |
| 7 | GACGTCAGTCATGATAATAATGGAAGAACCA (SEQ ID NO: 94) |
| 8 | GGTTAATGGTGGCACTTTTCGGGGAAATGTGC (SEQ ID NO: 95) |
| 9 | TGAGCTGGTTATTTTTCTAGGTACATCCGCATAAGTGTTTTTTTGTAGAAAG |
| | (SEQ ID NO: 96) |
| 10 | GTAGGAAGGTTAGAAGTTAGAAATTTTGACT (SEQ ID NO: 97) |
| 11 | CGGGTGTACACCTGTATCGGCCTTCAAACGG (SEQ ID NO: 98) |
| 12 | TTGCGCAGATCAGTACTATGCAGTAACCTCAG (SEQ ID NO: 99) |
| 13 | GATAGTTATGATACTCTATGTTCGCCCGCATT (SEQ ID NO: 100) |
| 14 | ATTCATTCATTTTTTTGTGCAATCCTCTAGATTAGAATTTTTGGCGTTGTTT |
| | (SEQ ID NO: 101) |
| 15 | CTATATTCACTTTAAAAGAAAAGAGGTTGATACGTATCTTCC (SEQ ID |
| | NO: 102) |
| 16 | CCGCTCATGAAAGCATGGTAGCAAGTCGTTAA (SEQ ID NO: 103) |
| 17 | TGACCCAAGAGACAATAACCCTGAATATGTAT (SEQ ID NO: 104) |
| 18 | TTGGTCAGTTATCGGACCACGTGTAAGGATTA (SEQ ID NO: 105) |
| 19 | GTGACGTAACTCCCGGGTTGGTTTCATGTTAC (SEQ ID NO: 106) |
| 20 | ATGACAGTACATCTAGTGATAACGCACACGT (SEQ ID NO: 107) |
| 21 | TGTGTTGCAGGTTTTTTAGCGACAGTAAATATCTTCATTTTTCGACGTAGCG |
| | (SEQ ID NO: 108) |
| 22 | ATTCACAAGTCTGGCAATAC (SEQ ID NO: 109) |
| 23 | CCCCAATCTCTTATTACCCTCGATTAGTCACA (SEQ ID NO: 110) |
| 24 | GTCTATTAATATGTACCCCGGTTGGCGTCAGA (SEQ ID NO: 111) |
| 25 | ATAATTATGGGTTCCCAGAGCTATCGAGATA (SEQ ID NO: 112) |
| 26 | GGGTTGAGATCAAAAGAATAGACATTCTGAC (SEQ ID NO: 113) |
| 27 | TATTAATAGAGAAGTGGCTAACGCTGACGGG (SEQ ID NO: 114) |
| 28 | GAAAGCCGCCCCGATTTAGAGCTAAGCAAGA (SEQ ID NO: 115) |
| 29 | AAGGGAGCGCGAACGTGGCGAGAAAGGAAGGG (SEQ ID NO: 116) |
| 30 | GCGCCCGGTTGCGCTCCTGCAGCATACCTATG (SEQ ID NO: 117) |
| 31 | ACAGGGCGCACTTTTTATTAATTGCGGTGGCTCAACATTTTTACAGAGAAT |
| | A (SEQ ID NO: 118) |
| 32 | GCGCCGCTCTGGCGAACTACTTACTCTAGCGCCGCGCTTAAT (SEQ ID |
| | NO: 119) |
| 33 | CACGCTGCGCGTAACCACCACACCCTTCCCGG (SEQ ID NO: 120) |
| 34 | CAACAATTAATAGACTGGATGGAGGTAGCGGT (SEQ ID NO: 121) |
| 35 | GCGGATAAAGGCTGGCAAGT (SEQ ID NO: 122) |
| 36 | GAAAGGAGCGGTTTTTGCGCTAGGGCTTGCAGGACCATTTTTCTTATGCGC |
| | T (SEQ ID NO: 123) |
| 37 | AAGAAAGCGTAAAGCACTAAATCGGAACCCTA (SEQ ID NO: 124) |
| 38 | GCCCTCCCGTATTTTTTCGTAGTTATGTTTTTTGGGGTTTTTTCGAGGTGCC |
| | (SEQ ID NO: 125) |
| 39 | CTAATCAACTACACGACGGGGAGTCAGGCGTGAACCATCACC (SEQ ID |
| | NO: 126) |
| 40 | TCTATCAGGGCGATGGCCCACTACAACTATGG (SEQ ID NO: 127) |
| 41 | ATGAACGAAATAGACAGATCGCTGAAAAACCG (SEQ ID NO:128) |
| 42 | AGATAGGTGCTCAAAGGGCG (SEQ ID NO: 129) |
| 43 | TAAATCAGCTCTTTTTATTTTTTAACTAAAGAACGTGTTTTTGACTCCAACG |
| | (SEQ ID NO: 130) |
| 44 | TCCACTATCAATAGGCCGAAATCGGCAAAATC (SEQ ID NO: 131) |
| 45 | CCTTATAATGTTGTTCCAGTTTGGAACAAGAG (SEQ ID NO: 132) |
| 46 | ATTTTTGTCGTGAGTTTTCGTTCCACTGATAATTCGCGTTAA (SEQ ID |
| | NO: 133) |
| 47 | CTCACTGATTATTTTTAGCATTGGTACTCATGACCAATTTTTAATCCCTTAA |
| | (SEQ ID NO: 134) |
| 48 | TAGGTGAAGATCCTTTTTGATAATACTGTCAG (SEQ ID NO: 135) |
| 49 | ACCAAGTTTACTCATATATACTTAAAAGGATC (SEQ ID NO: 136) |
| 50 | GGCCAACTTTCATTTTTAATTTAAGATTGAT (SEQ ID NO: 137) |
| 51 | TGCAGCACTGGGGCCAGATGGTAACGGCCCTT (SEQ ID NO: 138) |
| 52 | CCGGCTGGCTGGTTTATTGCTGATGGTATCAT (SEQ ID NO: 139) |
| 53 | CCCCGAAGAACTTTTTGTTTTCCAATCAACGTTGCGCTTTTTAAACTATTAA |
| | (SEQ ID NO: 140) |
| 54 | CCACGATGCCTGTAGCAATGGCAAGATGAGCA (SEQ ID NO: 141) |
| 55 | CTTTTAAAGTTCTGCTATGTGGCGGCGTGACA (SEQ ID NO: 142) |
| 56 | AATGAAGCCATACCAAACGACGACGGTATTA (SEQ ID NO: 143) |
| 57 | TCCCGTATTGACGCCGGGCAAGACGGAGCTG (SEQ ID NO: 144) |
| 58 | CGGAGGACCGATTTTTAGGAGCTAACCTCGCCTTGATTTTTTCGTTGGGAA |
| | CGCAACTCGGTCTTTTTGCCGCATACA (SEQ ID NO: 145) |
| 59 | AGCCGGTATGGGGGATCATGTAACGCTTTTT (SEQ ID NO: 146) |
| 60 | TGCACAACGAGCGTGGGTCTCGCAAATCTGG (SEQ ID NO: 147) |
| 61 | TTAAAACTACTTCTGACAACGATAACACTGC (SEQ ID NO: 148) |
| 62 | CAGTCACAGAATTTTTAAGCATCTTAGCTGCCATAACTTTTTCATGAGTGAT |
| | (SEQ ID NO: 149) |
| 63 | CTGGCGGAAGAGAATTATGCAGTCGGATGGC (SEQ ID NO: 150) |
| 64 | TTTTGCGTTGGTTGAGTACTCACCTATTCTC (SEQ ID NO: 151) |
| 65 | AGAATGACGCATTTTGCCTTCCTATTCCCTT (SEQ ID NO: 152) |
| 66 | AGAGTTTTCGAGTAAATTAC (SEQ ID NO: 153) |
| 67 | ATCTCAACAGCGGTAAGATCCTTGATGCGATG (SEQ ID NO: 154) |
| 68 | TGACCTTAAACGAATAGCCTCTCCCGAACTGG (SEQ ID NO: 155) |
| 69 | AGGGGACGGGGTGCACGAGTGGGTTACATACCCAAGAGTTTG (SEQ ID |
| | NO: 156) |
| 70 | AAAAGATGCTGTTTTTAAGATCAGTTACGACGAATTCTTTTTGACGAAAGG |
| | G (SEQ ID NO: 157) |
| 71 (replaced | TCACCCAGAAACGCTGGTGAAAGTGTATGAGT (SEQ ID NO: 158) |
| with RNA) | |
| 72 (replaced | ATTCAACATTTCCGTGTCGCCCTTGTTTTTGC (SEQ ID NO: 159) |
| with RNA) | |
| 73 | CCTATTTGTTTTTTTTATTTTTCTAAAATAATATTGATTTTTAAAAGGAAGA |
| | (SEQ ID NO: 160) |
| 74 | TAAATGCTTCATACATTCAA (SEQ ID NO: 161) |
| 75 | GCGGAACCCCTCGTGATACGCCTATTTTTATA (SEQ ID NO: 162) |

The following is the scaffold sequence:

(SEQ ID NO: 163)
GAGCGCAACGCAATTAATGTGCGCCCTGTAGCGGCGCATTAAGCGCGGCGG
GTGTGGTGGTTACGCGCAGCGTGACCGCTACACTTGCCAGCGCCCTAGCGC
CCGCTCCTTTCGCTTTCTTCCCTTCCTTTCTCGCCACGTTCGCCGGCTTTC
CCCGTCAAGCTCTAAATCGGGGGCTCCCTTTAGGGTTCCGATTTAGTGCTT
TACGGCACCTCGACCCCAAAAAACTTGATTAGGGTGATGGTTCACGTAGTG
GGCCATCGCCCTGATAGACGGTTTTTCGCCCTTTGACGTTGGAGTCCACGT
TCTTTAATAGTGGACTCTTGTTCCAAACTGGAACAACACTCAACCCTATCT
CGGTCTATTCTTTTGATTTATAAGGGATTTTGCCGATTTCGGCCTATTGGT
TAAAAAATGAGCTGATTTAACAAAAATTTAACGCGAATTACAACCGGGGTA
CATATGATTGGGGTCTGACGCTCAGTGGAACGAAAACTCACGTTAAGGGAT
TTTGGTCATGAGATTATCAAAAAGGATCTTCACCTAGATCCTTTTAAATTA
AAAATGAAGTTTTAAATCAATCTAAAGTATATATGAGTAAACTTGGTCTGA
CAGTTACCAATGCTTAATCAGTGAGGCACCTATCTCAGCGATCTGTCTATT
TCGTTCATCCATAGTTGCCTGACTCCCCGTCGTGTAGATAACTACGATACG
GGAGGGCTTACCATCTGGCCCCAGTGCTGCAATGATACCGCGAGACCCACG
CTCACCGGCTCCAGATTTATCAGCAATAAACCAGCCAGCCGGAAGGGCCGA
GCGCATAAGTGGTCCTGCAACTTTATCCGCCTCCATCCAGTCTATTAATTG
TTGCCGGGAAGCTAGAGTAAGTAGTTCGCCAGTTAATAGTTTGCGCAACGT
TGTTGCCATTGCTACAGGCATCGTGGTGTCACGCTCGTCGTTTGGTATGGC
TTCATTCAGCTCCGGTTCCCAACGATCAAGGCGAGTTACATGATCCCCCAT
GTTGTGCAAAAAAGCGGTTAGCTCCTTCGGTCCTCCGATCGTTGTCAGAAG
TAAGTTGGCCGCAGTGTTATCACTCATGGTTATGGCAGCACTGCATAATTC
TCTTACTGTCATGCCATCCGTAAGATGCTTTTCTGTGACTGGTGAGTACTC
AACCAAGTCATTCTGAGAATAGTGTATGCGGCGACCGAGTTGCTCTTGCCC
GGCGTCAATACGGGATAATACCGCGCCACATAGCAGAACTTTAAAAGTGCT
CATCATTGGAAAACGTTCTTCGGGGCGAAAACTCTCAAGGATCTTACCGCT
GTTGAGATCCAGTTCGATGTAACCCACTCGTGCACCCAACTGATCTTCAGC
ATCTTTTACTTTCACCAGCGTTTCTGGGTGAGCAAAAACAGGAAGGCAAAA
TGCCGCAAAAAAGGGAATAAGGGCGACACGGAAATGTTGAATACTCATACT
CTTCCTTTTTCAATATTATTGAAGCATTTATCAGGGTTATTGTCTCATGAG
CGGATACATATTTGAATGTATTTAGAAAAATAAACAAATAGGGGTTCCGCG
CACATTTCCCCGAAAAGTGCCACCTGACGTCTAAGAAACCATTATTATCAT
GACATTAACCTATAAAAATAGGCGTATCACGAGGCCCTTTCGTCGAATTCG
TCGTCGTCCCCTCAAACTCTTGGGTGGAGAGGCTATTCGTTTAAGGTCACA
TCGCATGTAATTTACTTATTCTCTGTTGTTGAGCCACCCGGGCGCCAGATT
TTGTTTAAAGCTTTGTCTCTTAGTTTGTATAGACAGATTCAGAGTGCAAGG
TTTCGTTCGCTCGTACCTGGTTTTCCCTGGTTCTTCACAGATAGGATTTGA
CTTTCTACAACACTTATGCGGCTTCCTACCCGTTTGAAGGCCGATACAGGT
GCTGCGCAAAATGCGGGCGAACATAGAGTATCAAAACAACGCCTTCTAATC
TAGGAATATAGGGAAGATACGTATTTGCTACCATGCTTTCTTGGGTCATTA
ACGACCAACCTCTTTTCTTTTAAAGTAGGATTGCACAATGAATGAATACAC
GTGGTCCGATAACTGACCAAGTAACATGGTTATCACTAGATGTCCGCCAGA
CGTGTGCAAACCAACCCGGGAGTTACGTCACTAATCCTTCGCTACGTCGTG
AAGATATTTACTTGTGAATATCGAGGGTAATAAGATAATAGACTGTGACTA
GTATTGCCAGACTGTCGCTACCTGCAACACATAACTATCCTGAGGTTACTG
CATAGTACTGATTACACCCGAGTCAAAATTTCTAACTTCTAACATGTACCT
AGTAACCAGCTCAATAATTATGTCAGAATATAGCTCTGGGAACCCTCGGAC
AATTATGATACACGGTATTAATATCTTGCTTGCGTTAGCCACTTCTCATCT
TTGGATACCGATTCTATTTTGCATAGCAGTTCCTTTTACACATATAAGAAT
TTCGCCATAGGTATGCTGCAG.

The DNA was amplified and purified by Qiagen, and transcribed using the NEB T7 HiScribe kit modified for short RNA production, and purified by PAGE purification. It was then folded with 50 mM HEPES-KOH pH 7.5, 300 mM KCl following the RNA/DNA hybrid folding protocol. The excess staples were purified away from the PB84 with a 100 kDa MWCO filter. A 2% high resolution agarose gel with 1×TAE buffer and 4 mM Mg-acetate was run at 4 C in the cold room at 65V for 180 minutes.

It was observed that the nanoparticle with the RNA staple presented a band at the same size as the all-DNA staple nanoparticle. Addition of 5×-molar equivalents of Cas9 showed the nanoparticle band shifted into the well, unlike the tetrahedron where the nanoparticle only shifted moderately but fluorescence showed co-localization of the Cas9 with the tetrahedron.

### BIBLIOGRAPHY

1. Li, F., Vijayasankaran, N., Shen, A. Y., Kiss, R. & Amanullah, A. Cell Culture Processes for Monoclonal Antibody Production. MAbs 2, 466-79 (2010).
2. Wurm, F. M. Production of Recombinant Protein Therapeutics in Cultivated Mammalian Cells. Nat Biotechnol 22, 1393-8 (2004).
3. Bhatia, S. N., Underhill, G. H., Zaret, K. S. & Fox, I. J. Cell and Tissue Engineering for Liver Disease. Sci Transl Med 6, 245sr2 (2014).
4. Griffith, L. G., Wells, A. & Stolz, D. B. Engineering Liver. Hepatology 60, 1426-34 (2014).
5. Powell, J. D., Hutchison, J. R., Hess, B. M. & Straub, T. M. Bacillus Anthracis Spores Germinate Extracellularly at Air-Liquid Interface in an in Vitro Lung Model under Serum-Free Conditions. J Appl Microbiol 119, 711-23 (2015).
6. Eklund, S. E. et al. Metabolic Discrimination of Select List Agents by Monitoring Cellular Responses in a Multianalyte Microphysiometer. Sensors 9, 2117-33 (2009).
7. Lee, J. S., Kallehauge, T. B., Pedersen, L. E. & Kildegaard, H. F. Site-Specific Integration in Cho Cells Mediated by Crispr/Cas9 and Homology-Directed DNA Repair Pathway. Sci Rep 5, 8572 (2015).
8. Weber, J. et al. Crispr/Cas9 Somatic Multiplex-Mutagenesis for High-Throughput Functional Cancer Genomics in Mice. Proc Natl Acad Sci USA 112, 13982-7 (2015).
9. Housden, B. E. et al. Identification of Potential Drug Targets for Tuberous Sclerosis Complex by Synthetic Screens Combining Crispr-Based Knockouts with Rnai. Sci Signal 8, rs9 (2015).
10. Wang, K. et al. Efficient Generation of Myostatin Mutations in Pigs Using the Crispr/Cas9 System. Sci Rep 5, 16623 (2015).
11. Ali, Z. et al. Efficient Virus-Mediated Genome Editing in Plants Using the Crispr/Cas9 System. Mol Plant 8, 1288-91 (2015).
12. Recillas-Targa, F. Multiple Strategies for Gene Transfer, Expression, Knockdown, and Chromatin Influence in Mammalian Cell Lines and Transgenic Animals. Mol Biotechnol 34, 337-54 (2006).
13. Jinek, M. et al. A Programmable Dual-Rna-Guided DNA Endonuclease in Adaptive Bacterial Immunity. Science 337, 816-21 (2012).
14. Dowdy, S. F. Overcoming Cellular Barriers for RNA Therapeutics. Nat Biotechnol 35, 222-229 (2017).
15. Kim, S, Kim, D., Cho, S. W., Kim, J. & Kim, J. S. Highly Efficient Rna-Guided Genome Editing in Human Cells Via Delivery of Purified Cas9 Ribonucleoproteins. Genome Res 24, 1012-9 (2014).
16. Savic, N. et al. Covalent Linkage of the DNA Repair Template to the Crispr/Cas9 Complex Enhances Homology-Directed Repair. bioRxiv (2017).
17. Ran, F. A. et al. In Vivo Genome Editing Using Staphylococcus Aureus Cas9. Nature 520, 186-91 (2015).
18. Yin, H. et al. Therapeutic Genome Editing by Combined Viral and Non-Viral Delivery of Crispr System Components in Vivo. Nat Biotechnol 34, 328-33 (2016).
19. Fu, Y. et al. High-Frequency Off-Target Mutagenesis Induced by Crispr-Cas Nucleases in Human Cells. Nat Biotechnol 31, 822-6 (2013).
20. Han, X. et al. Crispr-Cas9 Delivery to Hard-to-Transfect Cells Via Membrane Deformation. Sci Adv 1, e1500454 (2015).
21. Ramakrishna, S. et al. Gene Disruption by Cell-Penetrating Peptide-Mediated Delivery of Cas9 Protein and Guide Rna. Genome Res 24, 1020-7 (2014).
22. Zuris, J. A. et al. Cationic Lipid-Mediated Delivery of Proteins Enables Efficient Protein-Based Genome Editing in Vitro and in Vivo. Nat Biotechnol 33, 73-80 (2015).
23. Barbieri, E. M., Muir, P., Akhuetie-Oni, B. O., Yellman, C. M. & Isaacs, F. J. Precise Editing at DNA Replication Forks Enables Multiplex Genome Engineering in Eukaryotes. Cell (2017).
24. Zhang, Q. et al. DNA Origami as an in Vivo Drug Delivery Vehicle for Cancer Therapy. ACS Nano 8, 6633-43 (2014).
25. Lee, H. et al. Molecularly Self-Assembled Nucleic Acid Nanoparticles for Targeted in Vivo Sirna Delivery. Nat Nanotechnol 7, 389-93 (2012).
26. Rothemund, P. W. Folding DNA to Create Nanoscale Shapes and Patterns. Nature 440, 297-302 (2006).
27. Veneziano, R. et al. Designer Nanoscale DNA Assemblies Programmed from the Top Down. Science 352, 1534 (2016).
28. Veneziano, R. et al. Enzymatic Synthesis of Gene-Length Single-Stranded DNA. bioRxiv (2017).
29. Shepherd, T. R., Du, R. & Bathe, M. Bacterial Production of Pure Single-Stranded DNA of Arbitrary Sequence. In preparation (2017).
30. Yin, H. et al. Structure-Guided Chemical Modification of Guide RNA Enables Potent Non-Viral in Vivo Genome Editing. Nat Biotechnol (2017).
31. Johannssen, T. & Lepenies, B. Glycan-Based Cell Targeting to Modulate Immune Responses. Trends Biotechnol 35, 334-346 (2017).
32. Angata, T., Nycholat, C. M. & Macauley, M. S. Therapeutic Targeting of Siglecs Using Antibody- and Glycan-Based Approaches. Trends Pharmacol Sci 36, 645-60 (2015).
33. D'Souza, A. A. & Devarajan, P. V. Asialoglycoprotein Receptor Mediated Hepatocyte Targeting—Strategies and Applications. J Control Release 203, 126-39 (2015).
34. Onizuka, T. et al. NMR Study of Ligand Release from Asialoglycoprotein Receptor under Solution Conditions in Early Endosomes. FEBS J 279, 2645-56 (2012).
35. Hanske, J. et al. Intradomain Allosteric Network Modulates Calcium Affinity of the CType Lectin Receptor Langerin. J Am Chem Soc 138, 12176-86 (2016).
36. Schwartz, A. L., Bolognesi, A. & Fridovich, S. E. Recycling of the Asialoglycoprotein Receptor and the Effect of Lysosomotropic Amines in Hepatoma Cells. J Cell Biol 98, 732-8 (1984).
37. O'Reilly, M. K., Tian, H. & Paulson, J. C. Cd22 Is a Recycling Receptor That Can Shuttle Cargo between the Cell Surface and Endosomal Compartments of B Cells. J Immunol 186, 1554-63 (2011).
38. Esko, J. D., J, H. P. & Linhardt, R. J. Proteins That Bind Sulfated Glycosaminoglycans. in Essentials of Glycobiology (eds. Varki, A. et al.) (Cold Spring Harbor (N.Y.), 2015).
39. Guye, P. et al. Genetically Engineering Self-Organization of Human Pluripotent Stem Cells into a Liver Bud-Like Tissue Using Gata6. Nat Commun 7, 10243 (2016).
40. Dong, J. et al. Elucidation of a Universal Size-Control Mechanism in Drosophila and Mammals. Cell 130, 1120-33 (2007).
41. Gresch, 0. & Altrogge, L. Transfection of Difficult-to-Transfect Primary Mammalian Cells. Methods Mol Biol 801, 65-74 (2012).
42. Prakash, T. P. et al. Comprehensive Structure-Activity Relationship of Triantennary NAcetylgalactosamine Conjugated Antisense Oligonucleotides for Targeted Delivery to Hepatocytes. J Med Chem 59, 2718-33 (2016).
43. Sanhueza, C. A. et al. Efficient Liver Targeting by Polyvalent Display of a Compact Ligand for the Asialoglycoprotein Receptor. J Am Chem Soc 139, 3528-3536 (2017).
44. Huang, X., Leroux, J. C. & Castagner, B. Well-Defined Multivalent Ligands for Hepatocytes Targeting Via Asialoglycoprotein Receptor. Bioconjug Chem 28, 283-295 (2017).
45. Agarwal, R. et al. Mammalian Cells Preferentially Internalize Hydrogel Nanodiscs over Nanorods and Use Shape-Specific Uptake Mechanisms. Proc Natl Acad Sci USA 110, 17247-52 (2013).
46. Bujold, K. E. et al. Sequence-Responsive Unzipping DNA Cubes with Tunable Cellular Uptake Profiles. Chemical Science 5, 2449-2455 (2014).
47. Bechara, C. & Sagan, S. Cell-Penetrating Peptides: 20 Years Later, Where Do We Stand? FEBS Lett 587, 1693-702 (2013).
48. Taddeo, A. et al. Selection and Depletion of Plasma Cells Based on the Specificity of the Secreted Antibody. Eur J Immunol 45, 317-9 (2015).
49. Peng, W. & Paulson, J. C. Cd22 Ligands on a Natural N-Glycan Scaffold Efficiently Deliver Toxins to B-Lymphoma Cells. J Am Chem Soc 139, 12450-12458 (2017).
50. Lu, X. & Huang, X. Design and Syntheses of Hyaluronan Oligosaccharide Conjugates as Inhibitors of Cd44-Hyaluronan Binding. Glycoconj J 32, 549-56 (2015).
51. Pogson, M., Parola, C., Kelton, W. J., Heuberger, P. & Reddy, S. T. Immunogenomic Engineering of a Plug-and-(Dis)Play Hybridoma Platform. Nat Commun 7, 12535 (2016).
52. Bandaranayake, A. D. & Almo, S. C. Recent Advances in Mammalian Protein Production. FEBS Lett 588, 253-60 (2014).
53. Zhang, W. et al. Generation of Apoptosis-Resistant Hek293 Cells with Crispr/Cas Mediated Quadruple Gene Knockout for Improved Protein and Virus Production. Biotechnol Bioeng 114, 2539-2549 (2017).
54. Sacca, B. et al. Orthogonal Protein Decoration of DNA Origami. Angew Chem Int Ed Engl 49, 9378-83 (2010).
55. Sacca, B. & Niemeyer, C. M. Functionalization of DNA Nanostructures with Proteins. Chemical Society Reviews 40, 5910-5921 (2011).
56. Meyer, R., Sacca, B. & Niemeyer, C. M. Site-Directed, on-Surface Assembly of DNA Nanostructures. Angew Chem Int Ed Engl 54, 12039-43 (2015).
57. Conway, J. W., McLaughlin, C. K., Castor, K. J. & Sleiman, H. DNA Nanostructure Serum Stability: Greater Than the Sum of Its Parts. Chem Commun (Camb) 49, 1172-4 (2013).
58. Zetsche, B. et al. Cpf1 Is a Single Rna-Guided Endonuclease of a Class 2 Crispr-Cas System. Cell 163, 759-71 (2015).
59. Zetsche, B. et al. Multiplex Gene Editing by Crispr-Cpf1 Using a Single Crrna Array. Nat Biotechnol 35, 31-34 (2017).
60. Ran, F. A. et al. Double Nicking by Rna-Guided Crispr Cas9 for Enhanced Genome Editing Specificity. Cell 154, 1380-9 (2013).
61. Ponnuswamy, N. et al. Oligolysine-Based Coating Protects DNA Nanostructures from Low-Salt Denaturation and Nuclease Degradation. Nat Commun 8, 15654 (2017).
62. Bedell, V. M. et al. In Vivo Genome Editing Using a High-Efficiency Talen System. Nature 491, 114-8 (2012).
63. Urnov, F. D., Rebar, E. J., Holmes, M. C., Zhang, H. S. & Gregory, P. D. Genome Editing with Engineered Zinc Finger Nucleases. Nat Rev Genet 11, 636-46 (2010).
64. Nyberg, S. L. et al. Primary Hepatocytes Outperform Hep G2 Cells as the Source of Biotransformation Functions in a Bioartificial Liver. Ann Surg 220, 59-67 (1994).
65. Renz, M., Daniels, B. R., Vamosi, G., Arias, I. M. & Lippincott-Schwartz, J. Plasticity of the Asialoglycoprotein Receptor Deciphered by Ensemble Fret Imaging and Single-Molecule Counting Palm Imaging. Proc Natl Acad Sci USA 109, E2989-97 (2012).
66. Schmidt, K. et al. Characterizing the Effect of Galnac and Phosphorothioate Backbone on Binding of Antisense Oligonucleotides to the Asialoglycoprotein Receptor. Nucleic Acids Res 45, 2294-2306 (2017).
67. Boulant, S., Kural, C., Zeeh, J. C., Ubelmann, F. & Kirchhausen, T. Actin Dynamics Counteract Membrane Tension During Clathrin-Mediated Endocytosis. Nat Cell Biol 13, 1124-31 (2011).
68. Castro, C. E. et al. A Primer to Scaffolded DNA Origami. Nat Methods 8, 221-9 (2011).
69. Pan, K., Bricker, W. P., Ratanalert, S. & Bathe, M. Structure and Conformational Dynamics of Scaffolded DNA Origami Nanoparticles. Nucleic Acids Res 45, 6284-6298 (2017).
70. Guo, S.-M. et al. Multiplexed Confocal and Super-Resolution Fluorescence Imaging of Cytoskeletal and Neuronal Synapse Proteins. bioRxiv (2017).
71. Wang, Y. et al. Rapid Sequential in Situ Multiplexing with DNA Exchange Imaging in Neuronal Cells and Tissues. Nano Lett 17, 6131-6139 (2017).
72. Jungmann, R. et al. Multiplexed 3d Cellular Super-Resolution Imaging with DNA-Paint and Exchange-Paint. Nat Methods 11, 313-8 (2014).
73. Canton, I. & Battaglia, G. Endocytosis at the Nanoscale. Chem Soc Rev 41, 2718-39 (2012).
74. Grant, B. D. & Donaldson, J. G. Pathways and Mechanisms of Endocytic Recycling. Nat Rev Mol Cell Biol 10, 597-608 (2009).
75. Liu, B. R. et al. Endocytic Trafficking of Nanoparticles Delivered by Cell-Penetrating Peptides Comprised of Nona-Arginine and a Penetration Accelerating Sequence. PLoS One 8, e67100 (2013).
76. Lonn, P. et al. Enhancing Endosomal Escape for Intracellular Delivery of Macromolecular Biologic Therapeutics. Sci Rep 6, 32301 (2016).
77. Frohlich, E. The Role of Surface Charge in Cellular Uptake and Cytotoxicity of Medical Nanoparticles. Int J Nanomedicine 7, 5577-91 (2012).
78. Alkilany, A. M. & Murphy, C. J. Toxicity and Cellular Uptake of Gold Nanoparticles: What We Have Learned So Far? J Nanopart Res 12, 2313-2333 (2010).
79. Vives, E., Brodin, P. & Lebleu, B. A Truncated Hiv-1 Tat Protein Basic Domain Rapidly Translocates through the Plasma Membrane and Accumulates in the Cell Nucleus. J Biol Chem 272, 16010-7 (1997).
80. Staahl, B. T. et al. Efficient Genome Editing in the Mouse Brain by Local Delivery of Engineered Cas9 Ribonucleoprotein Complexes. Nat Biotechnol 35, 431-434 (2017).
81. Lee, B. L. & Barton, G. M. Trafficking of Endosomal Toll-Like Receptors. Trends Cell Biol 24, 360-9 (2014).
82. Wittrup, A. et al. Visualizing Lipid-Formulated Sirna Release from Endosomes and Target Gene Knockdown. Nat Biotechnol 33, 870-6 (2015).
83. Nielsen, P. E., Egholm, M. & Buchardt, O. Peptide Nucleic Acid (PNA). A DNA Mimic with a Peptide Backbone. Bioconjug Chem 5, 3-7 (1994).
84. Ziegler, A., Nervi, P., Durrenberger, M. & Seelig, J. The Cationic Cell-Penetrating Peptide Cpp(Tat) Derived from the Hiv-1 Protein Tat Is Rapidly Transported into Living Fibroblasts: Optical, Biophysical, and Metabolic Evidence. Biochemistry 44, 138-48 (2005).
85. Zhang, W. et al. Design of Acid-Activated Cell Penetrating Peptide for Delivery of Active Molecules into Cancer Cells. Bioconjug Chem 22, 1410-5 (2011).
86. Cazenave, C., Frank, P., Toulme, J. J. & Busen, W. Characterization and Subcellular Localization of Ribonuclease H Activities from Xenopus Laevis Oocytes. J Biol Chem 269, 25185-92 (1994).
87. Glaser A, McColl B, and Vadolas J. GFP to BFP Conversion: A Versatile Assay for the Quantification of CRISPR/Cas9-mediatedGenome Editing. Mol Ther Nucleic Acids. 5(7):e334 (2016).
88. Camorani S, Esposito C L, Rienzo A, Catuogno S, Iaboni M, Condorelli G, de Franciscis V, and Cerchia L. Inhibition of receptor signaling and of glioblastoma-derived tumor growth by a novel PDGFRβ aptamer. Mol Ther. 22(4):828-41 (2014).
89. Catuogno S, Rienzo A, Di Vito A, Esposito C L, de Franciscis V. Selective delivery of therapeutic single strand antimiRs by aptamer-based conjugates. J Control Release., 210:147-59 (2015).
90. Filonov G S, Moon J D, Svensen N, Jaffrey S R. Broccoli: rapid selection of an RNA mimic of green fluorescent protein by fluorescence-based selection and directed evolution. J Am Chem Soc., 136(46):16299-308 (2014).
91. Tyson R. Shepherd, Rebecca R. Dul, Hellen Huang, Eike-Christian Wamhoff, Mark Bathe, Bioproduction of single-stranded DNA from isogenic miniphage, bioRxiv (2019).
92. Tyson R. Shepherd, Rebecca R. Dul, Hellen Huang, Eike-Christian Wamhoff, Mark Bathe, Bioproduction of pure, kilobase-scale single-stranded DNA, Sci Rep. 9: 6121 (2019).

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a,” “an,” and “the” include plural reference unless the context clearly dictates otherwise. Thus, for example, reference to “a nucleic acid assembly” includes a plurality of such nucleic acid assemblies, reference to “the nucleic acid assembly” is a reference to one or more nucleic acid assemblies and equivalents thereof known to those skilled in the art, and so forth.

Throughout the description and claims of this specification, the word “comprise” and variations of the word, such as “comprising” and “comprises,” means “including but not limited to,” and is not intended to exclude, for example, other additives, components, integers or steps.

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. It should be understood that all of the individual values and sub-ranges of values contained within an explicitly disclosed range are also specifically contemplated and should be considered disclosed unless the context specifically indicates otherwise. Finally, it should be understood that all ranges refer both to the recited range as a range and as a collection of individual numbers from and including the first endpoint to and including the second endpoint. In the latter case, it should be understood that any of the individual numbers can be selected as one form of the quantity, value, or feature to which the range refers. In this way, a range describes a set of numbers or values from and including the first endpoint to and including the second endpoint from which a single member of the set (i.e. a single number) can be selected as the quantity, value, or feature to which the range refers. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and the right to challenge the accuracy and pertinency of the cited documents is reserved. It will be clearly understood that, although a number of publications are referred to herein, such reference does not constitute an admission that any of these documents forms part of the common general knowledge in the art.

Although the description of materials, compositions, components, steps, techniques, etc. may include numerous options and alternatives, this should not be construed as, and is not an admission that, such options and alternatives are equivalent to each other or, in particular, are obvious alternatives. Thus, for example, a list of different cargo molecules does not indicate that the listed cargo molecules are obvious one to the other, nor is it an admission of equivalence or obviousness.

Every composition disclosed herein is intended to be and should be considered to be specifically disclosed herein. Further, every subgroup that can be identified within this disclosure is intended to be and should be considered to be specifically disclosed herein. As a result, it is specifically contemplated that any composition, or subgroup of compositions can be either specifically included for or excluded from use or included in or excluded from a list of compositions.

Those skilled in the art will recognize, or be able to ascertain using no more than routine experimentation, many equivalents to the specific embodiments of the method and compositions described herein. Such equivalents are intended to be encompassed by the following claims.

We claim:

1. A composition comprising:
(a) a nucleic acid assembly comprising one or more nucleic acid molecules, the nucleic acid assembly comprising physiochemical properties that:
(i) enhance trafficking and/or targeting of the composition to one or more types of cells, tissues, organs, or microenvironments relative to other types of cells, tissues, organs, or microenvironments in vivo;
(ii) enhance stability and/or half-life of the composition in vivo; and/or
(iii) reduce immunogenicity of the composition; and
(b) cargo comprising two or more cargo molecules enclosed and/or protected by the nucleic acid assembly in a defined stoichiometric ratio.

2. The composition of claim 1, wherein the one or more nucleic acid molecules comprising the nucleic acid assembly hybridize together.

3. The composition of claim 1 or 2, wherein the one or more nucleic acid molecules comprising the nucleic acid assembly comprise RNA.

4. The composition of any one of claims 1 to 3, further comprising a plurality of bridging molecules, wherein each bridging molecule is part of or directly or indirectly attached to either or both the nucleic acid assembly and one or more of the cargo molecules, wherein the cargo molecules and bridging molecules that are part of or attach to each other are said to correspond to each other,
whereby the bridging molecules collectively attach the cargo molecules to the nucleic acid assembly in the defined stoichiometric ratio.

5. The composition of claim 4, wherein two or more of the bridging molecules constitute one or more pairs of bridging molecules that specifically bind to the other bridging molecule in the pair,
wherein, for each pair, one bridging molecule of the pair is part of or is directly or indirectly attached to the nucleic acid assembly and the other bridging molecule of the pair is part of or directly or indirectly attached to a corresponding cargo molecule,
whereby specific binding of the pair of bridging molecules specifically attaches the corresponding cargo molecule to the nucleic acid assembly.

6. The composition of claim 4 or 5, wherein at least one of the bridging molecules is part of the nucleic acid assembly, wherein the bridging molecule that is part of the nucleic acid assembly attaches directly or indirectly to a corresponding cargo molecule,
whereby the bridging molecule that is part of the nucleic acid assembly specifically attaches the corresponding cargo molecule to the nucleic acid assembly.

7. The composition of any one of claims 4 to 6, wherein at least one of the bridging molecules is part of a cargo molecule, wherein the bridging molecule that is part of the cargo molecule attaches directly or indirectly to the nucleic acid assembly,
whereby the bridging molecule that is part of the cargo molecule specifically attaches the cargo molecule to the nucleic acid assembly.

8. The composition of any one of claims 4 to 7, wherein at least one of the bridging molecules is part of a cargo molecule, wherein the bridging molecule that is part of the cargo molecule is part of the nucleic acid assembly,
whereby the bridging molecule that is part of the cargo molecule specifically attaches the cargo molecule to the nucleic acid assembly.

9. The composition of any one of claims 4 to 8, wherein direct attachments of bridging molecules to the nucleic acid assembly and/or cargo molecules each comprise a covalent bond, a non-covalent bond, or both a covalent bond and a non-covalent bond.

10. The composition of claim 9, wherein a plurality of the non-covalent bonds are involved in nucleic acid hybridization, whereby specificity of the attachment of the cargo molecule to the nucleic acid assembly is provided by the specificity of the nucleic acid hybridization.

11. The composition of claim 9 or 10, wherein at least one of the covalent bonds is formed by a click chemistry reaction, whereby specificity of the attachment of the cargo molecule to the nucleic acid assembly is provided by the specificity of the click chemistry reaction.

12. The composition of any one of claims 9 to 11, wherein at least one of the non-covalent bonds is formed by specific binding molecules in a specific binding molecule pair, whereby specificity of the attachment of the cargo molecule to the nucleic acid assembly is provided by the specificity of the specific binding molecule pair.

13. The composition of any one of claims 1 to 12, wherein the defined stoichiometric ratio of the cargo molecules is based on the stoichiometric ratio at which the cargo molecules function together.

14. The composition of any one of claims 1 to 12, wherein the defined stoichiometric ratio of the cargo molecules is based on a desired relative effect of the cargo molecules.

15. The composition of any one of claims 1 to 14, wherein the physiochemical properties are selected from structural properties, electric properties, biological properties, or a combination thereof.

16. The composition of any one of claims 1 to 15, wherein the cargo comprises one or more components of one or more CRISPR-Cas systems.

17. The composition of claim 16, wherein the cargo comprises one or more CRISPR-Cas effector proteins.

18. The composition of claim 17, wherein the cargo further comprises one or more guide molecules and/or one or more template oligonucleotides.

19. The composition of claim 18, wherein one or more of the guide molecules is part of the nucleic acid assembly.

20. The composition of claim 18 or 19, wherein one or more of the template oligonucleotides is part of the nucleic acid assembly.

21. The composition of any one of claims 18 to 20, wherein one or more of the template oligonucleotides is an HDR template.

22. The composition of any one of claims 18 to 20, wherein one or more of the template oligonucleotides is an mRNA.

23. The composition of any one of claims 18 to 22, wherein the cargo comprises two or more CRISPR-Cas effector proteins, two or more guide molecules, two or more template oligonucleotides, or a combination thereof.

24. The composition of any one of claims 16 to 23, wherein at least one of the one or more CRISPR-Cas systems is a Cas9 system, a Cas12 system, a Cas13 system, a dCas system, a nickase system, a paired nickase system, an Alt-R CRISPR system, a proxy-CRISPR system, an Alt-R dCas system, an Alt-R nickase system, an Alt-R paired nickase system, an Alt-R proxy-CRISPR system, a proxy-dCas system, a proxy-nickase system, a proxy-paired nickase system, an Alt-R proxy-dCas system, an Alt-R proxy-nickase system, or an Alt-R proxy-paired nickase system.

25. The composition of any one of claims 16 to 24, wherein at least one of the one or more CRISPR-Cas systems comprises one or more CRISPR-Cas effector proteins, wherein at least one of the CRISPR-Cas effector proteins is SpCas9, dCas9, Cas nickase, Cas9 nickase, FnCas9, StCas9, SaCas9, LpCas9, FnCas12, Cas12 nickase, AsCas12, LbCas12, Cas12a, Cas12b, Cas12c, Cas13, or Cas13d.

26. The composition of any one of claims 16 to 25, wherein at least one of the one or more CRISPR-Cas systems comprises a paired Cas9 nickase system, a paired Cas9 nickase system, a dCas/Cas proxy-CRISPR system, a dCas9/Cas9 proxy-CRISPR system, or an SpdCas9/FnCas9 proxy-CRISPR system.

27. The composition of any one of claims 24 to 26, wherein the proxy-CRISPR system comprises two first dCas ribonucleoproteins targeted to different sites flanking and on opposite sides of a main target site and a Cas ribonucleoprotein targeted to the main target site.

28. The composition of claim 27, wherein the proxy-CRISPR system further comprises two additional dCas ribonucleoproteins targeted to different sites flanking and on opposite sides of the main target site, wherein the sites to which the additional dCas ribonucleoproteins are targeted are not the same as the target sites to which the first dCas ribonucleoproteins are targeted.

29. The composition of any one of claims 24 to 28, wherein the Alt-R CRISPR system comprises a separate, shortened gRNA, a separate, shortened tracrRNA, a Cas9 protein.

30. The composition of any one of claims 24 to 29, wherein the paired nickase system leaves 5′ overhangs.

31. The composition of any one of claims 16 to 30, wherein the cargo comprises one or more components of two or more CRISPR-Cas systems.

32. The composition of claim 23, wherein the cargo comprises three or more CRISPR-Cas effector proteins, three or more guide molecules, three or more template oligonucleotides, or a combination thereof.

33. The composition of any one of claims 1 to 15, wherein the cargo does not comprise a CRISPR-Cas effector protein, guide molecule, or HDR template.

34. The composition of any one of claims 1 to 33, wherein the cargo comprises an anti-sense nucleic acid, mRNA, miRNA, piRNA, siRNA, or a combination thereof.

35. The composition of any one of claims 1 to 34, further comprising one or more targeting molecules that specifically targets the nucleic acid assembly to one or more types of cells, tissues, organs, or microenvironments relative to other types of cells, tissues, organs, or microenvironments in vivo.

36. The composition of claim 35, wherein the targeting molecules are selected from the group consisting of DNA aptamers, RNA aptamers, antibodies, nanobodies, lectins, small molecule binding compounds, protein binding domains, protein toxin subunits, peptides, and viral coat proteins.

37. The composition of any one of claims 1 to 36, wherein the nucleic acid assembly forms a container.

38. The composition of claim 37, wherein the cargo is inside the container.

39. The composition of any one of claims 1 to 38, wherein the nucleic acid assembly comprises one or more RNA/DNA hybrid regions.

40. The composition of any one of claims 4 to 39, wherein one or more of the bridging molecules or bound bridging molecule pairs comprises an RNA/DNA hybrid region.

41. The composition of claim 39 or 40, wherein one or more of the RNA/DNA hybrid regions facilitates release of one or more cargo molecules in the presence of an RNA/DNA hybrid specific nuclease.

42. The composition of any one of claims 1 to 41, wherein the nucleic acid assembly comprises a plurality of effector molecules, wherein the effector molecules produce or contribute to the physiochemical properties.

43. The composition of claim 42, wherein the effector molecules comprise polyethylene glycol molecules, lipids, polar groups, charged groups, amphipathic groups, albumin binding molecules, zwitterions, polyamines, RNA intercollators, DNA intercollators, backbone-modified nucleic acids, base-modified nucleic acids, or combinations thereof.

44. A method of delivering cargo to a cell, the method comprising bringing into contact a cell of interest and a composition of any one of claims 1 to 43.

45. The method of claim 44, wherein bringing into contact is accomplished by administering the composition to a subject, wherein the subject harbors the cell.

46. The method of claim 45, wherein the cell is in a tissue of interest, organ of interest, or microenvironment of interest.

47. A method of producing a composition of any one of claims 1 to 43, the method comprising bringing into contact the one or more nucleic acid molecules and the cargo under conditions that facilitate assembly of the nucleic acid molecules and the cargo to form the composition.


SEQUENCE LISTING (200 sequences)

SEQ ID NO: 1 - RNA, 821 nt, Artificial Sequence
  EGFP mRNA
gguagcuaaggagguaaauaauggugagcaagggcgaggagcuguucaccgggguggugcccauccuggucgagcuggacggcgacguaaacggccacaaguucagcguguccggcgagggcgagggcgaugccaccuacggcaagcugacccugaaguucaucugcaccaccggcaagcugcccgugcccuggcccacccucgugaccacccugaccuacggcgugcagugcuucagccgcuaccccgaccacaugaagcagcacgacuucuucaaguccgccaugcccgaaggcuacguccaggagcgcaccaucuucuucaaggacgacggcaacuacaagacccgcgccgaggugaaguucgagggcgacacccuggugaaccgcaucgagcugaagggcaucgacuucaaggaggacggcaacauccuggggcacaagcuggaguacaacuacaacagccacaacgucuauaucauggccgacaagcagaagaacggcaucaaggugaacuucaagauccgccacaacaucgaggacggcagcgugcagcucgccgaccacuaccagcagaacacccccaucggcgacggccccgugcugcugcccgacaaccacuaccugagcacccaguccgcccugagcaaagaccccaacgagaagcgcgaucacaugguccugcuggaguucgugaccgccgccgggaucacucucggcauggacgagcuguacaaguaacugcaggcaugcaagcuuggcguaaucauggucauagcuguuuccugugugaaauuguuauccgcucacaauuccacacaa

SEQ ID NO: 2 - RNA, 792 nt, Artificial Sequence
  Synthetic scaffold sequence
aagggcgaggagcuguucaccgggguggugcccauccuggucgagcuggacggcgacguaaacggccacaaguucagcguguccggcgagggcgagggcgaugccaccuacggcaagcugacccugaaguucaucugcaccaccggcaagcugcccgugcccuggcccacccucgugaccacccugaccuacggcgugcagugcuucagccgcuaccccgaccacaugaagcagcacgacuucuucaaguccgccaugcccgaaggcuacguccaggagcgcaccaucuucuucaaggacgacggcaacuacaagacccgcgccgaggugaaguucgagggcgacacccuggugaaccgcaucgagcugaagggcaucgacuucaaggaggacggcaacauccuggggcacaagcuggaguacaacuacaacagccacaacgucuauaucauggccgacaagcagaagaacggcaucaaggugaacuucaagauccgccacaacaucgaggacggcagcgugcagcucgccgaccacuaccagcagaacacccccaucggcgacggccccgugcugcugcccgacaaccacuaccugagcacccaguccgcccugagcaaagaccccaacgagaagcgcgaucacaugguccugcuggaguucgugaccgccgccgggaucacucucggcauggacgagcuguacaaguaacugcaggcaugcaagcuuggcguaaucauggucauagcuguuuccugugugaaauuguuauccgcucacaauuccacacaa

SEQ ID NO: 3 - DNA, 44 bp, Artificial Sequence
  Staple Sequence
ggtcggggtagagctcctcgcccttttgtgtggctgcttcatgt

SEQ ID NO: 4 - DNA, 44 bp, Artificial Sequence
  Staple Sequence
ctgcacgccgtgatgggcaccaccccggtgaaccggctgaagca

SEQ ID NO: 5 - DNA, 81 bp, Artificial Sequence
  Staple Sequence
tggtagtggtctttttggcgagctgcatggcggacttgtttttaagaagtcgtgaattgtgagcgtttttgataacaattt

SEQ ID NO: 6 - DNA, 44 bp, Artificial Sequence
  Staple Sequence
cagctatgaccgtcgccgatgggggtgttctgccacacaggaaa

SEQ ID NO: 7 - DNA, 44 bp, Artificial Sequence
  Staple Sequence
aagcttgcatgtgtcgggcagcagcacggggccatgattacgcc

SEQ ID NO: 8 - DNA, 33 bp, Artificial Sequence
  Staple Sequence
ccagcttgacgttgtggctgttgtagtctttgc

SEQ ID NO: 9 - DNA, 33 bp, Artificial Sequence
  Staple Sequence
tcagggcgtcgcgcttctcgttggggttgtact

SEQ ID NO: 10 - DNA, 22 bp, Artificial Sequence
  Staple Sequence
tgccccaggatccatgatatag

SEQ ID NO: 11 - DNA, 81 bp, Artificial Sequence
  Staple Sequence
atgccgttctttttttctgcttgtcgggttgccgtccttttttccttgaagtcgcgcgggtcttgttttttagttgccgtc

SEQ ID NO: 12 - DNA, 44 bp, Artificial Sequence
  Staple Sequence
tcgatgttgtgtcctggacgtagccttcgggcacgctgccgtcc

SEQ ID NO: 13 - DNA, 44 bp, Artificial Sequence
  Staple Sequence
agttcaccttggtccttgaagaagatggtgcgcgcggatcttga

SEQ ID NO: 14 - DNA, 81 bp, Artificial Sequence
  Staple Sequence
gaactccagcatttttggaccatgtgagactgggtgcttttttcaggtagtggtcctgcagttactttttttgtacagctc

SEQ ID NO: 15 - DNA, 33 bp, Artificial Sequence
  Staple Sequence
ctgaacttatcgccctcgccctcgccgccggcg

SEQ ID NO: 16 - DNA, 33 bp, Artificial Sequence
  Staple Sequence
gcggtcacgtccatgccgagagtgatcgacacg

SEQ ID NO: 17 - DNA, 22 bp, Artificial Sequence
  Staple Sequence
gtggccgtttaccgtaggtggc

SEQ ID NO: 18 - DNA, 81 bp, Artificial Sequence
  Staple Sequence
gatgaacttcatttttgggtcagcttgcgtcgccgtcctttttagctcgaccagaggtcagggtgtttttgtcacgagggt

SEQ ID NO: 19 - DNA, 33 bp, Artificial Sequence
  Staple Sequence
ccctcgaactcgatgcggttcaccaggttgccg

SEQ ID NO: 20 - DNA, 33 bp, Artificial Sequence
  Staple Sequence
gtggtgcagggccagggcacgggcagcgtgtcg

SEQ ID NO: 21 - DNA, 22 bp, Artificial Sequence
  Staple Sequence
cttcacctcggatgcccttcag

SEQ ID NO: 22 - DNA, 18 bp, Artificial Sequence
  T7 promoter
taatacgactcactatag

SEQ ID NO: 23 - DNA, 1008 bp, Artificial Sequence
  Scaffold Sequence
cgactcactataggtctcgctggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattcgagctcggtacccggggatcctctagagtcgacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatacggtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagatctctcaaaaatagctaccctctccggca

SEQ ID NO: 24 - DNA, 42 bp, Artificial Sequence
  Staple Sequence
gatgaacggtccatcaaaaataattcgcgtcacaagagaatc

SEQ ID NO: 25 - DNA, 42 bp, Artificial Sequence
  Staple Sequence
tatcaggtcatcatcaacattaaatgtgagctctacaaaggc

SEQ ID NO: 26 - DNA, 78 bp, Artificial Sequence
  Staple Sequence
gagtaacaacctttttcgtcggattcagcttgcatgctttttctgcaggtcgagggtagctattttttttttgagaga

SEQ ID NO: 27 - DNA, 78 bp, Artificial Sequence
  Staple Sequence
tataagcaaattttttatttaaattgattttttaacctttttaataggaacgaatcgtaaaacttttttagcatgtca

SEQ ID NO: 28 - DNA, 42 bp, Artificial Sequence
  Staple Sequence
aaacggcggagtaaaacgacggccagtgccatccgtgggaac

SEQ ID NO: 29 - DNA, 42 bp, Artificial Sequence
  Staple Sequence
gatgggcgcagattaagttgggtaacgccagacgttggtgta

SEQ ID NO: 30 - DNA, 42 bp, Artificial Sequence
  Staple Sequence
tccccgggtactatagtgagtcgtgccggagactctagagga

SEQ ID NO: 31 - DNA, 42 bp, Artificial Sequence
  Staple Sequence
gtttcctgtgcgtattgggcgccagggtggttggtcatagct

SEQ ID NO: 32 - DNA, 78 bp, Artificial Sequence
  Staple Sequence
aatgagtgagcttttttaactcacatcgcgcggggagtttttaggcggtttgtgaaattgttattttttccgctcaca

SEQ ID NO: 33 - DNA, 42 bp, Artificial Sequence
  Staple Sequence
aatcggccaataattgcgttgcgctcactgcctgcattaatg

SEQ ID NO: 34 - DNA, 32 bp, Artificial Sequence
  Staple Sequence
gtaaagccacatacgagccggaagtcggtgcg

SEQ ID NO: 35 - DNA, 32 bp, Artificial Sequence
  Staple Sequence
ggcctcttctgttgggaagggcgacataaagt

SEQ ID NO: 36 - DNA, 42 bp, Artificial Sequence
  Staple Sequence
cagctggcgattcgccattcaggctgcgcaacgctattacgc

SEQ ID NO: 37 - DNA, 49 bp, Artificial Sequence
  Staple Sequence
tgctaggatggccgttgcagtcggggaaaagccccaaaaacccccggtt

SEQ ID NO: 38 - DNA, 20 bp, Artificial Sequence
  Staple Sequence
aggaagattgatcatatgta

SEQ ID NO: 39 - DNA, 32 bp, Artificial Sequence
  Staple Sequence
gataatcaaaacctgtcgtgccagccgctttc

SEQ ID NO: 40 - DNA, 32 bp, Artificial Sequence
  Staple Sequence
tttttgtttattttgttaaaattccgcactcc

SEQ ID NO: 41 - DNA, 42 bp, Artificial Sequence
  Staple Sequence
cttctggtgctttgaggggacgacgaccgtattccggcaccg

SEQ ID NO: 42 - DNA, 32 bp, Artificial Sequence
  Staple Sequence
agccagcttcggcctcaggaagatgcgttaaa

SEQ ID NO: 43 - DNA, 78 bp, Artificial Sequence
  Staple Sequence
tcgtaaccgtgtttttcatctgccagcggaaaccaggtttttcaaagcgccaaagggggatgttttttgctgcaaggc

SEQ ID NO: 44 - DNA, 20 bp, Artificial Sequence
  Staple Sequence
aaatcagctctaaacgttaa

SEQ ID NO: 45 - DNA, 20 bp, Artificial Sequence
  Staple Sequence
tggggtgcctattccacaca

SEQ ID NO: 46 - DNA, 42 bp, Artificial Sequence
  Staple Sequence
ttcgtaatcattttcttttcaccagcgagacccgagctcgaa

SEQ ID NO: 47 - DNA, 42 bp, Artificial Sequence
  Staple Sequence
tctggagcaatggccttcctgtagccagcttttgcctgagag

SEQ ID NO: 48 - DNA, 63 bp, Artificial Sequence
  Staple Sequence
cagtcacgacgttttgaccgtaatgggataggttggttttccgggcccggaccggggtctttg

SEQ ID NO: 49 - DNA, 59 bp, Artificial Sequence
  Staple Sequence
acggccatcctagcatcctgagcacccagtccgccctgagcaaagaccccggtccgggc

SEQ ID NO: 50 - DNA, 24 bp, Artificial Sequence
  Staple Sequence
ctcagggcggactgggtgctcagg

SEQ ID NO: 51 - DNA, 19 bp, Artificial Sequence
  Synthetic sequence
tctaccttgcagtggctac

SEQ ID NO: 52 - RNA, 62 nt, Artificial Sequence
  VEGFA-targeting crRNA
gaauuucuacuguuuguagaucaaagcccauucccucuuuagccguagccacugcaagguag

SEQ ID NO: 53 - RNA, 179 nt, Artificial Sequence
  Synthetic sequence
gagcacggggccgucgccgauguuuuagagcuaugcuguugagacggucggguccagauauucguaucugucgaguagagugugggcucaacagcauagcaaguuaaaauaaggcuaguccguuaucaacuugaaaaaguggcaccgagucggugcuucgacuucuucucggucgcauc

SEQ ID NO: 54 - DNA, 89 bp, Artificial Sequence
  HER2 aptamer staple
tcctagatgaataagtaaattacatgcgaatcttccctatattttttgcagcggtgtgggggcagcggtgtgggggcagcggtgtgggg

SEQ ID NO: 55 - DNA, 87 bp, Artificial Sequence
  ER aptamer staple
tcctagatgaataagtaaattacatgcgaatcttccctatattttttcccggcatggttgcggagcaggagtataacactaccattg

SEQ ID NO: 56 - DNA, 59 bp, Artificial Sequence
  Synthetic Sequence
aagtggctaacgcgcgtcgactagtacaaccttaaagatgagtgatatcggtaccctgc

SEQ ID NO: 57 - DNA, 99 bp, Artificial Sequence
  EGFP to BFP HDR Sequence
gtcgtgctgcttcatgtggtcggggtagcggctgaagcactgcacgccgtggctcagggtggtcacgagggtgggccagggcacgggcagcttgccggt

SEQ ID NO: 58 - DNA, 22 bp, Artificial Sequence
  Synthetic Sequence
accggcaagctagcagcacgac

SEQ ID NO: 59 - DNA, 33 bp, Artificial Sequence
  Synthetic Sequence
gcgtgcagtgcttcgtgaccaccctgagccacg

SEQ ID NO: 60 - DNA, 31 bp, Artificial Sequence
  Synthetic Sequence
cgtaatacgactcactataggaccggcaagc

SEQ ID NO: 61 - DNA, 31 bp, Artificial Sequence
  Synthetic Sequence
cgtaatacgactcactataggcgtgcagtgc

SEQ ID NO: 62 - DNA, 76 bp, Artificial Sequence
  Synthetic Sequence
tgtcgaattgcatttactcgatgccccacgacagtcgtgctgctagcttgccggtcctatagtgagtcgtattacg

SEQ ID NO: 63 - DNA, 87 bp, Artificial Sequence
  Synthetic Sequence
gtgagcctcctgtcgaattgaggcgattgatcatcgtggctcagggtggtcacgaagcactgcacgcctatagtgagtcgtattacg

SEQ ID NO: 64 - DNA, 60 bp, Artificial Sequence
  Staple Sequence
gctgttgagcccacactctactcgacagatacgaatatctggacccgaccgtctcaacag

SEQ ID NO: 65 - DNA, 60 bp, Artificial Sequence
  Synthetic Sequence
cgtaatacgactcactataggaccggcaagctagcagcacgacctgttgagacggtcggg

SEQ ID NO: 66 - DNA, 70 bp, Artificial Sequence
  Synthetic Sequence
cgtaatacgactcactataggcgtgcagtgcttcgtgaccaccctgagccacgctgttgagacggtcggg

SEQ ID NO: 67 - DNA, 178 bp, Artificial Sequence
  Synthetic Sequence
cgtaatacgactcactatagctgaagcactgcacgccgtgttttagagctatgctgttgaaaaacagcatagcaagttaaaataaggctagtccgttatcaacttgaaaaagtggcaccgagtcggtgctttttctggcccaccctcagccgctaccccgaccacatgagcccgtgcc

SEQ ID NO: 68 - DNA, 113 bp, Artificial Sequence
  Synthetic Sequence
cgtaatacgactcactataggcgtgcagtgcttcgtgaccaccctgagccacgctgttgagacggtcgggtccagatattcgtatctgtcgagtagagtgtgggctcaacagc

SEQ ID NO: 69 - DNA, 42 bp, Artificial Sequence
  Staple Sequence
cggctgaagcgctcctcgcccttggaaacagggtcggggtag

SEQ ID NO: 70 - DNA, 42 bp, Artificial Sequence
  Staple Sequence
ggtgaacaactgcacgccgtaggtcagggtgggcaccacccc

SEQ ID NO: 71 - DNA, 84 bp, Artificial Sequence
  Staple Sequence
ttcagggtcagtttttttcttgccgtagcgccgtccagcttttttttcgaccaggatggtcacgaggtttttttgtgggccagg

SEQ ID NO: 72 - DNA, 20 bp, Artificial Sequence
  Staple Sequence
ccgtttacgtgtggcatcgc

SEQ ID NO: 73 - DNA, 32 bp, Artificial Sequence
  Staple Sequence
acttgtggcctcgccctcgccggacatgtgat

SEQ ID NO: 74 - DNA, 32 bp, Artificial Sequence
  Staple Sequence
cgcgcttccgaactccagcaggaccacgctga

SEQ ID NO: 75 - DNA, 32 bp, Artificial Sequence
  Staple Sequence
agatgaacgcacgggcagcttgcccttcacct

SEQ ID NO: 76 - DNA, 32 bp, Artificial Sequence
  Staple Sequence
cggcgcgggggtgtcgccctcgaaggtggtgc

SEQ ID NO: 77 - DNA, 84 bp, Artificial Sequence
  Staple Sequence
gccgtcctcgattttttttgttgtggcgagaagtcgtgcttttttttgcttcatgtctatgaccatgtttttttattacgccaa

SEQ ID NO: 78 - DNA, 42 bp, Artificial Sequence
  Staple Sequence
tcaccttgatcttcgggcatggcggacttgagatcttgaagt

SEQ ID NO: 79 - DNA, 42 bp, Artificial Sequence
  Staple Sequence
gacgtagcgccgttcttctgcttgtcggctggtgcgctcctg

SEQ ID NO: 80 - DNA, 84 bp, Artificial Sequence
  Staple Sequence
catgatatagatttttttcgttgtggcttcgatgccctttttttttcagctcgatgtgccgtcgtcctttttttttgaagaaga

SEQ ID NO: 81 - DNA, 20 bp, Artificial Sequence
  Staple Sequence
gtcttgtagtcggttcacca

SEQ ID NO: 82 - DNA, 20 bp, Artificial Sequence
  Staple Sequence
ctccttgaaggttgtagttg

SEQ ID NO: 83 - DNA, 32 bp, Artificial Sequence
  Staple Sequence
ttgccgtctactccagcttgtgccggtagtgg

SEQ ID NO: 84 - DNA, 32 bp, Artificial Sequence
  Staple Sequence
ttgtcggggcggactgggtgctcaccaggatg

SEQ ID NO: 85 - DNA, 42 bp, Artificial Sequence
  Staple Sequence
tgcagttactgtggtcggcgagctgcacgctgcttgcatgcc

SEQ ID NO: 86 - DNA, 42 bp, Artificial Sequence
  Staple Sequence
tgctggtatgtacagctcgtccatgccgagatgggggtgttc

SEQ ID NO: 87 - DNA, 84 bp, Artificial Sequence
  Staple Sequence
tcgttggggtcttttttttttgctcaggcagcagcacggtttttttggccgtcgccgagtgatcccgtttttttgcggcggtca

SEQ ID NO: 88 - DNA, 209 bp, Artificial Sequence
  Synthetic Sequence
gaattctaatacgactcactataggctgatgaggccgcacatggttttagagctatgctgttcuucggaacagcatagcaagttaaaataaggctagtccgttatcaacttgaaaaagtggcaccgagtcggtgcaaaaatcacccagaaacgctggtgaaagtgtatgagtattcaacatttccgtgtcgcccttgtttttgctgcag

SEQ ID NO: 89 - DNA, 32 bp, Artificial Sequence
  Staple Sequence
gcgaaattagacaaagctttaaacaaaatctg

SEQ ID NO: 90 - DNA, 31 bp, Artificial Sequence
  Staple Sequence
aaactaagcttatatgtgtaaaaggaactgc

SEQ ID NO: 91 - DNA, 31 bp, Artificial Sequence
  Staple Sequence
tatgcaaattgcactctgaatctgtctatac

SEQ ID NO: 92 - DNA, 78 bp, Artificial Sequence
  Staple Sequence
caggtacgagctttttgaacgaaaccatagaatcggttttttatccaaagatccgtgtatcattttttaattgtccga

SEQ ID NO: 93 - DNA, 31 bp, Artificial Sequence
  Staple Sequence
gggaaaactcaaatcctatctgtgtttctta

SEQ ID NO: 94 - DNA, 31 bp, Artificial Sequence
  Staple Sequence
gacgtcagtcatgataataatggaagaacca

SEQ ID NO: 95 - DNA, 32 bp, Artificial Sequence
  Synthetic Sequence
ggttaatggtggcacttttcggggaaatgtgc

SEQ ID NO: 96 - DNA, 52 bp, Artificial Sequence
  Staple Sequence
tgagctggttatttttctaggtacatccgcataagtgtttttttgtagaaag

SEQ ID NO: 97 - DNA, 31 bp, Artificial Sequence
  Staple Sequence
gtaggaaggttagaagttagaaattttgact

SEQ ID NO: 98 - DNA, 31 bp, Artificial Sequence
  Staple Sequence
cgggtgtacacctgtatcggccttcaaacgg

SEQ ID NO: 99 - DNA, 32 bp, Artificial Sequence
  Staple Sequence
ttgcgcagatcagtactatgcagtaacctcag

SEQ ID NO: 100 - DNA, 32 bp, Artificial Sequence
  Staple Sequence
gatagttatgatactctatgttcgcccgcatt

SEQ ID NO: 101 - DNA, 52 bp, Artificial Sequence
  Staple Sequence
attcattcatttttttgtgcaatcctctagattagaatttttggcgttgttt

SEQ ID NO: 102 - DNA, 42 bp, Artificial Sequence
  Staple Sequence
ctatattcactttaaaagaaaagaggttgatacgtatcttcc

SEQ ID NO: 103 - DNA, 32 bp, Artificial Sequence
  Staple Sequence
ccgctcatgaaagcatggtagcaagtcgttaa

SEQ ID NO: 104 - DNA, 32 bp, Artificial Sequence
  Staple Sequence
tgacccaagagacaataaccctgaatatgtat

SEQ ID NO: 105 - DNA, 32 bp, Artificial Sequence
  Staple Sequence
ttggtcagttatcggaccacgtgtaaggatta

SEQ ID NO: 106 - DNA, 32 bp, Artificial Sequence
  Staple Sequence
gtgacgtaactcccgggttggtttcatgttac

SEQ ID NO: 107 - DNA, 31 bp, Artificial Sequence
  Staple Sequence
atgacagtacatctagtgataacgcacacgt

SEQ ID NO: 108 - DNA, 52 bp, Artificial Sequence
  Staple Sequence
tgtgttgcaggttttttagcgacagtaaatatcttcatttttcgacgtagcg

SEQ ID NO: 109 - DNA, 20 bp, Artificial Sequence
  Staple Sequence
attcacaagtctggcaatac

SEQ ID NO: 110 - DNA, 32 bp, Artificial Sequence
  Staple Sequence
ccccaatctcttattaccctcgattagtcaca

SEQ ID NO: 111 - DNA, 32 bp, Artificial Sequence
  Staple Sequence
gtctattaatatgtaccccggttggcgtcaga

SEQ ID NO: 112 - DNA, 31 bp, Artificial Sequence
  Staple Sequence
ataattatgggttcccagagctatcgagata

SEQ ID NO: 113 - DNA, 31 bp, Artificial Sequence
  Staple Sequence
gggttgagatcaaaagaatagacattctgac

SEQ ID NO: 114 - DNA, 31 bp, Artificial Sequence
  Staple Sequence
tattaatagagaagtggctaacgctgacggg

SEQ ID NO: 115 - DNA, 31 bp, Artificial Sequence
  Staple Sequence
gaaagccgccccgatttagagctaagcaaga

SEQ ID NO: 116 - DNA, 32 bp, Artificial Sequence
  Staple Sequence
aagggagcgcgaacgtggcgagaaaggaaggg

SEQ ID NO: 117 - DNA, 32 bp, Artificial Sequence
  Staple Sequence
gcgcccggttgcgctcctgcagcatacctatg

SEQ ID NO: 118 - DNA, 52 bp, Artificial Sequence
  Staple Sequence
acagggcgcactttttattaattgcggtggctcaacatttttacagagaata

SEQ ID NO: 119 - DNA, 42 bp, Artificial Sequence
  Staple Sequence
gcgccgctctggcgaactacttactctagcgccgcgcttaat

SEQ ID NO: 120 - DNA, 32 bp, Artificial Sequence
  Staple Sequence
cacgctgcgcgtaaccaccacacccttcccgg

SEQ ID NO: 121 - DNA, 32 bp, Artificial Sequence
  Staple Sequence
caacaattaatagactggatggaggtagcggt

SEQ ID NO: 122 - DNA, 20 bp, Artificial Sequence
  Staple Sequence
gcggataaaggctggcaagt

SEQ ID NO: 123 - DNA, 52 bp, Artificial Sequence
  Staple Sequence
gaaaggagcggtttttgcgctagggcttgcaggaccatttttcttatgcgct

SEQ ID NO: 124 - DNA, 32 bp, Artificial Sequence
  Staple Sequence
aagaaagcgtaaagcactaaatcggaacccta

SEQ ID NO: 125 - DNA, 52 bp, Artificial Sequence
  Staple Sequence
gccctcccgtattttttcgtagttatgttttttggggttttttcgaggtgcc

SEQ ID NO: 126 - DNA, 42 bp, Artificial Sequence
  Staple Sequence
ctaatcaactacacgacggggagtcaggcgtgaaccatcacc

SEQ ID NO: 127 - DNA, 32 bp, Artificial Sequence
  Staple Sequence
tctatcagggcgatggcccactacaactatgg

SEQ ID NO: 128 - DNA, 32 bp, Artificial Sequence
  Staple Sequence
atgaacgaaatagacagatcgctgaaaaaccg

SEQ ID NO: 129 - DNA, 20 bp, Artificial Sequence
  Staple Sequence
agataggtgctcaaagggcg

SEQ ID NO: 130 - DNA, 52 bp, Artificial Sequence
  Staple Sequence
taaatcagctctttttattttttaactaaagaacgtgtttttgactccaacg

SEQ ID NO: 131 - DNA, 32 bp, Artificial Sequence
  Staple Sequence
tccactatcaataggccgaaatcggcaaaatc

SEQ ID NO: 132 - DNA, 32 bp, Artificial Sequence
  Staple Sequence
ccttataatgttgttccagtttggaacaagag

SEQ ID NO: 133 - DNA, 42 bp, Artificial Sequence
  Staple Sequence
atttttgtcgtgagttttcgttccactgataattcgcgttaa

SEQ ID NO: 134 - DNA, 52 bp, Artificial Sequence
  Staple Sequence
ctcactgattatttttagcattggtactcatgaccaatttttaatcccttaa

SEQ ID NO: 135 - DNA, 32 bp, Artificial Sequence
  Staple Sequence
taggtgaagatcctttttgataatactgtcag

SEQ ID NO: 136 - DNA, 32 bp, Artificial Sequence
  Staple Sequence
accaagtttactcatatatacttaaaaggatc

SEQ ID NO: 137 - DNA, 31 bp, Artificial Sequence
  Staple Sequence
ggccaactttcatttttaatttaagattgat

SEQ ID NO: 138 - DNA, 32 bp, Artificial Sequence
  Staple Sequence
tgcagcactggggccagatggtaacggccctt

SEQ ID NO: 139 - DNA, 32 bp, Artificial Sequence
  Staple Sequence
ccggctggctggtttattgctgatggtatcat

SEQ ID NO: 140 - DNA, 52 bp, Artificial Sequence
  Staple Sequence
ccccgaagaactttttgttttccaatcaacgttgcgctttttaaactattaa

SEQ ID NO: 141 - DNA, 32 bp, Artificial Sequence
  Staple Sequence
ccacgatgcctgtagcaatggcaagatgagca

SEQ ID NO: 142 - DNA, 32 bp, Artificial Sequence
  Staple Sequence
cttttaaagttctgctatgtggcggcgtgaca

SEQ ID NO: 143 - DNA, 31 bp, Artificial Sequence
  Staple Sequence
aatgaagccataccaaacgacgacggtatta

SEQ ID NO: 144 - DNA, 31 bp, Artificial Sequence
  Staple Sequence
tcccgtattgacgccgggcaagacggagctg

SEQ ID NO: 145 - DNA, 78 bp, Artificial Sequence
  Staple Sequence
cggaggaccgatttttaggagctaacctcgccttgattttttcgttgggaacgcaactcggtctttttgccgcataca

SEQ ID NO: 146 - DNA, 31 bp, Artificial Sequence
  Staple Sequence
agccggtatgggggatcatgtaacgcttttt

SEQ ID NO: 147 - DNA, 31 bp, Artificial Sequence
  Staple Sequence
tgcacaacgagcgtgggtctcgcaaatctgg

SEQ ID NO: 148 - DNA, 31 bp, Artificial Sequence
  Staple Sequence
ttaaaactacttctgacaacgataacactgc

SEQ ID NO: 149 - DNA, 52 bp, Artificial Sequence
  Staple Sequence
cagtcacagaatttttaagcatcttagctgccataactttttcatgagtgat

SEQ ID NO: 150 - DNA, 31 bp, Artificial Sequence
  Staple Sequence
ctggcggaagagaattatgcagtcggatggc

SEQ ID NO: 151 - DNA, 31 bp, Artificial Sequence
  Staple Sequence
ttttgcgttggttgagtactcacctattctc

SEQ ID NO: 152 - DNA, 31 bp, Artificial Sequence
  Staple Sequence
agaatgacgcattttgccttcctattccctt

SEQ ID NO: 153 - DNA, 20 bp, Artificial Sequence
  Staple Sequence
agagttttcgagtaaattac

SEQ ID NO: 154 - DNA, 32 bp, Artificial Sequence
  Staple Sequence
atctcaacagcggtaagatccttgatgcgatg

SEQ ID NO: 155 - DNA, 32 bp, Artificial Sequence
  Staple Sequence
tgaccttaaacgaatagcctctcccgaactgg

SEQ ID NO: 156 - DNA, 42 bp, Artificial Sequence
  Staple Sequence
aggggacggggtgcacgagtgggttacatacccaagagtttg

SEQ ID NO: 157 - DNA, 52 bp, Artificial Sequence
  Staple Sequence
aaaagatgctgtttttaagatcagttacgacgaattctttttgacgaaaggg

SEQ ID NO: 158 - DNA, 32 bp, Artificial Sequence
  Staple Sequence
tcacccagaaacgctggtgaaagtgtatgagt

SEQ ID NO: 159 - DNA, 32 bp, Artificial Sequence
  Staple Sequence
attcaacatttccgtgtcgcccttgtttttgc

SEQ ID NO: 160 - DNA, 52 bp, Artificial Sequence
  Staple Sequence
cctatttgttttttttatttttctaaaataatattgatttttaaaaggaaga

SEQ ID NO: 161 - DNA, 20 bp, Artificial Sequence
  Staple Sequence
taaatgcttcatacattcaa

SEQ ID NO: 162 - DNA, 32 bp, Artificial Sequence
  Staple Sequence
gcggaacccctcgtgatacgcctatttttata

SEQ ID NO: 163 - DNA, 2520 bp, Artificial Sequence
  Scaffold Sequence
gagcgcaacgcaattaatgtgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattacaaccggggtacatatgattggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcataagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcgaattcgtcgtcgtcccctcaaactcttgggtggagaggctattcgtttaaggtcacatcgcatgtaatttacttattctctgttgttgagccacccgggcgccagattttgtttaaagctttgtctcttagtttgtatagacagattcagagtgcaaggtttcgttcgctcgtacctggttttccctggttcttcacagataggatttgactttctacaacacttatgcggcttcctacccgtttgaaggccgatacaggtgctgcgcaaaatgcgggcgaacatagagtatcaaaacaacgccttctaatctaggaatatagggaagatacgtatttgctaccatgctttcttgggtcattaacgaccaacctcttttcttttaaagtaggattgcacaatgaatgaatacacgtggtccgataactgaccaagtaacatggttatcactagatgtccgccagacgtgtgcaaaccaacccgggagttacgtcactaatccttcgctacgtcgtgaagatatttacttgtgaatatcgagggtaataagataatagactgtgactagtattgccagactgtcgctacctgcaacacataactatcctgaggttactgcatagtactgattacacccgagtcaaaatttctaacttctaacatgtacctagtaaccagctcaataattatgtcagaatatagctctgggaaccctcggacaattatgatacacggtattaatatcttgcttgcgttagccacttctcatctttggataccgattctattttgcatagcagttccttttacacatataagaatttcgccataggtatgctgcag

SEQ ID NO: 164 - protein, 7 aa, Artificial Sequence
  Homing Peptide
ATWLPPR

SEQ ID NO: 165 - protein, 9 aa, Artificial Sequence
  NLS Sequence
PAAKRVKLD

SEQ ID NO: 166 - protein, 27 aa, Artificial Sequence
  Mitochondrial NLS Sequence
PEDEIWLPEPESVDVPAKPISTSSMMM

SEQ ID NO: 167 - DNA, 76 bp, Artificial Sequence
  Synthetic Sequence
cgtaatacgactcactataggaccggcaagctagcagcacgactgtcgtggggcatcgagtaaatgcaattcgaca

SEQ ID NO: 168 - DNA, 87 bp, Artificial Sequence
  Synthetic Sequence
cgtaatacgactcactataggcgtgcagtgcttcgtgaccaccctgagccacgatgatcaatcgcctcaattcgacaggaggctcac

SEQ ID NO: 169 - DNA, 103 bp, Artificial Sequence
  Synthetic Sequence
cgtaatacgactcactataggaccggcaagctagcagcacgacctgttgagacggtcgggtccagatattcgtatctgtcgagtagagtgtgggctcaacagc

SEQ ID NO: 170 - DNA, 12 bp, Artificial Sequence
  Stemloop Sequence
acttgtttaagt

SEQ ID NO: 171 - DNA, 16 bp, Artificial Sequence
  Stemloop Sequence
ggcaccgagtcggtgc

SEQ ID NO: 172 - protein, 4 aa, Artificial Sequence
  GlySer linker
GGGS

SEQ ID NO: 173 - protein, 12 aa, Artificial Sequence
  GlySer linker
GGGSGGGSGGGS

SEQ ID NO: 174 - protein, 15 aa, Artificial Sequence
  Hydrophilic/Cationic CPP
RQIKWFQNRRMKWKK

SEQ ID NO: 175 - protein, 11 aa, Artificial Sequence
  Hydrophilic/Cationic CPP
YGRKKRRQRRR

SEQ ID NO: 176 - protein, 18 aa, Artificial Sequence
  Hydrophilic/Cationic CPP
RGGRLSYSRRRFSTSTGR

SEQ ID NO: 177 - protein, 10 aa, Artificial Sequence
  Hydrophilic/Cationic CPP
RRLSYSRRRF

SEQ ID NO: 178 - protein, 12 aa, Artificial Sequence
  Hydrophilic/Cationic CPP
PIRRRKKLRRLK

SEQ ID NO: 179 - protein, 12 aa, Artificial Sequence
  Hydrophilic/Cationic CPP
RRQRRTSKLMKR

SEQ ID NO: 180 - protein, 15 aa, Artificial Sequence
  Hydrophilic/Cationic CPP
RRRRNRTRRNRRRVR

SEQ ID NO: 181 - protein, 19 aa, Artificial Sequence
  Hydrophilic/Cationic CPP
KMTRAQRRAAARRNRWTAR

SEQ ID NO: 182 - protein, 13 aa, Artificial Sequence
  Hydrophilic/Cationic CPP
TRRQRTRRARRNR

SEQ ID NO: 183 - protein, 13 aa, Artificial Sequence
  Hydrophilic/Cationic CPP
GRKKRRQRRRPPQ

SEQ ID NO: 184 - protein, 13 aa, Artificial Sequence
  Hydrophilic/Cationic CPP
GRRRRRRRRRPPQ

SEQ ID NO: 185 - protein, 27 aa, Artificial Sequence
  Amphipathic CPP
GWTLNSAGYLLGKINLKALAALAKKIL

SEQ ID NO: 186 - protein, 17 aa, Artificial Sequence
  Amphipathic CPP
KLALKLALKLALALKLA

SEQ ID NO: 187 - protein, 27 aa, Artificial Sequence
  Amphipathic CPP
MGLGLHLLVLAAALQGAWSQPKKKRKV

SEQ ID NO: 188 - protein, 27 aa, Artificial Sequence
  Amphipathic CPP
GALFLGWLGAAGSTMGAWSQPKKKRKV

SEQ ID NO: 189 - protein, 27 aa, Artificial Sequence
  Amphipathic CPP
  MISC_FEATURE (1)..(1): Acetylated
  MISC_FEATURE (27)..(27): Cysteamide group
GALFLGFLGAAGSTMGAWSQPKKKRKV

SEQ ID NO: 190 - protein, 27 aa, Artificial Sequence
  Amphipathic CPP
  MISC_FEATURE (1)..(1): Acetylated
  MISC_FEATURE (27)..(27): Cysteamide group
GALFLGFLGAAGSTMGAWSQPKSKRKV

SEQ ID NO: 191 - protein, 21 aa, Artificial Sequence
  Amphipathic CPP
  MISC_FEATURE (1)..(1): Acetylated
  MISC_FEATURE (21)..(21): Cysteamide group
KETWWETWWTEWSQPKKKRKV

SEQ ID NO: 192 - protein, 21 aa, Artificial Sequence
  Amphipathic CPP
  MISC_FEATURE (1)..(1): Acetylated
  MISC_FEATURE (21)..(21): Cysteamide group
KETWFETWFTEWSQPKKKRKV

SEQ ID NO: 193 - protein, 25 aa, Artificial Sequence
  Synthetic Sequence
GLFHAIAHFIHGGWHGLIHGWYGGC

SEQ ID NO: 194 - protein, 4 aa, Artificial Sequence
  NLS Sequence
  MISC_FEATURE (2)..(2): Xaa = Lysine or Arginine
  MISC_FEATURE (3)..(3): Xaa = any amino acid
  MISC_FEATURE (4)..(4): Xaa = Lysine or Arginine
KXXX

SEQ ID NO: 195 - protein, 7 aa, Artificial Sequence
  NLS Sequence
PKKKRKV

SEQ ID NO: 196 - protein, 12 aa, Artificial Sequence
  NLS Sequence
PKKKRKVEDPYC

SEQ ID NO: 197 - protein, 33 aa, Artificial Sequence
  NLS Sequence
PKKKRKVGPKKKRKVGPKKKRKVGPKKKRKVGC

SEQ ID NO: 198 - protein, 12 aa, Artificial Sequence
  NLS Sequence
CYGRKKRRQRRR

SEQ ID NO: 199 - protein, 17 aa, Artificial Sequence
  NLS Sequence
CSIPPEVKFNKPFVYLI

SEQ ID NO: 200 - protein, 18 aa, Artificial Sequence
  NLS Sequence
DRQIKIWFQNRRMKVVKK